(19) United States
(12) Patent Application Publication
(10) Pub. No.: US 2026/0241167 A1
(43) Pub. Date: Aug. 20, 2026

(54) AUTOMATIC DETECTION-THERAPY SYSTEMS FOR MIGRAINE AND CLUSTER HEADACHE AND NEUROVASCULAR MONITORING SYSTEM

(71) Applicant: David C. Shaw, Torrance, CA (US)

(72) Inventors: David C. Shaw, Torrance, CA (US); Caroline Huang, Redondo Beach, CA (US); Julia Huang, Redondo Beach, CA (US)

(73) Assignee: David C. Shaw, Torrance, CA (US)

(21) Appl. No.: 19/647,337

(22) Filed: Apr. 14, 2026

Related U.S. Application Data
(63) Continuation-in-part of application No. 19/191,555, filed on Apr. 28, 2025, now Pat. No. 12,642,486.
(60) Provisional application No. 63/732,819, filed on Sep. 25, 2024.
(60) Provisional application No. 63/732,962, filed on Oct. 12, 2024.
(60) Provisional application No. 63/833,151, filed on Oct. 26, 2024.
(60) Provisional application No. 64/028,497, filed on Jan. 30, 2026.

(51) Int. Cl. A61N 1/36; A61B 5/00; A61B 5/0205; A61B 5/026; A61B 5/291; A61B 5/372; A61N 1/02

(52) U.S. Cl. CPC A61N 1/36021; A61B 5/0205; A61N 1/025; A61N 1/36031; A61N 1/36036; A61B 5/0261; A61B 5/291; A61B 5/372; A61B 5/6817

(57) ABSTRACT

An extended auricular electroencephalogram (EEG) monitoring system, an auricular Speckle Contrast Optical Spectroscope (SCOS) unit and photoplethysmography (PPG) unit are integrated to detect and guide therapy of migraine, impending migraine, cluster headache and impending cluster headache. An automatic detection-therapy system includes the auricular EEG monitoring system, the SCOS unit-PPG unit and neuromodulation units. When presence of EEG signals and blood flow data suggestive of migraine or cluster headache is detected by the processing unit, the neuromodulation unit is configured to automatically start neuromodulating electric stimulation immediately to at least one of: auricular branch of vagus nerve, auriculotemporal nerve, supraorbital nerve, infraorbital nerve, occipital nerve and greater auricular nerve. A neurovascular monitoring system, comprising an auricular EEG, SCOS and PPG to monitor EEG and cerebral blood flow, can monitor stroke, brain injury and warn impending stroke. These earbud-shaped systems are wearable, user-installable, user-removable and ambulatory.

### CROSS REFERENCE TO RELATED APPLICATIONS

This application is a continuation-in-part application of U.S. Non-Provisional application Ser. No. 19/191,555, filed on Apr. 28, 2025, entitled “Auricular Electroencephalogram (EEG) and Automatic Remedy Systems for Neuropsychiatric Disorders”, which claims priority to and the benefit of the filing date of: U.S. Provisional Application No. 63/732,819 filed on Sep. 25, 2024, entitled “Automatic auricular anti-seizure device”; U.S. Provisional Application No. 63/732,962, filed on Oct. 12, 2024, entitled “Automatic auricular detection-remedy system”; and U.S. Provisional Application No. 63/833,151, filed on Oct. 26, 2024, entitled “Automatic auriculardetection-remedy system for neuropsychiatric disorders”, which are all hereby incorporated by reference in their entirety. This application also claims priority to and the benefit of the filing date of U.S. Provisional Application No. 64/028,497, filed on Jan. 30, 2026, entitled “Automatic Detection and Therapy Systems for Migraine” with the first inventor David Shaw, which is hereby incorporated by reference in its entity.

### FIELD OF THE INVENTION

This patent specification relates to the field of migraine and cluster headache, electroencephalogram (EEG), automatic EEG analysis and the field of neurovascular monitoring. This invention also relates to the field of neuromodulation, including transcutaneous auricular vagus nerve stimulation (taVNS), trigeminal nerve stimulation, occipital nerve stimulation and greater auricular nerve stimulation which may be used in treatment of migraine and cluster headache.

### BACKGROUND

The human ear is very close to the brain and recent studies have shown that electrodes placed in the external ear canal can do electroencephalogram (EEG). Traditional EEG (full-scalp EEG) is done with multiple (mostly 21) electrodes attached to the scalp and connected with the EEG machine through multiple wires. It is very time-consuming to set up and to remove. It is quite non-ambulatory (extremely cumbersome and difficult for wearer to move around). In traditional full-scalp EEG, the electrodes are attached or glued to the scalp by certified technologists. Traditional EEG is usually for short-term use only (half an hour to a couple days) because the electrodes often become detached after a short time. There exists a great need to have a miniature nice-looking device which can record EEG on a long-term (weeks or months or even years) basis. Previously EEG has been used primarily in the medical field or biomedical research field. Nowadays, in the artificial intelligence and robotics era, EEG or modified EEG is also heavily used in the brain-computer interface (BCI). Advancement in electronics technology, miniaturization trend, and dry electrode technology, together with sophisticated EEG interpretation algorithms, have enormously widened the potential usefulness of EEG in various medical fields.

In recent years, wireless in-ear EEG had been reported. For example, as published on Aug. 2, 2024, in Nature Communications, Ryan Kaveh et al reported using wireless miniature dry ear electrodes for in-ear EEG, with wireless electronics and offline classification algorithms, to monitor drowsiness of pilots and drivers. They described the design of earpieces for EEG, the neural recording hardware, the electrode materials and multi-sensor array. The recorded EEG data are digitized and transmitted to a processing unit for offline processing. They also describe manufacturing methods for in-ear EEG sensors. Two contralaterally worn earpieces can provide up to 11 channels with a common reference. Either right or left cymba concha electrode can be used as a reference. Prior to this report, earpieces with wet (hydrogel coated) electrodes were often used for EEG. These in-ear EEGs have been shown to successfully monitor drowsiness, seizure and sleep etc.

Various EEG analysis algorithms for decoding and processing of EEG data have been developed and described in many studies. With these EEG analysis algorithms and long-term EEG monitoring, they were able to accurately detect seizure (seizure also called epilepsy), migraine, cluster headache, major depressive disorder, bipolar disorder, schizophrenia, obsessive-compulsive disorder, attention deficit hyperactivity disorder, autism spectrum disorder, post-traumatic stress disorder, anxiety disorder and panic disorder. They also found changes of EEG patterns suggestive of impending seizure, migraine, cluster headache, major depressive disorder, bipolar disorder, schizophrenia, obsessive-compulsive disorder, attention deficit hyperactivity disorder, autism spectrum disorder, post-traumatic stress disorder, anxiety disorder and panic disorder. Artificial intelligence, including machine learning (ML) and deep learning (DL) algorithms were applied to EEG data for processing.

In-ear EEG can overcome a lot of the limitations of traditional full-scalp EEG (limitations of full-scalp EEG includes: time-consuming to set up and to remove, bulky and non-ambulatory, short-time use, not suitable for long-term monitoring, requiring certified technologist to set up or remove, etc.). In-ear EEG (ear-EEG) has been developed in recent years with the help of advancement in electronic technology including the miniaturization trend and dry electrode technology. Like the traditional full-scalp EEG, the in-ear EEG can assess the electrical activities of the brain to monitor various neurological and psychological conditions. In-ear EEG has a very important advantage that it is much more suitable than traditional full-scalp EEG for long-term ambulatory monitoring. The newly developed sophisticated EEG interpretation algorithms have further widened the potential usefulness of the in-ear EEG.

As compared with traditional full-scalp EEG, in-ear EEG has been shown to provide very high performance for detecting seizures, particularly generalized seizures, temporal lobe seizures and focal-onset seizures, although it cannot fully match the sensitivity of full-scalp EEG in detecting frontal lobe seizures. In-ear EEG can detect ictal and interictal abnormalities for temporal lobe seizures with sensitivity matching that of full-scalp EEG. In-ear EEG is less effective in identifying frontal dominant interictal discharges. Overall, the sensitivity of in-ear EEG is around 86.4% to 94.5%, in detecting seizure. A National Institute of Health (NIH) study confirms that in-ear EEG offer a reliable alternative method to traditional scalp EEG. In-ear EEG is easier and much faster to setup and offer more comfort for patients than full-scalp EEG. The biggest advantage of in-ear EEG is that it is ambulatory and allow easy long-term monitoring. Long-term (or extended) EEG monitoring is highly effective for detecting and capturing seizures that routine full-scalp EEG might miss. By recording for 24 hours or more, it can identify abnormal EEG patterns and enable better seizure classification, localization and prediction of imminent seizure events minutes in advance. A study found that by analyzing prolonged EEG signals some models can predict up to 98% of seizures minutes in advance by identifying pre-ictal states. Long-term monitoring for 24-72 hours or longer will be very valuable for patients with suspected seizures or patients at high risk of status epilepticus. Long-term monitoring will also be very helpful to detect ictal and interictal epileptiform discharges during sleep. There are other studies showing that, for other neuropsychiatric disorders like migraine or cluster headache, the in-ear EEG offers reliable alternative to full-scalp EEG.

Various EEG analysis algorithms for decoding and processing of EEG data have been developed and described in many studies. With these EEG analysis algorithms and long-term EEG monitoring, they were able to accurately detect neuropsychiatric disorders, including migraine, cluster headache, seizure and several psychiatric disorders. They also found changes of EEG patterns suggestive of impending migraine, seizure and impending psychiatric disorders. Artificial intelligence, including machine learning (ML) and deep learning (DL) algorithms were applied to EEG data for processing. Several different methods with very high accuracy for seizure detection were described by Maham Saeidi, et al, in a published article in Brain Science on Nov. 18, 2021.

Migraine headache affects millions of people in the USA. There are many medicines which can help migraine. The triptans are one of the mainstream medicines being used for migraine. Triptans can be taken orally, intranasally or by subcutaneous injection. There are also several prophylactic medicines available. Even with all of these, migraine headaches still produce major impact on the patients' lives and huge economic loss.

Migraine can occur suddenly. A reliable method for pre-migraine management is in great need. Previously, migraine attacks were considered unpredictable making preemptive interventions not feasible. However, it was found that there are neurophysiological changes 24-48 hours before migraine attacks. These neurophysiological changes can be detected using long-term EEG monitoring. In 2020, Isabel Martin et al reported using EEG to predict future migraine attacks. They found that 24 hours before migraine onset, there was a statistically significant modulation of the EEG, with decrease of relative power in the delta waves and increase of beta wave frequency bands, at rest. There was also a notable reduction of the amplitude and coherence measures of an attention event-related brain potential (P300). There were other studies regarding migraine and cluster headache with EEG changes. In 2016 Cao Z. et al reported that resting state EEG power and coherence vary between migraine phases. In 2023, Ning Zhang et al reported a review of using modern EEG data processing and analysis for EEG-based migraine analysis. It would be very valuable if patients can be notified of impending migraine and can start taking prophylactic measures preemptively.

In 2024, Thomas Van den Hoek reported another review article regarding EEG and migraine. Traditional EEG did not help much in the management or prediction of migraine headache. One of the most important reasons is due to the fact that traditional EEG was mostly done only short-term on cross-sectional basis. Recently, it was found that a more long-term longitudinal EEG can make subtle changes in EEG much more detectable. Advancement in data processing and analysis also help to find EEG changes during migraine. Furthermore, it was also found that EEG changes can be detected even before onset of acute migraine attack. Longitudinal studies have been able to identify some pre-ictal changes in spontaneous EEG features compared to the inter-ictal phase, and also between migraine patients and controls. These differences include EEG slowing, alpha and theta band asymmetry, enhanced EEG spectral power, and coherence. The observation that EEG power and coherence were reduced during the acute migraine as compared to the before migraine period could be used to predict migraine before an attack. Raghuraman L. and Joshi S. reported in 2024 another review article regarding EEG and migraine. They reported EEG changes in migraine, including cortical hyperexcitability and habituation deficit to sensory stimuli and alpha oscillations. Spectral analysis of EEG waves often showed more reliable and consistent results than features read off the EEG directly. EEG microstate was found to be the most promising method showing characteristic identifiable features for diagnosis of migraine.

Tension headache generally does not show specific diagnostic changes detectable on regular EEG or extended EEG. Using extended EEG to detect migraine will greatly help patients to differentiate between migraine and tension headache.

After detecting migraine or impending migraine, it would be very valuable to have a novel wearable convenient device that can provide therapeutic actions automatically and instantly in response to the EEG detection of migraine or impending migraine.

Many neuro-psychiatric disorders or conditions can be detected with EEG, including migraine, cluster headache, seizure, major depressive disorder, bipolar disease, autism, schizophrenia, tinnitus, dyslexia, stroke, etc. In 2023 Hao Zhang et al published a review article regarding principles of EEG analysis methods in neuroscience and clinical neurology. Maham Saeidi et al and Hao Zhang et al, reported that EEG data analysis have leaded to detection of stroke, autism, dyslexia and tasks including emotion recognition, mental workload, motor imagery, neurodegenerative diseases, sleep stages scoring and seizure detection, etc.

In 2024, Thomas Van den Hoek reported another review article regarding EEG and migraine. Traditional EEG did not help much in the management or prediction of migraine. One of the most important reasons is due to the fact that traditional EEG was mostly done only short-term on cross-sectional basis. Recently, it was found that a more long-term longitudinal EEG can make subtle changes in EEG much more detectable. Advancement in data processing and analysis also help to find EEG changes during migraine. Furthermore, it was also found that EEG changes can be detected even before onset of acute migraine attack. Longitudinal studies have been able to identify some pre-ictal changes in spontaneous EEG features compared to the inter-ictal phase, and also between migraine patients and controls. These differences include EEG slowing, alpha and theta band asymmetry, enhanced EEG spectral power, and coherence. The observation that EEG power and coherence were reduced during the acute migraine as compared to the before migraine period could be used to predict migraine before an attack.

Raghuraman L. and Joshi S. reported in 2024 one more review article regarding EEG and migraine. They reported EEG changes in migraine, including cortical hyperexcitability and habituation deficit to sensory stimuli and alpha oscillations. Spectral analysis of EEG waves often showed more reliable and consistent results than features read off the EEG directly. EEG microstate was found to be the most promising method showing characteristic identifiable features for diagnosis of migraine.

Cluster headache is another type of headache which affects millions of people in USA. It is also called trigeminal autonomic cephalgia. Cluster headache is usually shorter (15 to 180 minutes) than migraine, but is much more intense and can occur more often (up to 8 times a day). The pain with cluster headache is so severe that patients are often incapacitated during the attack. It can keep recurring for weeks or even months. Cluster headache is extremely painful and is characterized by severe, unilateral head pain, often located behind one eye. Cluster headache often occurs in clusters daily for weeks or months and then followed by remission (pain-free) periods (possibly for few months). Each cluster headache attack can occur suddenly lasting 15 minutes to 3 hours. It could occur in the night and waking the patients up at night. Cluster headache is typically one-sided and on the same side patients often have a drooping eyelid (ptosis), tearing (watery eye), nasal congestion or runny nose, facial sweating, and a flushed face.

Cluster headache is quite different from migraine. Migraine sufferers usually prefer to lie down in dark rooms, whereas cluster headache suffers are often agitated, restless and pacing around. The exact cause of cluster headache is unknown. Cluster headaches are believed to be linked to hypothalamic dysfunction and the trigeminal nerve. They are not generally hereditary. Because of the extreme severity and the very rapid onset, standard pain-relieving medicines are rarely effective for cluster headache. High-flow oxygen through a facial mask is an effective treatment. Sumatriptan injection is another effective treatment. Greater occipital nerve block has also been found to be helpful. However, oxygen tank and injectable sumatriptan are often not immediately available for the suffers. Preventive medication, such as verapamil, may be useful to prevent or lessen future attacks.

It is possible to have both cluster headaches and migraines, a condition where both types of severe headaches co-exist, with studies suggesting around 10 to 17 percent of people with cluster headaches also experience migraines. While they have distinct characteristics-cluster headaches are typically short, extremely agonizing, and cause restlessness, whereas migraines are often longer, throbbing, and cause a need for stillness-a patient can suffer from both independently.

Previously, it was believed that there were not much significant EEG changes before or during cluster headache. Recent studies have reported EEG changes associated with cluster headaches. These changes often occur before or during headache episodes, indicating potential alterations in brain activity related to the condition. EEGs taken between attacks may show abnormalities, including focal slowing or spikes, which could indicate underlying pathophysiological processes. Some studies have noted increased theta wave activity in the frontal regions of the brain during cluster headache attacks, suggesting heightened cortical excitability.

More recent studies with extended EEG have found EEG changes before and during cluster headache, even though there were less reports in this regard as compared with migraine. Some studies suggest a slight increase of non-specific abnormalities or slow waves during pre-cluster headache periods. Some reports have observed focal EEG abnormalities like transient theta-delta slowing on the ipsilateral side during cluster headache. Unlike the clear cortical spreading depression (CSD) and related EEG changes in migraine, typical cluster headache attacks do not exhibit these specific cortical changes, though some overlap with migraine symptoms (like aura) has been reported. In 2022, Padmarathy N. et al reported using EEG to identify cluster headache and migraine.

Cluster headache is a primary neurovascular headache, typically accompanied by significant neurovascular changes, including dilation of blood vessels in the brain and around the eye. These neurovascular changes are driven by the activation of the trigeminal-autonomic reflex, which connects the trigeminal nerve to facial parasympathetic nerves. It causes vasodilatation leading to autonomic symptoms on the affected side, including red, watery eye (conjunctival injection and lacrimation), swollen eyelid, droopy eyelid (ptosis) and nasal congestion. The increased blood flow may also result in temperature changes.

Blood flow changes may occur before cluster headache onset. Studies suggest that both intracranial and extracranial blood flow alterations can precede the onset of cluster headaches. The extracranial blood flow changes often intensify during the cluster headache. Research indicates that there may be changes in intracranial (cerebral) blood flow, particularly in areas associated with pain processing and autonomic regulation. These changes could be linked to the trigeminal autonomic reflex, which is involved in cluster headaches. The extracranial blood flow increase with heat loss and swelling affects the scalp, orbit, forehead and temple. The extracranial blood flow changes may contribute to the characteristic symptoms of cluster headaches, such as unilateral pain and autonomic dysfunction.

Studies also suggest that blood flow changes are largely secondary to, rather than the cause of, cluster headache attacks. Key changes include extracranial vasodilation (increased blood flow) in the temple and eye area, and decreased common carotid artery blood flow (increased vascular resistance) during the peak of the headache. Studies have shown that the blood flow changes in the external ear and the external ear canal are similar to the blood flow changes in the scalp, including the temple area. The intracranial (cerebral) blood flow changes during cluster headache attack are less consistent, with some studies show increased cerebral blood flow during induced attacks, while others indicate no significant, consistent change.

There are also significant intracranial and extracranial blood flow changes before and during migraine. Typically, there is a pre-migraine reduction in cerebral blood flow (oligemia) followed by increased, often dilated, blood vessel activity during the migraine phase. During the prodromal (aura) phase of migraine, the regional cerebral blood flow often decreases (hypoperfusion), particularly in the occipital cortex for aura. During the migraine headache phase, the cerebral blood flow shifts to abnormally high (hyperperfusion), particularly on the side of the migraine. During the migraine pain phase, there is often dilatation of meningeal arteries and potentially the middle cerebral artery. For the extracranial portion, the superficial temporal arteries may dilate, contributing to the throbbing pain, with blood flow often increased on the painful phase of migraine.

Previously older theories suggested blood flow changes caused the pain; however, more recent understanding links these blood flow changes to the activation of the trigemino-vascular system, where dilated vessels trigger pain pathways. Vasoactive substances like calcitonin gene-related peptide (CGRP) and nitric oxide (NO) are released during the migraine attack, causing vasodilation and inflammation. Triptan medications work by causing vasoconstriction of these dilated vessels, which helps to relieve the pain,

There is distinct difference in the blood flow patterns between the prodromal (aura) phase and the headache phase of migraine. There is marked decrease (hypoperfusion) of regional cerebral blood flow during the prodromal or pending phase (aura), which then shift to increased blood flow (hyperperfusion) during the migraine phase. For migraine, the most dramatic blood flow changes are intracranial (cerebral) and this is often more pronounced on the ipsilateral side of the migraine. Migraine is also associated with extracranial vasodilation (in the scalp and orbit etc.) although this is often less dramatic as compared with the changes in cerebral blood flow. The extracranial vasodilatation also involves the external ear and external ear canal and this might offer a convenient location for monitoring of blood flow.

There are a few different methods to assess intracranial and extracranial blood flow. Ultrasound or doppler studies are well known. But the traditional ultrasound devices are usually not wearable. Transcranial Doppler Ultrasound (TCD) is a non-invasive technique for real-time monitoring of cerebral blood flow. It can detect abnormalities such as blockages or irregular blood flow patterns, which may indicate conditions like stroke or carotid artery disease. Recent advancements include wearable ultrasound devices that can monitor cerebral blood flow. For instance, a lightweight system developed by researchers at Caltech and USC accurately measures blood flow to the brain, distinguishing it from scalp blood flow. The transcranial doppler ultrasound can be combined with carotid duplex ultrasound to assess the blood flow in stroke patients and carotid stenosis. an emerging flexible doppler ultrasound device uses wearable sensors for long-term continuous monitoring of cerebral blood flow.

Another emerging technology (for example Nuroflux) uses hybrid devices placed on a wearable headband designed for the continuous, non-invasive monitoring of brain activity and blood flow in stroke patients. This is for in-patient use to monitor acute stroke patients because it is not designed for ambulatory use. The Nuroflux device uses a proprietary sensor array that simultaneously monitors electrical brain activity (via EEG) and cerebral blood flow (using a novel ECG-based metric). The device utilizes AI to identify subtle patterns in blood flow change and brain activity change that precede a stroke or worsening of an existing stroke. Another device called “Hybrid EEG-fNIRS Headbands” combined EEG with functional Near-Infrared Spectroscopy to monitor brain electrical activity by EEG and to monitor cerebral blood flow/volume with fNIRS. This type of devices is bulky and cumbersome for users to use and are primarily suitable for in-patient use. There exists a great need to have a miniature wearable and ambulatory monitoring device for brain's electrical activities and cerebral blood flow.

There are other devices designed to measure both cerebral and scalp blood flow. Recent advancements have led to the development of noninvasive optical devices that utilize laser technology to measure blood flow at different depth. These laser devices can differentiate between blood flow in the brain and the scalp. These devices employ optical spectroscopy systems to accurately assess blood dynamics at different depths, ensuring that measurements reflect cerebral blood flow rather than just scalp circulation. These devices can be worn on the forehead, making them user-friendly and suitable for clinical settings. Such devices could aid in monitoring conditions like stroke and traumatic brain injury.

New development in laser technology also provides convenient way to monitor cerebral and extracranial blood flow. There are a few laser speckle-based blood flow monitoring unit. One example is Speckle Contrast Optical Spectroscopy (SCOS) which is a non-invasive, camera-based diffuse optical technique that can measure deep-tissue microvascular blood flow and perfusion at different depths. By analyzing the blurring of laser speckle patterns caused by moving red blood cells, SCOS provides high signal-to-noise ratio assessments of blood flow dynamics. When tissue is illuminated by a laser, the backscattered laser light creates a random interference pattern known as speckle. Moving red blood cells cause this speckle pattern to blur. The SCOS measures the contrast (ratio of standard deviation to mean intensity) of these patterns to determine blood flow speed. The SCOS uses multi-exposure with multi-depth approaches to analyze how speckle contrast depends on the camera exposure time or the distance between the light source and light detector. It is primarily used to monitor cerebral blood flow (CBF) and brain function (in stoke patients or brain trauma patients), and deep tissue perfusion,

A similar and related laser speckle-based blood flow monitoring unit, called Diffuse Correlation Spectroscopy (DCS) has also been developed, DCS is a non-invasive, optical technique that uses near-infrared light to measure deep tissue blood flow and microvascular hemodynamics in real-time. It works by measuring the temporal fluctuations of speckle patterns in back-scattered light, which are caused by moving red blood cells. DCS applications include monitoring of cerebral blood flow and tumor oxygenation.

Another related laser speckle-based blood flow monitoring unit, called Laser Speckle Contrast Imaging (LSCI), is a non-invasive, high-resolution optical technique that can be used to map blood flow and perfusion in real-time. It works by illuminating tissue with a laser light, producing a speckle pattern that blurs where blood is moving, creating a contrast map that indicates blood flow velocity. When coherent laser light illuminates tissue, it creates a random speckle pattern. Moving red blood cells will cause this pattern to blur, with higher speeds of blood flow resulting in greater blurring. The technique calculates the contrast, or the ratio of the standard deviation to the mean intensity of the speckle pattern, with lower contrast values indicating higher blood flow speed.

The technology behind SCOS, DCS and LSCI is related and they use similar components. The core components for Speckle Contrast Optical Spectroscopy (SCOS), include a coherent light source (typically a laser diode at 785 nm or 850 nm), a high-sensitivity light detector (such as a CMOS or sCMOS camera, or SPAD array) for capturing speckle patterns, and a data processing unit to analyze speckle contrast, usually involving noise correction. The coherent light source is usually a laser diode (e.g., 785 nm or 850 nm) that can provide the necessary coherent illumination to produce speckle patterns. (Most SCOS may have one to three laser diodes). The light detector (camera) is usually a high-speed CMOS or Scientific CMOS (sCMOS) camera, or specialized SPAD array, to be used to capture speckle images, allowing for high-resolution measurement of speckle intensity fluctuations. (CMOS is a high-speed Complementary Metal-Oxide Semiconductor sensor). Other components may include fiber coupling/delivery to deliver the light via a fiber-coupled system to the tissue and scatted laser light can be collected either directly by the camera or through fiber bundles. The SCOS device may also include a modular probe or mount to be used to secure the laser and the camera on the scalp (or other tissue) to measure cerebral blood flow. The data processing unit uses software or hardware that calculates the speckle contrast and performs corrections for dark noise, read noise, and shot noise to ensure accurate flow measurements.

The human ear is the only location where the vagus nerve reaches the body surface (the skin), through auricular branch of vagus nerve. The cymba concha has almost exclusive vagal nerve innervation. Cavum concha (the lower, larger bowl-shaped part) is located around the opening of the external ear canal. Cavum concha also receives significant innervation from the auricular branch of vagus nerve. While cavum concha is largely innervated by the vagus, the anterior cavum concha receives mixed innervation from the auriculotemporal nerve (a branch of the mandibular division of the trigeminal nerve). The facial, glossopharyngeal, and cervical nerves also innervate part of the cavum concha. The inner and posterior tragus is innervated by vagus nerve, while the anterior outer part of tragus is innervated by trigeminal nerve (auriculotemporal nerve) and some from greater auricular nerve (which is from cervical nerve). The posterior and inferior walls of the external ear canal receive innervation from the vagus nerve, while the anterior and superior walls of the external ear canal is innervated by the auriculotemporal nerve. The vagus nerve innervated auricular skin includes inner tragus, cymba-concha, cavum-concha, posterior inferior walls of the external ear canal and small adjacent regions of the external ear. Thus, transcutaneous auricular vagus nerve stimulation (taVNS) can be placed on the vagus innervated auricular skin to stimulate the vagus nerve. The vagus nerve has huge influence on various human body functions, including brain, heart, breathing, emotions, blood pressure, GI system and metabolism etc. The cavum concha is heavily innervated by the auricular branch of vagus nerve although the auriculotemporal nerve (a branch of mandibular division of trigeminal nerve) also provides overlapping innervation for anterior superior part of cavum concha. The inner (medial) posterior tragus is primarily innervated by the vagal nerve although the anterior outer part of tragus is innervated by the auriculotemporal nerve.

The human ear is a convenient location to place a stimulating electrode for auriculotemporal nerve. The auriculotemporal nerve mainly supplies the anterior-superior part of the pinna (including anterior-superior helix), anterior outer tragus, part of the inner tragus (inner/medial surface of tragus, facing the external ear canal), anterior-superior region of cavum concha and anterior-superior walls of external ear canal. The auriculotemporal nerve innervated auricular skin includes anterior outer tragus, part of the inner tragus (inner/medial surface of tragus), anterior and superior part of pinna (including anterior-superior helix), anterior and superior walls of the external ear canal and the anterior-superior region of cavum concha.

The US Food and Drug Administration (FDA) approved vagus nerve stimulation only for left vagus nerve when stimulated at neck region, due to concern of possible bradycardia when right vagus nerve is stimulated. The practice of using transcutaneous auricular vagus nerve stimulation to treat seizures was reviewed by Yu Wang et al in 2020. They reviewed other studies and found that bilateral auricular vagus nerve stimulation is safe with no increase of side effects as compared with left-sided stimulation. They also stated that bilateral stimulation is more effective. From review of many studies, they found that the stimulation parameters for seizures have a large range of variation. The most common stimulation frequency is 20 Hz or 25 Hz (range 0.5-120 Hz). The common stimulation pulse width is 1 ms or 0.25 ms (range 0.02-1 ms). They did find that pulse width of 500 microsecond is the most biological active. They reported that the stimulation intensity is often adjusted by the patients according to their tolerance.

Vagus nerve stimulation (VNS) therapy has been approved by US Food and Drug Administration (FDA) as an adjunct treatment for drug-resistant epilepsy. The placement of a traditional VNS device requires a surgery. The VNS device is usually placed under the skin in the chest with a wire going to the neck to wrap around or attach to the left vagus nerve in the neck. The VNS can give electrical stimulation to the vagus nerve. Various settings of stimulating patterns, strength, duration, frequency and intervals have been studied. For example, a setting with stimulation given in cycles of 30 seconds on and 5 minutes off is commonly used. The stimulation could be given 3-4 times per day and each time 1-4 hours. The stimulation strength varies from low (around 0.25 mA) to high (around 1.75 mA). In some studies, patients were allowed to adjust the stimulation strength to maximum tolerable strength. The VNS has been shown to help prevent impending seizures before they start and help to stop or shorten them if they do. It also helps to decrease symptoms in the post-ictal phase. The VNS decreases seizures by sending regular mild pulse of electrical stimulation to the vagus nerve. If a person is aware of a seizure happening, the person can manually swipe a magnet on the VNS to send extra burst of electric stimuli and this often helps to stop the seizure or decrease the severity of seizure. Similarly, various settings of stimulating patterns, strength, duration, frequency and intervals of the extra burst of electric stimuli have been studied to find the most effective stimulating setting. However, there is a problem that the person might not be aware of occurrence of a breakthrough seizure, or might be unable to respond to it or too late to respond to it, or seizure occurring during sleep.

The trigeminal nerve has several branches. The most easily targeted and commonly used trigeminal nerve branches for neurostimulation include the supraorbital nerve (V1 or ophthalmic division) and infraorbital nerve (V2 or maxillary division) because they are superficial, making them easily accessible for electrode placement. The other good choice is the auriculotemporal nerve (V3 or mandibular division). The auriculotemporal nerve has a superficial temporal branch which provides sensory innervation to the skin of temple and an auricular branch which provides innervation to anterior-superior pinna (including tragus and anterior-superior helix), anterior part of cavum concha, the anterior and superior walls of external auditory canal and outer surface of the tympanic membrane. The auriculotemporal nerve has unique characteristics that its innervated auricular skin is in close proximity and overlapping with the vagus-innervated auricular skin. (The vagus-innervated auricular skin includes inner posterior portion of tragus, cymba-concha, cavum-concha, posterior inferior walls of the external ear canal and small adjacent regions of the external ear.) (The auriculotemporal nerve innervated auricular skin includes anterior outer tragus, anterior and superior part of pinna including anterior-superior helix, anterior and superior walls of the external ear canal and the anterior-superior part of cavum concha.)

Both trigeminal nerve stimulation (TNS) and transcutaneous auricular vagus nerve stimulation (taVNS) can help regulate the autonomic nervous system, improve blood flow to the brain and increase stability of large-scale functional brain networks. While direct, large-scale clinical trials combining TNS and taVNS for seizure control are quite limited, conceptually, these two methods could be synergistic since they target different, yet overlapping, afferent brain pathways (taVNS targets the solitary tract located in the brainstem, while TNS targets the trigeminal system which has extensive brainstem connections). They can also be combined with pharmacological treatments. An FDA-cleared device called “gammaCore” (vagus nerve stimulation at neck region) was found to be useful for migraine and cluster headache treatment and prevention. Another FDA-cleared device called “Cefaly” (supraorbital nerve stimulation) has been found to help migraine treatment and prevention. They act by influencing blood flow and neural activity to treat headaches.

Supraorbital nerve stimulation is a type of trigeminal nerves stimulation. The supraorbital nerve stimulation has been shown to help refractory seizures. Examples of transcutaneous supraorbital nerve stimulators include a Cefaly device which is FDA-approved for migraine. The Cafaly device is not yet FDA-cleared for seizure, although studies have shown that it is quite promising. The Cefaly device is a wearable, non-invasive therapy for seizure by stimulating the supraorbital nerve. The supraorbital nerve is a branch of the trigeminal nerve. The Cefaly device is mounted on the forehead and uses pre-determined electrical impulses on the forehead for neuromodulation. Studies have shown that supraorbital nerve stimulation can decrease the frequency of seizures by 36-57% of refractory seizures.

Auriculotemporal nerve (ATN) stimulation is another type of trigeminal nerve stimulation. An example of a ATN stimulation unit is a device called the Roo™ Therapy System by Spark Biomedical which is FDA-designated. The auriculotemporal nerve stimulation unit is sometimes combined with auricular vagus nerve stimulation unit (taVNS) due to anatomical proximity or overlapping of the auriculotemporal nerve and auricular branch of vagal nerve in the ear around tragus, cavum concha and external ear canal. The auriculotemporal nerve stimulation unit and taVNS unit may share a same housing with a same stimulating electrode. However, they often use their own optimized stimulating electrodes with their own distinct stimulating parameters. The primary targets for taVNS stimulating electrode are cymba concha (100% vagal), cavum concha, inner-posterior portion of tragus (mixed vagal/trigeminal) and posterior-inferior walls of external ear canal for autonomous nerve modulation. The primary targets for auriculotemporal nerve include anterior outer part of tragus, anterior-superior helix, anterior portion of cavum concha and anterior-superior walls of external ear canal. If the anterior-superior helix is selected as the target for the auriculotemporal nerve, the stimulating electrode may be attached via a clip electrode. The auriculotemporal nerve stimulation unit uses a setup similar to a transcutaneous electrical nerve stimulator (TENS). Both ATN stimulation and taVNS stimulation can be applied concurrently to the ear with about 30 minutes stimulation session. There are various stimulating parameters. For example, a common stimulating parameter for taVNS is 20-25 Hz frequency, 200-500 microsecond pulse width with intensity adjusted to a comfortable sensory level, usually below 5 mA. The stimulating parameters for ATN (auriculotemporal nerve stimulator) are often different, with higher frequency (around 100 Hz) to modulate different analgesic receptors. The stimulating strength for both of them can be titrated to the user's individual maximal tolerable level without pain. Combining these techniques can have synergistic effect to produce a more potent inhibitory effect on seizure.

The occipital nerve stimulator is an adjunct therapy for seizure and psychiatric disorders. The occipital nerve is not part of the trigeminal nerve because it arises from the C2 and C3 cervical spinal nerves. The stimulating electrode for occipital nerve stimulator is usually implanted surgically near the occipital nerves at the base of the skull. By impacting the convergence of cervical and trigeminal nerves, stimulation of occipital nerve can help to modulate the brain. The stimulating parameters for occipital nerve are often similar to or overlapping with those for the trigeminal nerve, focusing on delivering comfortable, non-painful stimulation that induces mild tingling (paresthesia). Occipital nerve stimulation (ONS) was found to be helpful for headaches and refractory seizures. Each occipital nerve is divided into three nerves that provide sensation to the back of the head: the greater occipital nerve (mostly C2), the lesser occipital nerve (mostly C2/C3), and the third occipital nerve (C3). The ONS is believe to help seizure by its neuromodulating processes at upper brainstem or diencephalon. It can help to reduce frequency and severity of seizure although ONS is not as common as other neuromodulation. Although ONS is primarily used for refractory pain control, there is some evidence indicating that it may help reduce seizures in refractory seizure patients as an adjunct therapy. Specific seizure reduction percentage by ONS is sparse, but related occipital-focused neurostimulation (RNS) has shown 60%-70% seizure reduction in some studies.

The greater auricular nerve (GAN) stimulation has also shown promise for various neuropsychiatric conditions, including migraine and cluster headache. GAN stimulation may help alleviate symptoms of depression by modulating neural circuits involved in mood regulation. GAN stimulation has shown potential to reduce anxiety symptoms and post-traumatic stress disorder (PTSD). GAN stimulation has also shown some therapeutic benefits in reducing seizure frequency. GAN stimulation can benefit chronic pain, including migraine and cluster headache. Studies have shown GAN stimulation has potential to enhance neural plasticity and improve cognitive functions. GAN stimulation is also found to enhance parasympathetic activity, which can lead to improved heart rate variability and reduced stress responses. This modulation of the autonomic nervous system may contribute to its therapeutic effects across various neuropsychiatric conditions

For migraine, cluster headache, seizure, and psychiatric disorders, the commonly used stimulation parameters for taVNS, supraorbital nerve stimulation unit, auriculotemporal nerve stimulation unit, occipital nerve stimulation unit, greater auricular nerve stimulation unit and infraorbital nerve stimulation unit may be somewhat different. Examples for these stimulation parameters are:

### Common Stimulation Parameters (General Approach)

(1). Intensity: Individually fitted to create a strong, “tingling” but non-painful sensation, often described as below the pain threshold.
(2). Pulse width: 250-500
(3). Frequency: 1 Hz (often used for modulation) or 20-30 Hz (commonly used for seizure/neurological).
(4). Waveform: Often dense-sparse waves.
   Examples for disease-specific parameters for taVNS (also sometimes used for auriculotemporal nerve stimulation unit):
(A). For Migraine and chronic headache:
   Frequency: 1 Hz (found to have better results for daily prevention) or 25 Hz.
   Duration: 4 hours/day (often split into 1-4 hours sessions).
   Intensity: Adjusted by the patient to a comfortable, tingling sensation.
(B). For seizure:
   Frequency: 20 Hz (most commonly used).
   Intensity: adjusted to the highest tolerable limit (4-12 mA range).
   Duty Cycle: 30 seconds on/5 minutes off, or 30 s on/30 s off to avoid habituation.
(C). For psychiatric disorders:
   Frequence: around 20 Hz.
   Duration: 20 min to 1 hour daily.
   Intensity: Maximum, yet non-painful (4-6 mA range reported).
      There are other reported examples of stimulation parameters for trigeminal nerve stimulation (TNS) (including supraorbital nerve stimulation unit, auriculotemporal nerve stimulation unit and infraorbital nerve stimulation unit) and occipital nerve stimulation unit: a high-frequency, low-intensity pulse is usually used, for example a 100-120 Hz frequency and a 250 microseconds pulse width. The typical stimulation parameters for trigeminal nerve stimulation are:
(1). Intensity/Strength: 2-4 mA (with a range of 1-10 mA, maximum of 16 mA) for migraine, adjusted for comfort to a mild tingling sensation without pain.
(2). Frequency: 60-120 Hz
(3). Pulse width (Duration) 200 to 250 microseconds.
(4). Stimulation cycle: commonly 30 seconds on/30 seconds off or continuous for 20-60 minutes depending on condition.
(5). Session duration: 7-9 hours (overnight) for seizure
(6). Waveform: usually biphasic pulses.

### BRIEF SUMMARY OF THE INVENTION

According to one aspect consistent with the principles of the invention, an auricular electroencephalogram (EEG) monitoring system is provided. In some embodiments, an auricular electroencephalogram (EEG) monitoring system may include an auricular EEG recording module configured to be linked to a wearer's first ear or the peri-auricular area around the first ear. The EEG recording module may include a plurality of (at least two, but preferably more than two) EEG sensor electrodes and an optional reference electrode. These EEG sensor electrodes and the optional reference electrode are configured to contact separate areas of the wearer's first ear or the peri-auricular area around the wearer's first ear. The areas of the ear or the peri-auricular area that the EEG sensor electrodes and the optional reference electrode are configured to contact may be selected from at least one of the following: an external portion of the wearer's first ear, an external ear canal of the wearer's first ear, and peri-auricular area around the wearer's first ear. The peri-auricular area refers to a portion of the head around the auricle (pinna) and this portion of the head is typically hairless. The peri-auricular area includes a portion of the head in front of the auricle (pre-auricular area) and a portion of the head above and behind the auricle (post-auricular area). The pre-auricular area is small, about one inch wide and about two inches long and curved along the anterior edge of the auricle. The post-auricular area is also small, approximately one inch wide and about three inches long and curved along the superior and posterior edges of the auricle (pinna). The post-auricular area is where a behind-the-ear hearing aid is usually located. These small pre-auricular area and post-auricular area together will be called “peri-auricular area” hereinafter. (Anterior, posterior, superior, in front of and behind etc. all refer to the directions relative to the wearer's head when the wearer is in an upright position.) The auricular EEG recording module may be configured to record EEG data of the wearer.

The auricular electroencephalogram (EEG) monitoring system may include a network interface which may be configured to generate a notification to a client device or a client device of the wearer's healthcare provider. A processing unit may be in electronic communication with the auricular EEG recording module and the network interface. The processing unit may be configured to analyze the EEG data recorded by the auricular EEG recording module to detect presence or cessation of EEG signals suggestive of migraine or cluster headache. The processing unit may be also configured to analyze the EEG data recorded by the auricular EEG recording module to detect presence or cessation of EEG signals suggestive of impending migraine or impending cluster headache. When the presence of EEG signals suggestive of migraine or cluster headache is detected, the processing unit may be configured to immediately send signals to the network interface to generate a notification to the client device. When the presence of EEG signals suggestive of impending migraine or impending cluster headache is detected, the processing unit may be configured to immediately send signals to the network interface to generate a notification to the client device. When the cessation of EEG signals suggestive of migraine or cluster headache is detected, the processing unit may be configured to immediately send signals to the network interface to generate a notification to the client device. Likewise, when the cessation of EEG signals suggestive of impending migraine or impending cluster headache is detected, the processing unit may be further configured to immediately send signals to the network interface to generate a notification to the client device.

According to one aspect consistent with the principles of the invention, a neurovascular monitoring system is provided. The neurovascular monitoring system comprises an auricular EEG monitoring system and a cephalic blood flow monitoring system to monitor intracranial and extracranial blood flow, such as a laser-speckle based blood flow monitoring unit, including a Speckle Contrast Optical Spectroscopy (SCOS) unit or an auricular SCOS unit. (Alternatively, photoplethysmography (PPG) may be used to monitor blood flow although PPG primarily monitor changes in blood volume and is less sensitive. PPG is mostly useful for superficial or skin level monitoring. Another alternative is ultrasound and doppler based blood flow monitoring.) The SCOS unit may be configured to assess the cerebral and extracranial blood flow data from the wearer's (user) scalp while an auricular SCOS unit may be configured to assess the cerebral and extracranial blood flow data from the wearer's ear. The neurovascular monitoring system further comprises a processing unit and a network interface. The processing unit is in electronic communication with the network interface, the auricular EEG monitoring system and the auricular SCOS unit. The processing unit may analyze the cerebral and extracranial blood flow data transmitted from the auricular SCOS unit to assess the wearer's intracranial and extracranial blood flow data. The processing unit may be configured to analyze the EEG data (transmitted from or recorded by the auricular EEG monitoring system) to detect presence of EEG data suggestive of migraine or cluster headache. The processing unit is also configured to analyze the blood flow data (transmitted from or recorded by the auricular SCOS unit) to detects presence or cessation of blood flow data suggestive of migraine or cluster headache. In addition, the processing unit may be configured to analyze the EEG data (transmitted from or recorded by the auricular EEG monitoring system) to detect presence of EEG data suggestive of impending migraine or impending cluster headache. The processing unit is also configured to analyze the blood flow data (transmitted from or recorded by the auricular SCOS unit) to detects presence or cessation of blood flow data suggestive of impending migraine or impending cluster headache. When the processing unit detects at least one of the following: presence of EEG signals suggestive of migraine or cluster headache and presence of blood flow data suggestive of migraine or cluster headache, the processing unit is configured to send signals to the network interface which may be configured to generate a notification to a client device or a client device of the wearer's healthcare provider. When the processing unit detects at least one of the following: presence of EEG signals suggestive of impending migraine or impending cluster headache and presence of blood flow data suggestive of impending migraine or impending cluster headache, the processing unit is configured to send signals to the network interface which may be configured to generate a notification to the client device or the client device of the wearer's healthcare provider. When the processing unit detects all of the following: cessation of EEG signals suggestive of migraine or cluster headache, cessation of blood flow data suggestive of migraine or cluster headache, cessation of the EEG signals suggestive of impending migraine or impending cluster headache and cessation of blood flow data suggestive of impending migraine or impending cluster headache, the processing unit may be further configured to send signals to the network interface to generate a notification to the client device. An auricular EEG monitoring system is usually enough to detect presence of EEG signals suggestive of migraine and impending migraine. A neurovascular monitoring system may further enhance the accuracy in detecting migraine and impending migraine. Whereas, an auricular EEG monitoring system may be only modestly enough to detect presence of EEG signals suggestive of cluster headache and impending cluster headache. A neurovascular monitoring system may significantly enhance the accuracy in detecting cluster headache and impending cluster headache. Besides useful for monitoring of migraine and cluster headache, the neurovascular monitoring system may be very useful for monitoring of stroke, impending stroke and traumatic brain injury, etc.

In a modified aspect consistent with the principles of the invention, a neurovascular monitoring system may be configured as an auricular neurovascular monitoring system. The auricular neuro-vascular monitoring system comprises an auricular EEG monitoring system and a cephalic blood flow monitoring system, such as an auricular speckle contrast optical spectroscopy unit (auricular SCOS unit). (Auricula SCOS unit is a SCOS unit housed in a wearer's ear, for example in the concha or external ear canal of the wearer's ear). The auricular SCOS unit may be configured to assess the cerebral and extracranial blood flow data from the wearer's ear. The auricular neurovascular monitoring system further comprises a processing unit and a network interface. The processing unit is in electronic communication with the network interface, the auricular EEG monitoring system and the auricular SCOS unit. The processing unit may analyze the SCOS data transmitted from (or recorded by) the auricular SCOS unit to assess the wearer's intracranial (cerebral) and extracranial blood flow data. The processing unit may be configured to analyze the EEG data (transmitted from or recorded by the auricular EEG monitoring system) to detect presence or cessation of EEG signals suggestive of migraine or cluster headache. The processing unit is configured to analyze the blood flow data (transmitted from or recorded by the auricular SCOS unit) to detect presence or cessation of blood flow data suggestive of migraine or cluster headache. In addition, the processing unit may be configured to analyze the EEG data (transmitted from or recorded by the auricular EEG monitoring system) to detect presence of EEG signals suggestive of impending migraine or impending cluster headache. The processing unit is also configured to analyze the blood flow data (transmitted from or recorded by the auricular SCOS unit) to detects presence or cessation of blood flow data suggestive of impending migraine or impending cluster headache. When the processing unit detects at least one of the following: presence of EEG signals suggestive of migraine or cluster headache and presence of blood flow data suggestive of migraine or cluster headache, the processing unit is configured to send signals to the network interface which may be configured to generate a notification to a client device or a client device of the wearer's healthcare provider. When the processing unit detects at least one of the following: presence of EEG signals suggestive of impending migraine or impending cluster headache and presence of blood flow data suggestive of impending migraine or impending cluster headache, the processing unit is configured to send signals to the network interface which may be configured to generate a notification to the client device or the client device of the wearer's healthcare provider. When the processing unit detects all of the following: cessation of EEG signals suggestive of migraine or cluster headache, cessation of blood flow data suggestive of migraine or cluster headache, cessation of the EEG signals suggestive of impending migraine or impending cluster headache and cessation of blood flow data suggestive of impending migraine or impending cluster headache, the processing unit may be further configured to send signals to the network interface to generate a notification to the client device. An auricular EEG monitoring system is usually enough to detect presence of EEG signals suggestive of migraine and impending migraine. An auricular neurovascular monitoring system may further enhance the accuracy in detecting migraine and impending migraine. Whereas, an auricular EEG monitoring system may be only modestly enough to detect presence of EEG signals suggestive of cluster headache and impending cluster headache. An auricular neurovascular monitoring system may significantly enhance the accuracy in detecting cluster headache and impending cluster headache. Besides useful for monitoring of migraine and cluster headache, the auricular neurovascular monitoring system may be very useful for monitoring of stroke, impending stroke and traumatic brain injury etc.

According to another aspect consistent with the principles of the invention, automatic detection-therapy systems for migraine or cluster headache are disclosed. An automatic detection-therapy system may include a first auricular electroencephalogram (EEG) monitoring system, a first neuromodulation unit and a processing unit. The processing unit is in electronic communication with the auricular EEG monitoring system and the neuromodulation unit. The auricular EEG monitoring system may comprise an auricular EEG recording module configured to be linked to a wearer's first ear or a peri-auricular area around the wearer's first ear. The EEG recording module may include a plurality of (at least two, but preferably more than two) EEG sensor electrodes and an optional reference electrode. These EEG sensor electrodes and the optional reference electrode are configured to contact separate areas of the wearer's first ear or peri-auricular area around the wearer's first ear. The areas that the EEG sensor electrodes and the optional reference electrode are configured to contact may be selected from at least one of the following: an external ear of the wearer's first ear, an external ear canal of the wearer's first ear, and a peri-auricular area around the wearer's first ear. The auricular EEG recording module may be configured to record EEG data of the wearer. The first neuromodulation unit comprises at least one of the following components: a first transcutaneous auricular vagus nerve stimulation unit (taVNS unit), a supraorbital nerve stimulation unit, a first auriculotemporal nerve stimulation unit, an occipital nerve stimulation unit, a first greater auricular nerve stimulation unit and an infraorbital nerve stimulation unit. As an example, a neuromodulation unit may comprise a first transcutaneous auricular vagus nerve stimulation unit (first taVNS unit). The first taVNS unit has a first taVNS stimulating electrode configured to contact vagus innervated auricular skin of the wearer's first ear. The vagus innervated auricular skin includes external ear canal, tragus, cymba-concha, cavum-concha and small adjacent areas. (More precisely, the vagus-innervated auricular skin includes inner posterior portion of tragus, cymba-concha, cavum-concha, posterior inferior walls of the external ear canal and small adjacent regions of the external ear.) The vagus innervated auricular skin that the first taVNS stimulating electrode is configured to contact may be selected from at least one of the following: tragus, cymba-concha, cavum-concha and the external ear canal of the wearer's first ear. A processing unit may be in electronic communication with the auricular EEG recording module and in electronic communication with the first taVNS unit. The processing unit may be configured to analyze the EEG data recorded by the EEG monitoring system with the help of advanced EEG analysis algorithms together with machine learning, deep learning and artificial intelligence to detect the presence or cessation of EEG signals suggestive of migraine or cluster headache. When the presence of EEG signals suggestive of migraine or cluster headache is detected by the processing unit, the processing unit may be configured to immediately send signals to the first taVNS unit to automatically start sending pre-determined neuromodulating electric stimulation to the vagus innervated auricular skin of the wearer's first ear to which the first taVNS stimulating electrode is in contact with. When cessation of EEG signals suggestive of migraine or cluster headache is detected by the processing unit, the processing unit may be further configured to immediately send signals to the first taVNS unit to automatically stop sending pre-determined electric stimulation to the vagus innervated auricular skin of the wearer's first ear. The processing unit may be further configured to analyze the EEG date recorded by the EEG monitoring system to detect presence or cessation of EEG signals suggestive of impending migraine or impending cluster headache. When the presence of EEG signals suggestive of impending migraine or impending cluster headache is detected by the processing unit, the processing unit may be configured to immediately send signals to the first taVNS unit to automatically start sending predetermined neuromodulating electric stimulation to the vagus innervated auricular skin of the wearer's first ear. When cessation of EEG signals suggestive of impending migraine or impending cluster headache is detected by the processing unit, the processing unit may be further configured to immediately send signals to the first taVNS unit to automatically stop sending pre-determined electric stimulation to the vagus innervated auricular skin of the wearer's first ear. In other examples, the neuromodulation unit may comprise at least one of: a taVNS unit, a supraorbital nerve stimulation unit, an auriculotemporal nerve stimulation unit, an occipital nerve stimulation unit, a greater auricular nerve stimulation unit, an infraorbital nerve stimulation unit and various combinations thereof, and their setups are similar to the aforementioned descriptions for taVNS. They will be described in more details hereinafter, including various combinations of them.

According to an additional aspect consistent with the principles of the invention, automatic detection-therapy systems for migraine or cluster headache are disclosed. In some embodiments, an automatic detection-therapy system for migraine or cluster headache may include a first neurovascular monitoring system, a first neuromodulation unit and a processing unit. The first neurovascular monitoring system may comprise an auricular EEG monitoring system and a cephalic blood flow monitoring unit. Examples of the cephalic blood flow monitoring unit include ultrasound doppler-based blood flow monitoring unit (such as transcranial doppler ultrasound, TCD) or laser speckle-based blood flow monitoring unit (such as speckle contrast optical spectroscopy unit, SCOS unit) or photoplethysmography (PPG). There are a few different (related) types of laser speckle-based blood flow monitoring unit. The laser speckle blood flow systems, used for real-time, full-field mapping of tissue perfusion, are most commonly referred to as laser speckle contrast imaging (LSCI). Other frequently used names include laser speckle imaging (LSI), laser speckle perfusion imaging (LSPI), laser speckle flowmetry (LSF) and laser speckle contrast analysis (LASCA). For simplicity purpose, a Speckle Contrast Optical Spectroscopy unit, (SCOS unit) will be described, although all of the other blood flow monitoring devices are within the scope of this invention. The SCOS unit may be configured to be housed in an auricular housing and will be called “auricular Speckle Contrast Optical Spectroscopy unit”, or “auricular SCOS unit” hereinafter. The processing unit is in electronic communication with the auricular EEG monitoring system, a first SCOS unit (or a first auricular SCOS unit) and the first neuromodulation unit. The auricular EEG monitoring system comprises a first auricular EEG recording module configured to be linked to a wearer's first ear or a peri-auricular area around the wearer's first ear. The first auricular EEG recording module may include a plurality of (at least two, but preferably more than two) EEG sensor electrodes and an optional reference electrode. These EEG sensor electrodes and the optional reference electrode are configured to contact separate areas of the wearer's first ear or peri-auricular area around the wearer's first ear. The areas that the EEG sensor electrodes and the optional reference electrode are configured to contact may be selected from at least one of the following: an external ear of the wearer's first ear, an external ear canal of the wearer's first ear, and a peri-auricular area around the wearer's first ear. The first auricular EEG recording module may be configured to record EEG data of the wearer. The first speckle contrast optical spectroscope unit (first SCOS unit) is configured to measure the wearer's intracranial (cerebral) blood flow and extracranial blood flow from the wearer's scalp. The first SCOS unit may be configured to be housed in an auricular housing to be placed in the wearer's first ear (such as concha or external ear canal of the wearer's first ear) to become an auricular SCOS unit. The auricular SCOS unit may be configured to measure the intracranial (cerebral) blood flow and the extracranial blood flow from the wearer's ear (such as concha or external ear canal). Combination and integration of an auricular EEG monitoring system and a SCOS unit is called “neurovascular monitoring system” hereinafter. Combination and integration of an auricular EEG monitoring system and an auricular SCOS unit is called “auricular neurovascular monitoring system” hereinafter. In some embodiments, an auricular SCOS unit may be housed in an auricular housing (housing refers to protective cover or protective structure) and the auricular EEG monitoring system may also be housed in the same auricular housing so that the auricular neurovascular monitoring system will exist as one device. The first neuromodulation unit may comprise at least one of the following components: a first transcutaneous auricular vagus nerve stimulation unit (taVNS unit), a supraorbital nerve stimulation unit, a first auriculotemporal nerve stimulation unit, an occipital nerve stimulation unit, a first greater auricular nerve stimulation unit and an infraorbital nerve stimulation unit. The processing unit may be in electronic communication with the first auricular EEG recording module and the first auricular neurovascular monitoring system (including the auricular EEG monitoring system and the first auricular SCOS unit). The processing unit may be configured to analyze the EEG data recorded by the EEG monitoring system with the help of advanced EEG analysis algorithms together with machine learning, deep learning and artificial intelligence to detect the presence or cessation of EEG signals suggestive of migraine or cluster headache. The processing unit may also be configured to analyze the cerebral (intracranial) and extracranial blood flow data recorded by the first auricular SCOS unit to detect the presence or cessation of blood flow data suggestive of migraine or cluster headache. In addition, the processing unit may be configured to analyze the EEG data recorded by the auricular EEG monitoring system with the help of advanced EEG analysis algorithms together with machine learning, deep learning and artificial intelligence to detect the presence or cessation of EEG signals suggestive of impending migraine or impending cluster headache. The processing unit may also be configured to analyze the cerebral (intracranial) and extracranial blood flow data recorded by the first auricular SCOS unit to detect the presence or cessation of blood flow data suggestive of impending migraine or impending cluster headache. When the processing unit detects one of the following: presence of EEG signals suggestive of migraine or cluster headache and presence of blood flow data suggestive of migraine or cluster headache, the processing unit may be configured to immediately send signals to the first neuromodulation unit to prompt it to automatically start sending pre-determined neuromodulating electric stimulation to at least one of the following nerves of the wearer, including the auricular branch of vagus nerve on the wearer's first ear, the supraorbital nerve, the auriculotemporal nerve on the wearer's first ear, the occipital nerve, the greater auricular nerve on the wearer's first ear and the infraorbital nerve. When the processing unit detects one of the following: presence of EEG signals suggestive of impending migraine or impending cluster headache and presence of blood flow data suggestive of impending migraine or impending cluster headache, the processing unit may be configured to immediately send signals to the first neuromodulation unit to prompt it to automatically start sending pre-determined neuromodulating electric stimulation to at least one of the following nerves of the wearer: the auricular branch of vagus nerve on the wearer's first ear, the supraorbital nerve, the auriculotemporal nerve on the wearer's first ear, the occipital nerve, the greater auricular nerve on the wearer's first ear and the infraorbital nerve. When the processing unit detects all of the following: cessation of EEG signals suggestive of migraine or cluster headache, cessation blood flow data suggestive of migraine or cluster headache, cessation of EEG signals suggestive of impending migraine or impending cluster headache and cessation blood flow data suggestive of impending migraine or impending cluster headache, the processing unit is configured to send signals to the first neuromodulation unit to prompt it to stop the neuromodulating electric stimulation to any of the nerves.

According to still another aspect consistent with the principles of the invention, a novel automatic detection-therapy system for migraine is disclosed. This system may include an auricular electroencephalogram (EEG) monitoring system, a first neuromodulation unit and a processing unit. The auricular EEG monitoring system and the first neuromodulation unit are essentially the same as aforementioned descriptions. The processing unit is in electronic communication with the auricular EEG monitoring system and the first neuromodulation unit. The first neuromodulation unit may comprise at least one of the following components: a first taVNS unit (located in the wearer's first ear), a supraorbital nerve stimulation unit (located at the wearer's midforehead), a first auriculotemporal nerve stimulation unit (located in the wearer's first ear), an occipital nerve stimulation unit (located at the wearer's mid-occipital region), a first greater auricular nerve stimulation unit (located in the wearer's first ear) and an infraorbital nerve stimulation unit (located at the wearer's mid-face). The auricular electroencephalogram (EEG) monitoring system may include a first auricular EEG recording module configured to be linked to a wearer's (or user's) first ear or a peri-auricular area around the first ear. The EEG recording module may include a plurality of (at least two, but preferably more than two) EEG sensor electrodes and an optional reference electrode. These EEG sensor electrodes and the optional reference electrode may be configured to contact separate areas of the wearer's first ear or the peri-auricular area around the wearer's first ear. The areas of the ear or the peri-auricular area that the EEG sensor electrodes and the optional reference electrode are configured to contact may be selected from at least one of the following: an external portion of the wearer's first ear, an external ear canal of the wearer's first ear, and peri-auricular area around the wearer's first ear. The EEG monitoring system is configured to record the wearer's EEG data. Optionally, the automatic detection-therapy system for migraine may further comprise a network interface in electronic communication with the processing unit. The processing unit may be configured to analyze the EEG data recorded by the auricular EEG monitoring system with the help of advanced EEG analysis algorithms together with machine learning, deep learning and artificial intelligence to detect presence or cessation of EEG signals suggestive of migraine or impending migraine. When the presence of EEG signals suggestive of migraine or impending migraine is detected, the processing unit may be configured to immediately send signals to the first neuromodulation unit to prompt the first neuromodulation unit to start pre-determined neuromodulating electric stimulation to at least one of the following nerves: auricular branch of vagus nerve on the wearer's first ear, supraorbital nerves, auriculotemporal nerve on the wearer's first ear, occipital nerves, greater auricular nerve on the wearer's first ear and infraorbital nerve. When cessation of EEG signals suggestive of migraine or impending migraine is detected, the processing unit may be further configured to immediately send signals to the first neuromodulation unit to prompt the first neuromodulation unit to stop neuromodulating electric stimuli to any of the aforementioned nerve.

According to yet another aspect consistent with the principles of the invention, a novel modified automatic detection-therapy system for migraine is disclosed. The modified automatic detection-therapy system for migraine may further include a laser speckle-based blood flow monitoring unit, such as a first speckle contrast optical spectroscopy unit (first SCOS unit) or a first auricular speckle contrast optical spectroscopy unit (first auricular SCOS unit), configured to record the wearer's intracranial (cerebral) and extracranial blood flow data. A neurovascular monitoring system may be configured as a combination or integration of an auricular EEG monitoring system and a SCOS unit; while an auricular neurovascular monitoring system may be configured as a combination or integration of an auricular EEG monitoring system and an auricular SCOS unit. The processing unit is configured to be in electronic communication with the auricular EEG monitoring system, the first SCOS unit (or the first auricular SCOS unit) and the first neuromodulation unit. The processing unit may be configured to use both the EEG signals and the blood flow data to detect presence or cessation of EEG signals and blood flow data suggestive of migraine or impending migraine. When the processing unit detects one of the following: presence of EEG signals suggestive of migraine and presence of blood flow data suggestive of migraine, the processing unit may be configured to immediately send signals to the first neuromodulation unit to prompt it to automatically start sending pre-determined neuromodulating electric stimulation to at least one of the following nerves of the wearer: the auricular branch of vagus nerve on the wearer's first ear, the supraorbital nerve, the auriculotemporal nerve on the wearer's first ear, the occipital nerve, the greater auricular nerve on the wearer's first ear and the infraorbital nerve. When the processing unit detects one of the following: presence of EEG signals suggestive of impending migraine and presence of blood flow data suggestive of impending migraine, the processing unit may be configured to immediately send signals to the first neuromodulation unit to prompt it to automatically start sending pre-determined neuromodulating electric stimulation to at least one of the following nerves of the wearer: the auricular branch of vagus nerve on the wearer's first ear, the supraorbital nerves, the auriculotemporal nerve on the wearer's first ear, the occipital nerves, the greater auricular nerve on the wearer's first ear and the infraorbital nerve. When the processing unit detects all of the following: cessation of EEG signals suggestive of migraine, cessation of blood flow data suggestive of migraine, cessation of EEG signals suggestive of impending migraine and cessation blood flow data suggestive of impending migraine, the processing unit is configured to send signals to the first neuromodulation unit to prompt it to stop the neuromodulating electric stimulation to any of the aforementioned nerve.

According to a further aspect consistent with the principles of the invention, a novel automatic detection-therapy system for cluster headache is disclosed. The automatic detection-therapy system for cluster headache may comprise an auricular EEG monitoring system, a laser speckle-based blood flow monitoring unit such as a first auricular SCOS unit (or a first SCOS unit), a first neuromodulation unit and a processing unit. The auricular EEG monitoring system, the first auricular SCOS unit, and the first neuromodulation unit are similar to the aforementioned descriptions. The processing unit is in electronic communication with the auricular EEG monitoring system, the first auricular SCOS unit, and the first neuromodulation unit. The first neuromodulation unit may comprise at least one of the following components: a first transcutaneous auricular vagus nerve stimulation unit (first taVNS unit) (located in the wearer's first ear), a supraorbital nerve stimulation unit (located at wearer's midforehead), a first auriculotemporal nerve stimulation unit (located in the wearer's first ear), an occipital nerve stimulation unit (located at wearer's mid-occipital region), a first greater auricular nerve stimulation unit (located in the wearer's first ear) and an infraorbital nerve stimulation unit. The processing unit is configured to analyze the EEG data recorded by the EEG monitoring system and the blood flow data recorded by the first auricular SCOS unit to detect the presence or cessation of EEG signals and blood flow data suggestive of cluster headache and impending cluster headache. When the processing unit detects one of the following: presence of EEG signals suggestive of cluster headache and presence of blood flow data suggestive of cluster headache, the processing unit may be configured to immediately send signals to the first neuromodulation unit to prompt it to automatically start sending pre-determined neuromodulating electric stimulation to at least one of the following nerves of the wearer: the auricular branch of vagus nerve on the wearer's first ear, the supraorbital nerves, the auriculotemporal nerve on the wearer's first ear, the occipital nerves, the greater auricular nerve on the wearer's first ear and the infraorbital nerve. When the processing unit detects one of the following: presence of EEG signals suggestive of impending cluster headache and presence of blood flow data suggestive of impending cluster headache, the processing unit may be configured to immediately send signals to the first neuromodulation unit to prompt it to automatically start sending pre-determined neuromodulating electric stimulation to at least one of the following nerves of the wearer: the auricular branch of vagus nerve on the wearer's first ear, the supraorbital nerve, the auriculotemporal nerve on the wearer's first ear, the occipital nerve, the greater auricular nerve on the wearer's first ear and the infraorbital nerve. When the processing unit detects all of the following: cessation of EEG signals suggestive of cluster headache, cessation of blood flow data suggestive of cluster headache, cessation of EEG signals suggestive of impending cluster headache and cessation blood flow data suggestive of impending cluster headache, the processing unit may be further configured to send signals to the first neuromodulation unit to prompt it to stop the neuromodulating electric stimulation to any of the aforementioned nerve.

For the automatic detection-therapy system for migraine, examples of the taVNS stimulating parameters for migraine and impending migraine are shown in Tables 1 and 2. Table 1 shows examples of stimulating parameters for the taVNS unit for migraine.

TABLE 1
Example of taVNS unit electric stimulation
output parameters for migraine:

| Output | Parameter | |
|---|---|---|
| Power supply | Direct current 3-9 volts | |
| Pulse width | 0.05-0.25 | ms |
| Frequency | 1-25 | Hz |

| | |
|---|---|
| Modes | Bi-phasic waveform |
| Intensity | 0.1-5.0 mA (Start at 0.1 mA, adjust at 0.1 |
| | mA increment till maximum tolerable |
| | intensity) |
| On/off time | 30 sec on/30 sec off |
| Sessions | 30-240 min/session, daily or 3 times/week, |
| | total duration 4-12 weeks |

For the automatic detection-therapy system for impending migraine, examples for the taVNS stimulating parameters are shown in Table 2.

TABLE 2
Example of taVNS unit electric stimulation
output parameters for impending migraine:

| Output | Parameter | |
|---|---|---|
| Power supply | Direct current 3-9 volts | |
| Pulse width | 0.05-0.25 | ms |
| Frequency | 1-25 | Hz |

| | |
|---|---|
| Modes | Bi-phasic waveform |
| Intensity | 0.1-3.0 mA (Start at 0.1 mA, adjust at 0.1 |
| | mA increment till maximum tolerable |
| | intensity) |
| On/off time | 30 sec on/30 sec off |
| Sessions | 30-200 min/session, daily or 3 times/week, |
| | total duration 4-8 weeks |

For the automatic detection-therapy system for cluster headache, examples of the taVNS stimulating parameters for cluster headache and impending cluster headache are shown in Tables 3 and 4. Table 3 shows examples of stimulating parameters for the taVNS unit for cluster headache

TABLE 3
Example of taVNS unit electric stimulation output
parameters for impending cluster headache:

| Output | Parameter | |
|---|---|---|
| Power supply | Direct current 3-9 volts | |
| Pulse width | 0.05-0.45 | ms |
| Frequency | 10-30 | Hz |
| Modes | Continuous wave or sparse-dense wave | |
| Intensity | 0.1-4.0 | mA |

| | |
|---|---|
| Sessions | 20-60 min/session, Duration 3 weeks |

For the automatic detection-therapy system for impending cluster headache, examples for the taVNS stimulating parameters are shown in Table 4.

TABLE 4
Example of taVNS unit electric stimulation output
parameters for impending cluster headache:

| Output | Parameter | |
|---|---|---|
| Power supply | Direct current 3-9 volts | |
| Pulse width | 0.05-0.45 | ms |
| Frequency | 10-30 | Hz |
| Modes | Continuous wave or sparse-dense wave | |
| Intensity | 0.1-4.0 | mA |

| | |
|---|---|
| Sessions | 20-60 min/session, Duration 3 weeks |

In some embodiments, an automatic detection-therapy system for migraine or cluster headache may include a neuromodulation unit that may comprise a supraorbital nerve stimulation unit. The supraorbital nerve is a branch of the ophthalmic division of the trigeminal nerve. There are two supraorbital nerves, one on each side of the forehead. (Some non-invasive transcutaneous supraorbital nerve stimulators, such as the CEFALY device, are placed on midforehead and are designed to deliver electrical stimulation to the supraorbital nerves on both sides of the forehead simultaneously.) The supraorbital nerve stimulation unit includes a supraorbital nerve stimulating electrode configured to contact the supraorbital nerve innervated area of the wearer's forehead skin. A processing unit is in electronic communication with the auricular EEG recording module and the supraorbital nerve stimulation unit. The processing unit is configured to analyze the EEG data recorded by the EEG monitoring system to detect the presence or cessation of EEG signals suggestive of migraine or cluster headache. When the presence of EEG signals suggestive of migraine or cluster headache is detected by the processing unit, the processing unit is configured to immediately send signals to the supraorbital nerve stimulation unit to prompt it to automatically start sending pre-determined neuromodulating electric stimulation to the wearer's supraorbital nerves. When cessation of EEG signals suggestive of migraine or cluster headache is detected by the processing unit, the processing unit is further configured to immediately send signals to the supraorbital nerve stimulation unit to prompt it to automatically stop sending neuromodulating electric stimulation to the wearer's supraorbital nerves. The processing unit is further configured to analyze the EEG data recorded by the EEG monitoring system to detect presence or cessation of EEG signals suggestive of impending migraine or impending cluster headache. When the presence of EEG signals suggestive of impending migraine or impending cluster headache is detected by the processing unit, the processing unit is configured to immediately send signals to the supraorbital nerve stimulation unit to prompt it to automatically start sending predetermined neuromodulating electric stimulation to the wearer's supraorbital nerves. When cessation of EEG signals suggestive of impending migraine or impending cluster headache is detected by the processing unit, the processing unit is further configured to immediately send signals to the supraorbital nerve stimulation unit to prompt it to automatically stop sending neuromodulating electric stimulation to the wearer's supraorbital nerves.

In some modified embodiments, an automatic detection-therapy system for migraine or cluster headache may further comprises a laser speckle-based blood flow monitoring unit such as a speckle contrast optical spectroscopy unit (SCOS unit) or an auricular SCOS unit. The automatic detection-therapy system for migraine or cluster headache includes a neuromodulation unit that may comprise a supraorbital nerve stimulation unit. The supraorbital nerve stimulation unit includes a supraorbital nerve stimulating electrode configured to contact the supraorbital nerve innervated area of the wearer's forehead skin. A processing unit is in electronic communication with the auricular EEG recording module, the auricular SCOS unit (or SCOS unit) and the supraorbital nerve stimulation unit. The processing unit is configured to analyze the EEG data recorded by the EEG monitoring system to detect the presence or cessation of EEG signals suggestive of migraine or cluster headache. The processing unit is also configured to analyze the blood flow data recorded by the SCOS unit (or auricular SCOS unit) to detect the presence or cessation of blood flow data suggestive of migraine or cluster headache. When the processing unit detects one of the following: the presence of EEG signals suggestive of migraine or cluster headache and presence of blood flow data suggestive of migraine or cluster headache, the processing unit is configured to immediately send signals to the supraorbital nerve stimulation unit to prompt it to automatically start sending pre-determined neuromodulating electric stimulation to the wearer's supraorbital nerves. The processing unit is configured to analyze the EEG data recorded by the EEG monitoring system to detect presence or cessation of EEG signals suggestive of impending migraine or impending cluster headache. The processing unit is also configured to analyze the blood flow data recorded by the auricular SCOS unit (or SCOS unit) to detect presence or cessation of blood flow data suggestive of impending migraine or impending cluster headache. When the processing unit detects one of the following: the presence of EEG signals suggestive of impending migraine or impending cluster headache and presence of blood flow data suggestive of impending migraine or impending cluster headache, the processing unit is configured to immediately send signals to the supraorbital nerve stimulation unit to prompt it to automatically start sending predetermined neuromodulating electric stimuli to the wearer's supraorbital nerves. When the processing unit detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit is further configured to immediately send signals to the supraorbital nerve stimulation unit to prompt it to automatically stop sending neuromodulating electric stimuli to the wearer's supraorbital nerves.

In some embodiments, an automatic detection-therapy system for migraine or cluster headache may include a neuromodulation unit that may comprise an auriculotemporal nerve stimulation unit. The auriculotemporal nerve is a branch of the mandibular division of the trigeminal nerve. The auriculotemporal nerve stimulation unit includes an auriculotemporal nerve stimulating electrode configured to contact the auriculotemporal nerve innervated area of the wearer's ear. The auriculotemporal nerve innervated area includes the anterior-outer part of tragus, upper anterior part of the helix, anterior portion of cavum concha, anterior and superior walls of external ear canal, outer surface of the tympanic membrane and area immediately in front of the tragus. The auriculotemporal nerve innervated area is anatomically adjacent to or overlapping with the vagus-innervated auricular skin. (For comparison: The vagus-innervated auricular skin includes: inner posterior portion of tragus, cymba-concha, cavum-concha, posterior and inferior walls of the external ear canal and small adjacent regions of the external ear. The auriculotemporal nerve innervated auricular skin includes: anterior outer part of tragus, the anterior-superior part of cavum concha, anterior and superior walls of the external ear canal and, anterior and superior part of pinna including anterior-superior helix,) Because of this, it is feasible for the auriculotemporal nerve stimulation unit and the taVNS unit to share a housing device or even share a stimulating electrode. However, it is usually preferred to have separate devices (could be integrated into one housing) with separate stimulating electrodes and separate stimulating parameters because their optimal stimulating parameters are often different. A processing unit is in electronic communication with the auricular EEG recording module and the auriculotemporal nerve stimulation unit. The processing unit is configured to analyze the EEG data recorded by the EEG monitoring system to detect the presence or cessation of EEG signals suggestive of migraine or cluster headache. When the presence of EEG signals suggestive of migraine or cluster headache is detected by the processing unit, the processing unit is configured to immediately send signals to the auriculotemporal nerve stimulation unit to prompt it to automatically start sending pre-determined neuromodulating electric stimulation to the wearer's auriculotemporal nerve. When cessation of EEG signals suggestive of migraine or cluster headache is detected by the processing unit, the processing unit is further configured to immediately send signals to the auriculotemporal nerve stimulation unit to prompt it to automatically stop sending neuromodulating electric stimulation to the wearer's auriculotemporal nerve. The processing unit is further configured to analyze the EEG data recorded by the EEG monitoring system to detect presence or cessation of EEG signals suggestive of impending migraine or impending cluster headache. When the presence of EEG signals suggestive of impending migraine or impending cluster headache is detected by the processing unit, the processing unit is configured to immediately send signals to the auriculotemporal nerve stimulation unit to prompt it to automatically start sending predetermined neuromodulating electric stimulation to the wearer's auriculotemporal nerve. When cessation of EEG signals suggestive of impending migraine or impending cluster headache is detected by the processing unit, the processing unit is further configured to immediately send signals to the auriculotemporal nerve stimulation unit to prompt it to automatically stop sending neuromodulating electric stimuli to the wearer's auriculotemporal nerve.

In some modified embodiments, an automatic detection-therapy system for migraine or cluster headache may further comprises a laser speckle-based blood flow monitoring unit such as a speckle contrast optical spectroscopy unit (SCOS unit) or an auricular SCOS unit. The automatic detection-therapy system for migraine or cluster headache includes a neuromodulation unit that may comprise an auriculotemporal nerve stimulation unit. The auriculotemporal nerve stimulation unit includes an auriculotemporal nerve stimulating electrode configured to contact the auriculotemporal nerve innervated area of the wearer's auricular skin. A processing unit is in electronic communication with the auricular EEG recording module, the auricular SCOS unit (or SCOS unit) and the auriculotemporal nerve stimulation unit. The processing unit is configured to analyze the EEG data recorded by the EEG monitoring system to detect the presence or cessation of EEG signals suggestive of migraine or cluster headache. The processing unit is also configured to analyze the blood flow data recorded by the SCOS unit (or auricular SCOS unit) to detect the presence or cessation of blood flow data suggestive of migraine or cluster headache. When the processing unit detects one of the following: the presence of EEG signals suggestive of migraine or cluster headache and presence of blood flow data suggestive of migraine or cluster headache, the processing unit is configured to immediately send signals to the auriculotemporal nerve stimulation unit to prompt it to automatically start sending pre-determined neuromodulating electric stimulation to the wearer's auriculotemporal nerve. The processing unit is configured to analyze the EEG data recorded by the EEG monitoring system to detect presence or cessation of EEG signals suggestive of impending migraine or impending cluster headache. The processing unit is also configured to analyze the blood flow data recorded by the auricular SCOS unit (or SCOS unit) to detect presence or cessation of blood flow data suggestive of impending migraine or impending cluster headache. When the processing unit detects one of the following: the presence of EEG signals suggestive of impending migraine or impending cluster headache and presence of blood flow data suggestive of impending migraine or impending cluster headache, the processing unit is configured to immediately send signals to the auriculotemporal nerve stimulation unit to prompt it to automatically start sending predetermined neuromodulating electric stimulation to the wearer's auriculotemporal nerve. When the processing unit detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit is further configured to immediately send signals to the auriculotemporal nerve stimulation unit to prompt it to automatically stop sending neuromodulating electric stimulation to the wearer's auriculotemporal nerve.

In some embodiments, an automatic detection-therapy system for migraine or cluster headache may include a neuromodulation unit that may comprise an occipital nerve stimulation unit. The occipital nerve stimulation unit may deliver stimulation to unilateral occipital nerve or bilateral occipital nerves, depending on user's condition. (An occipital nerve stimulator placed at mid-occipital area could be used to reach bilateral occipital nerves.) (Traditionally, an occipital nerve stimulator is placed by surgery under the skin in occipital region. Nowadays, transcutaneous occipital nerve stimulator is available.) The occipital nerve stimulation unit includes one or two stimulating electrodes configured to contact the occipital nerves innervated areas of the wearer's occipital region of the head. A processing unit is in electronic communication with the auricular EEG recording module and the occipital nerve stimulation unit. The processing unit is configured to analyze the EEG data recorded by the EEG monitoring system to detect the presence or cessation of EEG signals suggestive of migraine or cluster headache. When the presence of EEG signals suggestive of migraine or cluster headache is detected by the processing unit, the processing unit is configured to immediately send signals to the occipital nerve stimulation unit to prompt it to automatically start sending pre-determined neuromodulating electric stimulation to the wearer's occipital nerves. When cessation of EEG signals suggestive of migraine or cluster headache is detected by the processing unit, the processing unit is further configured to immediately send signals to the occipital nerve stimulation unit to prompt it to automatically stop sending neuromodulating electric stimulation to the wearer's occipital nerves. The processing unit is further configured to analyze the EEG data recorded by the EEG monitoring system to detect presence or cessation of EEG signals suggestive of impending migraine or impending cluster headache. When the presence of EEG signals suggestive of impending migraine or impending cluster headache is detected by the processing unit, the processing unit is configured to immediately send signals to the occipital nerve stimulation unit to prompt it to automatically start sending predetermined neuromodulating electric stimulation to the wearer's occipital nerves. When cessation of EEG signals suggestive of impending migraine or impending cluster headache is detected by the processing unit, the processing unit is further configured to immediately send signals to the occipital nerve stimulation unit to prompt it to automatically stop sending neuromodulating electric stimulation to the wearer's occipital nerves.

In some modified embodiments, an automatic detection-therapy system for migraine or cluster headache may further comprises a laser speckle-based blood flow monitoring unit such as a speckle contrast optical spectroscopy unit (SCOS unit) or an auricular SCOS unit. The automatic detection-therapy system for migraine or cluster headache includes a neuromodulation unit that may comprise an occipital nerve stimulation unit. The occipital nerve stimulation unit includes an occipital nerve stimulating electrode configured to contact the occipital nerve innervated area of the wearer's occipital region. A processing unit is in electronic communication with the auricular EEG recording module, the auricular SCOS unit (or SCOS unit) and the occipital nerve stimulation unit. The processing unit is configured to analyze the EEG data recorded by the EEG monitoring system to detect the presence or cessation of EEG signals suggestive of migraine or cluster headache. The processing unit is also configured to analyze the blood flow data recorded by the SCOS unit (or auricular SCOS unit) to detect the presence or cessation of blood flow data suggestive of migraine or cluster headache. When the processing unit detects one of the following: the presence of EEG signals suggestive of migraine or cluster headache and presence of blood flow data suggestive of migraine or cluster headache, the processing unit is configured to immediately send signals to the occipital nerve stimulation unit to prompt it to automatically start sending pre-determined neuromodulating electric stimulation to the wearer's occipital nerves. The processing unit is configured to analyze the EEG data recorded by the EEG monitoring system to detect presence or cessation of EEG signals suggestive of impending migraine or impending cluster headache. The processing unit is also configured to analyze the blood flow data recorded by the auricular SCOS unit (or SCOS unit) to detect presence or cessation of blood flow data suggestive of impending migraine or impending cluster headache. When the processing unit detects one of the following: the presence of EEG signals suggestive of impending migraine or impending cluster headache and presence of blood flow data suggestive of impending migraine or impending cluster headache, the processing unit is configured to immediately send signals to the occipital nerve stimulation unit to prompt it to automatically start sending predetermined neuromodulating electric stimulation to the wearer's occipital nerves. When the processing unit detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit is further configured to immediately send signals to the occipital nerve stimulation unit to prompt it to automatically stop sending neuromodulating electric stimulation to the wearer's occipital nerves.

In some embodiments, an automatic detection-therapy system for migraine or cluster headache may include a neuromodulation unit that may comprise a greater auricular nerve stimulation unit. The greater auricular nerve (GAN) is a pure sensory nerve originated from the cervical spinal cord (C2, C3) and provides sensory innervation to the skin over the parotid gland, the mastoid process, and the lower two-thirds of the outer ear (pinna). The greater auricular nerve (GAN) innervated auricular skin includes skin on both surfaces of the pinna (including the lobule) and cavum concha (inferior/lower part of concha). The greater auricular nerve stimulation unit includes a greater auricular nerve stimulating electrode configured to contact the greater auricular nerve innervated area of the wearer's auricular skin. A processing unit is in electronic communication with the auricular EEG recording module and the greater auricular nerve stimulation unit. The processing unit is configured to analyze the EEG data recorded by the EEG monitoring system to detect the presence or cessation of EEG signals suggestive of migraine or cluster headache. When the presence of EEG signals suggestive of migraine or cluster headache is detected by the processing unit, the processing unit is configured to immediately send signals to the greater auricular nerve stimulation unit to prompt it to automatically start sending pre-determined neuromodulating electric stimulation to the wearer's greater auricular nerve. When cessation of EEG signals suggestive of migraine or cluster headache is detected by the processing unit, the processing unit is further configured to immediately send signals to the greater auricular nerve stimulation unit to prompt it to automatically stop sending neuromodulating electric stimulation to the wearer's greater auricular nerve. The processing unit is further configured to analyze the EEG data recorded by the EEG monitoring system to detect presence or cessation of EEG signals suggestive of impending migraine or impending cluster headache. When the presence of EEG signals suggestive of impending migraine or impending cluster headache is detected by the processing unit, the processing unit is configured to immediately send signals to the greater auricular nerve stimulation unit to prompt it to automatically start sending predetermined neuromodulating electric stimulation to the wearer's greater auricular nerve. When cessation of EEG signals suggestive of impending migraine or impending cluster headache is detected by the processing unit, the processing unit is further configured to immediately send signals to the greater auricular nerve stimulation unit to prompt it to automatically stop sending neuromodulating electric stimulation to the wearer's greater auricular nerve.

In some modified embodiments, an automatic detection-therapy system for migraine or cluster headache may further comprises a laser speckle-based blood flow monitoring unit such as a speckle contrast optical spectroscopy unit (SCOS unit) or an auricular SCOS unit. The automatic detection-therapy system for migraine or cluster headache includes a neuromodulation unit that may comprise a greater auricular nerve stimulation unit. The greater auricular nerve stimulation unit includes a greater auricular nerve stimulating electrode configured to contact the greater auricular nerve innervated area of the wearer's auricular skin. A processing unit is in electronic communication with the auricular EEG recording module, the auricular SCOS unit (or SCOS unit) and the greater auricular nerve stimulation unit. The processing unit is configured to analyze the EEG data recorded by the EEG monitoring system to detect the presence or cessation of EEG signals suggestive of migraine or cluster headache. The processing unit is also configured to analyze the blood flow data recorded by the SCOS unit (or auricular SCOS unit) to detect the presence or cessation of blood flow data suggestive of migraine or cluster headache. When the processing unit detects one of the following: the presence of EEG signals suggestive of migraine or cluster headache and presence of blood flow data suggestive of migraine or cluster headache, the processing unit is configured to immediately send signals to the greater auricular nerve stimulation unit to prompt it to automatically start sending pre-determined neuromodulating electric stimulation to the wearer's greater auricular nerve. The processing unit is configured to analyze the EEG data recorded by the EEG monitoring system to detect presence or cessation of EEG signals suggestive of impending migraine or impending cluster headache. The processing unit is also configured to analyze the blood flow data recorded by the auricular SCOS unit (or SCOS unit) to detect presence or cessation of blood flow data suggestive of impending migraine or impending cluster headache. When the processing unit detects one of the following: the presence of EEG signals suggestive of impending migraine or impending cluster headache and presence of blood flow data suggestive of impending migraine or impending cluster headache, the processing unit is configured to immediately send signals to the greater auricular nerve stimulation unit to prompt it to automatically start sending predetermined neuromodulating electric stimulation to the wearer's greater auricular nerve. When the processing unit detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit is further configured to immediately send signals to the greater auricular nerve stimulation unit to prompt it to automatically stop sending neuromodulating electric stimulation to the wearer's greater auricular nerve. In some modified embodiments, an automatic detection-therapy system for migraine or cluster headache may further comprises a laser speckle-based blood flow monitoring unit such as a speckle contrast optical spectroscopy unit (SCOS unit) or an auricular SCOS unit. The automatic detection-therapy system for migraine or cluster headache includes a neuromodulation unit that may comprise an infraorbital nerve stimulation unit 305. The setups and functions for the infraorbital nerve stimulation unit 305 is essentially the same as the aforementioned descriptions for the greater auricular nerve stimulation unit 304.

In modified embodiments, an automatic detection-therapy system for migraine or cluster headache may have a neuromodulation unit that comprises more than one component. For example, the neuromodulation unit may comprise a taVNS unit and an auriculotemporal nerve stimulation unit. When the processing unit detects one of the following: presence of EEG signals suggestive of migraine or cluster headache and presence of blood flow data suggestive of migraine or cluster headache, the processing unit may be configured to send signals to both the taVNS unit and the auriculotemporal nerve stimulation unit to prompt the taVNS unit to send pre-determined electric stimulation to the wearer's vagus-innervated auricular skin and also to prompt the auriculotemporal nerve stimulation unit to send pre-determined electric stimulation to the wearer's auriculotemporal nerve innervated auricular skin. When the processing unit detects one of the following: presence of EEG signals suggestive of impending migraine or impending cluster headache and presence of blood flow data suggestive of impending migraine or impending cluster headache, the processing unit may be configured to send signals to both the taVNS unit and the auriculotemporal nerve stimulation unit to prompt the taVNS unit to send pre-determined electric stimulation to the wearer's vagus-innervated auricular skin and also to prompt the auriculotemporal nerve stimulation unit to send pre-determined electric stimulation to the wearer's auriculotemporal nerve innervated auricular skin. When the processing unit detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit may be further configured to send signals to both the taVNS unit and the auriculotemporal nerve stimulation unit to prompt the taVNS unit to stop sending pre-determined electric stimulation to the wearer's vagus-innervated auricular skin and also to prompt the auriculotemporal nerve stimulation unit to stop sending pre-determined electric stimulation to the wearer's auriculotemporal nerve innervated auricular skin.

In another modified embodiment, the neuromodulation unit for the automatic detection-therapy system for migraine or cluster headache may include a combination of a taVNS unit and an occipital nerve stimulation unit. When the processing unit detects one of the following: presence of EEG signals suggestive of migraine or cluster headache and presence of blood flow data suggestive of migraine or cluster headache, the processing unit may be configured to send signals to both the taVNS unit and the occipital nerve stimulation unit to prompt the taVNS unit to send pre-determined electric stimulation to the wearer's vagus-innervated auricular skin and also to prompt the occipital nerve stimulation unit to send pre-determined electric stimulation to the wearer's occipital nerve innervated region. When the processing unit detects one of the following: presence of EEG signals suggestive of impending migraine or impending cluster headache and presence of blood flow data suggestive of impending migraine or impending cluster headache, the processing unit may be configured to send signals to both the taVNS unit and the occipital nerve stimulation unit to prompt the taVNS unit to send pre-determined electric stimulation to the wearer's vagus-innervated auricular skin and also to prompt the occipital nerve stimulation unit to send pre-determined electric stimulation to the wearer's occipital nerve innervated region. When the processing unit detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit may be further configured to send signals to both the taVNS unit and the occipital nerve stimulation unit to prompt the taVNS unit to stop sending electric stimulation to the wearer's vagus-innervated auricular skin and also to prompt the occipital nerve stimulation unit to stop sending electric stimulation to the wearer's occipital nerve innervated region.

In yet another modified embodiment, the neuromodulation unit for the automatic detection-therapy system for migraine or cluster headache may include a combination of a taVNS unit and a supraorbital nerve stimulation unit. When the processing unit detects one of the following: presence of EEG signals suggestive of migraine or cluster headache and presence of blood flow data suggestive of migraine or cluster headache, the processing unit may be configured to send signals to both the taVNS unit and the supraorbital nerve stimulation unit to prompt the taVNS unit to send pre-determined electric stimulation to the wearer's vagus-innervated auricular skin and also to prompt the supraorbital nerve stimulation unit to send pre-determined electric stimulation to the wearer's supraorbital nerve innervated forehead skin. When the processing unit detects one of the following: presence of EEG signals suggestive of impending migraine or impending cluster headache and presence of blood flow data suggestive of impending migraine or impending cluster headache, the processing unit may be configured to send signals to both the taVNS unit and the supraorbital nerve stimulation unit to prompt the taVNS unit to send pre-determined electric stimulation to the wearer's vagus-innervated auricular skin and also to prompt the supraorbital nerve stimulation unit to send pre-determined electric stimulation to the wearer's supraorbital nerve innervated forehead skin. When the processing unit detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit may be further configured to send signals to both the taVNS unit and the supraorbital nerve stimulation unit to prompt the taVNS unit to stop sending pre-determined electric stimulation to the wearer's vagus-innervated auricular skin and also to prompt the supraorbital nerve stimulation unit to stop sending pre-determined electric stimulation to the wearer's supraorbital nerve innervated forehead skin.

In still another modified embodiment, the neuromodulation unit for the automatic detection-therapy system for migraine or cluster headache may include a combination of an auriculotemporal nerve stimulation unit and a supraorbital nerve stimulation unit. The supraorbital nerve and the auriculotemporal nerve are different branches of the trigeminal nerve. Studies have shown that simultaneous stimulation of both branches of the trigeminal nerve may provide more comprehensive effects. When the processing unit detects one of the following: presence of EEG signal suggestive of migraine or cluster headache and presence of blood flow data suggestive of migraine or cluster headache, the processing unit may be configured to send signals to both the auriculotemporal nerve stimulation unit and the supraorbital nerve stimulation unit to prompt the auriculotemporal nerve stimulation unit to send pre-determined electric stimulation to the wearer's auriculotemporal nerve innervated auricular skin and also to prompt the supraorbital nerve stimulation unit to send pre-determined electric stimulation to the wearer's supraorbital nerve innervated forehead skin. When the processing unit detects one of the following: presence of EEG signals suggestive of impending migraine or impending cluster headache and presence of blood flow data suggestive of impending migraine or impending cluster headache, the processing unit may be configured to send signals to both the auriculotemporal nerve stimulation unit and the supraorbital nerve stimulation unit to prompt the auriculotemporal nerve stimulation unit to send pre-determined electric stimulation to the wearer's auriculotemporal nerve-innervated auricular skin and also to prompt the supraorbital nerve stimulation unit to send pre-determined electric stimulation to the wearer's supraorbital nerve innervated forehead skin. When the processing unit detects all of the following: cessation of EEG signal suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit may be further configured to send signals to both the auriculotemporal nerve stimulation unit and the supraorbital nerve stimulation unit to prompt the auriculotemporal nerve stimulation unit to stop sending pre-determined electric stimulation to the wearer's auriculotemporal nerve innervated auricular skin and also to prompt the supraorbital nerve stimulation unit to stop sending pre-determined electric stimulation to the wearer's supraorbital nerve innervated forehead skin.

In still yet another modified embodiment, the neuromodulation unit for the automatic detection-therapy system for migraine or cluster headache may include a combination of a taVNS unit and a greater auricular nerve stimulation unit. When the processing unit detects one of the following: presence of EEG signals suggestive of migraine or cluster headache and presence of blood flow data suggestive of migraine or cluster headache, the processing unit may be configured to send signals to both the taVNS unit and the greater auricular nerve stimulation unit to prompt the taVNS unit to send pre-determined electric stimulation to the wearer's vagus-innervated auricular skin and also to prompt the greater auricular nerve stimulation unit to send pre-determined electric stimulation to the wearer's greater auricular nerve innervated auricular skin. When the processing unit detects one of the following: presence of EEG signals suggestive of impending migraine or impending cluster headache and presence of blood flow data suggestive of impending migraine or impending cluster headache, the processing unit may be configured to send signals to both the taVNS unit and the greater auricular nerve stimulation unit to prompt the taVNS unit to send pre-determined electric stimulation to the wearer's vagus-innervated auricular skin and also to prompt the greater auricular nerve stimulation unit to send pre-determined electric stimulation to the wearer's greater auricular nerve innervated auricular skin. When the processing unit detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit may be further configured to send signals to both the taVNS unit and the greater auricular nerve stimulation unit to prompt the taVNS unit to stop sending pre-determined electric stimulation to the wearer's vagus-innervated auricular skin and also to prompt the greater auricular nerve stimulation unit to stop sending pre-determined electric stimulation to the wearer's greater auricular nerve innervated auricular skin.

Other nerve stimulation units of the neuromodulation unit may also be combined, similar to the aforementioned descriptions. In further modified embodiments, the neuromodulation unit for the automatic detection-therapy system for migraine or cluster headache may include a combination of 3 different nerve stimulation units, for example, a combination of a taVNS unit, an auriculotemporal nerve stimulation unit and a supraorbital nerve stimulation unit, or a combination of a taVNS unit, an auriculotemporal nerve stimulation unit and an occipital nerve stimulation unit or a combination of a taVNS unit, an auriculotemporal nerve stimulation unit and a greater auricular nerve stimulation unit. All of these various combinations are within the scope of this invention.

In some embodiments, an automatic detection-therapy system for migraine or cluster headache may include an auricular EEG monitoring system having two sets of EEG recording modules, including a first EEG recording module to be linked to a first ear of the wearer and a second EEG recording module to be linked to a second ear of the wearer. Both EEG recording modules are in electronic communication with the processing unit. The bilateral EEG will enhance the capability of this system to detect migraine or cluster headache and impending migraine or impending cluster headache. In some embodiments, an automatic detection-therapy system for migraine or cluster headache may include two neuromodulation units with the first neuromodulation unit linked to a first ear of the wearer (or part of the components of the first neuromodulation unit linked to the first ear of the wearer) and a second neuromodulation unit linked to a second ear of the wearer. The second neuromodulation unit may comprise at least one of: a second taVNS unit, a second auriculotemporal nerve stimulation unit and a second greater auricular nerve stimulation unit. (Note that: The supraorbital nerve stimulation unit is usually located at mid-forehead can stimulate unilateral or bilateral supraorbital nerves. The occipital nerve stimulation unit is usually located at mid-occipital region and can stimulate unilateral or bilateral occipital nerves. Similarly, the infraorbital nerve stimulation unit is usually located at mid-face and can stimulate unilateral or bilateral infraorbital nerves.) If, in rare situation, a wearer cannot tolerate a taVNS unit on the right ear (due to possible bradycardia), only one taVNS unit on the left ear will be utilized. Studies have shown that bilateral vagus nerve stimulation (through taVNS), preorbital nerve stimulation, auriculotemporal nerve stimulation, occipital nerve stimulation, greater auricular nerve stimulation and infraorbital nerve stimulation and various combinations thereof are more effective than unilateral stimulation of these nerves for migraine and cluster headache. (Trigeminal nerve stimulation can be given via the supraorbital nerve or the auriculotemporal nerve or the infraorbital nerve.)

### BRIEF DESCRIPTION OF THE DRAWINGS

Some embodiments of the present invention are illustrated as an example and are not limited by the figures of the accompanying drawings, in which like references may indicate similar elements and in which:

FIG. 1-FIG. 1 depicts a diagram of an example of an auricular electroencephalogram (EEG) monitoring system in a modified earbud housing, according to various embodiments described herein.

FIG. 2-FIG. 2 shows a diagram of an example of an automatic detection-therapy system in a modified earbud housing, according to various embodiments described herein.

FIG. 3-FIG. 3 depicts a diagram of another example of an automatic detection-therapy system in a modified in-the-ear housing, according to various embodiments described herein.

FIG. 4-FIG. 4 illustrates an elevation view of the anatomy of a human external ear, including the locations of tragus, cymba-concha, cavum-concha, triangular fossa, helix, lobule, external ear canal, etc.

FIG. 5-FIG. 5 shows a diagram of a further example of an automatic detection-therapy system in a behind-the-ear-hearing-aid-style housing, according to various embodiments described herein.

FIG. 6-FIG. 6 depicts a diagram of yet a further example of an automatic detection-therapy system in a modified in-the-ear housing, according to various embodiments described herein.

FIG. 7-FIG. 7 depicts a diagram of still another example of an automatic detection-therapy system having a modified earbud housing engaged to the ear of a wearer according to various embodiments described herein.

FIG. 8-FIG. 8 shows a diagram of an example of an auricular electroencephalogram (EEG) monitoring system having a modified earbud housing engaged or coupled to the ear of a wearer according to various embodiments described herein.

FIG. 9-FIG. 9 depicts a diagram of an example of an auricular electroencephalogram (EEG) monitoring system having a tubular-shaped structure engaged or coupled to the ear of a wearer according to various embodiments described herein.

FIG. 10-FIG. 10 illustrates a diagram of an example of an auricular electroencephalogram (EEG) monitoring system having a behind-the-ear-hearing-aid-style housing engaged or coupled to the ear of a wearer according to various embodiments described herein.

FIG. 11-FIG. 11 shows a diagram of an example of an automatic detection-therapy system having a modified earbud housing engaged or coupled to the ear of a wearer according to various embodiments described herein.

FIG. 12-FIG. 12 illustrates a diagram of an example of an automatic detection-therapy system having a modified in-the-ear housing engaged or coupled to the ear of a wearer according to various embodiments described herein.

FIG. 13-FIG. 13 depicts a diagram of an example of an automatic detection-therapy system having a behind-the-ear-hearing-aid-style housing engaged or coupled to the ear of a wearer according to various embodiments described herein.

FIG. 14-FIG. 14 illustrates a block diagram showing some exemplary elements of an example of a processing unit for an EEG monitoring system or an automatic detection-therapy system according to various embodiments described herein.

FIG. 15-FIG. 15 shows a block diagram showing some exemplary elements of an example of an auricular EEG monitoring system according to various embodiments described herein.

FIG. 16-FIG. 16 depicts a block diagram illustrating some exemplary elements of an example of a transcutaneous auricular vagus nerve stimulation (taVNS) unit according to various embodiments described herein.

FIG. 17-FIG. 17 shows a block diagram illustrating some exemplary elements of an example of a client device according to various embodiments described herein.

FIG. 18-FIG. 18 illustrates a schematic diagram illustrating some exemplary components of auricular EEG monitoring system and some exemplary components of an automatic detection-therapy system according to various embodiments described herein.

FIG. 19-FIG. 19 illustrates a perspective diagram of an example of a tubular-shaped structure for housing of the EEG sensor electrodes and some exemplary locations for the EEG sensor electrodes according to various embodiments described herein.

FIG. 20-FIG. 20 illustrates a side elevation diagram of an example of a tubular-shaped structure for housing of the EEG sensor electrodes and some exemplary locations for the EEG sensor electrodes according to various embodiments described herein.

FIG. 21-FIG. 21 shows a sectional, through line 21-21 shown in FIG. 20, elevation view of an example of a tubular-shaped structure for housing of the EEG sensor electrodes and some exemplary locations for the EEG sensor electrodes according to various embodiments described herein.

FIG. 22-FIG. 22 depicts a sectional, through line 22-22 shown in FIG. 20, elevation view of an example of a tubular-shaped structure for housing of the EEG sensor electrodes and some exemplary locations for the EEG sensor electrodes according to various embodiments described herein.

FIG. 23-FIG. 23 shows a sectional, through line 23-23 shown in FIG. 20, elevation view of an example of a tubular-shaped structure for housing of the EEG sensor electrodes and some exemplary locations for the EEG sensor electrodes according to various embodiments described herein.

FIG. 24-FIG. 24 illustrates a sectional, through line 24-24 shown in FIG. 20, elevation view of an example of a tubular-shaped structure for housing of the EEG sensor electrodes and some exemplary locations for the EEG sensor electrodes according to various embodiments described herein.

FIG. 25-FIG. 25 depicts a sectional, through line 25-25 shown in FIG. 20, elevation view of an example of a tubular-shaped structure for housing of the EEG sensor electrodes and some exemplary locations for the EEG sensor electrodes according to various embodiments described herein.

FIG. 26-FIG. 26 shows a sectional, through line 26-26 shown in FIG. 20, elevation view of an example of a tubular-shaped structure for housing of the EEG sensor electrodes and some exemplary locations for the EEG sensor electrodes according to various embodiments described herein.

FIG. 27-FIG. 27 shows a diagram of a combined neuromodulation system in the tragus-concha bowl of a wearer's ear, according to various embodiments described herein.

FIG. 28-FIG. 28 shows a diagram of still a further example of an automatic detection-therapy system having a behind-the-ear-hearing-aid-style housing according to various embodiments described herein.

FIG. 29-FIG. 29 shows a diagram of a supraorbital nerve stimulation unit.

FIG. 30-FIG. 30 shows a diagram of an occipital nerve stimulation unit with a transparent view of the occipital nerves.

FIG. 31-FIG. 31 shows a block diagram illustrating some exemplary elements of an example of a trigeminal nerve stimulation unit. Examples of a trigeminal nerve stimulation unit includes a supraorbital nerve stimulation unit, an auriculotemporal nerve stimulation unit and an infraorbital nerve stimulation unit.

FIG. 32-FIG. 32 shows a block diagram illustrating some exemplary elements of an example of an occipital nerve stimulation unit.

FIG. 33-FIG. 33 shows a block diagram illustrating some exemplary elements of an example of a greater auricular nerve stimulation unit.

FIG. 34-FIG. 34 shows a block diagram illustrating some exemplary elements of an example of a speckle contrast optical spectroscope unit (SCOS unit).

FIG. 35-FIG. 35 shows a block diagram illustrating some exemplary elements of an example of a photoplethysmography unit (PPG unit).

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. Unless otherwise defined, all terms (including technical and scientific terms) used herein have the same meaning as commonly understood by one having ordinary skill in the art to which this invention belongs. It will be further understood that terms, such as those defined in commonly used dictionaries, may be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more of the other disclosed techniques. Accordingly, this description will refrain from repeating every possible combination of the individual steps in an unnecessary fashion. Nevertheless, the specification and claims may be read with the understanding that such combinations are entirely within the scope of the invention and the claims.

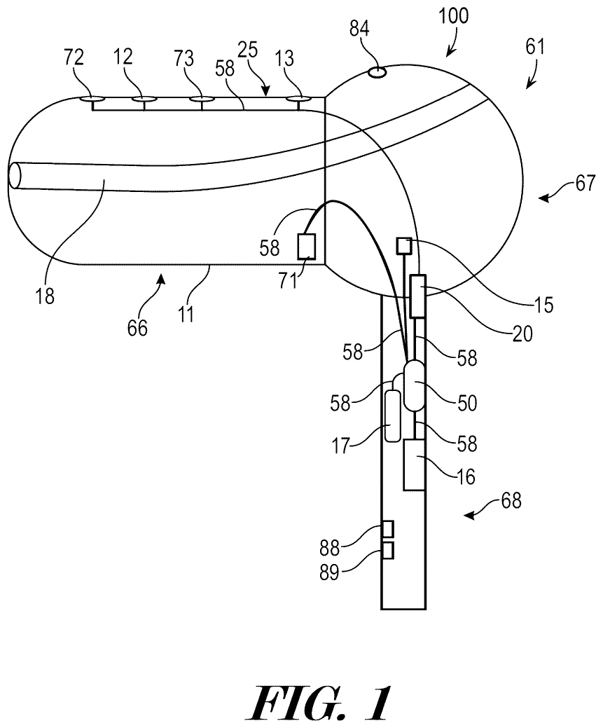

For purposes of description herein, the terms “upper”, “lower”, “left”, “right”, “rear”, “front”, “side”, “vertical”, “horizontal”, and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The term “client device” as used herein is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of client devices include: personal computers (PCs), workstations, servers, laptops, tablet PCs including the iPad, cell phones including iOS phones made by Apple Inc., Android OS phones, Microsoft OS phones, Blackberry phones, Apple iPads, Anota digital pens, smart watches (e.g., Apple Watch, Samsung Galaxy Watch, etc.), digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some nonlimiting examples of portable devices include: cell phones, smartphones, tablet computers, laptop computers, tablets, digital pens, wearable computers such as Apple Watch, other smartwatches, Fitbit, other wearable fitness trackers, Google Glasses, and the like.

As used herein the term “data network” or “network” shall mean an infrastructure capable of connecting two or more computers such as client devices either using wires or wirelessly allowing them to transmit and receive data. Non-limiting examples of data networks may include the internet or wireless networks or (i.e., a “wireless network”) which may include BLE (Bluetooth), LoRa and LoRaWAN (and other low-power, wide-area (LPWA) networking protocols), Wi-Fi, and cellular networks. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, a Zigbee network, or a voice-over-IP (VoIP) network.

Although the terms “first”, “second”, etc. are used herein to describe various elements, these elements may not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 15% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

A new auricular electroencephalogram (EEG) monitoring system and an automatic detection-therapy system are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below. The present invention will now be described by example and through referencing the appended figures representing preferred and alternative embodiments.

According to one embodiment consistent with the principles of the present invention, an auricular electroencephalogram (EEG) monitoring system (“the auricular EEG monitoring system”) 100 is disclosed (FIGS. 1, 8-10, and 18). In some embodiments, the auricular EEG monitoring system 100 may comprise one or two auricular EEG recording modules 20 (for example: a first EEG recording module 20 to be linked to a first ear of the wearer and configured to record EEG from the wearer's first ear 902 and a second EEG recording module 20 to be linked to a second ear 902 of the wearer and configured to record EEG from the wearer's second ear 902.) which may have a plurality of (at least two, but preferably more than two) wired or wireless EEG sensor electrodes 12, 13, 72, 73, 82, 83. Each auricular EEG recording module 20 may have a plurality of EEG sensor electrodes 12, 13, 72, 73, 82, 83. Optionally, the EEG recording module 20 may further comprises an optional wired or wireless reference electrode 84, to act as a baseline voltage reference for other active EEG sensor electrodes 12, 13, 72, 73, 82, 83. (The optional reference electrode 84 would be desirable but is not always needed. Instead, average of all of the EEG sensor electrodes 12, 13, 72, 73, 82, 83, can be used as a reference, i.e. common average reference). The EEG sensor electrodes 12, 13, 72, 73, 82, 83, and the optional reference electrode 84 are configured to contact separate areas of the wearer's ear 902 or peri-auricular area 903. The areas that the EEG sensor electrodes 12, 13, 72, 73, 82, 83, and the optional reference electrode 84 are configured to contact may be selected from at least one of the following: the external ear 902, external ear canal 904, and peri-auricular area 903. The peri-auricular area 903 refers to the portion of the head around the auricle. The peri-auricular area 903 is typically hairless. The peri-auricular area 903 includes a portion of the head in front of the auricle (preauricular area) and a portion of the head above and behind the auricle (post-auricular area). The pre-auricular area is small, about one inch wide and about two inches long and curved along the anterior edge of the auricle. The post-auricular area is also small and is about one inch wide and about three inches long and curved along the superior and posterior edges of the auricle. The post-auricular area is where a behind-the-ear hearing aid is usually attached to. The preauricular area and the post-auricular area together is called “peri-auricular area” herein. (Anterior, posterior, superior, in front of and behind etc. all refer to the directions relative to the wearer's head when the wearer is in an upright position.) The auricular EEG recording module 20 may be configured to record EEG data of the wearer 900 via electrical activities detected by the EEG sensor electrodes 12, 13 72, 73, 82, 83, and the optional reference electrode 84 (if incorporated). A network interface 53, 406, may be configured to generate a notification to a client device 400. A processing unit 50, 401, may be in electronic communication with the first auricular EEG recording module 20 and the network interface 53. Preferably, the processing unit 50, 401, may be configured to analyze the EEG data recorded by the auricular EEG recording module 20 to detect presence or cessation of EEG signals suggestive of migraine or cluster headache, and when the presence or cessation of EEG signals suggestive of migraine or cluster headache is detected, the processing unit 50, 401, may be further configured to immediately send signals to the network interface 53 to generate a notification to the client device 400, such as to a client device 400 of a wearer 900 or a client device 400 of the wearer's healthcare provider 950.

The auricular EEG recording module 20 may be configured to record EEG data of the wearer 900 via miniature EEG sensor electrodes 12, 13, 72, 73, 82, 83, and the optional reference electrode 84, e.g., wired or wireless miniature dry EEG electrodes (wired and wireless miniature dry EEG electrodes as known in the art). The recorded EEG data may be transmitted or otherwise electronically communicated wired or wirelessly to a processing unit 50, 401. With the help of various advanced EEG analysis algorithms together with machine learning, deep learning and artificial intelligence (as known in the art), the processing unit 50, 401, may be configured to analyze the EEG data to detect presence or cessation of EEG signals suggestive of migraine or cluster headache. The processing unit 50, 401, may also be configured to analyze the EEG data to detect presence or cessation of EEG signals suggestive of impending migraine or impending cluster headache.

In some embodiments, a processing unit 50, 401, of the auricular EEG monitoring system 100 may be configured to analyze the EEG data recorded by the auricular EEG recording module 20 to detect presence or cessation of EEG signals suggestive of migraine or cluster headache. When presence of EEG signals suggestive of migraine or cluster headache is detected, the processing unit 50, 401, may be further configured to immediately send signals to the network interface 53 to generate a notification to a client device 400, such as to a client device 400 of a wearer 900 or a client device 400 of the wearer's healthcare provider 950. In further embodiments, the processing unit 50, 401, may be configured to analyze the EEG data recorded by the auricular EEG recording module 20 to detect presence or cessation of EEG signals suggestive of impending migraine or impending cluster headache. When the presence of EEG signals suggestive of impending migraine or impending cluster headache is detected, the processing unit 50, 401, may be further configured to immediately send signals to the network interface 53 to generate a notification to the client device 400, such as to a client device 400 of a wearer 900 or a client device 400 of the wearer's healthcare provider 950.

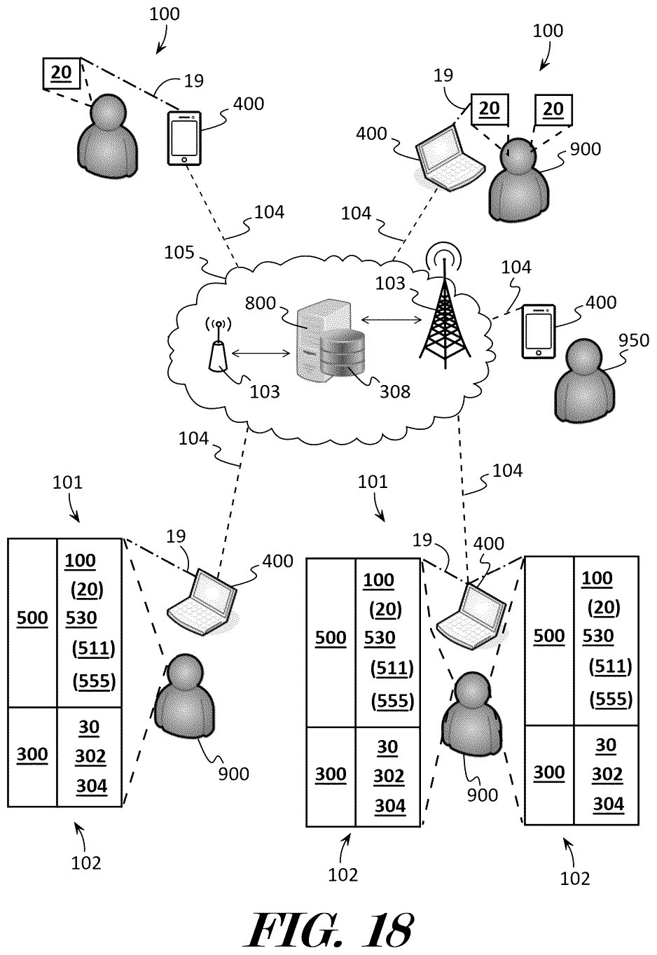

It should be understood that the auricular EEG monitoring system 100 may comprise one or more auricular EEG recording modules 20 as shown in FIG. 18. For example, the auricular EEG monitoring system 100 may comprise a first auricular EEG recording module 20 with its EEG sensor electrodes 12, 13, 72, 73, 82, 83, and the optional reference electrode 84, attached to the wearer's 900 right ear 902 or right peri-auricular area 903 and a second auricular EEG recording module 20 with its EEG sensor electrodes 12, 13, 72, 73, 82, 83, and the optional reference electrode 84 attached to the wearer's 900 left ear 902 or left peri-auricular area 903. The first auricular EEG recording module 20 may include a plurality of (at least two, but preferably more than two) EEG sensor electrodes, 12, 13, 72, 73, 82, 83, and the optional reference electrode 84 and all of these electrodes are configured to contact separate areas of the wearer's right ear 902 or right peri-auricular area 903. The areas that the EEG sensor electrodes 12, 13, 72, 73, 82, 83, and the optional reference electrode 84 may be configured to contact are selected from at least one of the following: the right external ear 902, right external ear canal 904, and right peri-auricular area 903. Likewise, the second auricular EEG recording module 20 may include a plurality of (at least two, but preferably more than two) EEG sensor electrodes 12, 13, 72, 73, 82, 83, and an optional reference electrode 84. These electrodes 12, 13, 72, 73, 82, 83, 84, may be configured to contact separate areas of the wearer's left ear 902 or left peri-auricular area 903. The areas that the EEG sensor electrodes 12, 13, 72, 73, 82, 83, and the optional reference electrode 84 may be configured to contact may be selected from at least one of the following: the left external ear 902, left external ear canal 904, and left peri-auricular area 903. The second auricular EEG recording module 20 may also be configured to record EEG data of the wearer 900, and the second EEG recording module 20 may also be in electronic communication with the processing unit 50 so that the processing unit 50 may use EEG data from the first auricular EEG recording module 20 and the second auricular EEG recording module 20 to detect the presence or cessation of EEG signals suggestive of migraine or cluster headache and/or to detect the presence or cessation of EEG signals suggestive of impending migraine or impending cluster headache.

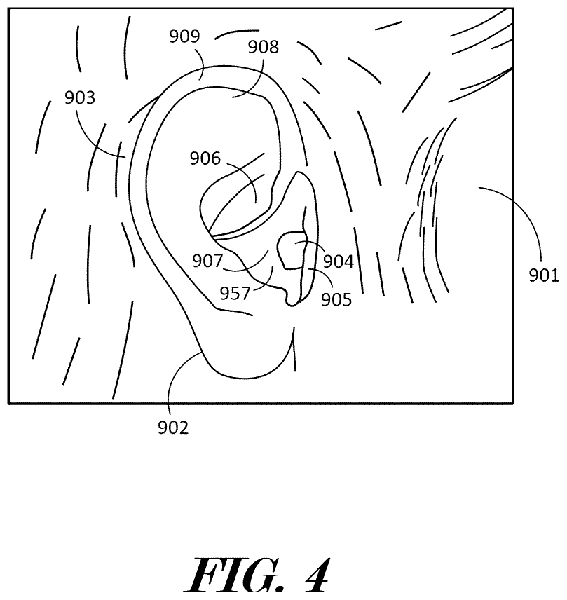

In preferred embodiments, the auricular EEG monitoring system 100 may comprise a miniature auricular EEG recording module 20 that may include a plurality of miniature EEG sensor electrodes 12, 13. 72, 73, 82, 83, and the optional reference electrode 84 that may be coupled to or contained in an auricular housing 11. The auricular housing 11 may be attached to the head 901, preferably to the external ear canal 904 or external ear 902 or the peri-auricular area 903. All of the EEG sensor electrodes 12, 13, 72, 73, 82, 83, and the optional reference electrode 84 are configured to contact separate areas selected from at least one of the following: external ear canal 904, external ear 902 and peri-auricular area 903. The locations for the EEG sensor electrodes 12, 13, 72, 73, 82, 83, and the optional reference electrode 84 to contact are separate from each other. (As referred to herein, the external ear canal 904 refers to the part of the ear 902 that connects the visible outer ear (pinna) to the middle ear, essentially the tube that carries sound waves to the eardrum). The anatomy of human external ear 902 and head 901 proximate to the ear 902 is shown in FIG. 4.

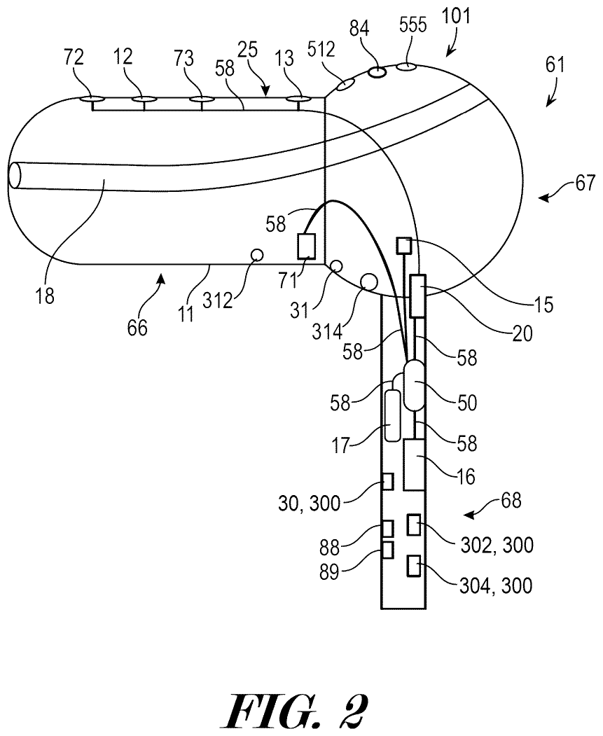

In some embodiments, an auricular housing 11 of an auricular EEG monitoring system 100 and/or an automatic detection-therapy system 101 may be configured in any size and shape that may be suitable for being attached to the external ear 902, the external ear canal 904, the tragus-concha bowl 957 or the peri-auricular area 903. In some embodiments, an auricular housing 11 may comprise a sound conduit 18 which may extend through the auricular housing 11 (such as shown in FIGS. 1 and 2) and which may facilitate the ability of sound to enter the ear 902. A sound conduit 18 may comprise an opening, channel, conduit, etc., which may extend through a portion of the auricular housing 11 so that sound waves may pass through the sound conduit 18 to facilitate or enable the wearer 900 to hear sounds in the environment. The sound conduit 18 may also help to equalize air pressure between the external ear canal 904 and the outside environment, reducing pressure buildup, improving comfort, and enhancing sound fidelity. As used herein, the term “tragus-concha bowl” 957 refers to an area surrounding the opening of the external ear canal 904 and this area is bounded by inner tragus 905 and concha 906, 907, forming a bowl-shaped cavity to funnel sound into the external ear canal. This bowl-shaped cavity is called tragus-concha bowl 957 herein (official name is concha auriculae and is sometimes referred to simply as the concha bowl).

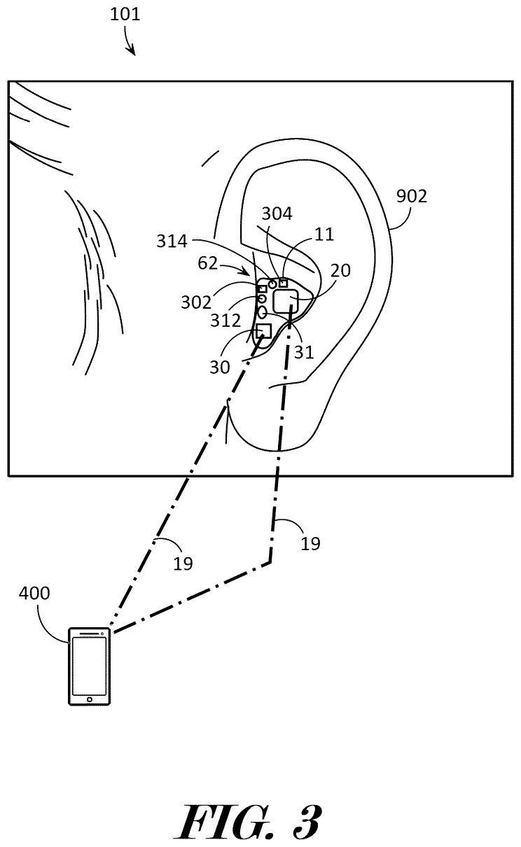

In some embodiments, an auricular housing 11 (housing refers to protective cover or protective structure) of an auricular EEG monitoring system 100 and/or an automatic detection-therapy system 101 may be shaped or configured as a modified in-the-ear housing 62. (A modified in-the-ear housing 62 may be modified from an in-the-ear (ITE) hearing aid to include a tubular-shaped structure 66 and a body-structure 67. The tubular-shaped structure 66 has an elongated tubular part that is inserted into a wearer's external ear canal 904 when in use. The body-structure 67 is located at the immediate opening of the external ear canal 904 and sits inside the tragus-concha bowl 957 when in use.) All or a majority of the auricular housing 11 may be inserted into a portion of the external ear canal 904 and the tragus-concha bowl 957, such as shown in FIGS. 3, 12. Example of a modified in-the-ear housing 62 is modified from a combination of an in-the-ear (ITE) hearing aid and an in-the-canal (ITC) hearing aid.

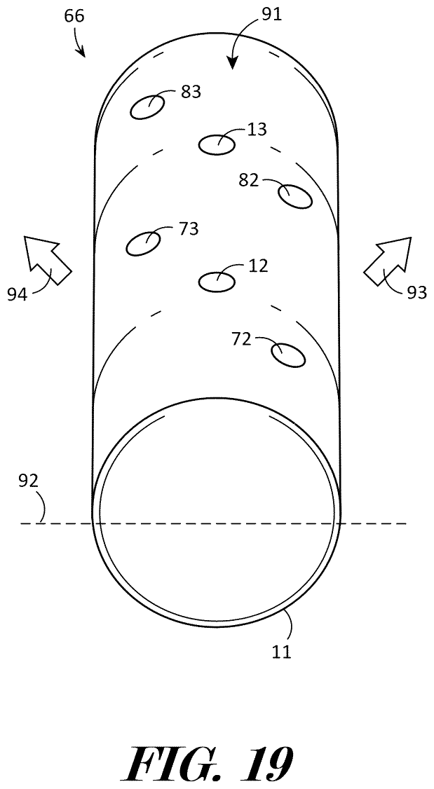

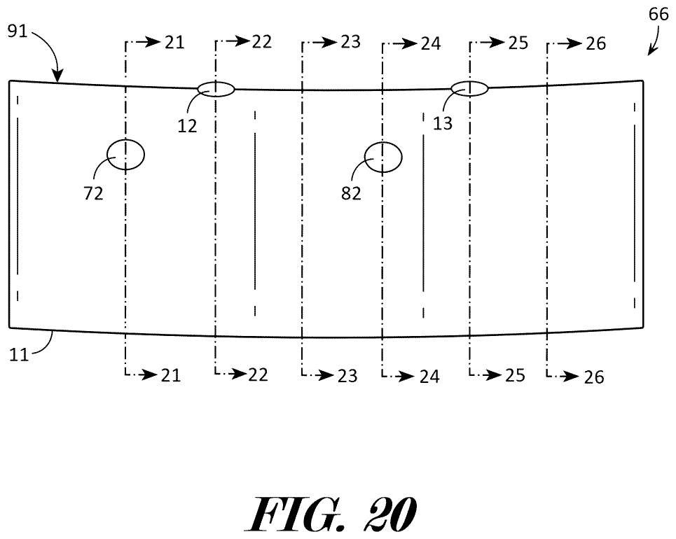

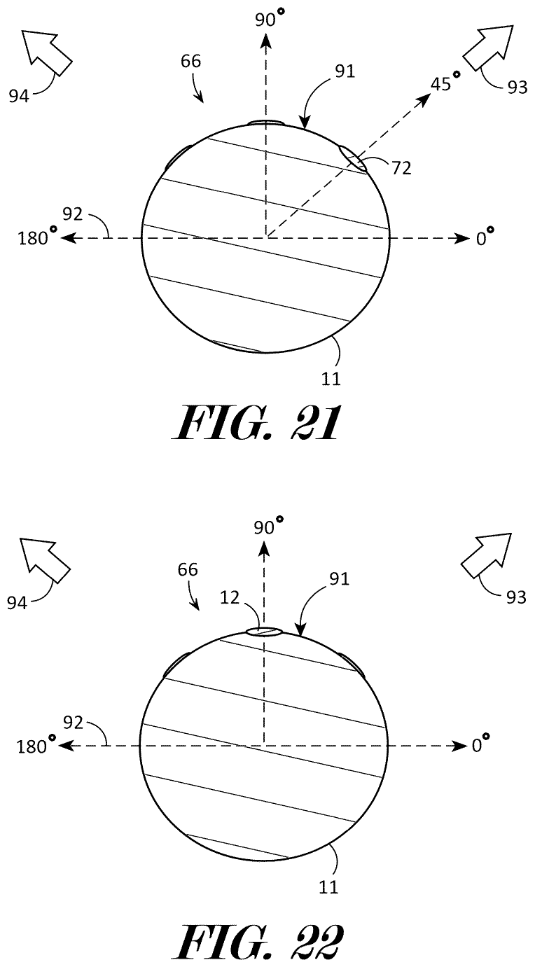

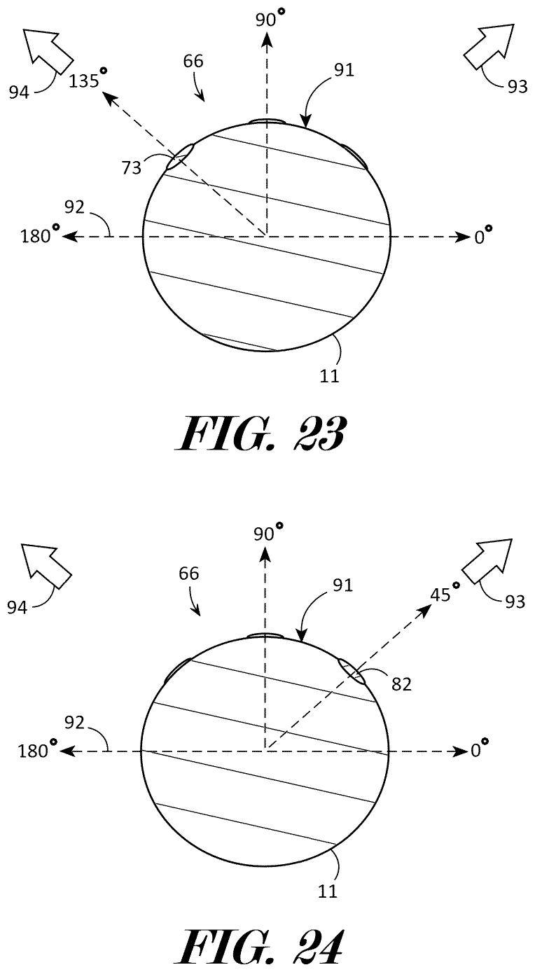

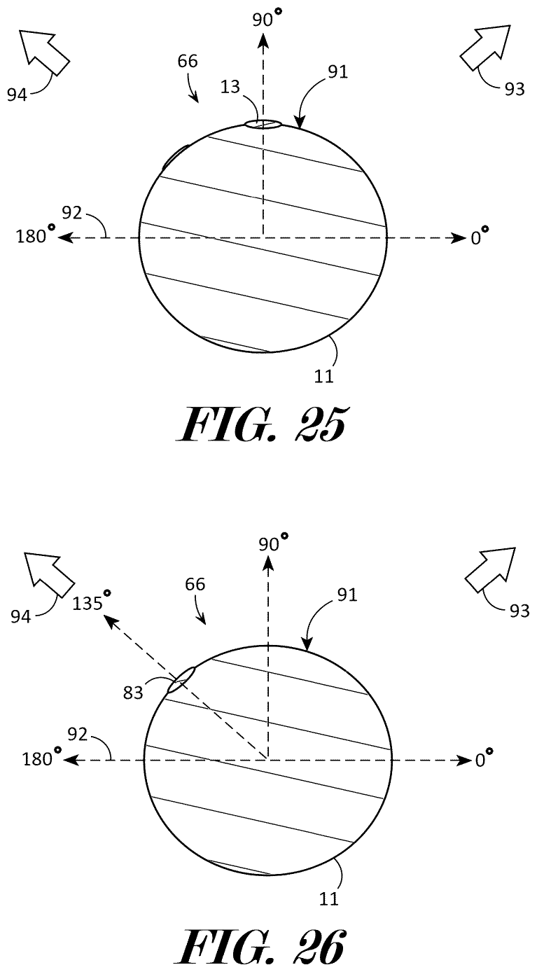

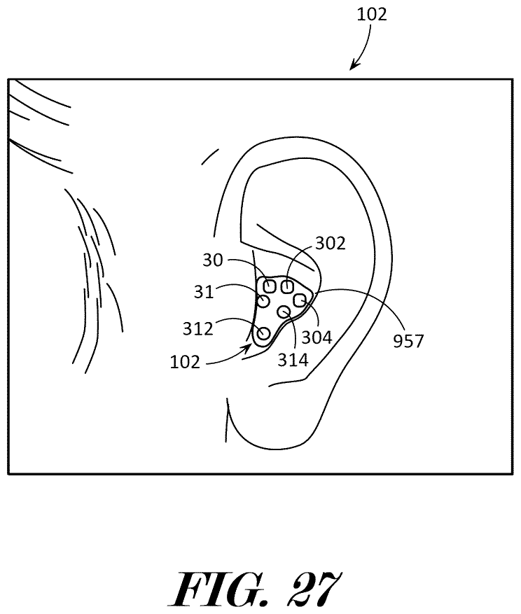

In some embodiments, an auricular housing 11 of an auricular EEG monitoring system 100 and/or an automatic detection-therapy system 101 may be configured to include a tubular-shaped structure 66. All of the EEG sensor electrodes 12, 13, 72, 73, 82, 83 are configured to be located at the surface 91 of the tubular-shaped structure 66 and so that all of the EEG sensor electrodes 12, 13, 72, 73, 82, 83 are housed in the tubular-shaped structure 66. Optionally, the other components for system 100 and/or system 101 may be configured to be located or housed in other portions of the auricular housing 11, to be described hereinafter. Alternatively, the tubular-shaped structure 66 may be configured as a standalone tubular-shaped structure 66 for housing of all of the EEG sensor electrodes 12, 13, 72, 73, 82, 83 and part of the other components of system 100 and/or system 101. The tubular-shaped structure 66 is configured to be inserted into a wearer's external ear canal 904 when in use. In preferred embodiments, one or more of the EEG sensor electrodes 12, 13, 72, 73, 82, 83 is/are configured to be located at the upper surface 91 (upper surface at approximately 90 degrees above horizontal level 92, as shown by electrodes 12, 13, in FIGS. 19, 22, 25) of the tubular-shaped structure 66. In preferred embodiments, one or more of the EEG sensor electrodes is/are configured to be located at between 0 and 90 degrees, and more preferably at approximately 45 degrees (plus or minus fifteen degrees, i.e. 30-60 degrees) above horizontal level 92 of the tubular structure 66 and is/are configured to face forward-upward direction 93 (e.g., as shown by electrodes 72, 82, in FIGS. 19, 21, 24). In preferred embodiments, one or more of the EEG sensor electrodes is/are configured to be located at between 90 and 180 degrees, and more preferably at approximately 135 degrees (plus or minus fifteen degrees, i.e. 120-150 degrees) above the horizontal level 92 of the tubular structure 66 and is/are configured to face backward-upward direction 94 (e.g., as shown by electrodes 73, 83, in FIGS. 19, 23, 26). (Upper surface, horizontal level, forward, backward and upward all refer to directions relative to the head 901 of the wearer 900 with the wearer in upright position after the tubular-shaped structure 66 has been inserted into the wearer's external ear canal 904.) This arrangement of the EEG sensor electrodes 12, 13, 72, 73, 82, 83, will provide one of the best locations and directions for these electrodes 12, 13, 72, 73, 82, 83, in the external ear canal 904 from where to record the wearer's EEG activities.

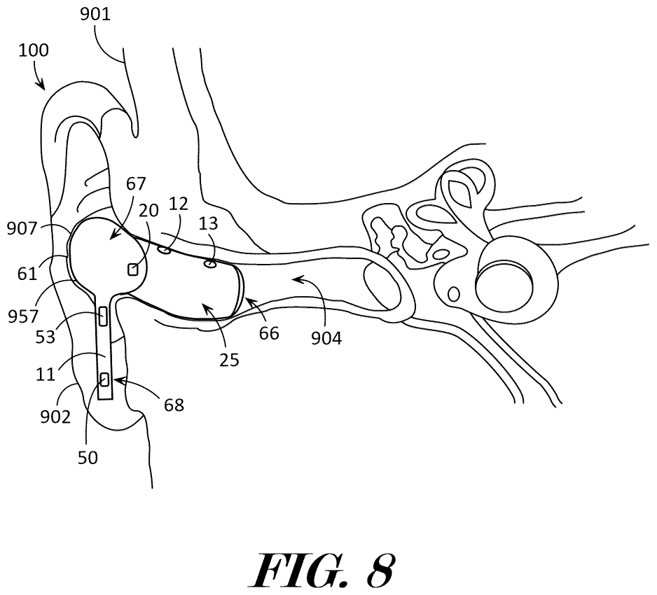

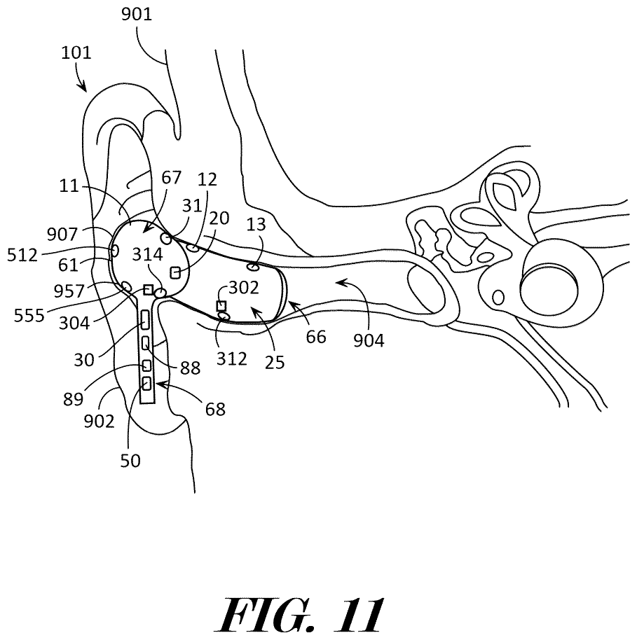

In some embodiments, an auricular housing 11 of an auricular EEG monitoring system 100 and/or an automatic detection-therapy system 101 may be shaped or configured as a modified earbud housing 61. The modified earbud housing 61 includes a tubular-body portion 25 and a stem portion (“stem” or “stalk” portion) 68. The tubular-body portion 25 comprises a tubular-shaped structure 66 and a body-structure 67. The tubular-shaped structure 66 is essentially an elongated version of an “ear-tip” and a “nozzle” of an earbud and the tubular-shaped structure 66 can be inserted into a wearer's external ear canal 904 when in use. The body-structure 67 is essentially similar to a “body” (or a “shell”) of an earbud. The body-structure 67 is placed at the opening of a wearer's external ear canal 904 and sit inside a tragus-concha bowl 957 when in use. The modified earbud housing 61 may be shaped and sized so that when the tubular-body portion 25 is placed in a wearer's external ear 902 when in use, the tubular-shaped structure 66 is inserted into the wearer's external ear canal 904 and the body-structure 67 is sitting inside the tragus-concha bowl 957 which is immediately outside the opening of the external ear canal 904, such as shown in FIGS. 8 and 11. Examples that a modified earbud housing 61 is modified from include: earbuds, ear phones, Apple AirPods®, in-ear monitors (IEM) and the like. As used herein, the term “tragus-concha bowl” 957 refers to an area surrounding the opening of the external ear canal 904 and this area is bounded by inner tragus 905 and concha 906, 907, forming a bowl-shaped cavity to funnel sound into the external ear canal. This bowl-shaped cavity is called tragus-concha bowl 957 herein (official name is concha auriculae and is sometimes referred to simply as the concha bowl).

In some embodiments of an auricular EEG monitoring system 100, an EEG recording module 20, a processing unit 50, and a network interface 53 may be housed or contained in a modified earbud housing 61 (FIGS. 1 and 8). The modified earbud housing 61 includes a tubular-body portion 25 and a stem portion 68. The tubular-body portion 25 includes a tubular-shaped structure 66 (to be inserted into a wearer's external ear canal 904 when in use) and a body-structure 67 (to be placed at opening of the external ear canal 904 and to sit or be placed inside a tragus-concha bowl 957 of the wearer's ear). All of the EEG sensor electrodes 12, 13, 72, 73, 82, 83, may be located on a surface and partially embedded in the surface with slight protrusion at the surface of the tubular-shaped structure 66. An optional reference electrode 84 may be located on a surface and partially embedded in the surface with protrusion at the surface of the body-structure 67. Preferably, the tubular-body portion 25 (including the tubular-shaped structure 66 and the body-structure 67) may comprise or may be made from an elastic flexible and adaptable material. The material of the tubular-body portion 25 of the modified earbud housing 61 is configured to have appropriate elasticity flexibility and adaptability so that all of the EEG sensor electrodes 12, 13, 72, 73, 82, 83, of the EEG recording module 20 are naturally in close contact (provided by the elastic flexible, and adaptable characteristics of the material so that it can conform to the contours of the external ear canal 904 and the tragus-concha bowl 957 and snugly fill the interior of the external ear canal 904 and the tragus-concha bowl 957) with the skin of the external ear canal 904 of the wearer's ear 902 and so that the optional reference electrode 84 is naturally in close contact with the skin of the tragus-concha bowl 957 of the wearer's ear 902 when the tubular-body portion 25 of the modified earbud housing 61 is placed in the wearer's ear 902.

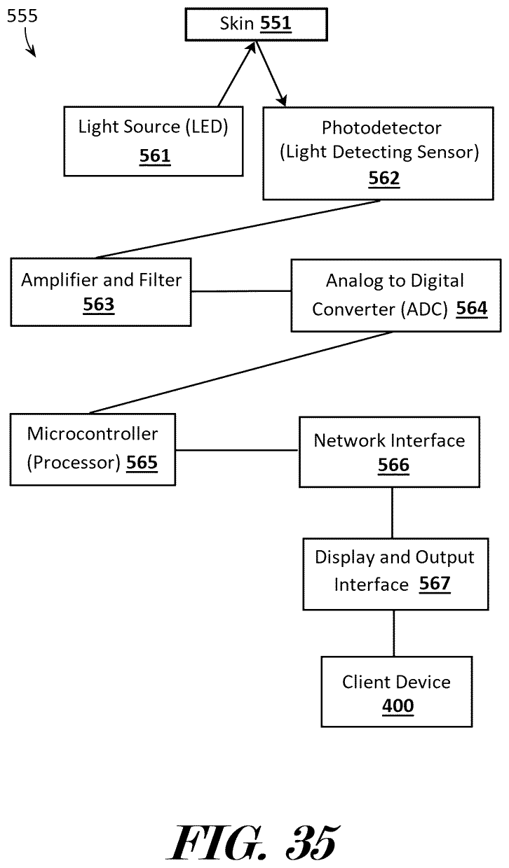

In some embodiments, an automatic detection-therapy system 101 may comprise a neurovascular monitoring system 500, a processing unit 50, a neuromodulation unit 300 and a network interface 53. The processing unit 50 is in electronic communication with the neurovascular monitoring system 500, the neuromodulation unit 300 and the network interface 53. The neurovascular monitoring system 500 comprises an auricular EEG monitoring system 100 and a cephalic blood flow monitoring system 530. The cephalic blood flow monitoring system 530 may comprise a laser speckle-based blood flow monitoring unit, such as a speckle contrast optical spectroscope unit (SCOS unit) 510 or an auricular speckle contrast optical spectroscopy unit (auricular SCOS unit) 511. Alternatively, the cephalic blood flow monitoring system 530 may comprise a photoplethysmography (PPG) unit 555 and the PPG unit 555 is in electronic communication with the processing unit 50, 401. (PPG is known in the art). The PPG unit 555 may be used to monitor blood flow and blood pressure (PPG primarily monitor changes in blood volume although it can also monitor blood flow). (PPG sensors use light to detect blood volume changes with algorithms, such as pulse wave analysis, PPG can analyze this waveform to estimate systolic and diastolic blood pressure.) In alternate embodiments, a cephalic blood flow monitoring system 530 may comprise a PPG unit 555 for blood flow and blood pressure monitoring. In other embodiments, a cephalic blood flow monitoring system 530 may comprise a SCOS unit 510 or a combination of both an auricular SCOS unit 511 and a PPG unit 555. Referring to FIG. 35, a PPG unit 555 as a blood flow and blood pressure meter typically includes the following components: a light source 561, a photodetector 562, amplifier and filter 563, analog-to-digital converter (ADC) 564, a microcontroller/processor 565, a network interface 566 and a display 567. The light source 561 is usually an LED that emit lights at a specific wavelength, commonly around 940 nm, to illuminate the skin 551. The photodetector (or light detecting sensor) 562 can detect the light reflected from the skin 551 and can capture the changes in blood volume. The photodetector 562 then transmits the signals to an amplifier and filter 563 for conditioning of the signals to enhance the quality of the PPG signals. These signals are then transmitted to an analog-to-digital converter (ADC) 564. The ADC 564 can convert the analog signals from the photodetector 562 into digital form for processing. The ADC 564 is in electronic communication with a microcontroller (processor) 565 that can process the digital signals to extract relevant information (such as blood flow metrics, heart rate and blood pressure, etc.) The network interface 566 is in electronic communication with a display and output interface 567 which then generate a display to a user (such as a display on a client device 400). The processing unit 50, 401, may be configured to analyze the data transmitted from the auricular SCOS unit 511 and the data transmitted from the PPG unit 555 to assess the wearer's cerebral and extracranial blood flow data. It is known that SCOS and PPG may provide complementary blood flow data and may be more useful than using SCOS or PPG alone. The PPG unit 555 can also provide estimate of the wearer's systolic and diastolic blood pressure. A PPG unit 555 may be placed on a surface of the auricular housing 11 (in either the tubular-shaped structure 66 or the body-structure 67) to monitor extracranial blood flow of the wearer 900. Ultrasound-doppler based blood flow monitoring device is another alternative for the cephalic blood flow monitoring system 530. The auricular SCOS unit 511 comprises an auricular SCOS sensor 512 configured to be in contact with the wearer's auricular skin when measuring the wearer's blood flow. The auricular SCOS sensor 512 is usually integrated with the SCOS camera 516. The neuromodulation unit 300 may include at least one of the following components: a taVNS unit 30, a supraorbital nerve stimulation unit 301, an auriculotemporal nerve stimulation unit 302, an occipital nerve stimulation unit 303, a greater auricular nerve stimulation unit 304 and an infraorbital nerve stimulation unit 305. Integration of taVNS unit 30, auriculotemporal nerve stimulation unit 302 and greater auricular nerve stimulation unit 304 into a single auricular housing 11, such as a modified earbud housing 61, for automatic detection-therapy system 101 would be preferred due to the proximity and overlapping of their target skin areas and their synergistic effects. The taVNS unit 30 comprises a taVNS stimulating electrode 31 that is configured to give pre-determined neuromodulating electric stimulation to vagus innervated auricular skin of the wearer 900 when activated or prompted. The auriculotemporal nerve stimulation unit 302 comprises an auriculotemporal nerve stimulation electrode 312 that is configured to give pre-determined neuromodulating electric stimulation to the wearer's auriculotemporal nerve innervated auricular skin when activated or prompted. The greater auricular nerve stimulation unit 304 comprises a greater auricular nerve stimulation electrode 314 that is configured to give pre-determined neuromodulating electric stimulation to the wearer's greater auricular nerve innervated auricular skin when activated or prompted.

In some embodiments, an automatic detection-therapy system 101 may be housed or contained in a modified earbud housing 61. Optionally, the auricular EEG recording module 20 may contain an optional reference electrode 84 (to act as a baseline voltage reference for other active EEG sensor electrodes 12, 13, 72, 73, 82, 83). All (or portion) of the following: all of the EEG sensor electrodes 12, 13, 72, 73, 82, 83, the optional reference electrode 84, the auricular SCOS sensor 512, the taVNS stimulating electrode 31, the auriculotemporal nerve stimulating electrode 312 and the greater auricular nerve stimulating electrode 314 are configured to be housed or contained in the modified earbud housing 61 (FIGS. 2, 7, and 11). The modified earbud housing 61 includes a tubular-body portion 25 and a stem portion 68. The tubular-body portion 25 includes a tubular-shaped structure 66 (to be inserted into a wearer's external ear canal when in use) and a body-structure 67 (to be placed at the immediate opening of the wearer's external ear canal 904 and to sit or be placed inside the wearer's tragus-concha bowl 957 when in use). The tubular-shaped structure 66 is equivalent to an elongated version of a “nozzle” and an “ear-tip” of an earbud. The body-structure 67 is equivalent to a “body” (or “shell”) of an earbud. All of the EEG sensor electrodes 12, 13, 72, 73, 82, 83, the optional reference electrode 84, the auricular SCOS sensor 512, the taVNS stimulating electrode 31, the auriculotemporal nerve stimulating electrode 312 and the greater auricular nerve stimulating electrode 314 are configured to be located on a surface of the tubular-body portion 25 of the modified earbud housing 61. All of the EEG sensor electrodes 12, 13, 72, 73, 82, 83, the optional reference electrode 84, the auricular SCOS sensor 512, the taVNS stimulating electrode 31, the auriculotemporal nerve stimulating electrode 312 and the greater auricular nerve stimulating electrode 314 may be configured to be partially embedded in the surface with protrusion at the surface of the tubular-body portion 25 of the modified earbud housing 61. Preferably all of the EEG sensor electrodes 12, 13, 72, 73, 82, 83, may be located on the surface 91 (and partially embedded in the surface with protrusion at the surface) of the tubular-shaped structure 66. Preferably the auricular SCOS sensor 512, the taVNS stimulating electrode 31, and the auriculotemporal nerve stimulating electrode 312 may be located on the surface (and partially embedded in the surface with protrusion at the surface) of one of, the body-structure 67 and the tubular-shaped structure 66. Preferably, the optional reference electrode 84 and the greater auricular nerve stimulating electrode 314 may be located on a surface (and partially embedded in the surface with protrusion at the surface) of the body-structure 67. The tubular-body portion 25 may comprise or may be made from an elastic flexible and adaptable material. The material for the tubular-body portion 25 of the modified earbud housing 61 is configured to have appropriate elasticity flexibility and adaptability so that when the tubular-body portion 25 is placed in the wearer's ear 902, all of the EEG sensor electrodes 12, 13, 72, 73, 82, 83 will be naturally and snugly in contact with the skin of the wearer's external ear canal 904; meanwhile, the greater auricular nerve stimulating electrode 314 and the optional reference electrode 84 will be naturally snugly in contact with the skin of the wearer's tragus-concha bowl 957; and, at the same time, the auricular SCOS sensor 512, the taVNS stimulating electrode 31 and the auriculotemporal nerve stimulating electrode 312 will be naturally snugly in close contact with the skin of wearer's tragus-concha bowl 957 or the skin of the wearer's external ear canal 904 (provided by the elastic, flexible, and adaptable characteristics of the material so that it can conform to the contour of the external ear canal 904 and the contour of the tragus-concha bowl 957 and snugly fill the interior of the external ear canal 904 and the interior of the tragus-concha bowl 957). Meanwhile, the taVNS stimulating electrode 31 will naturally snugly contacting vagus-innervated auricular skin, the auriculotemporal nerve (ATN) stimulating electrode 312 will naturally snugly contacting the ATN innervated auricular skin and the greater auricular nerve (GAN) stimulating electrode 314 will also naturally snugly contacting the GAN innervated auricular skin when the tubular-body portion 25 of the modified ear bud housing 61 is placed in the wearer's ear 902 when in use. (FIGS. 2, 11). Attaching and removing all of the EEG sensor electrodes 12, 13, 72, 73, 82, 83, the optional reference electrode 84, the auricular SCOS sensor 512, the taVNS stimulating electrode 31, the auriculotemporal nerve stimulating electrode 312 and the greater auricular nerve stimulating electrode 314 will be as easy as inserting and removing the modified earbud housing 61 from the wearer's ear 902. (For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve (ATN) innervated auricular skin includes: anterior outer part of tragus 905, anterior-superior part of cavum concha 907, anterior and superior walls of the external ear canal 904 and, anterior and superior part of pinna including anterior-superior helix 909. The greater auricular nerve (GAN) innervated auricular skin includes: cavum concha, lower two thirds of anterior and posterior pinna and the mastoid process.) From the above comparison, it is obvious that the tragus-concha 957 of a wearer's ear 902 received mixed and overlapped innervation from the auricular branch of vagus nerve, the auriculotemporal nerve (ATN) and the greater auricular nerve (GAN), while the external ear canal 904 received mixed and overlapped innervation from the auricular branch of vagus nerve and the ATN. In some embodiments, a neuromodulation unit 300 may have 3 components having a taVNS stimulating electrode 31, an auriculotemporal nerve stimulating electrode 312 and a greater auricular nerve stimulating electrode 314 and all of these electrodes (31, 312, 314) may be located on the body-structure 67. By carefully selecting the location for the taVNS stimulating electrode 31 on the body-structure 67 to match the aforementioned innervation locations of the vagus innervated skin on the tragus-concha bowl 957, the taVNS stimulating electrode 31 will automatically get in close contact with its target skin on the tragus-concha bowl 957 when the body-structure 67 is placed in the tragus-concha bowl 957. By carefully selecting the location for the auriculotemporal nerve stimulating electrode 312 on the body-structure 67 to match the aforementioned innervation locations of the auriculotemporal nerve on the tragus-concha bowl 957, the auriculotemporal nerve stimulating electrode 312 will automatically get in close contact with its target skin on the tragus-concha bowl 957 when the body-structure 67 is placed in the tragus-concha bowl 957. Similarly, by carefully selecting the location for the greater auricular nerve stimulating electrode 314 on the body-structure 67 to match the aforementioned innervation locations of the greater auricular nerve innervated skin on the tragus-concha bowl 957, the greater auricular nerve stimulating electrode 314 will automatically get in close contact with its target skin on the tragus-concha bowl 957 when the body-structure 67 is placed in the tragus-concha bowl 957. Alternatively, the taVNS stimulating electrode 31 and the auriculotemporal nerve stimulating electrode 312 may be located on the tubular-shaped structure 66. By carefully selecting the location for the taVNS stimulating electrode 31 on the tubular-shaped structure 66 to match its target skin locations on the external ear canal 904, the taVNS stimulating electrode 31 will be automatically get in close contact with its target skin in the external ear canal 904. Similarly, by carefully selecting the location for the auriculotemporal nerve stimulating electrode 312 on the tubular-shaped structure 66 to match its target skin locations on the external ear canal 904, the auriculotemporal nerve stimulating electrode 312 will be automatically get in close contact with its target skin in the external ear canal 904.

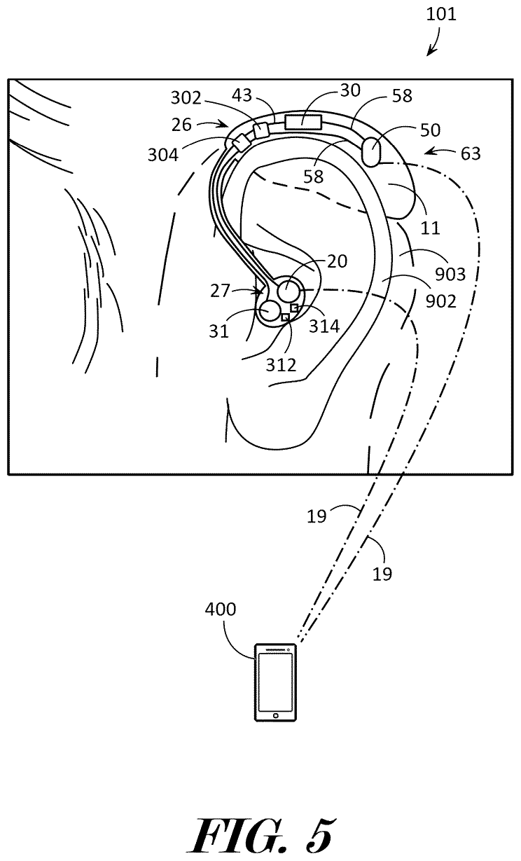

In some embodiments, an auricular housing 11 of an auricular EEG monitoring system 100 and/or an automatic detection-therapy system 101 may be shaped or configured as a behind-the-ear-hearing-aid-style housing 63 as shown in FIGS. 5, 10, and 13. The behind-the-ear-hearing-aid-style housing 63 may comprise a behind-the-ear portion 26 and an in-the-ear portion 27. Generally, a behind-the-ear-hearing-aid-style housing 63 may be shaped and sized so that all or a majority of the behind-the-ear portion 26 may be positioned behind the ear 902, such as to contact the post-auricular part of the peri-auricular area 903, as shown in FIGS. 5, 10, and 13. The in-the-ear portion 27 is essentially the same as the tubular-body portion 25 of a modified earbud housing 61. The in-the-ear portion 27 is also essentially the same as a modified in-the-ear housing 62, as aforementioned description. The in-the-ear portion 27 of the behind-the-ear-hearing-aid-style housing 63 may include a tubular-shaped structure 66 (to be placed inside the wearer's external ear canal 904 when in use) and a body-structure 67 (to be located immediately outside the opening of the wearer's external ear canal 904 and to sit or be placed inside the tragus-concha bowl 957 when in use). The in-the-ear portion 27 may be sized and shaped so that the tubular-shaped structure 66 can be inserted into the external ear canal 904 while the body-structure 67 will be located immediately at opening of the external ear canal 904 and be placed inside the tragus-concha bowl 957 when in use. Example of behind-the-ear-hearing-aid-style housing 63 include behind-the-ear (BTE), receiver-in-the-ear (RITE), CROS/BiCROS (CROS stands for “Contralateral Routing of Signals” and BiCROS stands for “Bilateral Contralateral Routing of Signals”), and the like.

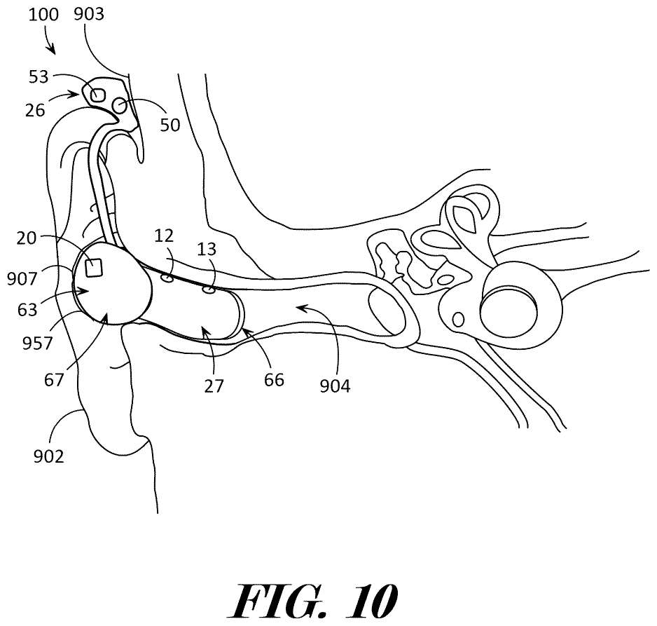

In some embodiments, an auricular EEG monitoring system 100 may be housed or contained in a behind-the-ear-hearing-aid-style housing 63. The auricular EEG monitoring system 100 may comprise an EEG recording module 20, a processing unit 50, and a network interface 53. All of the EEG sensor electrodes 12, 13, 72, 73, 82, 83, may be located on a surface 91 of the tubular-shaped structure 66 of the in-the-ear portion 27 of the behind-the-ear-hearing-aid-style housing 63. All of the EEG sensor electrodes 12, 13, 72, 73, 82, 83, may be configured to be partially embedded in the surface 91 with protrusion at the surface 91 of the tubular-shaped structure 66. Optionally, the auricular EEG recording module 20 may contain an optional reference electrode 84 (to act as a baseline voltage reference for other active EEG sensor electrodes 12, 13, 72, 73, 82, 83). Preferably all of the EEG sensor electrodes 12, 13, 72, 73, 82, 83, may be located on the surface 91 of the tubular-shaped structure 66. Preferably the optional reference electrode 84 may be located on the surface and partially embedded in the surface with protrusion at the surface of the body-structure 67. Preferably, the in-the-ear portion 27 of the behind-the-ear-hearing-aid-style housing 63 may comprise or may be made from an elastic flexible and adaptable material. The material for the in-the-ear portion 27 is configured to have appropriate elasticity flexibility and adaptability so that all of the EEG sensor electrodes 12, 13, 72, 73, 82, 83, of the EEG recording module 20 are naturally in close contact (provided by the elastic, flexible, and adaptable characteristics of the resilient material so that it can conform to the contours of the external ear canal 904 and snugly fill the interior of the external ear canal 904) with the skin of the external ear canal 904 of the wearer's ear 901, and meanwhile, the optional reference electrode 84 will be naturally in close contact with the skin of tragus-concha bowl 957 of the wearer's ear 902 when the in-the-ear portion 27 is placed in the wearer's ear 902. (FIG. 10).

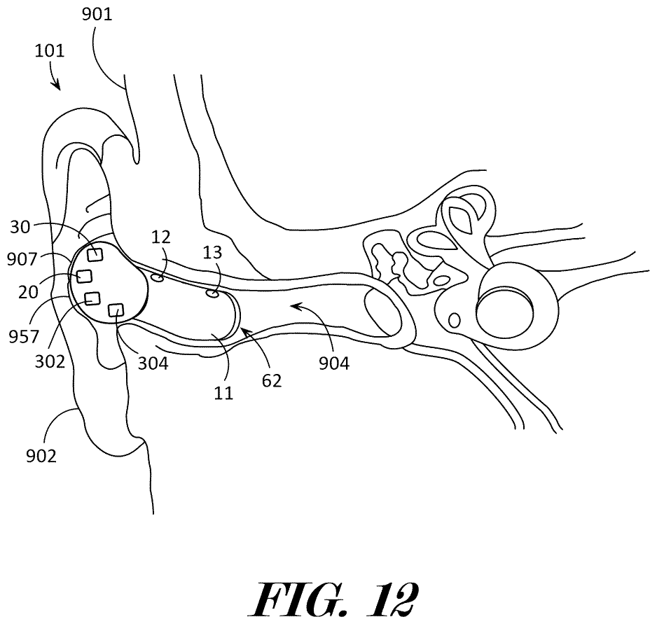

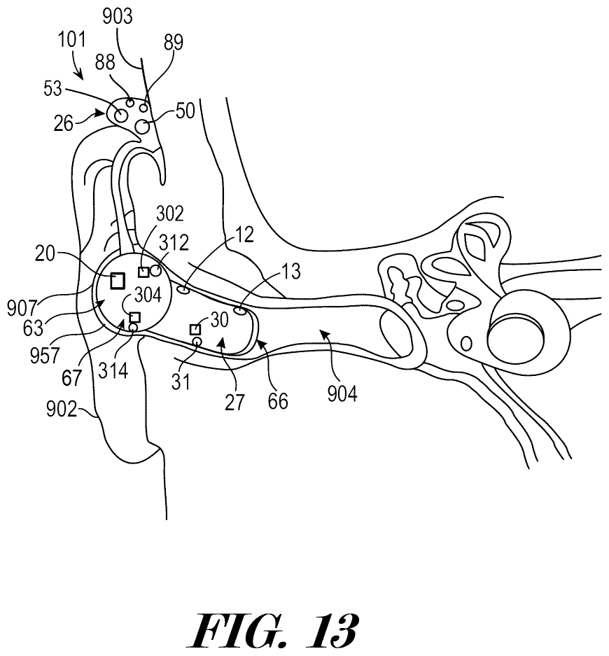

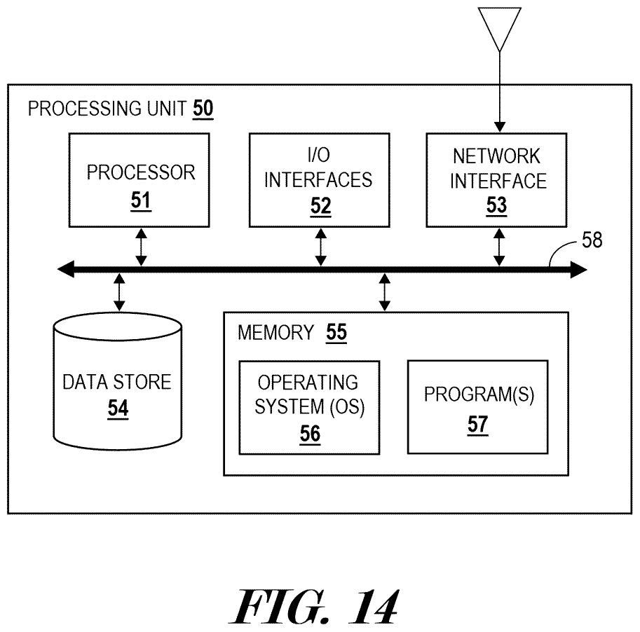

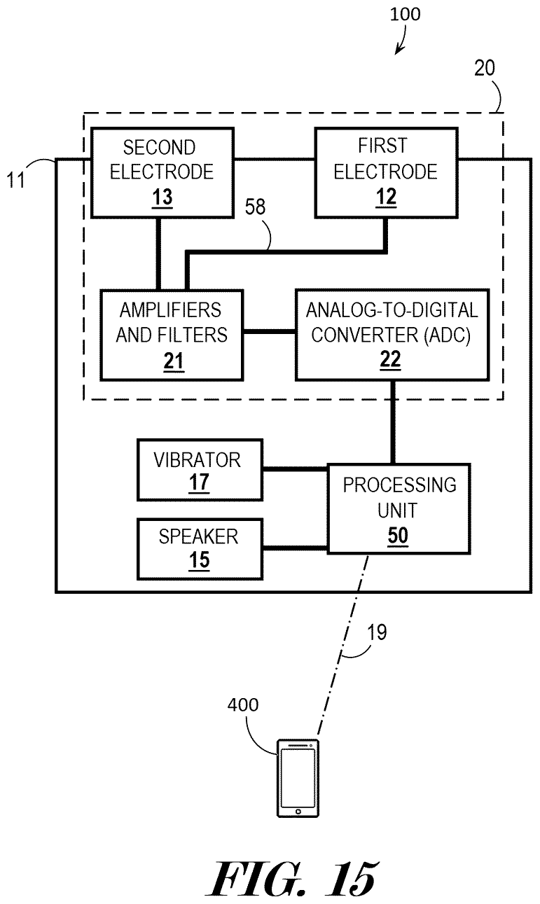

Similarly, in some embodiments, an automatic detection-therapy system 101 may be housed or contained in a behind-the-ear-hearing-aid-style housing 63. The automatic detection-therapy system 101 may comprise a neurovascular monitoring system 500, a processing unit 50, a neuromodulation unit 300 and a network interface 53. The neurovascular monitoring system 500 includes an auricular EEG monitoring system 100 and a cephalic blood flow monitoring system 530. The auricular EEG monitoring system 100 includes an EEG recording module 20, The cephalic blood flow monitoring system 530 may be configured as an auricular speckle contrast optical spectroscopy unit (auricular SCOS unit) 511. Alternatively, the cephalic blood flow monitoring system 530 may comprise an auricular SCOS unit 511 and a PPG unit 555. The processing unit 50 is in electronic communication with the auricular EEG monitoring system 100, the auricular SCOS unit 511, the neuromodulation unit 300 and the network interface 53. The neuromodulation unit 300 may include at least one of the following components: a taVNS unit 30, an auriculotemporal nerve stimulation unit 302, a supraorbital nerve stimulation unit 301, an occipital nerve stimulation unit 303, a greater auricular nerve stimulation unit 304 and an infraorbital nerve stimulation unit 305. Combination of taVNS unit 30, auriculotemporal nerve stimulation unit 302 and greater auricular nerve stimulation unit 304 into a single auricular housing 11, such as a behind-the-ear-hearing-aid-style housing 63, for automatic detection-therapy system 101 would be preferred due to the proximity of their target skin areas and their synergistic effects. The behind-the-ear-hearing-aid-style housing 63 includes an in-the-ear portion 27 and a behind-the-ear portion 26. The in-the-ear portion 27 includes a tubular-shaped structure 66 (to be inserted into a wearer's external ear canal 904 when in use) and a body-structure 67 (to be placed at immediate opening of the wearer's external ear canal and to sit or be placed inside the tragus-concha bowl 957 of the wearer's ear when in use). All of the EEG sensor electrodes 12, 13, 72, 73, 82, 83, may be located on a surface 91 of the tubular-shaped structure 66 of the in-the-ear portion 27 (the in-the-ear portion 27 is essentially the same as the tubular-body portion 25 of a modified earbud housing 61) of the behind-the-ear-hearing-aid-style housing 63. All of the EEG sensor electrodes 12, 13, 72, 73, 82, 83, may be configured to be partially embedded in the surface with slight protrusion at the surface of tubular-shaped structure 66 of the behind-the-ear-hearing-aid-style housing 63. Optionally, the auricular EEG recording module 20 may contain an optional reference electrode 84 (to act as a baseline voltage reference for other active EEG sensor electrodes 12, 13, 72, 73, 82, 83). Preferably all of the EEG sensor electrodes 12, 13, 72, 73, 82, 83, may be located on the surface 91 of the tubular-shaped structure 66. Preferably the optional reference electrode 84 and the greater auricular nerve stimulating electrode 314 may be located on a surface (and partially embedded in the surface with protrusion at the surface) of the body-structure 67. Preferably the auricular SCOS sensor 512, the taVNS stimulating electrode 31 and the auriculotemporal nerve stimulating electrode 312 may be located on the surface and partially embedded in the surface with slight protrusion at the surface of one of, the body-structure 67 and the tubular-shaped structure 66. Preferably, the in-the-ear portion 27 (same as a tubular-body portion 25 of a modified earbud housing 61) may comprise or may be made from an elastic flexible and adaptable material. The material for the in-the-ear portion 27 is configured to have appropriate elasticity flexibility and adaptability so that all of the EEG sensor electrodes 12, 13, 72, 73, 82, 83, are naturally snugly in contact with the skin of the wearer's external ear canal 904; so that the optional reference electrode 84 and the greater auricular nerve stimulating electrode 314 are naturally snugly in contact with the skin of the wearer's tragus-concha bowl 957; and so that the auricular SCOS sensor 512, the taVNS stimulating electrode 31, and the auriculotemporal nerve stimulating electrode 312 are naturally snugly in close contact with skin of the wearer's external ear canal 904 or skin of the wearer's tragus-concha bowl 957 (provided by the elastic, flexible, and adaptable characteristics of the resilient material so that it can conform to the contours of the external ear canal 904 and contour of tragus-concha bowl 957 and snugly fill the interior of the external ear canal 904 and interior of tragus-concha bowl 957) when the in-the-ear portion 27 is placed in the wearer's ear 902. (FIG. 13). At the same time, the taVNS stimulating electrode 31 will naturally contacting the wearer's vagus innervated auricular skin, the auriculotemporal nerve stimulating electrode 312 will naturally contacting the wearer's auriculotemporal nerve innervated auricular skin, and the greater auricular nerve stimulating electrode 314 will naturally contacting the greater auricular nerve innervated auricular skin of the wearer 900 when the in-the-ear portion is place in the wearer's ear 902. Installing and removing all of the EEG sensor electrodes 12, 13, 72, 73, 82, 83, the optional reference electrode 84, the auricular SCOS sensor, the taVNS stimulating electrode 31, the auriculotemporal nerve stimulating electrode 312 and the greater auricular nerve stimulating electrode 314 will be as easy as inserting and removing the in-the-ear portion 27 from the wearer's external ear 902. (FIGS. 5, 13, 28). This is feasible due to the proximity of the innervation patterns of the auricular branch of vagus nerve, the auriculotemporal nerve and the greater auricular nerve in the auricular area, and by careful selection of the locations for 31, 312, 314, to match the locations of their respective target skin on the wearer's ear, as described hereinbefore. From the aforementioned innervation patterns, it is obvious that the tragus-concha 957 of a wearer's ear 902 received mixed and overlapped innervation from the auricular branch of vagus nerve, the auriculotemporal nerve and the greater auricular nerve. In some embodiments, a neuromodulation unit 300 may have a taVNS stimulating electrode 31, an auriculotemporal nerve stimulating electrode 312 and a greater auricular nerve stimulating electrode 314 and all of these electrodes (31, 312, 314) may be located on a body-structure 67. By carefully selecting the location for the taVNS stimulating electrode 31 on the body-structure 67 to match the aforementioned innervation locations of the vagus innervated skin on the tragus-concha bowl 957, the taVNS stimulating electrode 31 will automatically get in close contact with its target skin on the tragus-concha bowl 957 when the body-structure 67 is placed in the tragus-concha bowl 957. By carefully selecting the location for the auriculotemporal nerve stimulating electrode 312 on the body-structure 67 to match the aforementioned innervation locations of the auriculotemporal nerve on the tragus-concha bowl 957, the auriculotemporal nerve stimulating electrode 312 will automatically get in close contact with its target skin on the tragus-concha bowl 957 when the body-structure 67 is placed in the tragus-concha bowl 957. Similarly, by carefully selecting the location for the greater auricular nerve stimulating electrode 314, on the body-structure 67 to match the aforementioned innervation locations of the greater auricular nerve innervated skin on the tragus-concha bowl 957, the greater auricular nerve stimulating electrode 314 will automatically get in close contact with its target skin on the tragus-concha bowl 957 when the body-structure 67 is placed in the tragus-concha bowl 957. Alternatively, the taVNS stimulating electrode 31 and the auriculotemporal nerve stimulating electrode 312 may be located on a tubular-shaped structure 66. By carefully selecting the location for the taVNS stimulating electrode 31 on the tubular-shaped structure 66 to match its target skin locations on the external ear canal 904, the taVNS stimulating electrode 31 will be automatically get in close contact with its target skin in the external ear canal 904. Similarly, by carefully selecting the location for the auriculotemporal nerve stimulating electrode 312 on the tubular-shaped structure 66 to match its target skin locations on the external ear canal 904, the auriculotemporal nerve stimulating electrode 312 will be automatically get in close contact with its target skin in the external ear canal 904.

Generally, an elastic flexible and adaptable material may be flexible to allow slight deformation and optionally resilient so as to return to its original shape after deformation. In preferred embodiments, all or portions of a modified earbud housing 61 (e.g., a tubular-body portion 25), all or portions of a modified in-the-ear housing 62, all or portions of a behind-the-ear-hearing-aid-style housing 63 (e.g., in-the-ear portion 27), all or portions of the tubular-shaped structure 66 may be made from or comprise an elastic flexible and adaptable material such as natural and/or synthetic rubber material such as latex rubber, silicone foam, silicone rubber or polysiloxanes, rubber foam, urethane foam, plastic foam, neoprene foam, latex foam rubber, polyurethane foam rubber, forms of the organic compound isoprene, Polyacrylate Rubber, Ethylene-acrylate Rubber, Polyester Urethane, flexible plastics, such as high-density polyethylene (HDPE), polyvinyl chloride (PVC), polypropylene (PP), Polystyrene (PS), Polycarbonate (PC), low density polyethylene (LDPE), or any other flexible material including combinations of materials.

In some embodiments, an auricular housing 11 may house one or more components, such as a processing unit 50, an EEG recording module 20, an auricular speckle contrast optical spectroscopy (auricular SCOS) 511, a PPG unit 555, EEG sensor electrodes 12, 13, 72, 73, 82, 83, an optional reference electrode 84, taVNS stimulating electrode 31, an auriculotemporal nerve stimulating electrode 312, a greater auricular nerve stimulating electrode 314, a speaker 15, a power source 16, a vibrator 17, etc., which may be communicatively coupled via a local interface 58. The local interface 58 can be, for example but not limited to, one or more buses, circuit boards, wiring harnesses, or other wired connections or wireless connections, as is known in the art. The local interface 58 can have additional elements, which are omitted for simplicity, such as controllers, buffers (caches), drivers, repeaters, and receivers, among many others, to enable communications. Further, the local interface 58 may include address, control, and/or data connections to enable appropriate communications among the aforementioned components. In some embodiments, an auricular EEG monitoring system 100 may comprise a processing unit 50 which may be contained in the auricular housing 11.

A processing unit 50 may include a processor 51 that may comprise a hardware device for executing software instructions. (FIG. 14). The processor 51 can be any custom made or commercially available processor, a central processing unit (CPU), an auxiliary processor among several processors associated with the processing unit 50, a semiconductor-based microprocessor (in the form of a microchip or chip set), or generally any device for executing software instructions. Optionally, when the processing unit 50 is in operation, the processor 51 may be configured to execute software stored within a memory 55, to communicate data to and from the memory 55, and to generally control one or more operations of the auricular EEG monitoring system 100 pursuant to the software instructions and/or from instructions. In an exemplary embodiment, the processor 51 may include a mobile optimized processor, such as optimized for power consumption and mobile applications.

In some embodiments, an auricular EEG monitoring system 100 may comprise one or more I/O interfaces 52 which can be used to provide user input and display system output data, such as operational status, from the auricular EEG monitoring system 100. The I/O interfaces 52 can include, for example, buttons, knobs, switches, LED indicator lights, LED display, LCD display, a serial port, a parallel port, a small computer system interface (SCSI), an infrared (IR) interface, a radio frequency (RF) interface, a universal serial bus (USB) interface, and the like. In some embodiments, an auricular EEG monitoring system 100 may comprise a network interface 53 which may be contained in the auricular housing 11 and which may enable wired and/or wireless communication between one or more components, such as EEG recording module 20, processing unit 50, etc., with one or more client devices 400. Preferably, a network interface 53 may comprise a radio that may operate via WiFi and/or Bluetooth communication standards. In further embodiments, a network interface 53 may comprise a radio that may operate on a cellular band and may communicate with or receive a Subscriber Identity Module (SIM) card or other wireless network identifier. Any number of suitable wireless data communication protocols, techniques, or methodologies can be supported by a network interface 53, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Near-Field Communication (NFC); Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication. In further embodiments, a network interface 53 may enable wired network communication and may include, for example, an Ethernet card or adapter (e.g., 10BaseT, Fast Ethernet, Gigabit Ethernet, 10GbE) or a wireless local area network (WLAN) card or adapter (e.g., 802.11a/b/g/n). The network interface 53 may include address, control, and/or data connections to enable appropriate communications on the network.

In some embodiments, a processing unit 50 may comprise a memory 55 that may include any volatile memory elements (e.g., random access memory (RAM, such as DRAM, SRAM, SDRAM, etc.)), nonvolatile memory elements (e.g., ROM, hard drive, etc.), and combinations thereof. Moreover, the memory 55 may incorporate electronic, magnetic, optical, and/or other types of storage media. Note that the memory 55 may have a distributed architecture, where various components are situated remotely from one another, but can be accessed by the processor 51. Optionally, memory 55 can include one or more software programs, each of which includes an ordered listing of executable instructions for implementing logical functions. Optionally, the software in the memory system 55 includes a suitable operating system (O/S) 56 and program(s) 57. The operating system 56 essentially controls the execution of input/output interface 52 and other element functions, and provides scheduling, input-output control, file and data management, memory management, and communication control and related services. The operating system 56 may be, for example, LINUX (or another UNIX variant), Android (available from Google), Symbian OS, Microsoft Windows CE, Microsoft Windows 7 Mobile, iOS (available from Apple, Inc.), webOS (available from Hewlett Packard), Blackberry OS (Available from Research in Motion), and the like. The programs 57 may include various applications, add-ons, etc. configured to provide end user functionality of the system 100.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The auricular EEG monitoring system 100 may comprise one or more auricular EEG recording modules 20. In some embodiments, an auricular EEG monitoring system 100 may comprise an auricular EEG recording module 20 that may be contained in an auricular housing 11. An auricular EEG recording module 20 may record the electrical activities of the brain (EEG signals) of the wearer 900 to generate EEG data. The EEG data may be analyzed by a processing unit 50, 401, to detect presence or cessation of EEG signals suggestive of migraine or cluster headache. The processing unit 50, 401 is further configured to analyze the EEG data to detect presence or cessation of EEG signals suggestive of impending migraine or impending cluster headache.

Generally, amplifiers and filters 21 of an auricular EEG recording module 20 may pick up the electrical activities of the wearer's 900 brain via the plurality of EEG sensor electrodes 12, 13, 72, 73, 82, 83 and the optional reference electrode 84. An amplifier of amplifiers and filters 21 is responsible for amplifying the weak electrical signals received from the electrodes 12, 13, 72, 73, 82, 83, 84. The brain's electrical signals are typically very faint, (often in the 5-30 microvolts range for in-ear electrodes and about 10-100 microvolts for scalp electrodes). The amplifier boosts these signals to a level that can be accurately recorded and displayed. Modern EEG machines use sophisticated amplifiers that minimize noise and ensure signal clarity. Filters of amplifiers and filters 21 are used to remove unwanted noise and interference from the electrical signals. Common sources of noise include muscle contractions, electrical interference from other devices, and movement artifacts. EEG machines use various filters, such as high-pass, low-pass, and notch filters, to clean the signals, ensuring that the resulting EEG trace is clear and interpretable. An analog-to-digital converter (ADC) 22 may transform the analog electrical signals from the brain into digital data. This digital conversion is essential for processing, storing, and displaying the EEG data on a screen or print out. The ADC 22 ensures that the data is accurately digitized, preserving the integrity of the original signals. The ADC 22 may be in communication with a processing unit 50.

According to another embodiment consistent with the principles of the present invention, an automatic detection-therapy system 101 is disclosed (FIGS. 2, 3, 5-7, 11-13, 18 and 28). In some embodiments, an automatic detection-therapy system 101 may comprise an auricular electroencephalogram (EEG) monitoring system 100 having one or more EEG recording modules 20 (for example, a first EEG recording module configured to record EEG from a wearer's first ear 902 and a second EEG recording module configured to record EEG from the wearer's second ear 902). Each EEG recording module 20 preferably has a plurality of miniature wired or wireless EEG sensor electrodes 12, 13, 72, 73, 82, 83, and an optional wired or wireless reference electrode 84 that are configured to be linked to an ear 902 of the wearer such as to contact or be attached to separate areas selected from at least one of the following: the external ear canal 904, external ear 902, or peri-auricular area 903, that may be configured to record EEG data of the wearer 900, such as discussed above. In some embodiments, an automatic detection-therapy system 101 may include one or two transcutaneous auricular vagus nerve stimulation units (taVNS units) 30, such as a first taVNS unit 30 and a second taVNS unit 30, with each taVNS unit 30 having a taVNS stimulating electrode 31 configured to contact vagus innervated auricular skin of one of the wearer's ears 902. The vagus innervated auricular skin includes external ear canal 904, tragus 905, cymba-concha 906, cavum-concha 907 and small adjacent areas. (More specifically, the vagus innervated auricular skin includes: posterior and inferior walls of external ear canal 904, inner/posterior portion of tragus 905, cymba-concha 906, and majority of cavum-concha 907, and part of the eardrum.) The vagus innervated auricular skin that the stimulating electrode 31 is configured to contact may be selected from at least one of the following: external ear canal 904, tragus 905, cymba-concha 906, and cavum-concha 907. The one or more EEG recording modules 20 and the one or two taVNS units 30 are in wired or wireless electronic communication with the processing unit 50, 401.

In some embodiments, an automatic detection-therapy system 101 may comprise one transcutaneous auricular vagus nerve stimulation (taVNS) unit, such as a first taVNS unit 30. The first taVNS unit 30 having a first taVNS stimulating electrode 31 configured to contact vagus innervated auricular skin of the wearer's first ear 902. The vagus innervated auricular skin includes: posterior and inferior walls of external ear canal 904, inner/posterior portion of tragus 905, cymba-concha 906, cavum-concha 907 and small adjacent areas. The vagus innervated auricular skin that the stimulating electrode 31 is configured to contact may be selected from at least one of the following: external ear canal 904, tragus 905, cymba-concha 906, and cavum-concha 907 of the wearer's ear 902. A processing unit 50, 401, may be in electronic communication with the auricular electroencephalogram (EEG) monitoring system 100 and with the first taVNS unit 30. The processing unit 50, 401, may be configured to analyze EEG data recorded by the EEG monitoring system 100 to detect the presence or cessation of EEG signals suggestive of migraine or cluster headache. When the presence of EEG signals suggestive of a migraine or cluster headache is detected by the processing unit 50, 401, the processing unit 50, 401, may be configured to immediately send signals to the first taVNS unit 30 to prompt the first taVNS unit 30 to automatically start sending pre-determined electric stimulation to the vagus innervated auricular skin of the wearer's ear 902 to which the first taVNS stimulating electrode 31 is in contact with. When cessation of EEG signals suggestive of the migraine or cluster headache is detected by the processing unit 50, 401, the processing unit 50, 401, may be further configured to immediately send signals to the first taVNS unit 30 to prompt (or activate) the first taVNS unit 30 to automatically stop sending pre-determined electric stimulation to the vagus innervated auricular skin of the wearer's first ear 902. The processing unit 50, 401, may be further configured to analyze the EEG data recorded by the EEG monitoring system 100 to detect the presence or cessation of EEG signals suggestive of impending migraine or impending cluster headache. When the presence of EEG signals suggestive of impending migraine or impending cluster headache is detected by the processing unit 50, 401, the processing unit 50, 401, may be configured to immediately send signals to the first taVNS unit 30 to prompt the first taVNS unit 30 to automatically start sending predetermined electric stimulation to the vagus innervated auricular skin of the wearer's first ear 902. When cessation of EEG signals suggestive of the impending migraine or impending cluster headache is detected by the processing unit 50, 401, the processing unit 50, 401, may be further configured to immediately send signals to the first taVNS unit 30 to prompt the first taVNS unit 30 to automatically stop sending electric stimulation to the vagus innervated auricular skin of the wearer's first ear 902.

In some embodiments, an automatic detection-therapy system 101 may comprise two transcutaneous auricular vagus nerve stimulation (taVNS) units 30 (“taVNS unit 30”) such as shown in FIG. 18. Preferably, an automatic detection-remedy system 101 may comprise a first taVNS unit (such as described hereinbefore) and a second taVNS unit 30. The function of the second taVNS unit 30 is similar to the aforementioned description for the first taVNS unit. 30

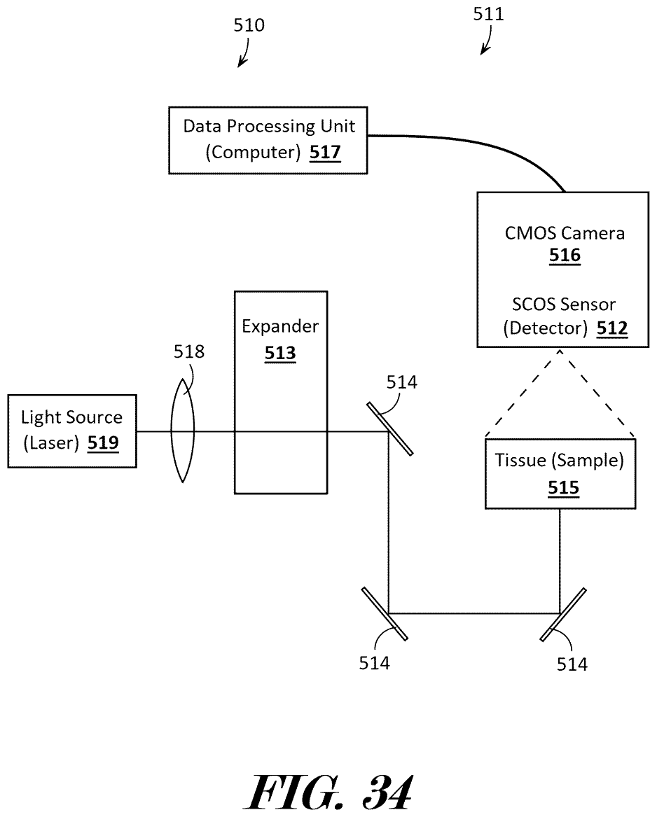

In some embodiments, an automatic detection-therapy system 101 may comprise a neurovascular monitoring system 500 having an auricular EEG monitoring system 100 and a cephalic blood flow monitoring system 530, such a speckle contrast optical spectroscopy unit (SCOS unit) 510. There are a few other laser speckle blood flow devices that can be used as alternate. Photoplethysmography (PPG) could be another alternative although PPG is usually used to monitor superficial blood flow. A novel auricular Speckle Contrast Optical Spectroscope unit (auricular SCOS unit) 511 is disclosed by placing a SCOS unit 510 in an auricular housing 11, such as a modified earbud housing 61 or an in-the-ear housing 62 or a behind-the-ear-hearing-aid-style housing 63. The auricular SCOS unit 511 comprises a light source (laser) 519 that pass through a focusing lens 518 and an expander 513 and mirrors 514 and then tissue (or sample) 515. The light is then collected by a SCOS sensor (detector) 512 that is integrated with a CMOS camera 516 and the data are transmitted to a data processing unit (computer) 517 for analysis and to get the blood flow data at different depths of the tissue. (FIG. 34). The auricular SCOS sensor 512 is located on a surface of a wearer's tragus-concha bowl 957 or the wearer's external ear canal 904 when in use to collect the wearer's cerebral (intracranial) and extracranial blood flow data. The neurovascular monitoring system 500 is configured to simultaneously record the wearer's EEG data (via the auricular EEG monitoring system 100) and the wearer's cerebral and extracranial blood flow data (via the cephalic blood flow monitoring system 530).

In preferred embodiments, an automatic detection-therapy system 101 may comprise a novel integration of a neurovascular monitoring system 500 and a neuromodulation unit 300. The neurovascular monitoring system 500 includes an auricular EEG monitoring system 100 and a cephalic blood flow monitoring system 530 such as an auricular speckle contrast optical spectroscope unit (auricular SCOS unit) 511. (Alternatively, the cephalic blood flow monitoring system 530 may comprise an integration of an auricular SCOS unit 511 and a PPG unit 555). The auricular SCOS unit 511 is configured to collect the intracranial (cerebral) and extracranial blood flow data of a wearer 900. The neurovascular monitoring system 500 is configured to simultaneously record the wearer's EEG data (via the auricular EEG monitoring system 100) and the wearer's cerebral and extracranial blood flow data (via the auricular SCOS unit 511). The neuromodulation unit 300 may include at least one of the following components: a transcutaneous auricular vagus nerve stimulation unit (taVNS unit) 30, a supraorbital nerve stimulation unit 301, an auriculotemporal nerve stimulation unit 302, an occipital nerve stimulation unit 303, a greater auricular nerve (GAN) stimulation unit 304 and an infraorbital nerve stimulation unit 305. In some embodiments, a neuromodulation unit 300 may include a taVNS unit 30. The taVNS unit 30 preferably has a miniature taVNS stimulating electrode 31 that is configured to contact or be attached to vagus nerve innervated auricular skin of the wearer 900. The EEG recording module 100 and the auricular SCOS unit 511 may be in electronic communication (e.g., through wire, Bluetooth, etc.) with a processing unit 50, 401. The taVNS unit 30 may also be in wired or wireless electronic communication with a processing unit 50, 401. When the processing unit 50, 401, detects one of the following: presence of EEG signals suggestive of migraine or cluster headache and presence of blood flow data suggestive of migraine or cluster headache, the processing unit 50, 401, may be configured to send signals to the taVNS unit 30 to prompt the taVNS unit 30 to start sending neuromodulating electric stimulation to the vagus nerve innervated auricular skin of the wearer 900. When the processing unit 50, 401, detects one of the following: presence of EEG signals suggestive of impending migraine or impending cluster headache and presence of blood flow data suggestive of impending migraine or impending cluster headache, the processing unit 50, 401, may be configured to send signals to the taVNS unit 30 to prompt the taVNS unit 30 to start sending neuromodulating electric stimulation to the vagus nerve innervated auricular skin of the wearer 900. When the processing unit 50, 401 detects all of the following: cessation of EEG signals suggestive of migraine or cluster headache, cessation of blood flow data suggestive of migraine or cluster headache, cessation of EEG signals suggestive of impending migraine or impending cluster headache, and cessation of blood flow data suggestive of impending migraine or impending cluster headache, the processing unit 50, 401, may be further configured to send signals to the taVNS unit 30 to stop the neuromodulating electric stimulation.

In some embodiments, an automatic detection-therapy system 101 may comprise a neuromodulation unit 300 that may include at least one of the following components: a transcutaneous auricular vagus nerve stimulation unit (taVNS unit) 30, a supraorbital nerve stimulation unit 301, an auriculotemporal nerve (ATN) stimulation unit 302, an occipital nerve stimulation unit 303, a greater auricular nerve (GAN) stimulation unit 304 and an infraorbital nerve stimulation unit 305. In some embodiments, a neuromodulation unit 300 may include a taVNS unit 30. The setups and the functions of the taVNS unit 30 are as described hereinbefore. The setups and functions of the supraorbital nerve stimulation unit 301, the auriculotemporal nerve (ATN) stimulation unit 302, the occipital nerve stimulation unit 303, the GAN stimulation unit 304 and the infraorbital nerve stimulation unit are also similar to the above descriptions for taVNS.

In some embodiments, an automatic detection-therapy system 101 may comprise a neuromodulation unit 300 that includes a supraorbital nerve stimulation unit 301. When prompted (by EEG signals and blood flow data as described hereinbefore), the supraorbital nerve stimulation unit 301 may be configured to start (or stop) neuromodulating electric stimulation to supraorbital nerve innervated skin at the wearer's 900 forehead via a supraorbital nerve stimulating electrode 311. When the processing unit 50, 401, detects one of the following: presence of EEG signals suggestive of migraine or cluster headache and presence of blood flow data suggestive of migraine or cluster headache, the processing unit 50, 401, may be configured to send signals to the supraorbital nerve stimulation unit 301 to prompt the supraorbital nerve stimulation unit 301 to start sending neuromodulating electric stimulation to the supraorbital nerve innervated forehead skin of the wearer 900. When the processing unit 50, 401, detects one of the following: presence of EEG signals suggestive of impending migraine or impending cluster headache and presence of blood flow data suggestive of impending migraine or impending cluster headache, the processing unit 50, 401, may be configured to send signals to the supraorbital nerve stimulation unit 301 to prompt the supraorbital nerve stimulation unit 301 to start sending neuromodulating electric stimulation to the supraorbital nerve innervated forehead skin of the wearer 900. When the processing unit 50, 401 detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit 50, 401, may be further configured to send signals to the supraorbital nerve stimulation unit 301 to stop the neuromodulating electric stimulation.

In some embodiments, an automatic detection-therapy system 101 may comprise a neuromodulation unit 300 that includes an auriculotemporal nerve stimulation unit 302. When prompted (by EEG signals and blood flow data as described hereinbefore), the auriculotemporal nerve stimulation unit 302 may be configured to start (or stop) neuromodulating electric stimulation to the auriculotemporal nerve innervated auricular skin of the wearer 900 via an auriculotemporal nerve stimulation electrode 312. When the processing unit 50, 401, detects one of the following: presence of EEG signals suggestive of migraine or cluster headache and presence of blood flow data suggestive of migraine or cluster headache, the processing unit 50, 401, may be configured to send signals to the auriculotemporal nerve stimulation unit 302 to prompt the auriculotemporal nerve stimulation unit 302 to start sending neuromodulating electric stimulation to the auriculotemporal nerve innervated skin of the wearer's ear 902. When the processing unit 50, 401, detects one of the following: presence of EEG signals suggestive of impending migraine or impending cluster headache and presence of blood flow data suggestive of impending migraine or impending cluster headache, the processing unit 50, 401, may be configured to send signals to the auriculotemporal nerve stimulation unit 302 to prompt the auriculotemporal nerve stimulation unit 302 to start sending neuromodulating electric stimulation to the auriculotemporal nerve innervated skin of the wearer's ear 902. When the processing unit 50, 401 detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit 50, 401, may be further configured to send signals to the auriculotemporal nerve stimulation unit 302 to stop the neuromodulating electric stimulation. The auriculotemporal nerve innervated auricular skin includes the anterior outer part of tragus 905, anterior superior portion of the cavum-concha 907, anterior-superior helix 909, anterior and superior walls of the external ear canal 904, anterior auricle and part of the outer tympanic membrane. Thus, the tragus-concha bowl 957 and the external ear canal 904 received mixed innervation from both the auricular branch of vagus nerve and the auriculotemporal nerve. Alternatively, the auriculotemporal nerve stimulation unit 302 may be configured to deliver electric stimulation via a clip electrode 319 for auriculotemporal nerve stimulation to auriculotemporal nerve innervated anterior-superior helix 909 of the wearer's ear 902 when prompted or activated.

In some embodiments, an automatic detection-therapy system 101 may comprise a neuromodulation unit 300 that includes an occipital nerve stimulation unit 303. When prompted (by EEG signals and blood flow data as described hereinbefore), the occipital nerve stimulation unit 303 may be configured to start (or stop) neuromodulating electric stimulation to occipital nerve innervated skin at occipital region of the wearer 900 via an occipital nerve stimulating electrode 313. When the processing unit 50, 401, detects one of the following: presence of EEG signals suggestive of migraine or cluster headache and presence of blood flow data suggestive of migraine or cluster headache, the processing unit 50, 401, may be configured to send signals to the occipital nerve stimulation unit 303 to prompt the occipital nerve stimulation unit 303 to start sending neuromodulating electric stimulation to the occipital nerve innervated occipital region of the wearer 900. When the processing unit 50, 401, detects one of the following: presence of EEG signals suggestive of impending migraine or impending cluster headache and presence of blood flow data suggestive of impending migraine or impending cluster headache, the processing unit 50, 401, may be configured to send signals to the occipital nerve stimulation unit 303 to prompt the occipital nerve stimulation unit 303 to start sending neuromodulating electric stimulation to the occipital nerve innervated occipital region of the wearer 900. When the processing unit 50, 401 detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit 50, 401, may be further configured to send signals to the occipital nerve stimulation unit 303 to stop the neuromodulating electric stimulation. In some embodiments, the supraorbital nerve stimulation unit 301 and the occipital nerve stimulation unit 303 may be installed manually by the wearer 900 when the wearer 900 received pertinent notification from the network interface 406. Alternatively, the supraorbital nerve stimulation unit 301 and the occipital nerve stimulation unit 303 may be pre-installed and may be configured to deliver neuromodulating electric stimuli automatically when prompted via wireless communication from the processing unit 50, 401. (It should be noted that, in preferred embodiments, the taVNS stimulating electrode 31, the auriculotemporal nerve stimulating electrode 312, the greater auricular nerve stimulating electrode 314, the auricular SCOS sensor 512, all of the EEG sensor electrodes 12, 13, 72, 73, 82, 83 and the optional reference electrode 84 may be configured to be housed in one auricular housing 11 and therefore will be automatically pre-installed when the wearer 900 places the auricular housing 11 into an ear 902 of the wearer 900.)

In some embodiments, an automatic detection-therapy system 101 may comprise a neuromodulation unit 300 that includes a greater auricular nerve (GAN) stimulation unit 304. When prompted (by EEG signals and blood flow data as described hereinbefore), the greater auricular nerve (GAN) stimulation unit 302 may be configured to start (or stop) neuromodulating electric stimulation to the GAN innervated auricular skin of the wearer 900 via a GAN stimulation electrode 314. When the processing unit 50, 401, detects one of the following: presence of EEG signals suggestive of migraine or cluster headache and presence of blood flow data suggestive of migraine or cluster headache, the processing unit 50, 401, may be configured to send signals to the GAN stimulation unit 304 to prompt the GAN stimulation unit 304 to start sending neuromodulating electric stimulation to the GAN innervated skin of the wearer's ear 902. When the processing unit 50, 401, detects one of the following: presence of EEG signals suggestive of impending migraine or impending cluster headache and presence of blood flow data suggestive of impending migraine or impending cluster headache, the processing unit 50, 401, may be configured to send signals to the GAN stimulation unit 304 to prompt the GAN stimulation unit 304 to start sending neuromodulating electric stimulation to the GAN innervated skin of the wearer's ear 902. When the processing unit 50, 401 detects all of the following: cessation of EEG signals suggestive of migraine or cluster headache, cessation of blood flow data suggestive of migraine or cluster headache, cessation of EEG signals suggestive of impending migraine or impending cluster headache and cessation of blood flow data suggestive of impending migraine or impending cluster headache, the processing unit 50, 401, may be further configured to send signals to the GAN stimulation unit 304 to stop the neuromodulating electric stimulation. The greater auricular nerve (GAN) is a pure sensory nerve originated from the cervical spinal cord (C2, C3) and provides sensory innervation to the skin over the parotid gland, the mastoid process, and the lower two-thirds of the outer ear (pinna). The greater auricular nerve (GAN) innervated auricular skin includes skin on both surfaces of the pinna (including the lobule) and cavum concha (inferior/lower part of concha). Thus, the tragus-concha bowl 957 received mixed innervation from the auricular branch of vagus nerve, the auriculotemporal nerve and the greater auricular nerve. Research indicates that neuromodulation techniques targeting the GAN may provide therapeutic relief for various neurological (including migraine, cluster headache, seizure) and psychiatric disorders.

In preferred embodiments, an automatic detection-therapy system 101 may comprise one or two neuromodulation units 300. A first neuromodulation unit 300 may be configured to include at least one of the following: a first transcutaneous vagus nerve stimulation unit (taVNS unit) 30, a supraorbital nerve stimulation unit 301, a first auriculotemporal nerve stimulation unit 302, an occipital nerve stimulation unit 303, a first greater auricular nerve stimulation unit 304 and an infraorbital nerve stimulation unit 305, as aforementioned description. The first taVNS unit 30, the first auriculotemporal nerve stimulation unit 302 and the first greater auricular nerve stimulation unit 304 may be configured to be located at the wearer's first ear 902. The supraorbital nerve stimulation unit 301 may be located at midforehead and be configured to stimulate unilateral or bilateral supraorbital nerves. The occipital nerve stimulation unit 303 may be located at mid-occipital region and be configured to stimulate unilateral or bilateral occipital nerves. The infraorbital nerve stimulation unit 305 may be located at mid-face and be configured to stimulate unilateral or bilateral infraorbital nerves. Optionally, a second neuromodulation unit 300 may include a second taVNS unit 30, a second auriculotemporal nerve stimulation unit 302 and a second greater auricular nerve stimulation unit 304, configured to be located at the wearer's second ear 902. The setups and functions of the second taVNS unit 30 are essentially the same as the first taVNS unit 30. The setups and functions of the second auriculotemporal nerve stimulation unit 302 are essentially the same as the first auriculotemporal nerve stimulation unit 302. Likewise, the setups and functions of the second greater auricular nerve stimulation unit 304 are essentially the same as the first greater auricular nerve stimulation unit 304. Studies have shown that bilateral neuromodulation is more effective than unilateral neuromodulation. Separately, studies have shown that simultaneous neuromodulation of two or three nerves on the same side is more effective than single nerve neuromodulation due to their synergistic effects. Double neuromodulation may consist of simultaneous neuromodulation of two nerves, such as simultaneous stimulation of auricular branch of vagus nerve and auriculotemporal nerve, or simultaneous neuromodulation of vagus nerve and greater auricular nerve or simultaneous neuromodulation of vagus nerve and occipital nerve, etc. Triple neuromodulation may consist of simultaneous neuromodulation of three nerves, such as simultaneous neuromodulating electric stimuli to auricular branch of vagus nerve, auriculotemporal nerve and greater occipital nerve, etc.

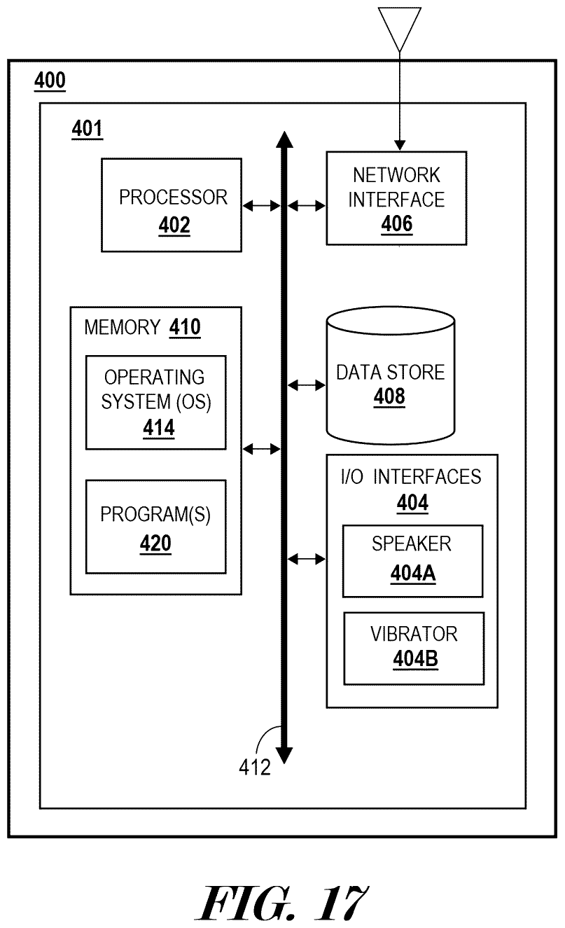

In some embodiments, an auricular EEG monitoring system 100 and/or an automatic detection-therapy system 101 may be in electronic communication with one or more client devices 400. In some embodiments, the auricular EEG monitoring system 100 may comprise one or more client devices 400. Referring to FIG. 17, in an exemplary embodiment, a block diagram illustrates a client device 400 of which may be a type of computing platform. A client device 400 can be a digital device that, in terms of hardware architecture, generally includes a processor 402, input/output (I/O) interfaces 404, a network interface 406, a data store 408, and memory 410. It may be appreciated by those of ordinary skill in the art that FIG. 17 depicts the client device 400 in an oversimplified manner, and a practical embodiment may include additional components and suitably configured processing logic to support known or conventional operating features that are not described in detail herein. The components (402, 404, 406, 408, and 410) are communicatively coupled via a local interface 412. The local interface 412 can be, for example but not limited to, one or more buses or other wired or wireless connections, as is known in the art. The local interface 412 can have additional elements, which are omitted for simplicity, such as controllers, buffers (caches), drivers, repeaters, and receivers, among many others, to enable communications.

In some embodiments, an auricular EEG monitoring system 100 and/or an automatic detection-therapy system 101 may comprise a processor 402. The processor 402 is a hardware device for executing software instructions. The processor 402 can be any custom made or commercially available processor, a central processing unit (CPU), an auxiliary processor among several processors associated with the client device 400, a semiconductor-based microprocessor (in the form of a microchip or chip set), or generally any device for executing software instructions. When the client device 400 is in operation, the processor 402 is configured to execute software stored within the memory 410, to communicate data to and from the memory 410, and to generally control operations of the client device 400 pursuant to the software instructions. In an exemplary embodiment, the processor 402 may include a mobile optimized processor such as optimized for power consumption and mobile applications.

In some embodiments, an auricular EEG monitoring system 100 and/or an automatic detection-therapy system 101 may comprise an I/O interface 404. The I/O interfaces 404 can be used to receive data and user input and/or for providing system output. User input can be provided via a plurality of I/O interfaces 404, such as a keypad, a touch screen, speaker 404A, a camera, a microphone, a scroll ball, a scroll bar, buttons, barcode scanner, voice recognition, eye gesture, and the like. System output can be provided via a display screen such as a liquid crystal display (LCD), touch screen, and the like. The I/O interfaces 404 can also include, for example, a global positioning service (GPS) radio, a serial port, a parallel port, a small computer system interface (SCSI), an infrared (IR) interface, a radio frequency (RF) interface, a universal serial bus (USB) interface, and the like. The I/O interfaces 404 can include a graphical user interface (GUI) that enables a user to interact with the client device 400. Additionally, the I/O interfaces 404 may be used to output notifications to a user and can include a speaker 404A or other sound emitting device configured to emit audio notifications, a vibrational device or vibrator 404B configured to vibrate, shake, or produce any other series of rapid and repeated movements to produce haptic notifications, and/or a light emitting diode (LED) or other light emitting element which may be configured to illuminate to provide a visual notification.

In some embodiments, an auricular EEG monitoring system 100 and/or an automatic detection-therapy system 101 may comprise a network interface 406. The network interface 406 enables wireless communication to an external access device or network. Any number of suitable wireless data communication protocols, techniques, or methodologies can be supported by the network interface 406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication.

In some embodiments, an auricular EEG monitoring system 100 and/or an automatic detection-therapy system 101 may comprise a data store 408. The data store 408 may be used to store data and is therefore a type of memory. The data store 408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 408 may incorporate electronic, magnetic, optical, and/or other types of storage media. In some embodiments, an auricular EEG monitoring system 100 and/or an automatic detection-therapy system 101 may comprise a memory 410. The memory 410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 410 may incorporate electronic, magnetic, optical, and/or other types of storage media. Note that the memory 410 may have a distributed architecture, where various components are situated remotely from one another, but can be accessed by the processor 402. The software in memory 410 can include software programs 420 that include an ordered listing of executable instructions for implementing logical functions. In the example of FIG. 17, the software in the memory system 410 includes a suitable operating system (O/S) 414 and programs 420.

In some embodiments, an auricular EEG monitoring system 100 and/or an automatic detection-therapy system 101 may comprise an operating system 414. The operating system 414 essentially controls the execution of other computer programs, and provides scheduling, input-output control, file and data management, memory management, and communication control and related services. The operating system 414 may be, for example, LINUX (or another UNIX variant), Android (available from Google), Symbian OS, Microsoft Windows CE, Microsoft Windows 7 Mobile, Microsoft Windows 10, iOS (available from Apple, Inc.), webOS (available from Hewlett Packard), Blackberry OS (Available from Research in Motion), and the like. In some embodiments, an auricular EEG monitoring system 100 and/or an automatic detection-therapy system 101 may comprise programs 420. The programs 420 may include various applications, add-ons, etc. configured to provide end user functionality with the client device 400. For example, exemplary programs 420 may include, but not limited to, a web browser, social networking applications, streaming media applications, games, mapping and location applications, electronic mail applications, financial applications, and the like.

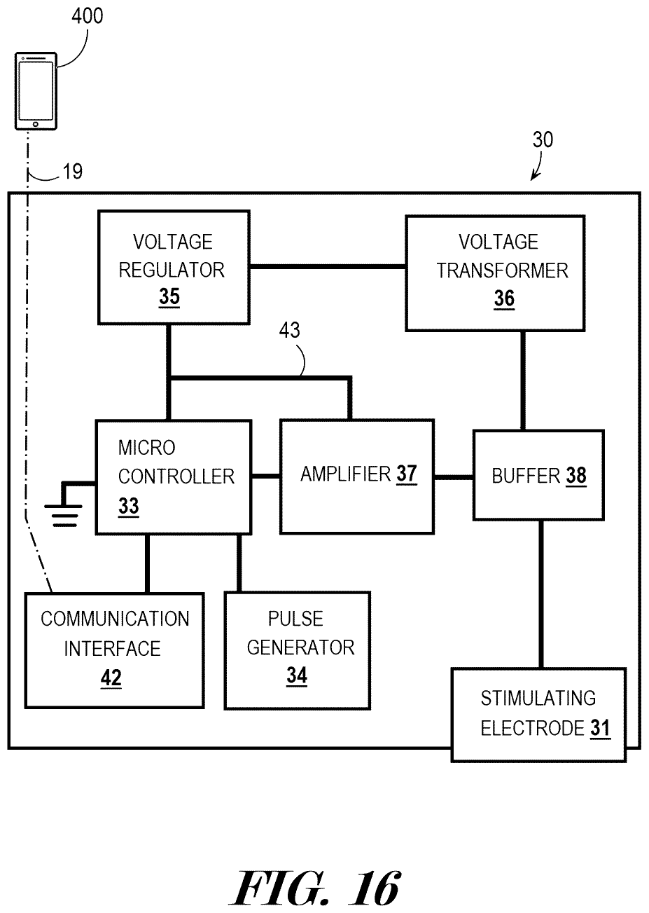

In some embodiments, an automatic detection-therapy system 101 may comprise a taVNS unit. The taVNS unit 30 may comprise any device that is able to provide transcutaneous auricular vagus nerve stimulation to a user's body when activated. As an example, and referring to FIG. 16, a taVNS unit 30 may comprise a microcontroller 33 that may be in communication with a pulse generator 34, voltage regulator 35, voltage transformer 36, amplifier 37, and buffer 38, and that may be configured to generate taVNS stimuli that may be transmitted to vagus nerve innervated auricular skin via a taVNS stimulating electrode 31.

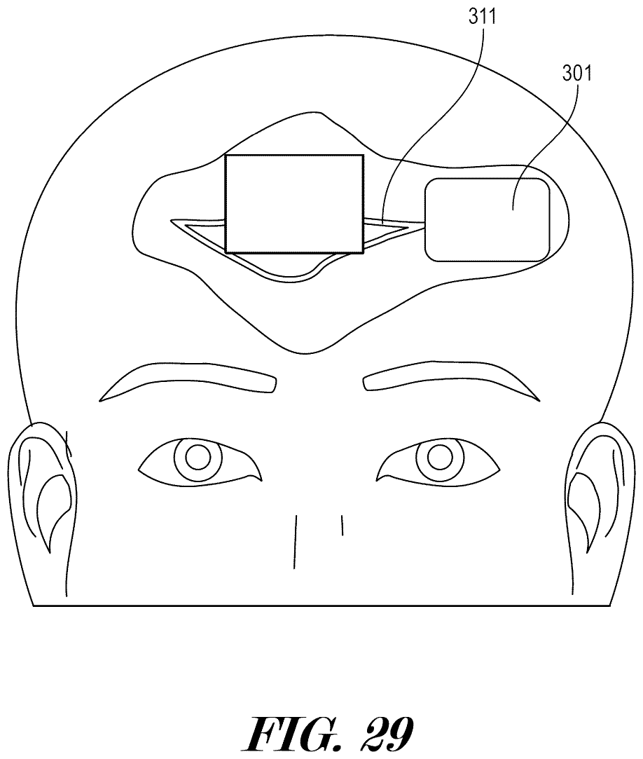

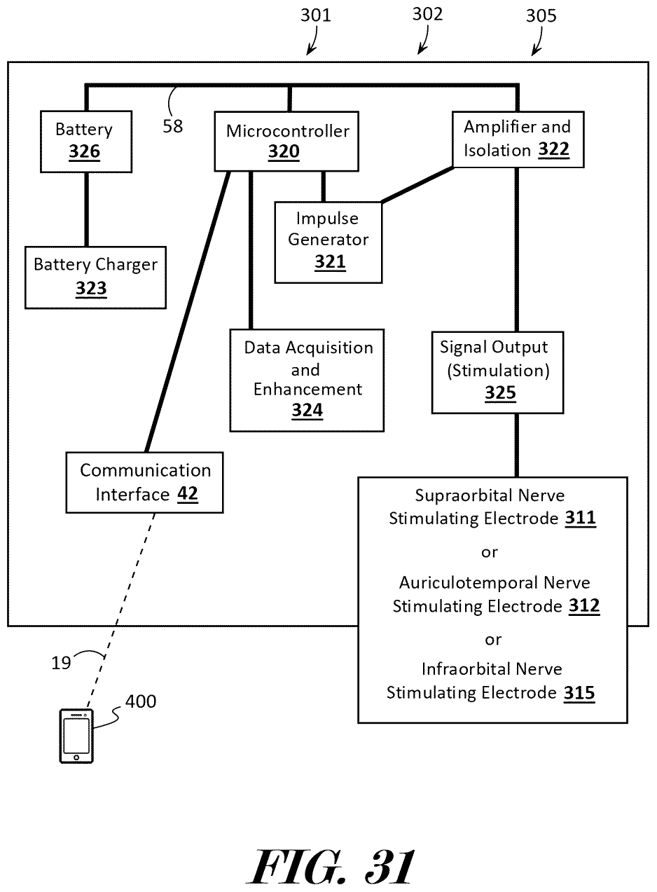

In some embodiments, an automatic detection-therapy system 101 may comprise a supraorbital nerve stimulation unit 301. The supraorbital nerve stimulation unit 301 may comprise any device that is able to provide transcutaneous supraorbital nerve stimulation to a user's body when activated. As an example, and referring to FIGS. 29, 31, a supraorbital nerve stimulation unit 301 may comprise a microcontroller 320 that may be in communication with an impulse generator 321, amplifier and isolation 322, data acquisition and enhancement 324, signal output (stimuli) 325, and supraorbital nerve stimulating electrode 311 to supraorbital nerve(s). The supraorbital nerve stimulation unit 301 further comprises a battery 326, a battery charger 323 and a communication interface 42 for communication to a client device 400. The supraorbital nerve stimulation unit 301 may be configured to generate electric stimulation that may be transmitted to supraorbital nerve innervated forehead skin via the supraorbital nerve stimulating electrode 311. Similarly, in some embodiments, an infraorbital nerve stimulation unit 305 may be included as another component of the neuromodulation unit 300. The infraorbital nerve stimulation unit 305 includes an infraorbital nerve stimulating electrode 315 configured to contact infraorbital nerve innervated midfacial region of the wearer 900. Infraorbital nerve is a branch from the second division of the trigeminal nerve. Infraorbital nerve stimulation has been found to be effective for conditions like trigeminal neuralgia, post-herpetic neuralgia, headache, facial pain, etc. However, infraorbital nerve stimulation is more complex to set up and sometimes requires invasive procedure (although transcutaneous unit 305 is available nowadays). The function of the infraorbital nerve stimulation unit 305 is essentially similar to that of the supraorbital nerve stimulation unit 301.

In some embodiments, an automatic detection-therapy system 101 may comprise an auriculotemporal nerve stimulation unit 302. The auriculotemporal nerve stimulation unit 302 may comprise any device that is able to provide transcutaneous auriculotemporal nerve stimulation to a user's body when activated. As an example, and referring to FIG. 31, an auriculotemporal nerve stimulation unit 302 may comprise a microcontroller 320 that may be in communication with an impulse generator 321, amplifier and isolation 322, data acquisition and enhancement 324, signal output (stimuli) 325, and auriculotemporal nerve stimulating electrode 312, 319, to auriculotemporal nerve. The auriculotemporal nerve stimulation unit 302 further comprises a battery 326, a battery charger 323 and a communication interface 42 for communication to a client device 400. The auriculotemporal nerve stimulation unit 302 may be configured to generate electric stimulation that may be transmitted to auriculotemporal nerve innervated auricular skin (such as external ear canal 904 or tragus-concha bowl 957) via the auriculotemporal nerve stimulating electrode 312. Alternatively, the auriculotemporal nerve stimulation unit 302 may be configured to generate electric stimulation that may be transmitted to auriculotemporal nerve innervated auricular skin at anterior-superior helix 909 of the wearer's ear 902 via a clip electrode 319 for auriculotemporal nerve stimulation.

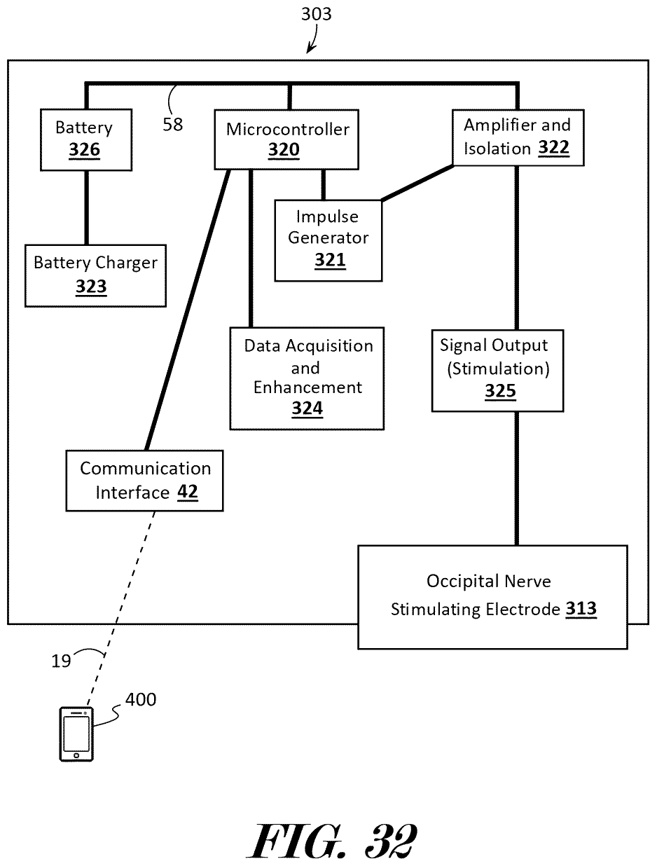

In some embodiments, an automatic detection-therapy system 101 may comprise an occipital nerve stimulation unit 303. The occipital nerve stimulation unit 303 may comprise any device that is able to provide occipital nerve stimulation to a user's body when activated. As an example, and referring to FIG. 32, an occipital nerve stimulation unit 303 may comprise a microcontroller 320 that may be in communication with an impulse generator 321, amplifier and isolation 322, data acquisition and enhancement 324, signal output (stimuli) 325, and occipital nerve stimulating electrode 313. The occipital nerve stimulation unit 303 further comprises a battery 326, a battery charger 323 and a communication interface 42 for communication with a client device 400. The occipital nerve stimulation unit 303 may be configured to generate electric stimulation that may be transmitted to occipital nerve innervated occipital region via the occipital nerve stimulating electrode 313.

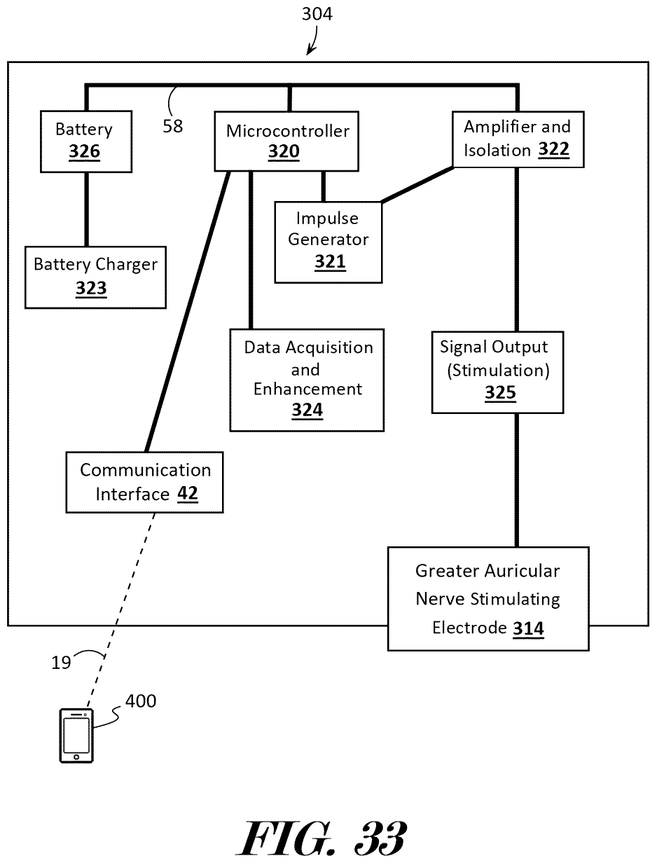

In some embodiments, an automatic detection-therapy system 101 may comprise a greater auricular nerve (GAN) stimulation unit 304. The GAN stimulation unit 304 may comprise any device that is able to provide GAN stimulation to a user's body when activated. As an example, and referring to FIG. 33, a GAN stimulation unit 304 may comprise a microcontroller 320 that may be in communication with an impulse generator 321, amplifier and isolation 322, data acquisition and enhancement 324, signal output (stimuli) 325, and greater auricular nerve stimulating electrode 314. The GAN stimulation unit 304 further comprises a battery 326, a battery charger 323 and a communication interface 42 for communication with a client device 400. The GAN stimulation unit 304 may be configured to generate electric stimulation that may be transmitted to greater auricular nerve innervated occipital region via the GAN stimulating electrode 314.

In some embodiments, an automatic detection-therapy system 101 may comprise a speckle contrast optical spectroscopy unit (SCOS unit) 510 or an auricular speckle contrast optical spectroscopy unit (auricular SCOS unit) 511. Referring to FIG. 34, a SCOS unit 510 or an auricular SCOS unit 511 comprises a light source (laser) 519, usually infrared laser diodes or near-infrared (NIR) laser diodes to ensure deep tissue penetration. They most commonly operating at wavelengths of 785 nm, 830 nm or 685 nm. These lasers often operate in continuous-wave (CW) mode with low power, focusing on detecting speckle pattern intensity fluctuations to measure blood flow. This type of laser light is effective because it can penetrate the skull and the brain, allowing for the measurement of blood flow and volume by analyzing the patterns of scattered light. The infrared light produces a back-scattered speckle pattern that varies with changes in blood flow and tissue oxygenation that pass through a focusing lens 518 and an expander 513 and mirrors 514 and then tissue (or sample) 515. The light is then collected by a SCOS sensor 512 that is integrated with a CMOS camera 516 and the data are transmitted to a data processing unit (computer) 517 for analysis and to get the blood flow data at different depths of the tissue. The auricular SCOS sensor 512 is configured to be located on a surface of a wearer's tragus-concha bowl 957 or the wearer's external ear canal 904 to collect the wearer's cerebral (intracranial) and extracranial blood flow data. There are two main types of SCOS designs: fiber-less (compact) design and fiber design. In fiber-less design, the CMOS camera sensor (or detector) is placed in direct contact with the user's skin (or scalp), whereas in fiber-based design, the sensor is remote and only the fiber optic bundle contacts the user's skin (or scalp). In standard SCOS setups, there is usually an air gap (for example, about 6 mm) between the laser and the skin to achieve the desired illumination spot size and to maintain safety standards. The fiber-less design is usually quite compact and wearable as compared with the traditional fiber optic cables which are bulky. (While the camera-based approach uses an air gap, a wearable fiber-less optical sensor for deep tissue measurement may be designed for direct contact or very minimal space between the sensor and the skin.) Fiber-less SCOS unit 511, with or without a small air gap, may be preferred for this invention.

In some embodiments, each component of a neuromodulation unit 300 of an automatic detection-therapy system 101 may comprise a communication interface 42. Optionally, a taVNS unit 30 may comprise a communication interface 42 which may enable electronic communication 19 (e.g., wired and/or wireless communication) between the taVNS unit 30 and another electronic device, such as a client device 400, an auricular EEG monitoring system 100, etc. Optionally, a supraorbital nerve stimulation unit 301 may comprise a communication interface 42 which may enable electronic communication 19 (e.g., wired and/or wireless communication) with another electronic device, such as a client device 400, an auricular EEG monitoring system 100, etc. Optionally, an auriculotemporal nerve stimulation unit 302 may comprise a communication interface 42 which may enable electronic communication 19 (e.g., wired and/or wireless communication) with another electronic device, such as a client device 400, an auricular EEG monitoring system 100, etc. Optionally, an occipital nerve stimulation unit 303 may comprise a communication interface 42 which may enable electronic communication 19 (e.g., wired and/or wireless communication) with another electronic device, such as a client device 400, an auricular EEG monitoring system 100, etc. Likewise, a greater auricular nerve stimulation unit 304 may comprise a communication interface 42 which may enable electronic communication 19 (e.g., wired and/or wireless communication) with another electronic device, such as a client device 400, an auricular EEG monitoring system 100, etc. Preferably, a communication interface 42 may comprise a radio that may operate via WiFi and/or Bluetooth communication standards. In further embodiments, a communication interface 42 may be configured as a network interface 53 described above so that it may operate on any wireless and/or wired electronic communication 19 protocol that a network interface 53 may use.

In some embodiments, an automatic detection-therapy system 101 may comprise or be contained in a housing. Preferably, an auricular housing 11 may be utilized for the housing purpose for any or part of the following: the taVNS unit 30, the auricular EEG monitoring system 100, the neurovascular monitoring system 500 and the neuromodulation unit 300. Preferably, the auricular housing 11 may be utilized to house all or nearly all of the following: all of the EEG sensor electrodes 12, 13, 72, 73, 82, 83, the optional reference electrode 84, the auricular SCOS sensor 512, the taVNS stimulating electrode 31, the auriculotemporal nerve stimulating electrode 312 and the greater auricular nerve stimulating electrode 314. The auricular housing 11 may be configured in any size and shape, and may be made from or comprise plastic, elastomer, silicone or any other material used in the field of personal medical devices. In some embodiments, an auricular housing 11 may be configured as a modified earbud housing 61, a modified in-the-ear housing 62, a behind-the-ear-hearing-aid-style housing 63 or a tubular-shaped structure 66 (standalone tubular-shaped structure 66). Optionally, all of the EEG sensor electrodes 12, 13, 72, 73, 82, 83, the optional reference electrode 84, the auricular SCOS sensor 512, the taVNS stimulating electrode 31, the auriculotemporal nerve stimulating electrode 312 and the greater auricular nerve stimulating electrode 314 may be configured to be all housed in a single auricular housing 11. Optionally, a taVNS unit 30 may be housed in a standalone housing that may be separated from the auricular housing 11. In some embodiments, the taVNS unit 30 may include a taVNS stimulating electrode 31 which may be built within the auricular housing 11 for the taVNS unit 30 (so that the taVNS housing and taVNS stimulating electrode 31 may be located within a single unit). In further embodiments, a taVNS stimulating electrode 31 may be connected with the taVNS unit 30 through a wire (so that the taVNS stimulating electrode 31 may be remote from the taVNS unit 30).

In some embodiments, an automatic detection-therapy system 101 may comprise a taVNS unit 30 and an auriculotemporal nerve stimulation unit 302. In preferred embodiments, the auriculotemporal nerve stimulation unit 302 and the taVNS unit 30 may be placed within a same housing. The taVNS unit 30 and the auriculotemporal nerve stimulation unit 302 may be intergraded into a single unit and they might share a common stimulating electrode. However, in preferred embodiments, the taVNS unit 30 and the auriculotemporal nerve stimulation unit 302 should be separate units with separate stimulating electrodes (a taVNS stimulating electrode 31 and an auriculotemporal nerve stimulating electrode 312) because it is preferred that the taVNS unit 30 and the auriculotemporal nerve stimulation unit 302 use its own optimal distinct stimulation parameters. They may share a same housing structure, such as one of a modified earbud housing 61, a modified in-the-ear housing and a behind-the-ear-hearing-aid-style housing 63. In preferred embodiments, all of the EEG electrodes 12, 13, 72, 73, 82, 83, the optional reference electrode 84, the auricular SCOS sensor, the taVNS stimulating electrode 31, the auriculotemporal nerve stimulating electrode 312 and the greater auricular nerve stimulating electrode 314 may be configured to be all placed within a same housing structure (an auricular housing 11), selected from one of the following: a modified earbud housing 61, a modified in-the-ear housing 62, a behind-the-ear-hearing-aid-style housing 63 and a tubular-shaped structure 66. This setup will be very convenient for the wearer (user). The tragus-concha bowl 957 and the external ear canal 904 are suitable locations for attachment of taVNS stimulating electrode 31. The tragus-concha bowl 957 and the external ear canal 904 are also suitable locations for attachment of the auriculotemporal nerve stimulating electrode; while the cavum concha 907 (tragus-concha bowl 957) is suitable for attachment of the greater auricular nerve stimulating electrode 314. (This is due to the fact that the auricular branch of vagus nerve and the auriculotemporal nerve innervate the external ear canal and the tragus-concha bowl; whereas the greater auricular nerve innervates the cavum concha.)

In preferred embodiments of an automatic detection-therapy system 101 for migraine, when the processing unit 50, 401 detects one of the following: EEG signals suggestive of migraine and blood flow data suggestive of migraine. the taVNS unit 30 may be configured to start sending neuromodulating electric stimulation to the auricular branch of vagus nerve via the taVNS stimulating electrode 31, utilizing pre-determined stimulation parameters, such as shown in Table 1.

TABLE 1
Example of taVNS unit electric stimulation
output parameters for migraine.

| Output | Parameter | |
|---|---|---|
| Power supply | Direct current 3-9 volts | |
| Pulse width | 0.05-0.25 | ms |
| Frequency | 1-25 | Hz |

| | |
|---|---|
| Modes | Bi-phasic waveform |
| Intensity | 0.1-5.0 mA (Start at 0.1 mA, adjust at 0.1 |
| | mA increment till maximum tolerable |
| | intensity) |
| On/off time | 30 sec on/30 sec off |
| Sessions | 30-240 min/session, daily or 3 times/week, |
| | total duration 4-12 weeks |

In preferred embodiments of an automatic detection-therapy system 101 for migraine, when the processing unit 50, 401 detects one of the following: EEG signals suggestive of impending migraine and blood flow data suggestive of impending migraine, the taVNS unit 30 may be configured to start sending neuromodulating electric stimulation to the auricular branch of vagus nerve via the taVNS stimulating electrode 31, utilizing pre-determined stimulation parameters, such as shown in Table 2:

TABLE 2
Example of taVNS unit electric stimulation
output parameters for impending migraine

| Output | Parameter | |
|---|---|---|
| Power supply | Direct current 3-9 volts | |
| Pulse width | 0.05-0.25 | ms |
| Frequency | 1-25 | Hz |

| | |
|---|---|
| Modes | Bi-phasic waveform |
| Intensity | 0.1-3.0 mA (Start at 0.1 mA, adjust at 0.1 |
| | mA increment till maximum tolerable |
| | intensity) |
| On/off time | 30 sec on/30 sec off |
| Sessions | 30-200 min/session, daily or 3 times/week, |
| | total duration 4-8 weeks |

In preferred embodiments of an automatic detection-therapy system 101 for cluster headache, when the processing unit 50, 401 detects one of the following: EEG signals suggestive of cluster and blood flow data suggestive of cluster headache. the taVNS unit 30 may be configured to start sending neuromodulating electric stimulation to the auricular branch of vagus nerve via the taVNS stimulating electrode 31, utilizing pre-determined stimulation parameters, such as shown in Table 3:

TABLE 3
Example of taVNS unit electric stimulation
output parameters for cluster headache.

| Output | Parameter | |
|---|---|---|
| Power supply | Direct current 3-9 volts | |
| Pulse width | 0.05-0.45 | ms |
| Frequency | 10-30 | Hz |
| Modes | Continuous wave or sparse-dense wave | |
| Intensity | 0.1-5 | mA |

| | |
|---|---|
| Sessions | 20-60 min/session, Duration 4 weeks |

In preferred embodiments of an automatic detection-therapy system 101 for cluster headache, when the processing unit 50, 401 detects one of the following: EEG signals suggestive of impending cluster headache and blood flow data suggestive of impending cluster headache. the taVNS unit 30 may be configured to start sending neuromodulating electric stimulation to the auricular branch of vagus nerve via the taVNS stimulating electrode 31, utilizing pre-determined stimulation parameters, such as shown in Table 4:

TABLE 4
Example of taVNS unit electric stimulation output
parameters for impending cluster headache:

| Output | Parameter | |
|---|---|---|
| Power supply | Direct current 3-9 volts | |
| Pulse width | 0.05-0.45 | ms |
| Frequency | 10-30 | Hz |
| Modes | Continuous wave or sparse-dense wave | |
| Intensity | 0.1-4.0 | mA |

| | |
|---|---|
| Sessions | 20-60 min/session, Duration 3 weeks |

In preferred embodiments of an automatic detection-therapy system 101, the neuromodulating unit 300 may comprise at least one of the following components: a taVNS unit 30, a supraorbital nerve stimulation unit 301, an auriculotemporal nerve stimulation unit 302, an occipital nerve stimulation unit 303, a greater auricular nerve stimulation unit 304 and an infraorbital nerve stimulation unit 305. Examples of the stimulation parameters for the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305 for migraine and cluster headache and impending migraine and impending cluster headache are as follows:
(1). Intensity/Strength: 2-4 mA (with a range of 1-10 mA, maximum of 16 mA), adjusted for comfort to a mild tingling sensation without pain.
(2). Frequency: 60-120 Hz
(3). Pulse width (Duration): 200 to 250 microseconds.
(4). Stimulation cycle: commonly 30 seconds on/30 seconds off or continuous for 20-60 minutes depending on condition.
(5). Session duration: up to 7-9 hours (overnight) as needed, depending on condition.
(6). Waveform: usually biphasic pulses.
   Other examples of stimulation parameters for neuropsychiatric disorders for the taVNS unit 30, the supraorbital nerve stimulation unit 301, the auriculotemporal nerve stimulation unit 302, the occipital nerve stimulation unit 303 and the greater auricular nerve stimulation unit 304, are similar to or may be modified from the aforementioned parameters, such as discussed in the Background section. In some embodiments, the processing unit 50, 401, may include a closed-loop control system (a “sensor-controller-actuator” setup) (or a feedback-controlled mechanism). The closed-loop control system within the processing unit 50, 401, may be in electronic communication with the neurovascular monitoring system 500 (including the auricular EEG monitoring system 100 and cephalic blood flow monitoring system 530) and the neuromodulation unit 300. The closed-loop control system receives real-time continuous input of the wearer's 900 EEG data from the auricular EEG monitoring system 100 and real-time continuous input of the wearer's cerebral and extracranial blood flow data from the cephalic blood flow monitoring system 530 and the closed-loop control system of the processing unit 50, 401, is configured to analyze these real-time EEG data and cerebral and extracranial blood flow data, using controlling algorithms, to continuously adjust the actuating outputs to the neuromodulation unit 300. The actuating outputs includes turning on or turning off at least one component of the neuromodulation unit 300 and adjusting the stimulating parameters (intensity, frequency, duration, cycle, wave form etc.) of the neuromodulating unit 300 during the time when the neuromodulating unit 300 is turned on. This will greatly enhance the effectiveness and safety of neuromodulation, especially trigeminal nerve stimulation. In trigeminal nerve neuromodulation, some high-frequency (e.g., 200 Hz) stimulation could produce pronounced effects on heart rate and blood pressure compared to lower frequencies. Studies have shown that high frequency (e.g. 200 Hz) trigeminal nerve electric stimulation can produce a rapid, transient decrease in heart rate (bradycardia) and, in some cases, a sudden, brief drop in blood pressure (hypotension) due to activation of the trigeminal cardiac reflex. Both taVNS and trigeminal nerve stimulation are known to be effective for migraine and cluster headache. Studies have shown that trigeminal nerve stimulation has stronger (FDA-cleared) evidence specifically for acute and preventive migraine treatment, while taVNS has particular strength in reducing migraine frequency and treating cluster headache. High frequency trigeminal nerve stimulation (in the 100-150 Hz range) is generally considered more effective for acute migraine and cluster headache treatment, while specific lower frequency combinations are used for prevention of migraine and cluster headache. Using controlled-loop control system together with blood flow monitoring from auricular SCOS unit 511 and blood pressure monitoring from the PPG unit 555 and EEG data from the auricular EEG monitoring system 100 can minimize the risk when high frequency is used for trigeminal nerve stimulation. When any of the following: the blood flow data, the blood pressure data and the EEG data fall out of desirable range (for example: blood flow decreased by 20% or more, blood pressure decreased by 20 mmHg or more and EEG relative delta power increase above a certain level or EEG delta/alpha ratio increase above a certain level) the stimulation may be immediately stopped or the stimulation parameters may be immediately altered. High frequency (around 100-160 Hz) occipital nerve stimulation has also been found to be more effective than low frequency for migraine and cluster headache. Alternating frequencies (2/100 Hz) occipital nerve stimulation has also been found to be effective.

In preferred embodiments for automatic detection-therapy system 101, the settings or parameters for neuromodulating electric stimulation by each component of the neuromodulation unit 300 (including: the taVNS unit 30, the supraorbital nerve stimulation unit, the auriculotemporal nerve stimulation unit 302, the occipital nerve stimulation unit 303, the greater auricular nerve stimulation unit 304 and the infraorbital nerve stimulation unit 305) are pre-determined to have the most effective parameter for migraine and cluster headache and for impending migraine and impending cluster headache regarding each component of the neuromodulation unit 300. Similar or different stimulation parameters may be utilized for migraine and cluster headache and for impending migraine and impending cluster headache. As disclosed herein, an automatic detection-therapy system 101 having the novel integration of a neurovascular monitoring system 500 and a neuromodulation unit 300 achieves very important dual functions, namely long-term EEG monitoring and blood flow monitoring from the ear 902 and automatic instant therapeutic intervention by at least one component of the neuromodulation unit 300 in response to specific EEG findings and blood flow data.

In preferred embodiments, for most patients whose migraine or cluster headache is usually on one side of the head 901, one neurovascular monitoring system 500 placed on the same side may be preferred. These patients might use one neuromodulation unit 500 on the same side (although, optionally, two neuromodulation units 500, with one unit on each side of the head, might be considered). In other embodiments, for some patients with migraine or cluster headache, two neurovascular monitoring systems 500, one on each side of the head 901, may be considered. For some patients with migraine or cluster headache, two neuromodulation units 300 may be considered, with one neuromodulation unit 300 on each side of the head 901. In some other embodiments, a second neuromodulation unit with a second taVNS unit 30, a second auriculotemporal nerve stimulation unit 302 and a second greater auricular nerve stimulation unit 304 will be utilized. (Bilateral neuromodulation units 300 with one taVNS unit 30, one auriculotemporal nerve stimulation unit 302 and one greater auricular nerve stimulation unit 304 placed in each external ear 902 of the wearer 900.) However, in rare situations when a patient cannot tolerate side effects from a taVNS unit 300 on one of the ears 902, a single taVNS unit 30 may be utilized in the other side/ear 902. This may be preferred if right-sided vagus nerve stimulation from taVNS unit 30 produces any bradycardia or other side effects. Bilateral vagal stimulation is usually more effective than unilateral vagal stimulation. However, vagal stimulation may be performed only on left side if the patient or wearer 900 has side effect from right-sided vagal stimulation.

In some embodiments, one or more elements of an automatic detection-therapy system 101 may be housed in a hearing aid style structure. The traditional hearing aids include in-the-ear-hearing-aid style and behind-the-ear-hearing-aid style and both styles have close contact with the skin of tragus 905, cavum-concha 907, and external ear canal 904. These areas are the optimal locations for attachment of the elements of the present invention. (FIG. 4 shows anatomy of external ear.) Combining elements of the present invention with a hearing aid will be a welcoming set-up for patients who need hearing aids and neuromodulation. For patients who do not need hearing aids, a modified earbud housing 61, a modified in-the-ear housing 62, or a behind-the-ear-hearing-aid-style housing 63 may be used for housing of an automatic detection-therapy system 101 of the present invention. These locations and structures provide inherently secure and stable attachment.

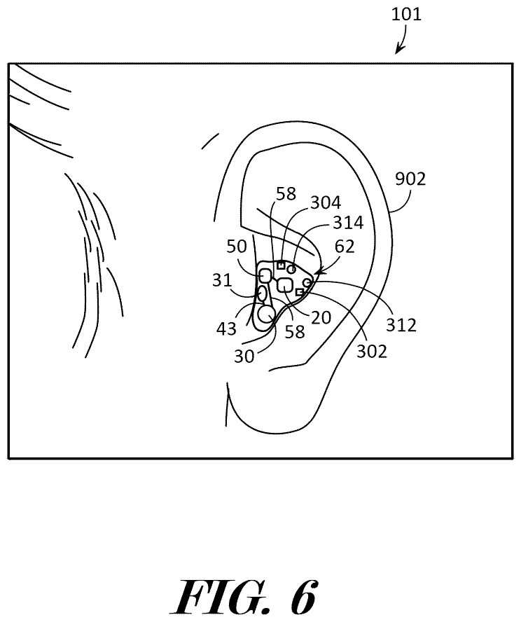

In some embodiments, a processing unit 50 can be incorporated within an auricular housing 11 of the automatic detection-therapy system 101 and housed in one of: a modified earbud housing 61, a modified in-the-ear housing 62, and a behind-the-ear-hearing-aid-style housing 63. (modified in-the-ear housing shown in FIGS. 3, 6). If the processing unit 50 is housed at a location very close to the auricular EEG recording module 20 and taVNS unit 30, they can be connected through wire type local interface 58, instead of through wireless communication. For example, if the processing unit 50 is housed within a behind-the-ear-hearing-aid-style housing 63, the processing unit 50 can be connected with the EEG recording module 20 and the taVNS 30 through wire type local interfaces 58. Alternatively, the processing unit 401 may use wireless communication with the EEG recording module 20, network interface 406 and the neuromodulation unit 300 (including one of the following components: a taVNS unit 30, a supraorbital nerve stimulation unit 301, an auriculotemporal nerve stimulation unit 302, an occipital nerve stimulation unit 303, a greater auricular nerve stimulation unit 304 and an infraorbital nerve stimulation unit 305) and the processing unit may be housed remotely as a component of a smart phone type of client device 400 or a smart watch or a health tracker with an app. The processing unit 50, 401, may also be an independent processing device, which is wearable or portable or handheld.

In some embodiments, an auricular EEG monitoring system 100 and/or an automatic detection-therapy system 101, may have an auricular housing 11 configured as one of the following: a modified earbud housing 61 (such as earphone, earbud or air-pod structures), a modified in-the-ear housing 62 (structure similar to an in-the ear hearing aid), a behind-the-ear-hearing-aid-style housing 63 (structure similar to a behind-the-ear hearing aid) and a tubular-shaped structure 66 (as part of the 61, 62 or 63 or as a standalone tubular-shaped structure 66, as further explained hereinafter). In some embodiments, an automatic detection-therapy system 101 may have an auricular housing 11. The auricular housing 11 may be configured to house all or portion of the following: all of the EEG sensor electrodes 12, 13, 72, 73, 82, 83, and an optional reference electrode 84 of an EEG recording module 20, an auricular SCOS sensor 512 of an auricular speckle contrast optical spectroscopy unit (auricular SCOS unit) 511, a taVNS stimulating electrode 31, an auriculotemporal nerve stimulation electrode 312 and a greater auricular nerve stimulating electrode 314. These housing structures are inherently stable for secure attachment. They can be easily removed temporarily for power source 16 (battery) re-charging and can be put back in place easily. Nowadays, earphones, air-pods and earbuds have become quite popular. They are nice looking and well accepted by most people. These housing structures enable easy and convenient long-term monitoring and automatic therapeutic intervention of migraine and cluster headache.

In preferred embodiments, an automatic detection-therapy system 101 may have an auricular housing 11 that may be configured as a modified earbud housing 61. The modified earbud housing 61 includes a tubular-body portion 25 and a stem portion 68. (FIG. 2). The stem portion 68 is equivalent to a “stem” (or a “stalk”) of an earbud. The tubular-body portion 25 includes a tubular-shaped structure 66 (to be placed inside a wearer's external ear canal 904 when in use) and a body-structure 67 (to be placed immediately at the opening of the wearer's external ear canal 904 and be placed inside the tragus-concha bowl 957 when in use). The tubular-shaped structure 66 is equivalent to a modified elongated version of an “ear-tip” and a “nozzle” of an earbud. The body-structure 67 is equivalent to a “body” (or a “shell”) of an earbud. The EEG recording module 20 may include an optional reference electrode 84. All of the EEG sensor electrodes 12, 13, 72, 73, 82, 83, may be configured to be placed on a surface 91 of the tubular-shaped structure 66. All of the EEG sensor electrodes 12, 13, 72, 73, 82, 83, are configured to be partially embedded in the surface 91 with slight protrusion at the surface 91 of the tubular-shaped structure 66. The auricular SCOS sensor 512, the taVNS stimulating electrode 31 and the auriculotemporal nerve stimulating electrode 312 may be configured to be placed on a surface and partially embedded in the surface with slight protrusion at the surface of one of: the body-structure 67 and the tubular-shaped structure 66. (Alternatively, the auricular SCOS sensor 512 may be placed slightly below the surface of the body-structure 67 or the tubular-shaped structure 66 so that there is a small air gap between the SCOS sensor 512 and the wearer's skin when the body-structure 67 and the tubular-shaped structure 66 are placed in the wearer's ear 902). The greater auricular nerve stimulating electrode 314 and the optional reference electrode 84 may be configured to be placed on the surface (and partially embedded in the surface with slight protrusion at the surface) of the body-structure 67. Preferably the tubular-body portion 25 (including the tubular-shaped structure 66 and the body-structure 67) of the modified earbud housing 61 may be made with or may comprise an elastic flexible and adaptable material (such as silicone), in which the material for the tubular-body portion 25 is configured to have appropriate elasticity, flexibility and adaptability so that the tubular-shaped structure 66 will naturally adapt to the contour of the wearer's external ear canal 904 and naturally fill the interior of the wearer's external ear canal 904 when the tubular-shaped structure 66 is inserted into the wearer's external ear canal 904. At the same time, the body-structure 67 will naturally adapt to the contour of the wearer's tragus-concha bowl 957 and naturally fill the interior of the wearer's tragus-concha bowl 957 when the body-structure 67 is placed in the wearer's ear 902 (in the tragus-concha bowl 957). This set-up and the elasticity, flexibility and adaptability of the material will allow all of the EEG sensor electrodes 12, 13, 72, 73, 82, 83, to be naturally in close contact with the skin of the wearer's external ear canal 904 when the tubular-shaped structure 66 is inserted into the wearer's external ear canal 904; at the same time, the auricular SCOS sensor 512, the taVNS stimulating electrode 31, and the auriculotemporal nerve stimulating electrode 312 will be naturally in close contact with the skin of the wearer's external ear canal 904 or skin of the wearer's tragus-concha bowl 957 when the tubular-body portion 25 is placed in the wearer's ear 902; and at the same time, the greater auricular nerve stimulation electrode 314 and the optional reference electrode 84 will be naturally in close contact with the skin of the wearer's tragus-concha bowl 957 when the tubular-body portion 25 in placed in the wearer's ear 902. Thus, at the same time, the taVNS stimulating electrode 31 is naturally and snugly in contact with its target skin of vagus-innervated auricular skin of the wearer's first ear 902, provided by carefully selecting a location for the taVNS stimulating electrode 31 on the tubular-shaped structure 66 or the body-structure 67 to match one of the innervation locations of the vagus innervated skin on the external ear canal 904 or the tragus-concha bowl 957, and, at the same time, the auriculotemporal nerves stimulating electrode 312 will be naturally in close contact with its target skin of auriculotemporal nerve innervated auricular skin provided by carefully selecting a location for the auriculotemporal nerve stimulating electrode 312 on the tubular-shaped structure 66 or the body-structure 67 to match one of the innervation locations of the auriculotemporal nerve innervated skin on the external ear canal 904 or the tragus-concha bowl 957, and furthermore, at the same time, the greater auricular nerve stimulating electrode 314 will be naturally in close contact with greater auricular nerve innervated auricular skin, provided by carefully selecting a location for the greater auricular nerve stimulating electrode 314 on the body-structure 67 to match one of the innervation locations of the greater auricular nerve innervated skin on tragus-concha bowl 957. (FIG. 11). (This is feasible due to the proximity of the innervation patterns of the auricular branch of vagus nerve, the auriculotemporal nerve and the greater auricular nerve in the auricular area, and by careful selection of the locations for 31, 312, 314, to match the locations of their respective target skin on the wearer's ear, as described hereinbefore.) For the wearer 900, attaching and removing these electrodes 12, 13, 72, 73, 82, 83, 84, 31, 312, 314, will be as easy as inserting and removing the tubular-body portion 25 of the modified earbud housing 61 from the wearer's external ear 902. There will be no need for a certified technologist to apply the electrodes. Applying adhesive material to secure these electrodes 12, 13, 72, 73, 82, 83, 84, 31, 312, 314, will also be unnecessary. This is feasible due to the unique anatomical features of the human external ear canal 904 and the cavity/bowl shape of the tragus-concha bowl 957 as illustrated in FIG. 11. This will create huge convenience for the wearer. Thus, the automatic detection-therapy system 101, is wearable, self-installable, self-removable, ambulatory and very convenient for wearers (users). This housing design will enable a very convenient multi-mode neuromodulation system to do double neuromodulation (simultaneous neuromodulation of two different nerves) or triple neuromodulation (simultaneous neuromodulation of three different nerves) since 2 or 3 components of the neuromodulation unit are conveniently housed together in a same auricular housing 11 (such as the modified earbud housing 61) as described herein.

These advantages can be similarly achieved when the automatic detection-therapy system 101 comprises a behind-the-ear-hearing-aid-style housing 63. In some embodiments, for example, an automatic detection-therapy system 101 may have an auricular housing 11 that may be configured as a behind-the-ear-hearing-aid-style housing 63. The behind-the-ear-hearing-aid-style housing 63 includes an in-the-ear portion 27 and a behind-the-ear portion 26. The in-the-ear portion 27 is essentially the same as the tubular-body portion 25 of a modified earbud housing 61. The in-the-ear portion 27 includes a tubular-shaped structure 66 (configured to be placed inside the wearer's external ear canal 904 when in use) and a body-structure 67 (configured to be placed at the immediate opening of the wearer's external ear canal 904 and be placed inside the tragus-concha bowl 957 when in use). The in-the-ear portion 27 may be configured to be made with elastic flexible and adaptable material (such as silicone), in which the material for the in-the-ear portion 27 is configured to have appropriate elasticity, flexibility and adaptability so that the in-the-ear portion 27 will naturally adapt to the contour of the wearer's external ear canal 904 and the contour of the wearer's tragus-concha bowl 957 and will naturally fill the interior of the external ear canal 904 and the interior of the tragus-concha bowl 957 when it is placed is the wearer's ear 902. All of the EEG sensor electrodes 12, 13, 72, 73, 82, 83, may be placed on a surface 91 of the tubular-shaped structure 66 of the in-the-ear portion 27. All of the EEG sensor electrodes 12, 13, 72, 73, 82, 83 may be partially embedded in the surface 91 with slight protrusion at the surface 91 of the tubular-shaped structure 66 of the in-the-ear portion 27 so that all of the EEG sensor electrodes 12, 13, 72, 73, 82, 83, may be naturally in close contact with the skin of the wearer's external ear canal 904 when the tubular-shaped structure 66 is inserted into the wearer's external ear canal 904. The EEG recording module 20 may further include an optional reference electrode 84. The auricular SCOS sensor 512, the PPG unit 555, the taVNS stimulating electrode 31 and the auriculotemporal nerve stimulating electrode 312 may be configured to be placed on a surface (and partially embedded in the surface with slight protrusion at the surface) of the in-the-ear portion 27 (in either the tubular-shaped structure 66 or the body-structure 67) so that when the in-the-ear portion 27 is placed in the wearer's external ear canal 904 and the tragus-concha bowl 957, the auricular SCOS sensor 512, the PPG unit 555, the taVNS stimulating electrode 31 and the auriculotemporal nerve stimulating electrode 312 will all be naturally in close contact with the skin of the wearer's external ear canal 904 or the skin of the wearer's tragus-concha bowl 957. (Alternatively, the auricular SCOS sensor 512 may be placed slightly below the surface of the body-structure 67 or tubular-shaped structure 66 so that there is a small air gap between the SCOS sensor 512 and the wearer's skin when the body-structure 67 and the tubular-shaped structure 66 are placed in the wearer's ear 902). The greater auricular nerve stimulating electrode 314 and the optional reference electrode 84 may be configured to be placed on the surface (and partially embedded in the surface with protrusion at the surface) of the body-structure 67 so that when the body-structure 67 is placed in the wearer's tragus-concha bowl 957 the greater auricular nerve stimulating electrode 314 and the optional reference electrode 84 will be naturally snugly in close contact with the skin of the wearer's tragus-concha bowl 957. At the same time, the taVNS stimulating electrode 31 will be naturally in close contact with vagus innervated auricular skin since external ear canal 904 and the tragus-concha bowl 957 are part of vagus innervated auricular skin. Meanwhile, the auriculotemporal nerve stimulating electrode 312 will also be naturally in close contact with auriculotemporal nerve innervated auricular skin since external ear canal 904 and the tragus-concha bowl 957 are also part of auriculotemporal nerve innervated auricular skin. Further, the greater auricular nerve stimulating electrode 314 will be naturally in close contact with the greater auricular nerve innervated auricular skin since cavum concha (part of tragus-concha bowl 957) is part of the greater auricular nerve innervated auricular skin. Thus, the taVNS stimulating electrode 31 will be naturally in close contact with the vagus innervated auricular skin, provided by carefully selecting a location for the taVNS stimulating electrode 31 on the tubular-shaped structure 66 or the body-structure 67 to match one of the innervation locations of the vagus innervated skin on the external ear canal 904 or the tragus-concha bowl 957, and, at the same time, the auriculotemporal nerves stimulating electrode 312 will be naturally in close contact with auriculotemporal nerve innervated auricular skin, provided by carefully selecting a location for the auriculotemporal nerve stimulating electrode 312 on the tubular-shaped structure 66 or the body-structure 67 to match one of the innervation locations of the auriculotemporal nerve innervated skin on the external ear canal 904 or the tragus-concha bowl 957, and, at the same time, the greater auricular nerve stimulating electrode 314 will be naturally in close contact with greater auricular nerve innervated auricular skin, provided by carefully selecting a location for the greater auricular nerve stimulating electrode 314 on the body-structure 67 to match one of the innervation locations of the greater auricular nerve innervated skin on tragus-concha bowl 957. (This is feasible due to the proximity of the innervation patterns of the auricular branch of vagus nerve, the auriculotemporal nerve and the greater auricular nerve in the auricular area, and by careful selection of the locations for 31, 312, 314, to match the locations of their respective target skin on the wearer's ear, as described hereinbefore.) For a wearer 900, installing and removing all of these electrodes 12, 13, 72, 73, 82, 83, 84, 31, 312, 314, 512 and PPG unit 555 will be as easy as placing and removing the in-the-ear portion 26 (of the behind-the-ear-hearing-aid-style housing 63) from the wearer's ear 902. There will be no need for a certified technologist to install all of these electrodes 12, 13, 72, 73, 82, 83, 84, 31, 312, 314, 512 and PPG unit 555. Applying adhesive material to secure these electrodes 12, 13, 72, 73, 82, 83, 31, 312, 314, will also be unneeded. Thus, this automatic detection-therapy system 101 is fully wearable, user-installable, user-removable, ambulatory and very convenient for wearers (users). (For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve innervated auricular skin includes: anterior outer part of tragus 905, anterior-superior part of cavum concha 907, anterior and superior walls of the external ear canal 904 and, anterior and superior part of pinna including anterior-superior helix 909.) This housing design will provide a novel multi-mode neuromodulation system when double neuromodulation (simultaneous neuromodulation of two different nerves) or triple neuromodulation (simultaneous neuromodulation of three different nerves) are housed in a same auricular housing 11, such as the behind-the-ear-hearing-aid-style housing 63, as described herein.

In some embodiments, the aforementioned advantages can also be similarly achieved when an auricular housing 11 of an automatic detection-therapy system 101 may be shaped or configured as a modified in-the-ear housing 62. The modified in-the-ear housing 62 is essentially similar to the in-the-ear portion 27 of a behind-the-ear-hearing-aid-style housing 63. The modified in-the-ear housing 62 is also essentially the same as a tubular-body portion 25 of a modified earbud housing 61. The modified in-the-ear housing 62 also includes a tubular-shaped structure 66 and a body-structure 67. The tubular-shaped structure 66 will be placed in a wearer's external ear canal 904 when in use. The body-structure 67 will be placed at the opening of the wearer's external ear canal 904 and be placed inside a tragus-concha bowl 957 of a wearer's ear 902 when in use. Preferably, all of the EEG sensor electrodes 12, 13, 72, 73, 82, 83, may be configured to be located at a surface 91 and partially embedded in the surface 91 with slight protrusion at the surface 91 of the tubular-shaped structure 66. Preferably, an auricular SCOS sensor 512, a taVNS stimulating electrode 31 and an auriculotemporal nerve stimulating electrode 312 may be configured to be located at a surface and partially embedded in the surface with slight protrusion at the surface of one of: the body-structure 67 and the tubular-shaped structure 66. (Alternatively, the auricular SCOS sensor 512 may be placed slightly below the surface of the body-structure 67 or the tubular-shaped structure 66 so that there is a small air gap between the SCOS sensor 512 and the wearer's skin when the body-structure 67 and the tubular-shaped structure 66 are placed in the wearer's ear 902). Preferably, a greater auricular nerve stimulating electrode 314 and an optional reference electrode 84 may be configured to be located at the surface (and partially embedded in the surface with protrusion at the surface) of the body-structure 67. Preferably, the modified in-the-ear housing 62 may be made with or may comprise elastic flexible and adaptable material (such as silicone), in which the material for the modified in-the-ear housing 62 is configured to have appropriate elasticity flexibility and adaptability so that when the modified in-the-ear housing 62 is placed in a wearer's external ear 902, the tubular-shaped structure 66 will naturally adapt to the contour of wearer's external ear canal 904 and will snugly fill the interior of the wearer's external ear canal 904 and, meanwhile, the body-structure 67 will naturally adapt to the contour of the wearer's tragus-concha bowl 957 and fill the interior of the tragus-concha bowl 957. This set-up and the elasticity flexibility and adaptability of the material will enable all of the EEG sensor electrodes 12, 13, 72, 73, 82, 83, to be naturally in close contact with the skin of the wearer's external ear canal 904; meanwhile the taVNS stimulating electrode 31, the auriculotemporal nerve stimulating electrode 312 and the auricular SCOS sensor 512 to be naturally in close contact with the skin of the wearer's external ear canal 904 or skin of the tragus-concha bowl 957, and, at the same time, the greater auricular nerve stimulating electrode 314 and the optional reference electrode 84 will be naturally in close contact with the skin of tragus-concha bowl 957, when the modified in-the-ear housing 62 is placed in the wearer's ear 902. At the same time, the taVNS stimulating electrode 31 will be naturally in close contact with the wearer's vagus innervated auricular skin and the auriculotemporal nerve stimulating electrode 312 will be naturally in close contact with the wearer's auriculotemporal nerve innervated auricular skin since the skin of the external ear canal 904 and skin of the tragus-concha bowl 957 received mixed innervation from the auricular branch of vagus nerve and the auriculotemporal nerve. Furthermore, the greater auricular nerve stimulating electrode 314 will be naturally in close contact with greater auricular nerve innervated auricular skin, similar to the aforementioned descriptions. Thus, this system 101 is wearable, user-installable, user-removable, fully ambulatory and very convenient for wearers (users). This housing design will provide a novel multi-mode neuromodulation system when double neuromodulation (simultaneous neuromodulation of two different nerves) or triple neuromodulation (simultaneous neuromodulation of three different nerves) are housed in a same auricular housing 11, such as the in-the-ear housing 62, as described herein.

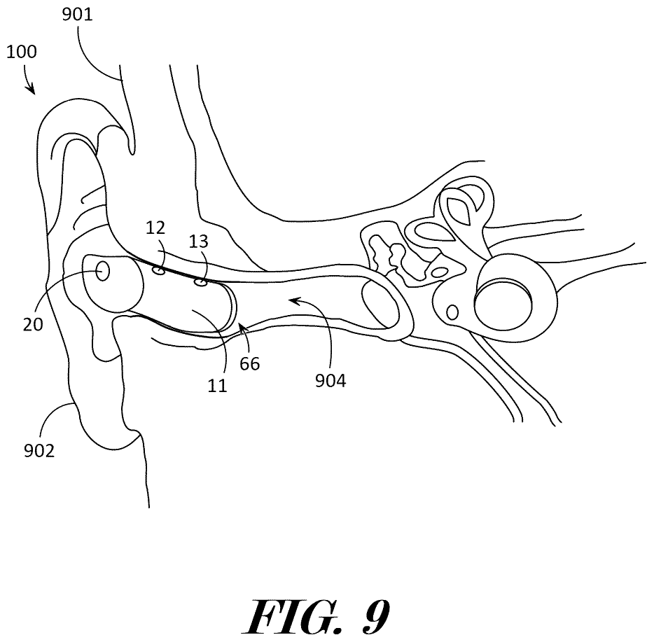

In some embodiments, a standalone tubular-shaped structure 66 may be configured as a housing choice for an auricular EEG monitoring system 100 and/or an automatic detection-therapy system 101. (FIG. 9). Obviously, the aforementioned modified earbud housing 61, modified in-the-ear housing 62 and behind-the-ear-hearing-aid-style housing 63 all comprise a tubular-shaped structure 66. Thus, all of the aforementioned descriptions for the tubular-shaped structure 66 can be applied to a standalone tubular-shaped structure 66 as a housing choice for system 100 and system 101. For an auricular EEG monitoring system 100, all of the EEG sensor electrodes 12, 13, 72, 73, 82, 83, may be configured to be located on a surface and partially embedded in the surface with slight protrusion at the surface of the tubular-shaped structure 66. For an automatic detection-therapy system 101, all of the EEG sensor electrodes 12, 13, 72, 73, 82, 83, an auricular SCOS sensor 512, a taVNS stimulating electrode 31 and an auriculotemporal nerve stimulating electrode 312 may be configured to be located on a surface and partially embedded in the surface with slight protrusion at the surface of the tubular-shaped structure 66, with the setups, functions and advantages similar to the aforementioned descriptions. There will be no need to have an optional reference electrode 84 when a standalone tubular-shaped structure 66 is used for housing for system 100 or system 101 because the external ear canal 904 may not be an ideal location for a reference electrode 84 (although it may be feasible to place a reference electrode 84 at an inferior surface of the tubular-shaped structure 66.). Instead, average of all of the EEG sensor electrodes 12, 13, 72, 73, 82, 83, can be used as a reference (common average reference). The greater auricular nerve stimulating electrode 314 cannot be placed in the wearer's external ear canal 904.

In some embodiments, a separate client device 400 may be used for housing of one or more of the components of an auricular EEG monitoring system 100 and/or an automatic detection-therapy system 101. For example, an EEG recording module 20 and the processing unit 401 may be housed remotely in a wearable client device 400, such as a smart watch-type device or a smart phone type device. The EEG sensor electrodes 12, 13, 72, 73, 82, 83, the optional reference electrode 84, the auricular SCOS unit 511, the taVNS stimulation unit 30, the auriculotemporal nerve stimulation unit 302 and the greater auricular nerve stimulation unit 304 may be housed in one of: a modified earbud housing 61, a modified in-the-ear housing 26 and in an in-the-ear portion 27 of a behind-the-ear-hearing-aid-style housing 63. Wireless EEG sensor electrodes may be used for all of the EEG sensor electrodes 12, 13, 72, 73, 82, 83 and the optional reference electrode 84. (Wireless dry electrodes for in-ear EEG such as that described by Ryan Kaveh et al. in Nature Communications on Aug. 2, 2024. Wireless dry EEG electrodes are also available from Zeto, Inc. headquarter in Santa Clara, California.) There are well-known wired or wireless EEG amplifiers available, such as EEG Electroencephalogram Smart Amplifier (Part #: EEG100D), or preferably BioNomadix 2Ch Wireless EEG Amplifier (Part #: BN-EEG2), both being made by the same company BIOPAC Systems, Inc. (Goleta, California). By using wireless EEG electrodes 12, 13, 72, 73, 82, 83, wireless optional reference electrode 84 and wireless EEG amplifier 21, the EEG recording module 20, together with the processing unit 401, may be housed remotely in a wearable client device 400 (such as a watch-type device or a smart phone type device) and communicate wirelessly with all of the EEG sensor electrodes 12, 13, 72, 73, 82, 83 and the optional reference electrode 84.

In some embodiments, an auricular EEG monitoring system 100 and/or an automatic detection-therapy system 101 may comprise a network interface 53. The network interface 53 is in electronic communication with a speaker 15, 404A, and/or a vibrator 17, 404B, which may be configured to generate an audible and/or tactile notification to a wearer 900, or the wearer's healthcare provider 950, or other individual.

A speaker 15, 404A, may comprise a sound emitting device which can provide audible notification function. A speaker of a speaker 15, 404A, may comprise a buzzer, a piezoelectric sound producing device, a dielectric elastomer sound producing device, a buzzer, a moving coil loudspeaker, an electrostatic loudspeaker, an isodynamic loudspeaker, a piezoelectric loudspeaker, or any other device capable of producing one or more sounds. In preferred embodiments, an auricular EEG monitoring system 100 and/or an automatic detection-therapy system 101 may comprise a speaker 15, 404A, that may be in electronic communication with a network interface 53, 406, and/or a processing unit 50, 401. The speaker 15, 404A, may be configured to generate an audible notification when the processing unit 50, 401, detects at least one of the following: presence of EEG signals suggestive of migraine or cluster headache, presence of blood flow data suggestive of migraine or cluster headache, presence of EEG signals suggestive of impending migraine or impending cluster headache and presence of blood flow data suggestive of impending migraine or impending cluster headache. The speaker 15, 404A, may be also configured to generate an audible notification when the processing unit detects all of the following: cessation of EEG signals suggestive of migraine or cluster headache, cessation of EEG signals suggestive of impending migraine or impending cluster headache, cessation of blood flow data suggestive of migraine or cluster headache, cessation of blood flow data suggestive of impending migraine or impending cluster headache.

A vibrator 17, 404B, may comprise a weight that may be rapidly moved by a long life brushless (BLDC) vibration motor, a coin or pancake vibration motor, an encapsulated vibration motor, an enclosed vibration motor, a pager motor, an eccentric rotating mass (ERM) motor, a linear resonant actuator (LRA), a printed circuit board (PCB) mounted vibration motor, or any other electrical device capable of producing a series of rapid and repeated movements. In preferred embodiments, an auricular EEG monitoring system 100 and/or an automatic detection-therapy system 101 may comprise a vibrator 17, 404B, that may be in electronic communication with a network interface 53, 406, and/or a processing unit 50, 401. The vibrator 17, 404B, may be configured to generate a tactile notification when the processing unit 50, 401, detects at least one of the following: presence of EEG signals suggestive of migraine or cluster headache, presence of blood flow data suggestive of migraine or cluster headache, presence of EEG signals suggestive of impending migraine or impending cluster headache and presence of blood flow data suggestive of impending migraine or impending cluster headache. The vibrator 17, 404B, may be also configured to generate a tactile notification when the processing unit detects all of the following: cessation of EEG signals suggestive of migraine or cluster headache, cessation of EEG signals suggestive of impending migraine or impending cluster headache, cessation of blood flow data suggestive of migraine or cluster headache and cessation of blood flow data suggestive of impending migraine or impending cluster headache.

In some embodiments, an auricular EEG monitoring system 100 and an automatic detection-therapy system 101 may comprises a switch 88 and a timer 89 to enable the wearer 900 to turn on the system 100 or system 101 manually and setting a duration, an interval or a schedule etc. Preferably, the switch 88 may be configured as a multi-mode switch 88 and the timer 89 may be configured as a multi-mode timer 89. In some embodiments, a multi-mode switch 88 may be configured to enable the wearer to set the system 100 and system 101 (including system 101 for migraine and system 101 for cluster headache) at an automatic mode or a manual mode. The functions and setups of the automatic mode for system 101 for migraine and system 101 for cluster headache are as described hereinbefore. In some embodiments for an automatic detection-therapy system 101, the multi-mode switch 88 may be configured to allow the wearer 900 to choose at least one of the following switch selections: turning on or off an auricular EEG monitoring system 100, turning on or off an auricular SCOS unit 511, turning on or off a taVNS unit 30, turning on or off a supraorbital nerve stimulation unit 301, turning on or off an auriculotemporal nerve stimulation unit 302, turning on or off an occipital nerve stimulation unit 303, turning on or off a greater auricular nerve stimulation unit 304, turning on or off an infraorbital nerve stimulation unit 305, or various combinations thereof: The multi-mode timer 89 may be configured to function together with the multi-mode switch 88 to set each switch selection at desirable timing courses such as duration, interval, countdown, daily/weekly schedule, 10 minute/30 minutes/60 minutes (or other duration) selection, continuous operation, manual turning on and manual shutdown etc. Furthermore, the multi-mode switch 88 and multi-mode timer 89 may be configured as a multifunctional switch-timer or as a programmable multifunctional timer-switch. In some embodiments, the programmable multifunctional timer-switch may be incorporated in a client device 400 of a wearer 900 to allow the wearer flexibility and ease in choosing manual or automatic control and choosing various time courses and various switch selections (selection of various combinations of the 6 components of the neuromodulation unit, including the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305.) In some embodiments, the multi-mode switch 88 function and the multi-mode timer 89 function may be configured to be incorporated into a wearer's client device 400 and can be easily set up as programmable (programmable setup as known in the art). In the manual mode, the wearer 900 can use the taVNS unit 30, the unit 301, unit 302, unit 303, unit 304, unit 305 or various combinations thereof for prophylactic purpose against neuropsychiatric disorders (including migraine and cluster headache) or for health maintenance purpose. Studies have shown that the taVNS unit 30 is effective not only for therapy, but also for prophylaxis, for various neuropsychiatric disorders. Studies have also shown that the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305 (and various combinations thereof) are effective not only for therapy, but also for prophylaxis, for various neuropsychiatric disorders. The aforementioned programmable set up for the system 101 will enable the system 101 to be used for prophylactic purpose and health maintenance purpose. The multi-mode switch 88, multi-mode timer 89 and the programmable design may enable the wearer the option to choose from one of the following: neuromodulation from one neuromodulation component (single neuromodulation), simultaneous neuromodulation from two neuromodulation components (double neuromodulation) and simultaneous neuromodulation from three neuromodulation components (triple neuromodulation). Thus, when prompted, the neuromodulation unit 300 is configured to generate neuromodulating electric stimuli to a wearer 900 according to the choice of the stimulation mode selected by the wearer 900. The choices of the stimulation mode include the following: single neuromodulation by one component of the neuromodulation unit 300, double neuromodulation by various combinations of two components of the neuromodulation unit 300, triple neuromodulation by various combinations of three components of the neuromodulation unit 300, etc. In alternate embodiments, an automatic detection-therapy system 101 for neuropsychiatric disorders may comprise a first and a second EEG recording modules 20 and a first and a second neuromodulation units 300 (as described hereinbefore) and the system 101 may further comprise a multi-mode switch 88 and a multi-mode timer 89 (or a programmable multi-functional switch-timer) that are configured to be linked to both the first and the second EEG recording modules 20 and the first and the second neuromodulation units 300, and are configured to enable the wearer 900 to select stimulation components from both the first and the second neuromodulation units 300 and to set time courses involving stimulation components in both the first and the second neuromodulation units 300.

As perhaps best shown by FIG. 18, an illustrative example of some of the physical components which may be used with an auricular EEG monitoring system 100 and/or an automatic detection-therapy system 101, according to some embodiments are presented. An auricular EEG monitoring system 100 and an automatic detection-therapy system 101 may be configured to facilitate the transfer of data and information between one or more access points 103, client devices 400, and servers 800 over a data network 105. Client devices 400 and servers 800 may send data to and receive data from the data network 105 through a network connection 104 with an access point 103. A data store 308 accessible by the server 800 may contain one or more databases. The data may comprise any data recorded and generated by an auricular EEG monitoring system 100 and/or an automatic detection-therapy system 101. In this example, an auricular EEG monitoring system 100 and/or an automatic detection-therapy system 101 may comprise or be in communication with at least one client device 400 (but preferably two or more than two client devices 400) configured to be operated by one or more users 900, 950. Client devices 400 may include mobile devices, such as laptops, tablet computers, personal digital assistants, smart phones, smart watches, and the like, that are equipped with a wireless network interface 406 capable of sending data to one or more servers 800 with access to one or more data stores 308 over a network 105, such as a wireless local area network (WLAN). Additionally, client devices 400 may include fixed devices, such as desktops, workstations, and the like, that are equipped with a wireless or wired network interface capable of sending data to one or more servers 800 with access to one or more data stores 308 over a wireless or wired local area network 105. The present invention may be implemented on at least one computing device, such as a client device 400 and/or server 800, programmed to perform one or more of the steps described herein. In some embodiments, more than one client device 400 and/or server 800 may be used, with each being programmed to carry out one or more steps of a method or process described herein.

According to yet another aspect consistent with the principles of this invention, a cephalic blood flow monitoring complex for migraine and cluster headache detection is disclosed. In some embodiments, a cephalic blood flow monitoring complex for migraine and cluster headache detection comprises a cephalic blood flow monitoring system 530, a network interface 53 and a processing unit 50, 401. The cephalic blood flow monitoring system 530 may be configured to record cerebral and extracranial blood flow data of a wearer. The processing unit 50, 401, may be configured to be in electronic communication with the cephalic blood flow monitoring system 530 and the network interface 53. The processing unit 50, 401, may be configured to analyze the cerebral and extracranial blood flow data recorded by the cephalic blood flow monitoring system 530 to detect presence or cessation of cerebral and extracranial blood flow data suggestive of at least one of: migraine and cluster headache. The processing unit 50, 401, may be also configured to analyze the cerebral and extracranial blood flow data recorded by the cephalic blood flow monitoring system 530 to detect presence or cessation of cerebral and extracranial blood flow data suggestive of at least one of: impending migraine and impending cluster headache. When the processing unit 50, 401, detects presence of cerebral and extracranial blood flow data suggestive of at least one of: migraine and cluster headache, the processing unit 50, 401, may be configured to generate signals to the network interface 53 to prompt the network interface 53 to send notification to at least one of: a client device 400 of the wearer 900 and a client device 400 of a healthcare provider 950 of the wearer. When the processing unit 50, 401, detects presence of cerebral and extracranial blood flow data suggestive of at least one of: impending migraine and impending cluster headache, the processing unit 50, 401, may be configured to generate signals to the network interface 53 to prompt the network interface 53 to send notification to at least one of: the client device 400 of the wearer 900 and the client device 400 of the healthcare provider 950 of the wearer. When the processing unit 50, 401, detects both of the following: cessation of cerebral and extracranial blood flow data suggestive of at least one of: migraine and cluster headache and cessation of cerebral and extracranial blood flow data suggestive of at least one of: impending migraine and impending cluster headache, the processing unit 50, 401, is further configured to generate signals to the network interface 53 to prompt the network interface 53 to send notification to at least one of: the client device 400 of the wearer 900 and the client device 400 of the healthcare provider 950 of the wearer. In some embodiments, a cephalic blood flow monitoring complex for migraine and cluster headache detection may include a cephalic blood flow monitoring system 530 that comprises at least one of the followings: a photoplethysmography (PPG) unit 555, a speckle contrast optical spectroscopy unit (SCOS unit) 510, an auricular speckle contrast optical spectroscopy unit (auricular SCOS unit) 511, ultrasound-doppler based blood flow monitoring devices (such as a transcranial doppler ultrasound TCD) and other laser-based blood flow monitoring devices such as laser speckle contrast imaging (LSCI), near-infrared spectroscopy (NIRS) and diffuse correlation spectroscopy (DCS), etc. In some embodiments, a cephalic blood flow monitoring complex for migraine and cluster headache detection may include a cephalic blood flow monitoring system 530 that may comprise a photoplethysmography (PPG) unit 555 and an auricular speckle contrast optical spectroscopy unit (auricular SCOS unit) 511. The processing unit 50, 401, may be in electronic communication with the PPG unit 555 and the auricular SCOS unit 511. The PPG unit 555 and the auricular SCOS unit 511 may be both configured to be housed in an auricular housing 11 (such as a housing in cymba concha 906 or cavum concha 907, or a housing in tragus-concha bowl 957, or a housing with a body-structure 67, for example a modified earbud housing 61 or a modified in-the-ear housing 62) to be placed in an ear 902 of the wearer 900 when in use. The PPG unit 555 is configured to record the wearer's 900 cerebral and extracranial blood flow data from the wearer's ear 902, and the auricular SCOS unit 511 is also configured to record the wearer's cerebral and extracranial blood flow data from the wearer's ear 902. The PPG unit 555 and the auricular SCOS unit 511 may be housed together in the auricular housing 11 (e.g. in tragus-concha bowl 957). The housing setups and the material for the auricular housing 11 are similar to the aforementioned descriptions so that the PPG unit 555 and the auricular SCOS unit 511 are user-installable and user-removable. In alternate embodiments, the PPG unit 555 and the SCOS unit 510 may be attached to near the wearer's orbits (eyes) or temple area because orbital area and temple area have significant blood flow changes before and during migraine and cluster headache (although being housed in an auricular housing 11 offers the important advantages of being aesthetic, wearable and suitable for long-term monitoring.)

### A First Preferred Embodiment

In preferred embodiments, an automatic detection-therapy system 101 for migraine may comprise an auricular EEG monitoring system 100 having an auricular electroencephalogram (EEG) recording module 20, a neuromodulation unit 300 and a processing unit 50, 401. The auricular EEG recording module 20 may have a plurality (at least two, but preferably more than two) of miniature wired or wireless EEG sensor electrodes 12, 13, 72, 73, 82, 83 and a wired or wireless EEG amplifier 21. Optionally, the EEG recording module 20 may include an optional reference electrode 84. (The optional reference electrode 84 would be desired but is not always necessary. Instead, average of all of the EEG sensor electrodes 12, 13, 72, 73, 82, 83, can be used as a reference, i.e. common average reference). The EEG sensor electrodes 12, 13, 72, 73, 82, 83, and the optional reference electrode 84 may be configured to contact separate areas selected from at least one of the following: external ear canal 904 of a first ear, external ear 902 of the first ear or peri-auricular area 903 around the first ear. The peri-auricular area 903 refers to the portion of the head around the auricle (pinna). The peri-auricular area 903 is typically hairless. The peri-auricular area includes a portion of the head in front of the auricle (pre-auricular area) and a portion of the head above and behind the auricle (post-auricular area). The pre-auricular area is small, about one inch wide and two inches long and curved along the anterior edge of the auricle. The post-auricular area is also small and is about one inch wide and about three inches long and curved along the superior and posterior edges of the auricle (pinna). The mastoid (mastoid process) is located toward the lower end of the post-auricular area. The post-auricular area is where a behind-the-ear hearing aid is usually attached to. The preauricular area and the post-auricular area together is called “peri-auricular area” herein. (Anterior, posterior, superior, in front of and behind etc. all refer to the directions relative to the wearer's head when the wearer is in an upright position.) The optional reference electrode 84 of the EEG recording module 20 may be placed in the tragus-concha bowl 957 or at the mastoid of the peri-auricular area 903. (Tragus-concha bowl 957 and mastoid are known to be good locations for EEG reference electrode 84.) The EEG recording module 20 may be configured to record and generate EEG data of the wearer 900 using electrical activities of the wearer 900 that are picked up via all of the EEG sensor electrodes 12, 13, 72, 73, 82, 83, and the optional reference electrode 84 (if the optional reference electrode is included) (FIGS. 2, 7, 11-13). The EEG recording module 20 is in electronic communication with the processing unit 50, 401. The processing unit 50, 401 is configured to converts raw EEG data into quantitative EEG (qEEG) data through digital signal processing (DSP) techniques that transform, analyze, and mathematically quantify brain electrical activity. The processing unit 50, 401, is configured to use the EEG and qEEG data to detect migraine and impending migraine, including detecting higher absolute power (especially high beta), increased relative theta power, and increased occipito-temporal asymmetry which is detectable 36-72 hours before a migraine attack (impending migraine). More specifically, the processing unit 50, 401, is configured to use the EEG and qEEG data to detect inter-ictal phase, pre-ictal phase and ictal phase of migraine by using the following: during inter-ictal phase of migraine, migraineurs often display higher absolute power (particularly in the high beta frequency range) and increased relative theta power in all cortical regions, along with higher delta activity in the fronto-central region; during the impending migraine phase (pre-ictal phase) significantly higher asymmetry in the occipitoparietal alpha and theta bands and higher EEG coherence in delta, theta, alpha, and beta bands compared to inter-ictal states are detectable 36 hours before a migraine attack; while during migraine attack (ictal phase) the qEEG often show heightened theta/delta power and reduced alpha power, indicating a shift from a baseline state. The processing unit 50, 401, is configured to analyze these EEG and qEEG data to detect impending migraine and confirm migraine to help the wearer to take appropriate action and to actuate the neuromodulation unit 300 to start or stop neuromodulation therapy under the regulation by the closed-loop control system.

In preferred embodiments, an automatic detection-therapy system 101 for migraine may comprise a neurovascular monitoring system 500, a first neuromodulation unit 300 and a processing unit 50, 401. The neurovascular monitoring system 500 comprises an auricular EEG monitoring system 100 and a cephalic blood flow monitoring system 530. The auricular EEG monitoring system 100 is as described hereinbefore. The cephalic blood flow monitoring system 530 is configured to assess the cerebral (intracranial) and extracranial blood flow. The cephalic blood flow monitoring system 530 may be configured as an ultrasound doppler-based unit or a photoplethysmography (PPG) unit 555 or a laser speckle-based unit. For example, a Transcranial Doppler Ultrasound (TCD) is a non-invasive technique for real-time monitoring of cerebral blood flow. It can detect abnormalities such as blockages or irregular blood flow patterns, which may indicate conditions like stroke or carotid artery disease. New development in laser technology also provides convenient way to monitor cerebral and extracranial blood flow. There are a few laser speckle-based blood flow monitoring units and examples include a Speckle Contrast Optical Spectroscope unit (SCOS unit) 510. A novel auricular Speckle Contrast Optical Spectroscope unit (auricular SCOS unit) 511 is disclosed by placing a SCOS unit 510 in an auricular housing 11, such as a modified earbud housing 61 or an in-the-ear housing 62 or a behind-the-ear-hearing-aid-style housing 63. Alternatively, the cephalic blood flow monitoring system 530 may comprise an auricular SCOS unit 511 and a PPG unit 555. There is evidence that combining SCOS and PPG together can enhance the accuracy of blood flow assessments compared to using either method alone, as SCOS captures high-frequency changes in blood flow while PPG provides volume data (complementary effects). In some embodiments, a cephalic blood flow monitoring system may comprise one of a SCOS unit 510 and an auricular SCOS unit 511 and may further comprises a photoplethysmography (PPG) unit 555. The PPG unit 555 is configured to record the wearer's cerebral and extracranial blood flow data and estimate the wearer's systolic and diastolic blood pressure. The PPG unit 555 is in electronic communication with the processing unit 50, 401. The processing unit 50, 401, is configured to analyze the cerebral and extracranial blood flow data recorded by the PPG unit 555 and one of: the SCOS unit 510 and the auricular SCOS unit 511 to assess the wearer's cerebral and extracranial blood flow data. The auricular SCOS unit 511 comprises a light source (laser) 519 that pass through a focusing lens 518 and an expander 513 and mirrors 514 and then tissue (or sample) 515. The light is then collected by a SCOS sensor (detector) 512 that is integrated with a Complementary Metal-Oxide Semiconductor camera (CMOS camera) 516 and the data are transmitted to a data processing unit (computer) 517 for analysis and to get the blood flow data at different depths of the tissue, such as cerebral blood flow data and extracranial blood flow data. (FIG. 34). In some embodiments, a first auricular SCOS sensor 512 is configured to be located on a surface of a tragus-concha bowl 957 of the wearer's first ear or the external ear canal 904 of the wearer's first ear when in use to collect the wearer's cerebral (intracranial) and extracranial blood flow data. In some embodiments, a cephalic blood flow monitoring system 530 may comprise an auricular SCOS unit 511 and a PPG unit 555 (both unit 511 and unit 555 may be located in the auricular housing 11) to enhance the capability to monitor the cerebral and extracranial blood flow. In some embodiments, a first neurovascular monitoring system 500 comprises a first auricular EEG monitoring system 100 (configured to record the wearer's EEG and qEEG data from a first ear 902 of the wearer) and a first auricular SCOS unit 511 (configured to record the wearer's cerebral and extracranial blood flow data from the wearer's first ear 902), configured to simultaneously record the wearer's EEG and qEEG data and cerebral (intracranial) and extracranial blood flow data.

In some embodiments, an auricular detection-therapy system for migraine 101 may comprise a cephalic blood flow monitoring system 530, a first neuromodulation unit 300 and a processing unit 50, 401. The cephalic blood flow monitoring system 530 is configured to record cerebral and extracranial blood flow data of the wearer 900. The first neuromodulation unit 300 is configured to give neuromodulating electric stimulation to the wearer 900 when activated. The neuromodulating electric stimulation parameters may be pre-determined (open-loop controlled) or feedback controlled (closed-loop controlled). The timing and when to turn on or turn off the first neuromodulation unit 300 may also be closed-loop controlled. The processing unit 50, 401, is in electronic communication with the cephalic blood flow monitoring system 530 and the first neuromodulation unit 300. The processing unit 50, 401 is configured to analyze the cerebral and extracranial blood flow data recorded by the cephalic blood flow monitoring system 530 to detect presence or cessation of cerebral and extracranial blood flow data suggestive of migraine. When the processing unit 50, 401 detects presence of cerebral and extracranial blood flow data suggestive of migraine, the processing unit 50, 401, is configured to immediately send signals to the first neuromodulation unit 300 to automatically start sending neuromodulating electric stimulation to the wearer 900. The processing unit 50, 401, is also configured to analyze the cerebral and extracranial blood flow data recorded by the cephalic blood flow monitoring system 530 to detect presence or cessation of cerebral and extracranial blood flow data suggestive of impending migraine. When the processing unit 50, 401, detects presence of cerebral and extracranial blood flow data suggestive of impending migraine, the processing unit 50, 401, is configured to immediately send signals to the first neuromodulation unit 300 to automatically start sending neuromodulating electric stimulation to the wearer 900. When the processing unit 50, 401, detects both of the following: cessation of cerebral and extracranial blood flow data suggestive of migraine and cessation of cerebral and extracranial blood flow data suggestive of impending migraine, the processing unit 50, 401, is further configured to immediately send signals to the first neuromodulation unit 300 to automatically stop sending neuromodulating electric stimulation to the wearer 900.

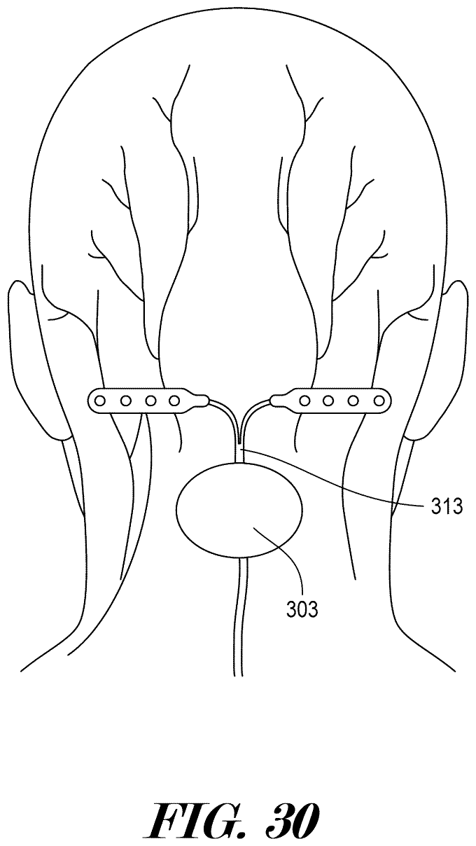

In preferred embodiments for an automatic detection-therapy system 101 for migraine, the first neuromodulation unit 300 may include at least one of the following components: a first transcutaneous auricular vagus nerve stimulation unit (first taVNS unit) 30, a supraorbital nerve stimulation unit 301, an infraorbital nerve stimulation unit 305, a first auriculotemporal nerve stimulation unit 302, an occipital nerve stimulation unit 303 and a first greater auricular nerve (GAN) stimulation unit 304. When any of these components (including unit 30, unit 301, unit 302, unit 303, unit 304, unit 305) is activated, neuromodulating electric stimulation will be sent to the wearer (user) 900. There are evidences showing the benefits of neuromodulating electric stimulation from taVNS unit 30. More recently, there are also evidences showing benefits of neuromodulating electric stimulation from non-vagus electric neuromodulation unit. As used herein, the term “non-vagus electric neuromodulation unit” refers to one of the following: a supraorbital nerve stimulation unit 301, an infraorbital nerve stimulation unit 305, an auriculotemporal nerve stimulation unit 302, an occipital nerve stimulation unit 303, a greater auricular nerve stimulation unit 304, and various combinations thereof: Non-vagus electric neuromodulation has significant benefits for migraine, cluster headache and other pain. Simultaneous stimulation from a taVNS unit 30 and a non-vagus electric neuromodulation unit has synergistic effects. The first taVNS unit 30 may comprise a miniature first taVNS stimulating electrode 31. The first taVNS stimulating electrode 31 may be configured to contact vagus innervated auricular skin of the wearer's 900 first ear. The vagus innervated auricular skin that the taVNS stimulating electrode 31 is configured to contact may be selected from at least one of the following: posterior and inferior walls of external ear canal 904, inner/posterior portion of tragus 905, cymba-concha 906, and majority of cavum-concha 907. (Part of the eardrum also receives vagus innervation but eardrum is not suitable for electrode placement). When prompted, the first taVNS unit 30 may be configured to give neuromodulating electric stimulation through the first taVNS stimulating electrode 31 to the vagus innervated auricular skin of the wearer's first ear in a way similar to transcutaneous electric nerve stimulation (TENS) (taVNS and TENS as known in the art). (FIGS. 2, 11-13). The supraorbital nerve stimulation unit 301 may comprise a supraorbital nerve stimulation electrode 311 to be attached to supraorbital nerve innervated forehead skin. The supraorbital nerve stimulation unit 301 is configured to give neuromodulating electric stimulation through the supraorbital nerve stimulating electrode 311 to a wearer's supraorbital nerve innervated forehead skin when prompted (or activated). (FIG. 29). The first auriculotemporal nerve stimulation unit 302 may comprise a first auriculotemporal nerve stimulation electrode 312 to be attached to the auriculotemporal nerve innervated skin of the wearer's first ear 902. The auriculotemporal nerve innervated auricular skin include anterior-superior helix 909, anterior outer part of tragus 905, anterior portion (or anterior superior part) of cavum-concha 907, anterior and superior walls of the external ear canal 904. (The anterior auricle and part of the outer tympanic membrane also receive auriculotemporal nerve innervation). Thus, the tragus-concha bowl 957 and the external ear canal 904 received mixed innervation from both the auricular branch of vagus nerve and the auriculotemporal nerve. The first auriculotemporal nerve stimulation unit 302 is configured to give neuromodulating electric stimulation through the first auriculotemporal nerve stimulating electrode 312 to a wearer's auriculotemporal nerve innervated skin of the wearer's first ear when prompted (or activated). (FIG. 31). The occipital nerve stimulation unit 303 may comprise an occipital nerve stimulation electrode 313 to be attached to occipital nerve innervated occipital region. The occipital nerve stimulation unit 303 is configured to give neuromodulating electric stimulation through the occipital nerve stimulating electrode 313 to a wearer's occipital nerve innervated occipital region when prompted (or activated). (FIGS. 30, 32). The first greater auricular nerve (GAN) stimulation unit 304 may comprise a first GAN stimulation electrode 313 to be attached to GAN innervated auricular skin of the wearer's first ear. The first GAN stimulation unit 304 is configured to give neuromodulating electric stimulation through the first GAN stimulating electrode 314 to the GAN innervated auricular skin of the wearer's first ear when prompted or activated. (FIG. 33). The infraorbital nerve stimulation unit 305 is configured to give neuromodulating electric stimulation through the infraorbital nerve stimulating electrode 315 to a wearer's infraorbital nerve innervated mid-facial skin when prompted (or activated). (FIG. 31). (For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve innervated auricular skin includes: anterior outer part of tragus 905, anterior-superior part of cavum concha 907, anterior and superior walls of the external ear canal 904 and, anterior and superior part of pinna including anterior-superior helix 909. The greater auricular nerve innervated auricular skin includes: cavum concha, lower two thirds of anterior and posterior pinna and the mastoid process.) From the above comparison, it is obvious that the tragus-concha 957 of a wearer's ear 902 received mixed and overlapped innervation from the auricular branch of vagus nerve, the auriculotemporal nerve (ATN) and the greater auricular nerve (GAN). When the taVNS stimulating electrode 31, the ATN stimulating electrode 312 and the GAN stimulating electrode 314 are located on a body-structure 67, by carefully selecting the locations for the taVNS stimulating electrode 31 the ATN stimulating electrode 312 and the GAN stimulating electrode 314 on the body-structure 67 to match the aforementioned innervation locations of the innervated skin on the tragus-concha bowl 957, these stimulating electrodes 31, 312, 314, will automatically get in close contact with their target skin on the tragus-concha bowl 957 when the body-structure 67 is placed in the tragus-concha bowl 957. Alternatively, when the taVNS stimulating electrode 31 and the ATN stimulating electrode 312 are located on a tubular-shaped structure 66, by carefully selecting the locations for the taVNS stimulating electrode 31 and the ATN stimulating electrode 312 on the tubular-shaped structure 66 to match the aforementioned innervation locations of the innervated skin on the external ear canal 904, these stimulating electrodes 31, 312, will automatically get in close contact with their target skin on the external ear canal 904 when the tubular-shaped structure 66 is placed in the external ear canal 904.)

In some embodiments for an automatic detection-therapy system 101 for migraine, the auricular EEG recording module 20 may be in electronic communication with the processing unit 50, 401, through Bluetooth, wired, wireless or other electronic connection means or methods. Each component of the neuromodulation unit 300 (including at least one of: the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305) may also be in electronic communication with the processing unit 50, 401, through Bluetooth, wired, wireless or other connection means. The EEG recording module 20 collects the wearer's EEG and qEEG data and these data are transmitted to the processing unit 50, 401. With the help of various advanced EEG analysis algorithms together with machine learning, deep learning and artificial intelligence (as known in the art), the processing unit 50, 401, is configured to analyze the EEG data (and converted to qEEG data) to detect presence of EEG signals and qEEG signals of wearer 900 suggestive of migraine and impending migraine. The processing unit 50, 401, may be also configured to detect cessation of EEG and qEEG signals of wearer 900 suggestive of migraine or cessation of EEG and qEEG signals suggestive of impending migraine. In some embodiments, an automatic detection-therapy system 101 for migraine may comprise a neurovascular monitoring system 500 that includes an auricular EEG monitoring system 100 and a cephalic blood flow monitoring system 530 that comprises an auricular speckle contrast optical spectroscopy unit (auricular SCOS unit) 511. (Alternatively, the cephalic blood flow monitoring system 530 may comprise an auricular SCOS unit 511 and a photoplethysmography (PPG) unit 555.) (PPG unit 555 may enhance the capability of SCOS unit 5111 in assessing cerebral and extracranial blood flow, especially the extracranial blood flow.) The auricular SCOS unit and the auricular EEG recording module 20 may be in electronic communication with the processing unit 50, 401, through Bluetooth, wired, wireless or other electronic connection means or methods. The auricular SCOS unit 511 may be configured to record the cerebral and extracranial blood flow data of the wearer 900 simultaneously during EEG data (and qEEG data) collection (recording) by the auricular EEG monitoring system 100.

When the processing unit 50, 401, detects one of the following: presence of EEG and qEEG signals suggestive of migraine and presence of blood flow data suggestive of migraine, the processing unit 50, 401, may be configured to automatically send signals to the neuromodulation unit 300 to actuate at least one of the following components: the taVNS unit 30, the supraorbital nerve stimulation unit 301, the auriculotemporal nerve stimulation unit 302, the occipital nerve stimulation unit 303, the greater auricular nerve stimulation unit 304 and infraorbital nerve stimulation unit 305 to start sending pre-determined neuromodulating electric stimulation immediately. When the processing unit 50, 401, detects one of the following: presence of EEG and qEEG signals suggestive of impending migraine and presence of blood flow data suggestive of impending migraine, the processing unit 50, 401, may be configured to automatically send signals to the neuromodulation unit 300 to actuate at least one of the following components: the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305 to start sending pre-determined neuromodulating electric stimulation immediately. When the processing unit detects all of the following: cessation of EEG and qEEG signals suggestive of migraine, cessation of blood flow data suggestive of migraine, cessation of EEG and qEEG signals suggestive of impending migraine and cessation of blood flow data suggestive of impending migraine, the processing unit 50, 401, may be further configured to automatically send signals to the neuromodulation unit 300 to stop sending pre-determined electric stimuli from any of the following: the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305.

For the taVNS unit 30, the parameters of the electric stimulation for migraine, including the stimulating patterns, strength, duration, frequency and intervals, are predetermined, such as shown in Table 1. For the taVNS unit 30, the parameters of the electric stimulation for impending migraine, including the stimulating patterns, strength, duration, frequency and intervals, are also predetermined, such as shown in Table 2. Some examples of stimulus parameters for other components of the neuromodulation unit 300 (such as supraorbital nerve stimulation unit 301, auriculotemporal nerve stimulation unit 302, occipital nerve stimulation unit 303, greater auricular nerve stimulation unit 304 and infraorbital nerve stimulation unit 305) for migraine and impending migraine were described hereinbefore and by various articles in the Background section of this invention. In some embodiments, the processing unit 50, 401, may include a closed-loop control system (a “sensor-controller-actuator” setup) (or a feedback-controlled mechanism). The closed-loop control system within the processing unit 50, 401, may be in electronic communication with the neurovascular monitoring system 500 (including the auricular EEG monitoring system 100 and cephalic blood flow monitoring system 530) and the neuromodulation unit 300. The closed-loop control system receives real-time continuous input of the wearer's 900 EEG data (and qEEG data) from the auricular EEG monitoring system 100 and real-time continuous input of the wearer's cerebral and extracranial blood flow data from the cephalic blood flow monitoring system 530 and the closed-loop control system of the processing unit 50, 401, is configured to analyze these real-time EEG data and qEEG data and cerebral and extracranial blood flow data, using controlling algorithms, to continuously adjust the actuating outputs to the neuromodulation unit 300. The actuating outputs includes turning on or turning off at least one component of the neuromodulation unit 300 and adjusting the stimulating parameters (intensity, frequency, duration, cycle, wave form etc.) of the neuromodulating unit 300 during the time when the neuromodulating unit 300 is turned on. This will greatly enhance the effectiveness and safety of neuromodulation, especially trigeminal nerve stimulation. In trigeminal nerve neuromodulation, some high-frequency (e.g., 200 Hz) stimulation could produce pronounced effects on heart rate and blood pressure compared to lower frequencies. Studies have shown that high frequency (e.g. 200 Hz) trigeminal nerve electric stimulation can produce a rapid, transient decrease in heart rate (bradycardia) and, in some cases, a sudden, brief drop in blood pressure (hypotension) due to activation of the trigeminal cardiac reflex. Both taVNS and trigeminal nerve stimulation are known to be effective for migraine and cluster headache. Studies have shown that trigeminal nerve stimulation has stronger (FDA-cleared) evidence specifically for acute and preventive migraine treatment, while taVNS has particular strength in reducing migraine frequency and treating cluster headache. High frequency trigeminal nerve stimulation (in the 100-150 Hz range) is generally considered more effective for acute migraine and cluster headache treatment, while specific lower frequency combinations are used for prevention of migraine and cluster headache. Using controlled-loop control system together with blood flow monitoring from auricular SCOS unit 511, blood pressure monitoring from the PPG unit 555 and EEG data and qEEG data from the auricular EEG monitoring system 100 can minimize the risk when high frequency is used for trigeminal nerve stimulation. When any of the following: a decrease of blood flow more than a predetermined level (e.g. a decrease of blood flow of 20% or more), a decrease of blood pressure more than a predetermined level (e.g. at least one of: a decrease of systolic blood pressure of 20 mmHg or more and a decrease of diastolic blood pressure of 20 mmHg or more) and the EEG and qEEG data changes more than a predetermined level (e.g. at least one of: an increase of relative delta power of 20% or more and an increase of delta/alpha ratio of 20% or more) (or significant changes of other quantitative data from qEEG), the stimulation may be immediately stopped or the stimulation parameters may be immediately altered. High frequency (around 100-160 Hz) occipital nerve stimulation has also been found to be more effective than low frequency for migraine and cluster headache. Alternating frequencies (2/100 Hz) occipital nerve stimulation has also been found to be effective.

In preferred embodiments, for most patients whose migraine is usually on one side of the head 901, one neurovascular monitoring system 500 placed on the same side may be preferred. These patients might use one neuromodulation unit 500 on the same side (although, optionally, two neuromodulation units 500, with one unit on each side of the head, might be considered). In other embodiments, for some patients with migraine, two neurovascular monitoring systems 500, one on each side of the head 901, may be considered. For some patients with migraine, two neuromodulation units 300 may be considered, with one neuromodulation unit 300 on each side of the head 901. Studies have shown that migraine is often unilateral, causing throbbing pain on one side of the head. However, in many cases of migraine, the pain can also be bilateral with throbbing on both sides of the head. There are evidences that up to 50% of migraine patients may experience bilateral pain, or pain that starts on one side and then spreads to both sides. In some embodiments, an auricular detection-therapy system 101 for migraine may comprise two neuromodulation units 300, including two taVNS units 30. However, in rare situations if a patient is unable to tolerate a taVNS unit 30 in right ear due to bradycardia or other side effects, only one taVNS unit 30 may be utilized and be placed in the left ear. For some patients with migraine, two auriculotemporal nerve stimulation units 302 might be considered, with one auriculotemporal nerve stimulation unit 302 on each side of the head 901 coupled to the tragus-concha bowl 957 or external ear canal 904 of each ear 902. Similarly, for some patients with migraine, two greater auricular nerve stimulation units 302 might be considered, with one greater auricular nerve stimulation unit 302 on each side of the head 901 coupled to the tragus-concha bowl 957 (cavum concha 907) of each ear 902. In some embodiments, an automatic detection-therapy system 101 for migraine may comprise a neurovascular monitoring system 500 that includes an auricular EEG monitoring system 100 having a first EEG recording module 20 and a second EEG recording module 20 and a cephalic blood flow monitoring system 530 having a first auricular SCOS unit 511 and a second auricular SCOS unit 511. (Alternatively, a cephalic blood flow monitoring system 530 may comprise a first SCOS unit 511 and a first PPG unit 555 linked to a first ear 902 of the wearer and a second SCOS unit 511 and a second PPG unit 555 linked to a second ear 902 of the wearer.) The first EEG recording module 20 and the first auricular SCOS unit 511 may be configured to be linked to a wearer's first ear 902, while the second EEG recording module 20 and the second auricular SCOS unit 511 may be configured to be linked to a second ear 902 of the wearer 900. The automatic detection-therapy system 101 for migraine may further comprise two neuromodulation units 300, with the first neuromodulation unit 300 located in the wearer's first ear 902 (although some components of the first neuromodulation unit 300 are located at mid-forehead, mid-face and mid-occipital region) and the second neuromodulation unit 300 located in the wearer's second ear 902. The first neuromodulation unit 300 comprises at least one of: a first taVNS unit 30, a supraorbital nerve stimulation unit 301, an infraorbital nerve stimulation unit 305, a first auriculotemporal nerve stimulation unit 302, an occipital nerve stimulation unit 303, and a first greater auricular nerve stimulation unit 304. The second neuromodulation unit 300 may comprise at least one of: a second taVNS unit 30, a second auriculotemporal nerve stimulation unit 302 and a second greater auricular nerve stimulation unit 304. The second taVNS unit 30 includes a second taVNS stimulating electrode 31 configured to contact vagus innervated auricular skin of the wearer's second ear 902. The second auriculotemporal nerve stimulation unit 302 includes a second auriculotemporal nerve stimulating electrode 312 configured to contact auriculotemporal nerve innervated auricular skin of the wearer's second ear 902, while the second greater auricular nerve stimulation unit 304 includes a second greater auricular nerve stimulating electrode 314 configured to contact greater auricular nerve innervated auricular skin of the wearer's second ear 902. The processing unit 50, 401, is configured to analyze the EEG data recorded by the auricular EEG monitoring system 100, including EEG data and qEEG data recorded by the first EEG recording module 20 and EEG data and qEEG data recorded by the second EEG recording module 20, to detect presence or cessation of EEG signals and qEEG signals suggestive of migraine. The processing unit 50, 401, is further configured to analyze the EEG data and qEEG data recorded by the auricular EEG monitoring system 100, including EEG data and qEEG data recorded by the first EEG recording module 20 and EEG data and qEEG data recorded by the second EEG recording module 20, to detect presence or cessation of EEG signals and qEEG signals suggestive of impending migraine. The processing unit 50, 401, is also configured to analyze the cerebral and extracranial blood flow data recorded by the cephalic blood flow monitoring system 530, including cerebral and extracranial blood flow data recorded by the first auricular SCOS unit 511 (and the first PPG unit 555) and cerebral and extracranial blood flow data recorded by the second auricular SCOS unit 511 (and the second PPG unit 555), to detect presence or cessation of cerebral and extracranial blood flow data suggestive of migraine. The processing unit 50, 401, is further configured to analyze the cerebral and extracranial blood flow data recorded by the cephalic blood flow monitoring system 530, including cerebral and extracranial blood flow data recorded by the first auricular SCOS unit 511 (and the first PPG unit 555) and cerebral and extracranial blood flow data recorded by the second auricular SCOS unit 511 (and the second PPG unit 555), to detect presence or cessation of cerebral and extracranial blood flow data suggestive of impending migraine. When the processing unit 50, 401, detects at least one of the following: presence of EEG and qEEG signals suggestive of migraine by analyzing the EEG and qEEG data recorded by the first EEG recording module 20, presence of EEG and qEEG signals suggestive of migraine by analyzing the EEG and qEEG data recorded by the second EEG recording module 20, presence of cerebral and extracranial blood flow data suggestive of migraine by analyzing cerebral and extracranial blood flow data recorded by the first auricular SCOS unit 511 (and the first PPG unit 555), and presence of cerebral and extracranial blood flow data suggestive of migraine by analyzing cerebral and extracranial blood flow data recorded by the second auricular SCOS unit 511 (and the second PPG unit 555), the processing unit 50, 401, is configured to start sending predetermined neuromodulating electric stimulation from at least one of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303 (including various combinations thereof). When the processing unit 50, 401, detects at least one of the following: presence of EEG and qEEG signals suggestive of impending migraine by analyzing EEG and qEEG data recorded by the first EEG recording module 20, presence of EEG and qEEG signals suggestive of impending migraine by analyzing EEG and qEEG data recorded by the second EEG recording module 20, presence of cerebral and extracranial blood flow data suggestive of impending migraine by analyzing cerebral and extracranial blood flow data recorded by the first auricular SCOS unit 511 (and the first PPG unit 555), and presence of cerebral and extracranial blood flow data suggestive of impending migraine by analyzing cerebral and extracranial blood flow data recorded by the second auricular SCOS unit 511 (and the second PPG unit 555), the processing unit 50, 401, is configured to start sending predetermined neuromodulating electric stimulation from at least one of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 302, the infraorbital nerve stimulation unit 305 and the occipital nerve stimulation unit 303 (including various combinations thereof). When the processing unit detects all of the following: cessation of EEG and qEEG signals suggestive of migraine by analyzing EEG and qEEG data recorded by the first EEG recording module 20, cessation of EEG and qEEG signals suggestive of impending migraine by analyzing EEG and qEEG data recorded by the first EEG recording module 20, cessation of cerebral and extracranial blood flow data suggestive of migraine by analyzing cerebral and extracranial blood flow data recorded by the first auricular SCOS unit 511 (and the first PPG unit 555), cessation of cerebral and extracranial blood flow data suggestive of impending migraine by analyzing cerebral and extracranial blood flow data recorded by the first auricular SCOS unit 511 (and the first PPG unit 555), cessation of EEG and qEEG signals suggestive of migraine by analyzing EEG and qEEG data recorded by the second EEG recording module 20, cessation of EEG and qEEG signals suggestive of impending migraine by analyzing EEG and qEEG data recorded by the second EEG recording module 20, cessation of cerebral and extracranial blood flow data suggestive of migraine by analyzing cerebral and extracranial blood flow data recorded by the second auricular SCOS unit 511 (and the second PPG unit 555), and cessation of cerebral and extracranial blood flow data suggestive of impending migraine by analyzing cerebral and extracranial blood flow data recorded by the second auricular SCOS unit 511 (and the second PPG unit), the processing unit is further configured to stop sending neuromodulating electric stimulation from any of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303.

In some embodiments for an automatic detection-therapy system 101 for migraine, the auricular EEG recording module 20, the auricular SCOS unit 511, the PPG unit 555, the taVNS unit 30, the auriculotemporal nerve stimulation unit 302, the greater auricular nerve stimulation unit 304 and the processing unit 50, 401, may be housed together as a single structure and placed in the external ear canal 904, tragus 905, concha 906, 907, and/or peri-auricular area 903. Alternatively, the taVNS unit 30, the auricular SCOS unit 511, the PPG unit 555, the auriculotemporal nerve stimulation unit 302, the greater auricular nerve stimulation unit 304, and the EEG recording module 20 may be in separate housings and may be located adjacent to each other with each of them attached to one or more of the following: the external ear canal 904, the tragus-concha bowl 957, and the peri-auricular area 903. In some embodiments, all of the EEG sensor electrodes 12, 13, 72, 73, 82, 83, the optional reference electrode 84 (if incorporated), the auricular SCOS sensor 512, the PPG unit 555, the taVNS stimulating electrode 31, the auriculotemporal nerve stimulating electrode 312 and the greater auricular nerve stimulating electrode 314 may be all housed together sharing a same auricular housing 11. The auricular housing 11 may be selected from one of the following: a modified earbud housing 61, a modified in-the-ear housing 62, and a behind-the-ear-hearing-aid-style housing 63, as described hereinbefore. All of these housings 61, 62, 63, include a tubular-shaped structure 66 (configured to be inserted into an external ear canal 904 of a wearer when in use) and a body-structure 67 (configured to be placed at the opening of the external ear canal 904 and to sit or be placed inside a tragus-concha bowl 957 of the wearer's ear when in use). All of the EEG sensor electrodes 12, 13, 72, 73, 82, 83, may be configured to be located on a surface 91 of the tubular-shaped structure 66, while the auricular SCOS sensor 512, the PPG unit 555, the taVNS stimulating electrode 31 and the auriculotemporal nerve stimulating electrode 312 are configured to be housed on a surface of one of: the tubular-shaped structure 66 and the body-structure 67. The greater auricular nerve (GAN) stimulating electrode 314 and the optional reference electrode 84 are configured to be located at a surface of the body-structure 67. All of the EEG sensor electrodes 12, 13, 72, 73, 82, 83, the optional reference electrode 84, the auricular SCOS sensor 512, the PPG unit 555, the taVNS stimulating electrode 31, the auriculotemporal nerve stimulating electrode 312 and the GAN stimulating electrode 512 are configured to be partially embedded in the surface with slight protrusion at the surface of one of: the tubular-shaped structure 66 and the body-structure 67. (Alternatively, the auricular SCOS sensor 512 may be placed slightly below the surface of the tubular-shaped structure 66 or the body-structure 67 so that there is a small air gap between the SCOS sensor 512 and the wearer's skin when the tubular-shaped structure 66 or the body-structure 67 are placed in the wearer's ear 902.) The tubular-shaped structure 66 and the body-structure 67 may be configured to comprise a flexible elastic and adaptable material and the flexible elastic and adaptable material is configured to have appropriate flexibility, elasticity and adaptability so that the tubular-shaped structure 66 will naturally adapt to the contour of the external ear canal 904 and snugly fill the interior of the wearer's external ear canal 904 when the tubular-shaped structure 66 is inserted into the external ear canal 904 of the wearer's ear 902 and, meanwhile, the body-structure 67 will naturally adapt to the contour of the tragus-concha bowl 957 of the wearer's ear 902 and snugly fill the interior of the tragus-concha bowl 957 of the wearer's ear 902 when the body-structure 67 is placed inside the tragus-concha bowl 957 of the wearer's ear 902, so that all of the EEG sensor electrodes 12, 13, 72, 73, 82, 83, are naturally snugly in contact with the skin of the external ear canal 904 of the wearer's ear 902, and, at the same time, the auricular SCOS sensor 512, the PPG unit 555, the taVNS stimulating electrode 31 and the auriculotemporal nerve stimulating electrode 312 are naturally and snugly in contact with the skin of the tragus-concha bowl 957 of the wearer's ear 902 or the skin of the external ear canal 904 of the wearer's ear 902, while the GAN stimulating electrode 314 and the optional reference electrode 84 are naturally snugly in contact with the skin of the wearer's cavum concha 907 (part of the tragus-concha bowl 957), and so that the taVNS stimulating electrode 31 is naturally and snugly in contact with its target skin of vagus-innervated auricular skin of the wearer's first ear 902, provided by carefully selecting a location for the taVNS stimulating electrode 31 on the tubular-shaped structure 66 or the body-structure 67 to match one of the innervation locations of the vagus innervated skin on one of: the external ear canal 904 and the tragus-concha bowl 957, and, at the same time, the auriculotemporal nerves stimulating electrode 312 will be naturally in close contact with its target skin of auriculotemporal nerve innervated auricular skin, provided by carefully selecting a location for the auriculotemporal nerve stimulating electrode 312 on the tubular-shaped structure 66 or the body-structure 67 to match one of the innervation locations of the auriculotemporal nerve innervated skin on the external ear canal 904 or the tragus-concha bowl 957, and, at the same time, the greater auricular nerve stimulating electrode 314 will be naturally in close contact with greater auricular nerve innervated auricular skin, provided by carefully selecting a location for the greater auricular nerve stimulating electrode 314 on the body-structure 67 to match one of the innervation locations of the greater auricular nerve innervated skin on tragus-concha bowl 957. As described hereinbefore, by comparing the innervation patterns, it is obvious that the tragus-concha 957 of a wearer's ear 902 received mixed and overlapped innervation from the auricular branch of vagus nerve, the auriculotemporal nerve and the greater auricular nerve. In some embodiments, a neuromodulation unit 300 may have 3 components having a taVNS stimulating electrode 31, an auriculotemporal nerve stimulating electrode 312 and a greater auricular nerve stimulating electrode 314 and all of these electrodes (31, 312, 314) may be located on the body-structure 67. By carefully selecting the location for the taVNS stimulating electrode 31 on the body-structure 67 to match the innervation locations of the vagus innervated skin on the tragus-concha bowl 957, the taVNS stimulating electrode 31 will automatically get in close contact with its target skin on the tragus-concha bowl 957 when the body-structure 67 is placed in the tragus-concha bowl 957. By carefully selecting the location for the auriculotemporal nerve stimulating electrode 312 on the body-structure 67 to match the innervation locations of the auriculotemporal nerve on the tragus-concha bowl 957, the auriculotemporal nerve stimulating electrode 312 will automatically get in close contact with its target skin on the tragus-concha bowl 957 when the body-structure 67 is placed in the tragus-concha bowl 957. Similarly, by carefully selecting the location for the greater auricular nerve stimulating electrode 314, on the body-structure 67 to match the innervation locations of the greater auricular nerve innervated skin on the tragus-concha bowl 957, the greater auricular nerve stimulating electrode 314 will automatically get in close contact with its target skin on the tragus-concha bowl 957 when the body-structure 67 is placed in the tragus-concha bowl 957. Alternatively, the taVNS stimulating electrode 31 and the auriculotemporal nerve stimulating electrode 312 may be located on the tubular-shaped structure 66. By carefully selecting the location for the taVNS stimulating electrode 31 on the tubular-shaped structure 66 to match its target skin locations on the external ear canal 904, the taVNS stimulating electrode 31 will be automatically get in close contact with its target skin in the external ear canal 904. Similarly, by carefully selecting the location for the auriculotemporal nerve stimulating electrode 312 on the tubular-shaped structure 66 to match its target skin locations on the external ear canal 904, the auriculotemporal nerve stimulating electrode 312 will be automatically get in close contact with its target skin in the external ear canal 904.

Preferably, the automatic detection-therapy system 101 for migraine further comprises a network interface 53, 406, in electronic communication with the processing unit 50, 401. The network interface 53, 406, may be configured to generate a notification (such as an audible notification via a speaker 15, 404A, or a tactile notification via a vibrator 17, 404B). When migraine or an impending migraine is detected (by EEG and qEEG signals and blood flow data as described hereinbefore), the network interface 53, 406, may be configured to automatically generate a notification to a client device 400, such as to the client device 400 of the wearer 900 and/or the client device 400 of the wearer's healthcare provider 950 so that the wearer 900 or the wearer's healthcare provider 950 can take appropriate actions. Besides that, the neuromodulation unit 300 may be automatically actuated to start giving pre-determined neuromodulating stimulation to the wearer. The neuromodulation unit 300 includes at least one of the following components: a taVNS unit 30, a supraorbital nerve stimulation unit 301, an auriculotemporal nerve stimulation unit 302, an occipital nerve stimulation unit 303, a greater auricular nerve stimulation unit 304 and an infraorbital nerve stimulation unit 305. Thus, when migraine or impending migraine is detected, in addition to receiving at least one neuromodulation therapy automatically, the wearer 900 can take extra dose of medications or take other actions to alleviate the condition. An example of the notification device of a client device 400 is a speaker 15, 404A, configured to generate audible warning notification (or a vibrator 17, 404 B, configured to generate tactile notification) when presence of EEG and qEEG signals or blood flow data suggestive of migraine or impending migraine is detected by the processing unit 50, 401 (as described hereinbefore). The notification function is well-known. Most smart phones, smart watches and health trackers have notification function.

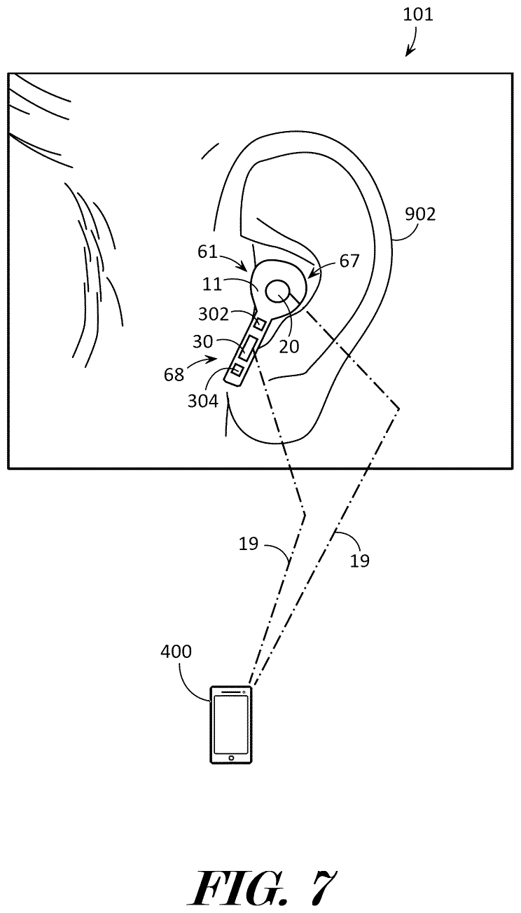

In some embodiments, an automatic detection-therapy system 101 for migraine may comprise an auricular EEG recording module 20, a processing unit 50, 401, an auricular SCOS sensor 512, a PPG unit 555, a taVNS unit 30, an auriculotemporal nerve stimulation unit 302, a greater auricular nerve (GAN) stimulation unit 304 and a network interface 53 and all or part of them (or part of their components) may be configured to be housed in a shared auricular housing 11. The auricular housing 11 may be selected from one of the following: a modified earbud housing 61, a modified in-the-ear housing 62, a behind-the-ear-hearing-aid-style housing 63 and a tubular-shaped structure 66. (FIGS. 7, 11, 12, 13). The modified earbud housing 61 may include a tubular-body portion 25 and a stem portion 68. (FIGS. 2, 11). The stem portion 68 is equivalent to a “stem” (or a “stalk”) of an earbud. The “stem” hangs down from the wearer's ear. The tubular-body portion 25 may include a tubular-shaped structure 66 and a body-structure 67. The tubular-shaped structure 66 is equivalent to an elongated and modified “ear-tip” and “nozzle” of an earbud (or an AirPod) and will be inserted into a wearer's external ear canal 904 when in use. The body-structure 67 is equivalent to a “body” (or a “shell”) of an earbud (or an AirPod). The body-structure 67 may be placed immediately at the opening of the wearer's external ear canal 904 and be placed inside the wearer's tragus-concha bowl 957 when in use. All of the EEG sensor electrodes 12, 13, 72, 73, 82, 83, may be configured to be placed on a surface 91 and partially embedded in the surface 91 with slight protrusion at the surface 91 of the tubular-shaped structure 66. The taVNS stimulating electrode 31, the auricular SCOS sensor 512, the PPG unit 555, and the auriculotemporal nerve stimulating electrode 312 may be configured to be placed on a surface and partially embedded in the surface with slight protrusion at the surface of one of: the tubular-shaped structure 66 and the body-structure 67. (Alternatively, the auricular SCOS sensor 512 may be located slightly below the surface 91 of the tubular-shaped structure 66 or body-structure 67 so that there is a tiny air gap between the auricular SCOS sensor 512 and the skin of the wearer's external ear canal 904 or the skin of the wearer's tragus-concha bowl 957 when the tubular-shaped structure 66 and the body-structure 67 are placed in the wearer's ear 902.) The greater auricular nerve (GAN) stimulating electrode 314 and the optional reference electrode 84 may be placed in a surface (and partially embedded in the surface with slight protrusion at the surface) of the body-structure 67. Both the tubular-shaped structure 66 and the body-structure 67 are configured to be made of elastic flexible and adaptable material. The material of the tubular-shaped structure 66 is configured to have appropriate elasticity flexibility and adaptability so that when the tubular-shaped structure 66 is inserted into the wearer's external ear canal 904, the tubular-shaped structure 66 will naturally adapt to the contour of the wearer's external ear canal 904 and snugly fill the interior of the wearer's external ear canal 904. Similarly, the material of the body-structure 67 is configured to have appropriate elasticity flexibility and adaptability so that when the body-structure 67 is placed inside the wearer's tragus-concha bowl 957, the body-structure 67 will naturally adapt to the contour of the wearer's tragus-concha bowl 957 and snugly fill the interior of the wearer's tragus-concha bowl 957. Thus, all of the EEG sensor electrodes 12, 13, 72, 73, 82, 83, will be naturally in close contact with the skin of the wearer's external ear canal 904; while the taVNS stimulating electrode 31, the auricular SCOS sensor 512, the PPG unit 555, and the auriculotemporal nerve stimulating electrode 312 will be naturally in close contact with the skin of the wearer's external ear canal 904 or the skin of the wearer's tragus-concha bowl 957; and, at the same time, the GAN stimulating electrode 314 and the optional reference electrode 84 will be naturally in close contact with the skin of the wearer's tragus-concha bowl 957. At the same time, the taVNS stimulating electrode 31 will be naturally in close contact with the skin of the wearer's vagus innervated auricular skin since the skin of the external ear canal 904 and the skin of the tragus-concha bowl 957 are part of the vagus innervated auricular skin. Likewise, at the same time, the auriculotemporal nerve stimulating electrode 312 will be naturally in close contact with the skin of the wearer's auriculotemporal nerve innervated auricular skin since the skin of the external ear canal 904 and the skin of the tragus-concha bowl 957 are part of the auriculotemporal nerve innervated auricular skin. Meanwhile, the GAN stimulating electrode 314 will be naturally in close contact with the skin of the wearer's cavum concha 907 (part of the tragus-concha bowl 957) since the skin of cavum concha 907 is part of GAN innervated auricular skin. (This is feasible due to the proximity of the innervation patterns of the auricular branch of vagus nerve, the auriculotemporal nerve and the greater auricular nerve in the auricular area, and by careful selection of the locations for 31, 312, 314 on the tubular-shaped structure 66 or the body-structure 67 to match the locations of their respective target skin on the wearer's ear, as described hereinbefore.) This unique design and set-up will allow the wearer 900 to install or to remove all of the EEG sensor electrodes 12, 13, 72, 73, 82, 83, the optional reference electrode 84 (if incorporated), the auricular SCOS sensor 512, the PPG unit 555, the taVNS stimulating electrodes 31, the auriculotemporal nerve stimulating electrode 312 and the GAN stimulating electrode 314 as easily as placing or removing the tubular-body portion 25 of the modified earbud housing 61 in (or from) the wearer's 900 external ear canal 904 and the tragus-concha bowl 957. Thus, this automatic detection-therapy system 101 is fully wearable, self-installable, self-removable, freely ambulatory and very convenient for wearers (users).

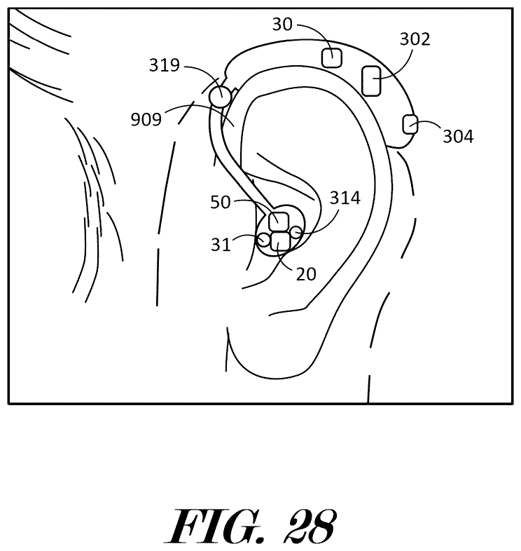

In other embodiments of an automatic detection-therapy system 101 for migraine, all or part of the following (or part of their components): an auricular EEG recording module 20, an auricular SCOS unit 511, a PPG unit 555, a processing unit 50, 401, a taVNS unit 30, an auriculotemporal nerve stimulation unit 302, a GAN stimulating electrode 314 and a network interface 53 may be configured to be housed in an auricular housing 11. The auricular housing 11 may be selected from a behind-the-ear-hearing-aid-style housing 63. The behind-the-ear-hearing-aid-style housing includes an in-the-ear portion 27 and a behind-the-ear portion 26. The in-the-ear portion 27 is essentially the same as the tubular-body portion 25 of a modified earbud housing 61 as aforementioned. (FIG. 13). The in-the-ear portion 27 (of the behind-the-ear-hearing-aid-style housing 63) includes a tubular-shaped structure 66 and a body-structure 67. The tubular-shaped structure 66 is equivalent to an elongated modified “ear-tip” and “nozzle” of an earbud (or an AirPod) and will be inserted into a wearer's external ear canal 904 when in use. The body-structure 67 is equivalent to a “body” (or a “shell”) of an earbud (or an AirPod). The body-structure 67 will be placed immediately at the opening of the wearer's external ear canal 904 and be placed inside the wearer's tragus-concha bowl 957 when in use. All of the EEG sensor electrodes 12, 13, 72, 73, 82, 83, may be configured to be placed on a surface 91 and partially embedded in the surface 91 with slight protrusion at the surface 91 of the tubular-shaped structure 66. The taVNS stimulating electrode 31, the auricular SCOS sensor 512, the PPG unit 555, and the auriculotemporal nerve stimulating electrode 312 may be configured to be placed on the surface and partially embedded in the surface with slight protrusion at the surface of one of: the tubular-shaped structure 66 and the body-structure 67. (Alternatively, the auricular SCOS sensor 512 may be located slightly below the surface 91 of the tubular-shaped structure 66 or body-structure 67 so that there is a tiny air gap between the auricular SCOS sensor 512 and the skin of the wearer's external ear canal 904 or the skin of the wearer's tragus-concha bowl 957 when the tubular-shaped structure 66 and the body-structure 67 are placed in the wearer's ear 902.) The GAN stimulating electrode 314 and the optional reference electrode 84 are configured to be placed at a surface (and partially embedded in the surface with slight protrusion at the surface) of the body-structure 67. Both the tubular-shaped structure 66 and the body-structure 67 are configured to be made of elastic flexible and adaptable material. The material of the tubular-shaped structure 66 is configured to have appropriate elasticity flexibility and adaptability so that when the tubular-shaped structure 66 is inserted into the wearer's external ear canal 904, the tubular-shaped structure 66 will naturally adapt to the contour of the wearer's external ear canal 904 and snugly fill the interior of the wearer's external ear canal 904. Similarly, the material of the body-structure 67 is configured to have appropriate elasticity flexibility and adaptability so that when the body-structure 67 is placed inside the wearer's tragus-concha bowl 957, the body-structure 67 will naturally adapt to the contour of the wearer's tragus-concha bowl 957 and snugly fill the interior of the wearer's tragus-concha bowl 957. Thus, all of the EEG sensor electrodes 12, 13, 72, 73, 82, 83, will be naturally in close contact with the skin of the wearer's external ear canal 904; while the taVNS stimulating electrode 31, the auricular SCOS sensor 512, the PPG unit 555 and the auriculotemporal nerve stimulating electrode 312 will be naturally in close contact with the skin of the wearer's external ear canal 904 or the skin of the wearer's tragus-concha bowl 957; and, at the same time, the GAN stimulating electrode 314 and the optional reference electrode 84 will be naturally in close contact with the skin of the wearer's tragus-concha bowl 957. At the same time, the taVNS stimulating electrode 31 will be naturally in close contact with the skin of the wearer's vagus innervated auricular skin since the skin of the external ear canal 904 and the skin of the tragus-concha bowl 957 are part of the vagus innervated auricular skin. Likewise, at the same time, the auriculotemporal nerve stimulating electrode 312 will be naturally in close contact with the skin of the wearer's auriculotemporal nerve innervated auricular skin since the skin of the external ear canal 904 and the skin of the tragus-concha bowl 957 are part of the auriculotemporal nerve innervated auricular skin. Similarly, at the same time, the GAN stimulating electrode 314 will be naturally in close contact with the skin of the wearer's tragus-concha bowl 957 since the skin of cavum concha 907 (part of the tragus-concha bowl 957) is part of the GAN innervated auricular skin. (This is feasible due to the proximity of the innervation patterns of the auricular branch of vagus nerve, the auriculotemporal nerve and the greater auricular nerve in the auricular area, and by careful selection of the locations for 31, 312, 314, to match the locations of their respective target skin on the wearer's ear, as described hereinbefore.) This unique design and set-up will allow the wearer 900 to attach or to remove all of the EEG sensor electrodes 12, 13, 72, 73, 82, 83, the optional reference electrode 84, the auricular SCOS sensor 512, the PPG unit 555, the taVNS stimulating electrodes 31, the auriculotemporal nerve stimulating electrode 312 and the GAN stimulating electrode 314 as easily as placing or removing the in-the-ear portion 27 of the behind-the-ear-hearing-aid-style housing 63 in (or from) the wearer's 900 external ear canal 904 and the tragus-concha bowl 957. These features will enable the automatic detection-therapy system 101 be fully wearable, user-installable, user-removable, freely ambulatory and very convenient for wearers (users). Alternatively, the auriculotemporal nerve stimulation unit 302 may have a clip electrode 319 to be clipped manually to the wearer's anterior-superior helix 909 of the ear 902 for the auriculotemporal nerve stimulating unit 902 to deliver the electric stimuli via the clip electrode 319 (FIG. 28).

In some embodiments for an automatic detection-therapy system 101 for migraine, all or part of the following (or part of their components): an auricular EEG recording module 20, a processing unit 50, 401, an auricular SCOS unit 511, a PPG unit 555, a taVNS unit 30, an auriculotemporal nerve stimulation unit 302, a greater auricular nerve (GAN) stimulation unit 304, and a network interface 53 may be configured to be housed in an auricular housing 11. The auricular housing 11 may comprise a modified in-the-ear-housing 62 that includes a tubular-shaped structure 66 (to be inserted into a wearer's external ear canal 904 when in use) and a body-structure 67 (to be placed in the tragus-concha bowl 957 of a wearer's ear when in use). (As aforementioned, the modified earbud housing 61 includes a tubular-shaped structure 66 and a body-structure 67. The behind-the-ear-hearing-aid-style housing 63 also includes a tubular-shaped structure 66 and a body-structure 67.) In some embodiments, the modified in-the-ear housing 62 may be made with or may comprise elastic flexible and adaptable material. The material for the modified in-the-ear housing 62 is configured to have appropriate elasticity, flexibility and adaptability so that when it is placed in the wearer's external ear canal 904 and the tragus-conch bowl 957, it will naturally adapt to the contours of the wearer's external ear canal 904 and the contour of the wearer's tragus-concha bowl 957 and snugly fill the interior of the wearer's external ear canal 904 and the interior of the tragus-concha bowl 957. All of the EEG sensor electrodes 12, 13, 72, 73, 82, 83, may be configured to be placed on a surface 91 and partially embedded in the surface 91 with slight protrusion at the surface 91 of the tubular-shaped structure 66. The taVNS stimulating electrode 31, the auricular SCOS sensor 512, the PPG unit 555 and the auriculotemporal nerve stimulating electrode 312 may be configured to be placed on the surface and partially embedded in the surface with slight protrusion at the surface of one of: the tubular-shaped structure 66 and the body-structure 67. (Alternatively, the auricular SCOS sensor 512 may be located slightly below the surface 91 of the tubular-shaped structure 66 or body-structure 67 so that there is a tiny air gap between the auricular SCOS sensor 512 and the skin of the wearer's external ear canal 904 or the skin of the wearer's tragus-concha bowl 957 when the tubular-shaped structure 66 and the body-structure 67 are placed in the wearer's ear 902.) The GAN stimulating electrode 314 and the optional reference electrode 84 are configured to be placed at the surface (and partially embedded in the surface with slight protrusion at the surface) of the body-structure 67. The material of the modified in-the-ear housing 62 (including the tubular-shaped structure 66 and the body structure 67) is configured to have appropriate elasticity, flexibility and adaptability so that when the modified in-the-ear housing 62 is placed in a wearer's external ear canal 904 and the tragus-concha bowl 957, all of the EEG sensor electrodes 12, 13, 72, 73, 82, 83, will be naturally in close contact with the skin of the wearer's external ear canal 904, while the optional reference electrode 84 and the greater auricular nerve (GAN) stimulating electrode 314 will be naturally in close contact with the skin of the wearer's tragus-concha bowl 957, and, at the same time, the auricular SCOS sensor 512, the PPG unit 555, the taVNS stimulating electrode 31 and the auriculotemporal nerve stimulating electrode 312 will be naturally in close contact with the skin of the wearer's external ear canal 904 or skin of the wearer's tragus-concha bowl 957. Furthermore, at the same time, the taVNS stimulating electrode 31 will be naturally in close contact with the skin of the wearer's vagus innervated auricular skin since the skin of the external ear canal 904 and the skin of the tragus-concha bowl 957 are part of the vagus innervated auricular skin. Likewise, at the same time, the auriculotemporal nerve stimulating electrode 312 will be naturally in close contact with the skin of the wearer's auriculotemporal nerve innervated auricular skin since the skin of the external ear canal 904 and the skin of the tragus-concha bowl 957 are part of the auriculotemporal nerve innervated auricular skin. Similarly, at the same time, the GAN stimulating electrode 314 will be naturally in close contact with the skin of the wearer's tragus-concha bowl 957 since the skin of cavum concha 907 (part of the tragus-concha bowl 957) is part of the GAN innervated auricular skin. This unique design and set-up will allow the wearer 900 to attach or to remove all of the EEG sensor electrodes 12, 13, 72, 73, 82, 83, the optional reference electrode 84, the auricular SCOS sensor 512, the PPG unit 555, the taVNS stimulating electrodes 31, the auriculotemporal nerve stimulating electrode 312 and the GAN stimulating electrode 314 as easily as placing or removing the modified in-the-ear housing 62 in (or from) the wearer's 900 external ear canal 904 and the tragus-concha bowl 957.

In some embodiments for an auricular EEG monitoring system 100 and/or an automatic detection-therapy system 101, a standalone tubular-shaped structure 66 may be used for housing purpose. The housing setups and arrangements for the standalone tubular-shaped structure 66 are essentially the same as the tubular-shaped structure 66 of the modified earbud housing 61 (or the behind-the-ear-hearing-aid-style housing 63 or the modified in-the-ear housing 62) described hereinbefore.

As aforementioned, the auricular housing 11 (including all of these 3 types of housing, i.e. a modified earbud housing 61, a behind-the-ear-hearing-aid-style housing 63 and a modified in-the-ear housing 62) comprises a tubular-shaped structure 66. In preferred embodiments for an auricular EEG monitoring system 100 and/or an automatic detection-therapy system 101, all of the EEG sensor electrodes 12, 13, 72, 73, 82, 83, may be housed at the surface 91 of the tubular-shaped structure 66 and partially embedded with slight protrusion at the surface 91 of the tubular-shaped structure 66. One or more of the EEG sensor electrodes 12, 13, 72, 73, 82, 83, is/are configured to be located at the upper surface 91 (upper surface at approximately 90 degrees above horizontal level 92, as shown by electrodes 12, 13, in FIGS. 19, 22, 25) of the tubular-shaped structure 66. In preferred embodiments, one or more of the EEG sensor electrodes 12, 13, 72, 73, 82, 83, is/are configured to be located at between 0 and 90 degrees, and more preferably at approximately 45 degrees (plus or minus fifteen degrees or 30-60 degrees) above the horizontal level 92 of the tubular-shaped structure 66 and is/are configured to face forward-upward direction 93 (e.g., as shown by electrodes 72, 82, in FIGS. 19, 21, 24). In preferred embodiments, one or more of the EEG sensor electrodes is/are configured to be located at between 90 and 180 degrees, and more preferably at approximately 135 degrees (plus or minus fifteen degrees or 120-150 degrees) above the horizontal level 92 of the tubular-shaped structure 66 and is/are configured to face backward-upward direction 94 (e.g., as shown by electrodes 73, 83, in FIGS. 19, 23, 26). These arrangements will enable the EEG sensor electrodes 12, 13, 72, 73, 82, 83, to have one of the best locations and directions from the external ear canal 904 to record the wearer's EEG activities. (Upper surface, horizontal level, forward, backward, and upward all refer to directions relative to the head 901 of the wearer 900 with the wearer in an upright position after the tubular-shaped structure 66 has been inserted into a wearer's external ear canal 904.)

In some embodiments for an automatic detection-therapy system 101 for migraine, a separate client device 400 may be used for housing of some of the components of the automatic detection-therapy system 101. All of the EEG sensor electrodes 12, 13, 72, 73, 82, 83, may be configured as wireless EEG sensor electrodes. The optional reference electrode 84 may be configured as wireless optional reference electrode 84. The EEG recording module 20 may be configured to comprise a wireless EEG amplifier 21. Thus, all of the wireless EEG sensor electrodes 12, 13, 72, 73, 82, 83 and the optional wireless reference electrode 84 may be housed in an auricular housing 11 while the EEG recording module 20 and the processing unit may be configured to be housed remotely in one of: a watch-type client device, a smart phone-type client device or a tablet-type client device. Wireless EEG sensor electrodes may be used for all of the EEG sensor electrodes 12, 13, 72, 73, 82, 83, and the optional reference electrode 84. (Wireless dry EEG electrodes such as that described by Ryan Kaveh et al. in Nature Communications on Aug. 2, 2024. Wireless dry EEG electrodes are also available from Zeto, Inc. headquarter in Santa Clara, California.) There are well-known wired or wireless EEG amplifiers available, such as EEG Electroencephalogram Smart Amplifier (Part #: EEG100D), or preferably BioNomadix 2Ch Wireless EEG Amplifier (Part #: BN-EEG2), both being made by the same company BIOPAC Systems, Inc. (Goleta, California). By using wireless EEG sensor electrodes 12, 13, 72, 73, 82, 83, wireless optional reference electrode 84 and wireless EEG amplifier 21, the EEG recording module 20 together with the processing unit 50, 401, may be housed remotely in a watch-type, smart phone-type or tablet-type client device 400 and communicate with all of the EEG sensor electrodes 12, 13, 72, 73, 82, 83, and the optional reference electrode 84 wirelessly.

In some embodiments, an automatic detection-therapy system 101 for migraine may comprises a switch 88 and a timer 89 to enable the wearer 900 to turn on the system 101 manually and setting a duration, an interval or a schedule, etc. Preferably, the switch 88 may be configured as a multi-mode switch 88 and the timer 89 may be configured as a multi-mode timer 89. In some embodiments, a multi-mode switch 88 may be configured to enable the wearer to set the system 101 at an automatic mode or a manual mode. The functions and setups of the automatic mode for system 101 are as described hereinbefore. In some embodiments for an automatic detection-therapy system 101 for migraine, the multi-mode switch 88 may be configured to allow the wearer 900 to choose at least one of the following switch selections: turning on or off an auricular EEG monitoring system 100, turning on or off an auricular SCOS unit 511 turning on or off a PPG unit 555, turning on or off a taVNS unit 30, turning on or off an auriculotemporal nerve (ATN) stimulation unit 302, turning on or off a greater auricular nerve (GAN) stimulation unit 304, turning on or off a supraorbital nerve stimulation unit 301, turning on or off an occipital nerve stimulation unit 303, turning on or off an infraorbital nerve stimulation unit 305, or a combination thereof: The multi-mode timer 89 may be configured to function together with the multi-mode switch 88 to set each switch selection at a desirable timing course such as duration, interval, countdown, daily/weekly schedule, 10 minute/30 minutes/60 minutes (or other duration) selection, continuous operation, manual turning on and manual shutdown etc. Furthermore, the multi-mode switch 88 and multi-mode timer 89 may be configured as programmable. In some embodiments, the multi-mode switch 88 functions and the multi-mode timer 89 functions may be configured to be incorporated into a wearer's client device 400 and can be easily set up as programmable. In the manual mode, the wearer 900 can use the taVNS unit 30 or the ATN stimulation unit 302 or the GAN stimulation unit 304, or supraorbital nerve stimulation unit 301, or occipital nerve stimulation unit 303, or infraorbital nerve stimulation unit 305, or various combinations thereof for prophylactic purpose against migraine or for health maintenance purpose. Studies have shown that the taVNS unit 30 is effective not only for therapy of migraine and impending migraine, but also for prophylaxis of migraine. Studies have also shown that neuromodulating electric stimulation from the taVNS unit 30, the supraorbital nerve stimulation unit 301, the ATN stimulation unit 302, the occipital nerve stimulation unit 303 and GAN stimulation unit 304 (and combination thereof) are effective not only for therapy of migraine, impending migraine, and migraine prophylaxis but also for other neuropsychiatric diseases and for health maintenance. The aforementioned programmable set up for the system 101 will enable the system 101 to be used for prophylactic purpose and health maintenance purpose. The multi-mode switch 88, multi-mode timer 89 and the programmable design may enable the wearer the option to choose from one of the following: neuromodulation from one neuromodulation component (single neuromodulation), simultaneous neuromodulation from two neuromodulation components (double neuromodulation) and simultaneous neuromodulation from three neuromodulation components (triple neuromodulation). Thus, when prompted or activated, the neuromodulation unit 300 is configured to generate neuromodulating electric stimulation to a wearer 900 according to the choice of the stimulation mode selected by the wearer 900. The choices of the stimulation mode include the following: single neuromodulation by one component of the neuromodulation unit 300, double neuromodulation by various combinations of two components of the neuromodulation unit 300, triple neuromodulation by various combinations of three components of the neuromodulation unit 300, etc.

There are many conventional smart watches or health trackers that contain monitoring devices 71 to monitor various body functions, including electrocardiogram (ECG), heart rate, blood oxygen, sleep, body temperature, and motion etc. (as known in the art). However, none of them can monitor the most important part of the human body, which is the brain. The auricular EEG monitoring system 100 and/or the automatic detection-therapy system 101 can easily combine with other monitoring devices 71 by placing their monitoring sensors in the external ear 902 or the external ear canal 904. Thus, system 100 and/or system 101 can monitor not only the brain (through EEG), it can also monitor other body functions, such as ECG, heart rate, blood oxygen, motion, sleep and body temperature, etc.

### A Second Preferred Embodiment

In preferred embodiments, an automatic detection-therapy system 101 for cluster headache may comprise an auricular EEG monitoring system 100 having an auricular electroencephalogram (EEG) recording module 20, a neuromodulation unit 300 and a processing unit 50, 401. The auricular EEG recording module 20 may have a plurality (at least two, but preferably more than two) of miniature wired or wireless EEG sensor electrodes 12, 13, 72, 73, 82, 83 and a wired or wireless EEG amplifier 21. Optionally, the EEG recording module 20 may include an optional reference electrode 84. (The optional reference electrode 84 would be desirable but is not always necessary. Instead, average of all of the EEG sensor electrodes 12, 13, 72, 73, 82, 83, can be used as a reference, i.e. common average reference). The EEG sensor electrodes 12, 13, 72, 73, 82, 83, and the optional reference electrode 84 may be configured to contact separate areas selected from at least one of the following: external ear canal 904 of a first ear, external ear 902 of the first ear or peri-auricular area 903 around the first ear. The optional reference electrode 84 of the EEG recording module 20 may be placed in the tragus-concha bowl 957 or at the mastoid of the peri-auricular area 903. (Tragus-concha bowl 957 and mastoid are known to be good locations for EEG reference electrode.) The EEG recording module 20 may be configured to record and generate EEG data of the wearer 900 using electrical activities of the wearer 900 that are picked up via all of the EEG sensor electrodes 12, 13, 72, 73, 82, 83, and the optional reference electrode 84 (if the 84 is incorporated). (FIGS. 2, 7, 11-13). The EEG recording module 20 is in electronic communication with the processing unit 50, 401. The processing unit 50, 401 is configured to converts raw EEG data into quantitative EEG (qEEG) data through digital signal processing (DSP) techniques that transform, analyze, and mathematically quantify brain electrical activity. The processing unit 50, 401, is configured to use the qEEG data to detect cluster headache and impending cluster headache, including detecting increased theta and delta wave activities (particularly in temporal and frontal lobes) during cluster headache and changes in thalamus and hypothalamus which may be detected through the auricular EEG monitoring system 100. The processing unit 50, 401, is configured to analyze these qEEG data to detect cluster headache and impending cluster headache to help the wearer to take appropriate action and to actuate the neuromodulation unit 300 to start or stop neuromodulation therapy under the regulation by the closed-loop control system. It should be noted that, as compared with migraine, cluster headache produces only subtle changes in EEG and qEEG during the pre-ictal and ictal phases. (Please note that cluster headache produces only subtle changes in cerebral cortex and thus the traditional scalp EEG can detect only subtle changes during cluster headache. Cluster headache does produce significant changes in thalamus and hypothalamus and these regions are more easily assessable by in-ear EEG. Data from qEEG are valuable when using closed-loop control system to regulate the neuromodulation unit 300.) (Please also note that blood flow changes are more pronounced in cluster headache. Monitoring of blood flow is more important for detecting cluster headache and impending cluster headache.)

In preferred embodiments, an automatic detection-therapy system 101 for cluster headache may comprise a neurovascular monitoring system 500, a first neuromodulation unit 300 and a processing unit 50, 401. The neurovascular monitoring system 500 comprises an auricular EEG monitoring system 100 and a cephalic blood flow monitoring system 530. The auricular EEG monitoring system 100 is as described hereinbefore. The cephalic blood flow monitoring system 530 is configured to assess the cerebral (intracranial) and extracranial blood flow. The cephalic blood flow monitoring system 530 may be configured as an ultrasound doppler-based unit or a photoplethysmography (PPG) unit 555 or a laser speckle-based unit. For example, a Transcranial Doppler Ultrasound (TCD) is a non-invasive technique for real-time monitoring of cerebral blood flow. It can detect abnormalities such as blockages or irregular blood flow patterns, which may indicate conditions like stroke or carotid artery disease. New development in laser technology also provides convenient way to monitor cerebral and extracranial blood flow. There are a few laser speckle-based blood flow monitoring units. Examples include a Speckle Contrast Optical Spectroscope unit (SCOS unit) 510. A novel auricular Speckle Contrast Optical Spectroscope unit (auricular SCOS unit) 511 is disclosed by placing a SCOS unit 510 in an auricular housing 11, such as a modified earbud housing 61 or an in-the-ear housing 62 or a behind-the-ear-hearing-aid-style housing 63. The auricular SCOS unit 511 comprises a light source (laser) 519 that pass through a focusing lens 518 and an expander 513 and mirrors 514 and then tissue (or sample) 515. The light is then collected by a SCOS sensor (detector) 512 that is integrated with a Complementary Metal-Oxide Semiconductor camera (CMOS camera) 516 and the data are transmitted to a data processing unit (computer) 517 for analysis and to get the blood flow data at different depths of the tissue, including the cerebral and extracranial blood flow data. (FIG. 34). In some embodiments, a first auricular SCOS sensor 512 is configured to be located on a surface of a tragus-concha bowl 957 of the wearer's first ear or the external ear canal 904 of the wearer's first ear when in use to collect the wearer's cerebral (intracranial) and extracranial blood flow data. In some embodiments, a cephalic blood flow monitoring system 530 may comprise an auricular SCOS unit 511 and a PPG unit 555 (both unit 511 and unit 555 may be located in the auricular housing 11) to enhance the capability to monitor the cerebral and extracranial blood flow. In some embodiments, a first neurovascular monitoring system 500 comprises a first auricular EEG monitoring system 100 (configured to record the wearer's EEG and qEEG data from a first ear 902 of the wearer) and a first auricular SCOS unit 511 (configured to record cerebral and extracranial blood flow data from the wearer's first ear 902), configured to simultaneously record (collect) the wearer's EEG and qEEG data and cerebral (intracranial) and extracranial blood flow data.

In some embodiments, an auricular detection-therapy system for cluster headache 101 may comprise a cephalic blood flow monitoring system 530, a first neuromodulation unit 300 and a processing unit 50, 401. The cephalic blood flow monitoring system 530 is configured to record cerebral and extracranial blood flow data of the wearer 900. The first neuromodulation unit 300 is configured to give neuromodulating electric stimulation to the wearer 900 when activated. The neuromodulating electric stimulation parameters may be pre-determined (open-loop controlled) or feedback controlled (closed-loop controlled). The timing and when to turn on or turn off the first neuromodulation unit 300 may also be closed-loop controlled. The processing unit 50, 401, is in electronic communication with the cephalic blood flow monitoring system 530 and the first neuromodulation unit 300. The processing unit 50, 401 is configured to analyze the cerebral and extracranial blood flow data recorded by the cephalic blood flow monitoring system 530 to detect presence or cessation of cerebral and extracranial blood flow data suggestive of cluster headache. When the processing unit 50, 401 detects presence of cerebral and extracranial blood flow data suggestive of cluster headache, the processing unit 50, 401, is configured to immediately send signals to the first neuromodulation unit 300 to automatically start sending neuromodulating electric stimulation to the wearer 900. The processing unit 50, 401, is also configured to analyze the cerebral and extracranial blood flow data recorded by the cephalic blood flow monitoring system 530 to detect presence or cessation of cerebral and extracranial blood flow data suggestive of impending cluster headache. When the processing unit 50, 401, detects presence of cerebral and extracranial blood flow data suggestive of impending cluster headache, the processing unit 50, 401, is configured to immediately send signals to the first neuromodulation unit 300 to automatically start sending neuromodulating electric stimulation to the wearer 900. When the processing unit 50, 401, detects both of the following: cessation of cerebral and extracranial blood flow data suggestive of cluster headache and cessation of cerebral and extracranial blood flow data suggestive of impending cluster headache, the processing unit 50, 401, is further configured to immediately send signals to the first neuromodulation unit 300 to automatically stop sending neuromodulating electric stimulation to the wearer 900.

In preferred embodiments for an automatic detection-therapy system 101 for cluster headache, the first neuromodulation unit 300 may include at least one of the following components: a first transcutaneous auricular vagus nerve stimulation unit (first taVNS unit) 30, a supraorbital nerve stimulation unit 301, an infraorbital nerve stimulation unit 305, a first auriculotemporal nerve stimulation unit 302, an occipital nerve stimulation unit 303 and a first greater auricular nerve (GAN) stimulation unit 304. When any of these components (including unit 30, unit 301, unit 302, unit 303, unit 304, unit 305) is activated, neuromodulating electric stimulation will be sent to the wearer (user) 900. There are evidences showing the benefits of neuromodulating electric stimulation from taVNS unit 30. More recently, there are also evidences showing benefits of neuromodulating electric stimulation from non-vagus electric neuromodulation unit. As used herein, the term “non-vagus electric neuromodulation unit” refers to one of the following: a supraorbital nerve stimulation unit 301, an infraorbital nerve stimulation unit 305, an auriculotemporal nerve stimulation unit 302, an occipital nerve stimulation unit 303, a greater auricular nerve stimulation unit 304, and various combinations thereof: Non-vagus electric neuromodulation has significant benefits for cluster headache. Simultaneous stimulation from a taVNS unit 30 and a non-vagus electric neuromodulation unit has synergistic effects. The first taVNS unit 30 may comprise a miniature first taVNS stimulating electrode 31. The first taVNS stimulating electrode 31 may be configured to contact vagus innervated auricular skin of the wearer's 900 first ear. The vagus innervated auricular skin that the taVNS stimulating electrode 31 is configured to contact may be selected from at least one of the following: posterior and inferior walls of external ear canal 904, inner/posterior portion of tragus 905, cymba-concha 906, and majority of cavum-concha 907. (Part of the eardrum also receives vagus innervation but eardrum is not suitable for electrode placement). When prompted, the first taVNS unit 30 may be configured to give neuromodulating electric stimulation through the first taVNS stimulating electrode 31 to the vagus innervated auricular skin of the wearer's first ear in a way similar to transcutaneous electric nerve stimulation (TENS) (taVNS and TENS as known in the art). (FIGS. 2, 11-13). The supraorbital nerve stimulation unit 301 may comprise a supraorbital nerve stimulating electrode 311 to be attached to supraorbital nerve innervated forehead skin. The supraorbital nerve stimulation unit 301 is configured to give neuromodulating electric stimulation through the supraorbital nerve stimulating electrode 311 to a wearer's supraorbital nerve innervated forehead skin when prompted. (FIG. 29). The infraorbital nerve is a branch of the second division of the trigeminal nerve and the function of the infraorbital nerve stimulation unit 305 is essentially similar to that of the supraorbital nerve stimulation unit 301. The first auriculotemporal nerve stimulation unit 302 may comprise a first auriculotemporal nerve stimulating electrode 312 to be attached to the auriculotemporal nerve innervated skin of the wearer's first ear 902. The auriculotemporal nerve innervated auricular skin include anterior-superior helix 909, anterior outer part of tragus 905, anterior portion (or anterior superior part) of cavum-concha 907, anterior and superior walls of the external ear canal 904. (The anterior auricle and part of the outer tympanic membrane also receive auriculotemporal nerve innervation). Thus, the tragus-concha bowl 957 and the external ear canal 904 received mixed innervation from both the auricular branch of vagus nerve and the auriculotemporal nerve. The first auriculotemporal nerve stimulation unit 302 is configured to give neuromodulating electric stimulation through the first auriculotemporal nerve stimulating electrode 312 to a wearer's auriculotemporal nerve innervated skin of the wearer's first ear when prompted. (FIG. 31). The occipital nerve stimulation unit 303 may comprise an occipital nerve stimulating electrode 313 to be attached to occipital nerve innervated occipital region. The occipital nerve stimulation unit 303 is configured to give neuromodulating electric stimulation through the occipital nerve stimulating electrode 313 to a wearer's occipital nerve innervated occipital region when prompted. (FIG. 30). The first greater auricular nerve (GAN) stimulation unit 304 may comprise a first GAN stimulating electrode 313 to be attached to GAN innervated auricular skin of the wearer's first ear. The first GAN stimulation unit 304 is configured to give neuromodulating electric stimulation through the first GAN stimulating electrode 314 to the GAN innervated auricular skin of the wearer's first ear when prompted. (FIG. 33). (For comparison: The vagus-innervated auricular skin includes: inner posterior portion of tragus 905, cymba-concha 906, cavum-concha 907, posterior and inferior walls of the external ear canal 904 and small adjacent regions of the external ear. The auriculotemporal nerve innervated auricular skin includes: anterior outer part of tragus 905, anterior-superior part of cavum concha 907, anterior and superior walls of the external ear canal 904 and, anterior and superior part of pinna including anterior-superior helix 909. The greater auricular nerve innervated auricular skin includes: cavum concha, lower two thirds of anterior and posterior pinna and the mastoid process.) From the above comparison, it is obvious that the tragus-concha 957 of a wearer's ear 902 received mixed and overlapping innervation from the auricular branch of vagus nerve, the auriculotemporal nerve (ATN) and the greater auricular nerve (GAN). When the taVNS stimulating electrode 31, the ATN stimulating electrode 312 and the GAN stimulating electrode 314 are located on the body-structure 67, by carefully selecting the locations for the taVNS stimulating electrode 31 the ATN stimulating electrode 312 and the GAN stimulating electrode 314 on the body-structure 67 to match the aforementioned innervation locations of the innervated skin on the tragus-concha bowl 957, these stimulating electrodes 31, 312, 314, will automatically get in close contact with their respective target skin on the tragus-concha bowl 957 when the body-structure 67 is placed in the tragus-concha bowl 957. Alternatively, when the taVNS stimulating electrode 31 and the ATN stimulating electrode 312 are located on the tubular-shaped structure 66, by carefully selecting the locations for the taVNS stimulating electrode 31 and the ATN stimulating electrode 312 on the tubular-shaped structure 66 to match the aforementioned innervation locations of the innervated skin on the external ear canal 904, these stimulating electrodes 31, 312, will automatically get in close contact with their target skin on the external ear canal 904 when the tubular-shaped structure 66 is placed in the external ear canal 904.

In some embodiments for an automatic detection-therapy system 101 for cluster headache, the auricular EEG recording module 20 may be in electronic communication with the processing unit 50, 401, through Bluetooth, wired, wireless or other electronic connection means or methods. Each component of the neuromodulation unit 300 (including at least one of: the taVNS unit 30, the supraorbital nerve stimulation unit 301, the infraorbital nerve stimulation unit 305, the auriculotemporal nerve stimulation unit 302, the occipital nerve stimulation unit 303 and the greater auricular nerve stimulation unit 304) may also be in electronic communication with the processing unit 50, 401, through Bluetooth, wired, wireless or other connection means. The EEG recording module 20 collects or records the wearer's EEG and qEEG data and these data are transmitted to the processing unit 50, 401. With the help of various advanced EEG analysis algorithms together with machine learning, deep learning and artificial intelligence (as known in the art), the processing unit 50, 401, is configured to analyze the EEG and qEEG data to detect presence of EEG and qEEG signals of wearer 900 suggestive of cluster headache and impending cluster headache. The processing unit 50, 401, may be also configured to detect cessation of EEG and qEEG signals of wearer 900 suggestive of cluster headache or cessation of EEG and qEEG signals suggestive of impending cluster headache. In some embodiments, an automatic detection-therapy system 101 for cluster headache may comprise a neurovascular monitoring system 500 that includes an auricular EEG monitoring system 100 and an auricular Speckle Contrast Optical Spectroscopy unit (auricular SCOS unit) 511. Alternatively, the cephalic blood flow monitoring system 530 may comprise an auricular SCOS unit 511 and a photoplethysmography (PPG) unit 555. (PPG unit 555 may enhance the capability of SCOS unit 5111 in assessing cerebral and extracranial blood flow, especially the extracranial blood flow. PPG unit 555 can also help to estimate the wearer's systolic and diastolic blood pressure.) (There is evidence that combining SCOS and PPG together can enhance the accuracy of blood flow assessments compared to using either method alone, as SCOS captures high-frequency changes in blood flow while PPG provides volume data. i.e. complementary effect). In some embodiments, a cephalic blood flow monitoring system may comprise one of a SCOS unit 510 and an auricular SCOS unit 511 and may further comprises a photoplethysmography (PPG) unit 555. The PPG unit 555 is configured to record the wearer's cerebral and extracranial blood flow data. The PPG unit 555 is in electronic communication with the processing unit 50, 401. The processing unit 50, 401, is configured to analyze the cerebral and extracranial blood flow data recorded by the PPG unit 555 and one of: the SCOS unit 510 and the auricular SCOS unit 511 to assess the wearer's cerebral and extracranial blood flow data. The auricular SCOS unit 511 and the auricular EEG monitoring system 100 may be in electronic communication with the processing unit 50, 401, through Bluetooth, wired, wireless or other electronic connection means or methods. The auricular EEG monitoring system 100 and the auricular SCOS unit 511 are configured to function at the same time for simultaneous recording of EEG and qEEG data and recording of cerebral and extracranial blood flow data of the wearer 900.

When the processing unit 50, 401, detects one of the following: presence of EEG and qEEG signals suggestive of cluster headache and presence of blood flow data suggestive of cluster headache, the processing unit 50, 401, may be configured to automatically send signals to the neuromodulation unit 300 to actuate (or activate) at least one of the following components: the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305 to start sending pre-determined neuromodulating electric stimulation immediately. When the processing unit 50, 401, detects one of the following: presence of EEG and qEEG signals suggestive of impending cluster headache and presence of blood flow data suggestive of impending cluster headache, the processing unit 50, 401, may be configured to automatically send signals to the neuromodulation unit 300 to actuate at least one of the following components: the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305 to start sending pre-determined neuromodulating electric stimulation immediately. When the processing unit detects all of the following: cessation of EEG and qEEG signals suggestive of cluster headache, cessation of blood flow data suggestive of cluster headache, cessation of EEG and qEEG signals suggestive of impending cluster headache and cessation of blood flow data suggestive of impending cluster headache, the processing unit 50, 401, may be further configured to automatically send signals to the neuromodulation unit 300 to stop sending pre-determined electric stimulation from any of the following: the taVNS unit 30, the supraorbital nerve stimulation unit 301, the auriculotemporal nerve stimulation unit 302, the occipital nerve stimulation unit 303, the greater auricular nerve stimulation unit 304 and the infraorbital nerve stimulation unit 305.

For the taVNS unit 30, the parameters of the electric stimulation for cluster headache, including the stimulating patterns, strength, duration, frequency and intervals, are predetermined, such as shown in Table 3. For the taVNS unit 30, the parameters of the electric stimulation for impending cluster headache, including the stimulating patterns, strength, duration, frequency and intervals, are also predetermined, such as shown in Table 4. Some examples of stimulus parameters for other components of the neuromodulation unit 300 (such as supraorbital nerve stimulation unit 301, auriculotemporal nerve stimulation unit 302, occipital nerve stimulation unit 303, greater auricular nerve stimulation unit 304 and infraorbital nerve stimulation unit 305) for cluster headache and impending cluster headache were described hereinbefore and by various articles in the Background section of this invention. In some embodiments, the processing unit 50, 401, may include a closed-loop control system (a “sensor-controller-actuator” setup) (or a feedback-controlled mechanism). The closed-loop control system within the processing unit 50, 401, may be in electronic communication with the neurovascular monitoring system 500 (including the auricular EEG monitoring system 100 and cephalic blood flow monitoring system 530) and the neuromodulation unit 300. The closed-loop control system receives real-time continuous input of the wearer's 900 EEG and qEEG data from the auricular EEG monitoring system 100 and real-time continuous input of the wearer's cerebral and extracranial blood flow data from the cephalic blood flow monitoring system 530 and the closed-loop control system of the processing unit 50, 401, is configured to analyze these real-time EEG and qEEG data and cerebral and extracranial blood flow data, using controlling algorithms, to continuously adjust the actuating outputs to the neuromodulation unit 300. The actuating outputs includes turning on or turning off at least one component of the neuromodulation unit 300 and adjusting the stimulating parameters (intensity, frequency, duration, cycle, wave form etc.) of the neuromodulating unit 300 during the time when the neuromodulating unit 300 is turned on. This will greatly enhance the effectiveness and safety of neuromodulation, especially trigeminal nerve stimulation. In trigeminal nerve neuromodulation, some high-frequency (e.g., 200 Hz) stimulation could produce pronounced effects on heart rate and blood pressure compared to lower frequencies. Studies have shown that high frequency (e.g. 200 Hz) trigeminal nerve electric stimulation can produce a rapid, transient decrease in heart rate (bradycardia) and, in some cases, a sudden, brief drop in blood pressure (hypotension) due to activation of the trigeminal cardiac reflex. Both taVNS and trigeminal nerve stimulation are known to be effective for migraine and cluster headache. Studies have shown that trigeminal nerve stimulation has stronger (FDA-cleared) evidence specifically for acute and preventive migraine treatment, while taVNS has particular strength in reducing migraine frequency and treating cluster headache. High frequency trigeminal nerve stimulation (in the 100-150 Hz range) is generally considered more effective for acute migraine and cluster headache treatment, while specific lower frequency combinations are used for prevention of migraine and cluster headache. Using controlled-loop control system together with blood flow monitoring from auricular SCOS unit 511, blood pressure monitoring from the PPG unit 555 and EEG and qEEG data from the auricular EEG monitoring system 100 can minimize the risk when high frequency is used for trigeminal nerve stimulation. When any of the following: a decrease of blood flow more than a predetermined level (e.g. a decrease of blood flow of 20% or more), a decrease of blood pressure more than a predetermined level (e.g. at least one of: a decrease of systolic blood pressure of 20 mmHg or more and a decrease of diastolic blood pressure of 20 mmHg or more) and the EEG and qEEG data changes more than a predetermined level (e.g. at least one of: an increase of relative delta power of 20% or more and an increase of delta/alpha ratio of 20% or more) (or significant changes of other quantitative data from qEEG), the stimulation may be immediately stopped or the stimulation parameters may be immediately altered. High frequency (around 100-160 Hz) occipital nerve stimulation has been found to be more effective than low frequency for migraine and cluster headache. Alternating frequencies (2/100 Hz) occipital nerve stimulation has also been found to be effective.

In preferred embodiments, for most patients whose cluster headache is usually on one side of the head 901, one neurovascular monitoring system 500 placed on the same side may be preferred. These patients may use one neuromodulation unit 500 on the same side (although, optionally, two neuromodulation units 500, with one unit on each side of the head, might be considered). In other embodiments, for some patients with cluster headache, two neurovascular monitoring systems 500, one on each side of the head 901 linked to each ear 902, may be considered. For some patients with cluster headache, two neuromodulation units 300 may be considered, with one neuromodulation unit 300 on each side of the head 901. There are evidences that in most cases, cluster headaches occur on the same side of the head. During a cluster period, the pain is typically unilateral, affecting only one side during each episode. However, it is also known that in rare situations, cluster headache can switch sides between different cluster episodes. If a patient is unable to tolerate a taVNS unit 30 in right ear due to bradycardia or other side effects, only one taVNS unit 30 may be utilized and be placed in the left ear. Likewise, for some patients with cluster headache, two auriculotemporal nerve stimulation unit 302 might be considered, with one auriculotemporal nerve stimulation unit 302 on each side of the head 901 coupled to the tragus-concha bowl 957 or external ear canal 904 of each ear 902. Similarly, for some patients with cluster headache, two greater auricular nerve stimulation unit 304 might be considered, with one greater auricular nerve stimulation unit 304 on each side of the ear 902 coupled to the tragus-concha bowl 957 (cavum concha 907) of each ear 902. In some embodiments, an automatic detection-therapy system 101 for cluster headache may comprise a neurovascular monitoring system 500 that includes an auricular EEG monitoring system 100 having a first EEG recording module 20 and a second EEG recording module 20 and a cephalic blood flow monitoring system 530 having a first auricular SCOS unit 511 and a second auricular SCOS unit 511. (Alternatively, a cephalic blood flow monitoring system 530 may comprise a first SCOS unit 511 and a first PPG unit 555 linked to a first ear 902 of the wearer and a second SCOS unit 511 and a second PPG unit 555 linked to a second ear 902 of the wearer.) The first EEG recording module 20 and the first auricular SCOS unit 511 may be configured to be linked to a wearer's first ear 902, while the second EEG recording module 20 and the second auricular SCOS unit 511 may be configured to be linked to a second ear 902 of the wearer 900. The automatic detection-therapy system 101 for cluster headache may further comprise two neuromodulation units 300, with some of the components (units 30, 302, 304) of the first neuromodulation unit 300 being located in the wearer's first ear 902 while the second neuromodulation unit 300 being located in the wearer's second ear 902. The first neuromodulation unit 300 comprises at least one of: a first taVNS unit 30, a supraorbital nerve stimulation unit 301 (to be located at wearer's mid-forehead), an infraorbital nerve stimulation unit 305 (to be located at wearer's mid-face), a first auriculotemporal nerve stimulation unit 302, an occipital nerve stimulation unit 303 (to be located at wearer's mid-occipital region) and a first greater auricular nerve stimulation unit 304. The second neuromodulation unit 300 may comprise at least one of: a second taVNS unit 30, a second auriculotemporal nerve stimulation unit 302 and a second greater auricular nerve stimulation unit 304. The second taVNS unit 30 includes a second taVNS stimulating electrode 31 configured to contact vagus innervated auricular skin of the wearer's second ear 902. The second auriculotemporal nerve stimulation unit 302 includes a second auriculotemporal nerve stimulating electrode 312 configured to contact auriculotemporal nerve innervated auricular skin of the wearer's second ear 902, while the second greater auricular nerve stimulation unit 304 includes a second greater auricular nerve stimulating electrode 314 configured to contact greater auricular nerve innervated auricular skin of the wearer's second ear 902. The processing unit 50, 401, is configured to analyze the EEG and qEEG data recorded by the auricular EEG monitoring system 100, including EEG and qEEG data recorded by the first EEG recording module 20 and EEG and qEEG data recorded by the second EEG recording module 20, to detect presence or cessation of EEG and qEEG signals suggestive of cluster headache. The processing unit 50, 401, is further configured to analyze the EEG and qEEG data recorded by the auricular EEG monitoring system 100, including EEG and qEEG data recorded by the first EEG recording module 20 and EEG and qEEG data recorded by the second EEG recording module 20, to detect presence or cessation of EEG and qEEG signals suggestive of impending cluster headache. The processing unit 50, 401, is also configured to analyze the cerebral and extracranial blood flow data recorded by the cephalic blood flow monitoring system 530, including cerebral and extracranial blood flow data recorded by the first auricular SCOS unit 511 and the first PPG unit 555 and cerebral and extracranial blood flow data recorded by the second auricular SCOS unit 511 and the second PPG unit 555, to detect presence or cessation of cerebral and extracranial blood flow data suggestive of cluster headache. The processing unit 50, 401, is further configured to analyze the cerebral and extracranial blood flow data recorded by the cephalic blood flow monitoring system 530, including cerebral and extracranial blood flow data recorded by the first auricular SCOS unit 511 and the first PPG unit 555 and cerebral and extracranial blood flow data recorded by the second auricular SCOS unit 511 and the second PPG unit 555, to detect presence or cessation of cerebral and extracranial blood flow data suggestive of impending cluster headache. When the processing unit 50, 401, detects at least one of the following: presence of EEG and qEEG signals suggestive of cluster headache by analyzing the EEG and qEEG data recorded by the first EEG recording module 20, presence of EEG and qEEG signals suggestive of cluster headache by analyzing the EEG and qEEG data recorded by the second EEG recording module 20, presence of cerebral and extracranial blood flow data suggestive of cluster headache by analyzing cerebral and extracranial blood flow data recorded by the first auricular SCOS unit 511 and the first PPG unit 555, and presence of cerebral and extracranial blood flow data suggestive of cluster headache by analyzing cerebral and extracranial blood flow data recorded by the second auricular SCOS unit 511 and the second PPG unit 555, the processing unit 50, 401, is configured to start sending predetermined neuromodulating electric stimulation from at least one of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303 (including various combinations thereof). When the processing unit 50, 401, detects at least one of the following: presence of EEG and qEEG signals suggestive of impending cluster headache by analyzing EEG and qEEG data recorded by the first EEG recording module 20, presence of EEG and qEEG signals suggestive of impending cluster headache by analyzing EEG and qEEG data recorded by the second EEG recording module 20, presence of cerebral and extracranial blood flow data suggestive of impending cluster headache by analyzing cerebral and extracranial blood flow data recorded by the first auricular SCOS unit 511 and the first PPG unit 555, and presence of cerebral and extracranial blood flow data suggestive of impending cluster headache by analyzing cerebral and extracranial blood flow data recorded by the second auricular SCOS unit 511 and the second PPG unit 555, the processing unit 50, 401, is configured to start sending predetermined neuromodulating electric stimulation from at least one of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 302, the infraorbital nerve stimulation unit 305 and the occipital nerve stimulation unit 303 (including various combinations thereof). When the processing unit detects all of the following: cessation of EEG and qEEG signals suggestive of cluster headache by analyzing EEG and qEEG data recorded by the first EEG recording module 20, cessation of EEG and qEEG signals suggestive of impending cluster headache by analyzing EEG and qEEG data recorded by the first EEG recording module 20, cessation of cerebral and extracranial blood flow data suggestive of cluster headache by analyzing cerebral and extracranial blood flow data recorded by the first auricular SCOS unit 511 and the first PPG unit 555, cessation of cerebral and extracranial blood flow data suggestive of impending cluster headache by analyzing cerebral and extracranial blood flow data recorded by the first auricular SCOS unit 511 and the first PPG unit 555, cessation of EEG and qEEG signals suggestive of cluster headache by analyzing EEG and qEEG data recorded by the second EEG recording module 20, cessation of EEG and qEEG signals suggestive of impending cluster headache by analyzing EEG and qEEG data recorded by the second EEG recording module 20, cessation of cerebral and extracranial blood flow data suggestive of cluster headache by analyzing cerebral and extracranial blood flow data recorded by the second auricular SCOS unit 511 and the second PPG unit 555, and cessation of cerebral and extracranial blood flow data suggestive of impending cluster headache by analyzing cerebral and extracranial blood flow data recorded by the second auricular SCOS unit 511 and the second PPG unit 555, the processing unit is further configured to stop sending neuromodulating electric stimulation from any of the following: the first taVNS unit 30, the first auriculotemporal nerve stimulation unit 302, the first greater auricular nerve stimulation unit 304, the second taVNS unit 30, the second auriculotemporal nerve stimulation unit 302, the second greater auricular nerve stimulation unit 304, the supraorbital nerve stimulation unit 301, the infraorbital nerve stimulation unit 305 and the occipital nerve stimulation unit 303.

In some embodiments for an automatic detection-therapy system 101 for cluster headache, the auricular EEG recording module 20, the auricular SCOS unit 511, the PPG unit 555, the taVNS unit 30, the auriculotemporal nerve stimulation unit 302, the greater auricular nerve stimulation unit 304 and the processing unit 50, 401, may be housed together as a single structure and placed in the external ear canal 904, tragus 905, concha 906, 907, and/or peri-auricular area 903. Alternatively, the taVNS unit 30, the auricular SCOS unit 511, the PPG unit 555, the auriculotemporal nerve stimulation unit 302, the greater auricular nerve stimulation unit 304, and the EEG recording module 20 may be in separate housings and may be located adjacent to each other with each of them attached to one or more of the following: the external ear canal 904, the tragus-concha bowl 957, and the peri-auricular area 903. In some embodiments, all of the EEG sensor electrodes 12, 13, 72, 73, 82, 83, the optional reference electrode 84, the auricular SCOS sensor 512, the PPG unit 555, the taVNS stimulating electrode 31, the auriculotemporal nerve stimulating electrode 312 and the greater auricular nerve stimulating electrode 314 may be all housed together sharing a same auricular housing 11. The auricular housing 11 may be selected from one of the following: a modified earbud housing 61, a modified in-the-ear housing 62, and a behind-the-ear-hearing-aid-style housing 63, as described hereinbefore. All of these housings 61, 62, 63, include a tubular-shaped structure 66 (configured to be inserted into an external ear canal 904 of a wearer when in use) and a body-structure 67 (configured to be placed at the opening of the external ear canal 904 and to sit or to be placed inside a tragus-concha bowl 957 of the wearer's ear when in use). All of the EEG sensor electrodes 12, 13, 72, 73, 82, 83, may be configured to be located on a surface 91 of the tubular-shaped structure 66, while the auricular SCOS sensor 512, the PPG unit 555, the taVNS stimulating electrode 31 and the auriculotemporal nerve stimulating electrode 312 are configured to be housed on a surface of one of: the tubular-shaped structure 66 and the body-structure 67. The greater auricular nerve (GAN) stimulating electrode 314 and the optional reference electrode 84 are configured to be located at the surface of the body-structure 67. All of the EEG sensor electrodes 12, 13, 72, 73, 82, 83, the optional reference electrode 84, the auricular SCOS sensor 512, the PPG unit 555, the taVNS stimulating electrode 31, the auriculotemporal nerve stimulating electrode 312 and the GAN stimulating electrode 512 are configured to be partially embedded in the surface with slight protrusion at the surface of one of: the tubular-shaped structure 66 and the body-structure 67. (Alternatively, the auricular SCOS sensor 512 may be located slightly below the surface 91 of the tubular-shaped structure 66 or body-structure 67 so that there is a tiny air gap between the auricular SCOS sensor 512 and the skin of the wearer's external ear canal 904 or the skin of the wearer's tragus-concha bowl 957 when the tubular-shaped structure 66 and the body-structure 67 are placed in the wearer's ear 902.) The tubular-shaped structure 66 and the body-structure 67 may be configured to comprise a flexible elastic and adaptable material and the flexible elastic and adaptable material is configured to have appropriate flexibility, elasticity and adaptability so that the tubular-shaped structure 66 will naturally adapt to the contour of the external ear canal 904 and snugly fill the interior of the wearer's external ear canal 904 when the tubular-shaped structure 66 is inserted into the external ear canal 904 of the wearer's ear 902 and, meanwhile, the body-structure 67 will naturally adapt to the contour of the tragus-concha bowl 957 of the wearer's ear 902 and snugly fill the interior of the tragus-concha bowl 957 of the wearer's ear 902 when the body-structure 67 is placed inside the tragus-concha bowl 957 of the wearer's ear 902, so that all of the EEG sensor electrodes 12, 13, 72, 73, 82, 83, are naturally snugly in contact with the skin of the external ear canal 904 of the wearer's ear 902, and, at the same time, the auricular SCOS sensor 512, the PPG unit 555, the taVNS stimulating electrode 31 and the auriculotemporal nerve stimulating electrode 312 are naturally and snugly in contact with the skin of the tragus-concha bowl 957 of the wearer's ear 902 or the skin of the external ear canal 904 of the wearer's ear 902, while the GAN stimulating electrode 314 and the optional reference electrode 84 are naturally snugly in contact with the skin of the wearer's cavum concha 907 (or tragus-concha bowl 957), and so that the taVNS stimulating electrode 31 is naturally and snugly in contact with its target skin of vagus-innervated auricular skin of the wearer's first ear 902, provided by carefully selecting a location for the taVNS stimulating electrode 31 on the tubular-shaped structure 66 or the body-structure 67 to match one of the innervation locations of the vagus innervated skin on the external ear canal 904 or the tragus-concha bowl 957, and, at the same time, the auriculotemporal nerves stimulating electrode 312 will be naturally in close contact with its target skin of auriculotemporal nerve innervated auricular skin, provided by carefully selecting a location for the auriculotemporal nerve stimulating electrode 312 on the tubular-shaped structure 66 or the body-structure 67 to match one of the innervation locations of the auriculotemporal nerve innervated skin on the external ear canal 904 or the tragus-concha bowl 957, and, at the same time, the greater auricular nerve stimulating electrode 314 will be naturally in close contact with greater auricular nerve innervated auricular skin, provided by carefully selecting a location for the greater auricular nerve stimulating electrode 314 on the body-structure 67 to match one of the innervation locations of the greater auricular nerve innervated skin on tragus-concha bowl 957. As described hereinbefore, by comparing the innervation patterns, it is obvious that the tragus-concha 957 of a wearer's ear 902 received mixed and overlapping innervation from the auricular branch of vagus nerve, the auriculotemporal nerve and the greater auricular nerve. In some embodiments, a neuromodulation unit 300 may have a taVNS stimulating electrode 31, an auriculotemporal nerve stimulating electrode 312 and a greater auricular nerve stimulating electrode 314 and all of these electrodes (31, 312, 314) may be located on the body-structure 67. By carefully selecting the location for the taVNS stimulating electrode 31 on the body-structure 67 to match the innervation locations of the vagus innervated skin on the tragus-concha bowl 957, the taVNS stimulating electrode 31 will automatically get in close contact with its target skin on the tragus-concha bowl 957 when the body-structure 67 is placed in the tragus-concha bowl 957. By carefully selecting the location for the auriculotemporal nerve stimulating electrode 312 on the body-structure 67 to match the innervation locations of the auriculotemporal nerve on the tragus-concha bowl 957, the auriculotemporal nerve stimulating electrode 312 will automatically get in close contact with its target skin on the tragus-concha bowl 957 when the body-structure 67 is placed in the tragus-concha bowl 957. Similarly, by carefully selecting the location for the greater auricular nerve stimulating electrode 314, on the body-structure 67 to match the innervation locations of the greater auricular nerve innervated skin on the tragus-concha bowl 957, the greater auricular nerve stimulating electrode 314 will automatically get in close contact with its target skin on the tragus-concha bowl 957 when the body-structure 67 is placed in the tragus-concha bowl 957. Alternatively, the taVNS stimulating electrode 31 and the auriculotemporal nerve stimulating electrode 312 may be located on the tubular-shaped structure 66. By carefully selecting the location for the taVNS stimulating electrode 31 on the tubular-shaped structure 66 to match its target skin locations on the external ear canal 904, the taVNS stimulating electrode 31 will be automatically get in close contact with its target skin in the external ear canal 904. Similarly, by carefully selecting the location for the auriculotemporal nerve stimulating electrode 312 on the tubular-shaped structure 66 to match its target skin locations on the external ear canal 904, the auriculotemporal nerve stimulating electrode 312 will be automatically get in close contact with its target skin in the external ear canal 904.

Preferably, the automatic detection-therapy system 101 for cluster headache further comprises a network interface 53, 406, in electronic communication with the processing unit 50, 401. The network interface 53, 406, may be configured to generate a notification (such as an audible notification via a speaker 15, 404A, or a tactile notification via a vibrator 17, 404B). When cluster headache or impending cluster headache is detected (by EEG signals and blood flow data as described hereinbefore), the network interface 53, 406, may be configured to automatically generate a notification to a client device 400, such as to the client device 400 of the wearer 900 and/or the client device 400 of the wearer's healthcare provider 950 so that the wearer 900 or the wearer's healthcare provider 950 can take appropriate actions. Besides that, the neuromodulation unit 300 may be automatically actuated to start giving pre-determined neuromodulating electric stimulation to the wearer. The neuromodulation unit 300 includes at least one of the following components: a taVNS unit 30, a supraorbital nerve stimulation unit 301, an auriculotemporal nerve stimulation unit 302, an occipital nerve stimulation unit 303, a greater auricular nerve stimulation unit 304 and infraorbital nerve stimulation unit 305. Thus, when cluster headache or impending cluster headache is detected, in addition to receiving at least one neuromodulation therapy automatically, the wearer 900 can take extra dose of medications or take other actions to alleviate the condition. An example of the notification device of a client device 400 is a speaker 15, 404A, configured to generate audible warning notification (or a vibrator 17, 404 B, configured to generate tactile notification) when presence of EEG and qEEG signals or blood flow data suggestive of cluster headache or impending cluster headache is detected by the processing unit 50, 401 (as described hereinbefore). The notification function is well-known. Most smart phones, smart watches and health trackers have notification function.

In some embodiments, an automatic detection-therapy system 101 for cluster headache may comprise an auricular EEG recording module 20, a processing unit 50, 401, an auricular SCOS unit 511, a PPG unit 555, a taVNS unit 30, an auriculotemporal nerve stimulation unit 302, a greater auricular nerve (GAN) stimulation unit 304 and a network interface 53 and all or part of them (or part of their components) may be configured to be housed in an auricular housing 11. The auricular housing 11 may be selected from one of the following: a modified earbud housing 61, a modified in-the-ear housing 62, a behind-the-ear-hearing-aid-style housing 63 and a tubular-shaped structure 66. (FIGS. 7, 11, 12, 13). The modified earbud housing 61 may include a tubular-body portion 25 and a stem portion 68. (FIGS. 2, 11). The stem portion 68 is equivalent to a “stem” (or a “stalk”) of an earbud. The “stem” hangs down from the wearer's ear. The tubular-body portion 25 may include a tubular-shaped structure 66 and a body-structure 67. The tubular-shaped structure 66 is equivalent to an elongated and modified “ear-tip” and “nozzle” of an earbud (or an AirPod) and will be inserted into a wearer's external ear canal 904 when in use. The body-structure 67 is equivalent to a “body” (or a “shell”) of an earbud (or an AirPod). The body-structure 67 may be placed immediately at the opening of the wearer's external ear canal 904 and sit inside the wearer's tragus-concha bowl 957 when in use. All of the EEG sensor electrodes 12, 13, 72, 73, 82, 83, may be configured to be placed on a surface 91 and partially embedded in the surface 91 with slight protrusion at the surface 91 of the tubular-shaped structure 66. The taVNS stimulating electrode 31, the auricular SCOS sensor 512, the PPG unit 555, and the auriculotemporal nerve stimulating electrode 312 may be configured to be placed on a surface and partially embedded in the surface with slight protrusion at the surface of one of: the tubular-shaped structure 66 and the body-structure 67. (Alternatively, the auricular SCOS sensor 512 may be located slightly below the surface 91 of the tubular-shaped structure 66 or body-structure 67 so that there is a tiny air gap between the auricular SCOS sensor 512 and the skin of the wearer's external ear canal 904 or the skin of the wearer's tragus-concha bowl 957 when the tubular-shaped structure 66 and the body-structure 67 are placed in the wearer's ear 902.) The greater auricular nerve (GAN) stimulating electrode 314 and the optional reference electrode 84 may be placed in a surface (and partially embedded in the surface with slight protrusion at the surface) of the body-structure 67. In some embodiments, both the tubular-shaped structure 66 and the body-structure 67 are configured to be made of elastic flexible and adaptable material. The material of the tubular-shaped structure 66 is configured to have appropriate elasticity flexibility and adaptability so that when the tubular-shaped structure 66 is inserted into the wearer's external ear canal 904, the tubular-shaped structure 66 will naturally adapt to the contour of the wearer's external ear canal 904 and snugly fill the interior of the wearer's external ear canal 904. Similarly, the material of the body-structure 67 is configured to have appropriate elasticity flexibility and adaptability so that when the body-structure 67 is placed inside the wearer's tragus-concha bowl 957, the body-structure 67 will naturally adapt to the contour of the wearer's tragus-concha bowl 957 and snugly fill the interior of the wearer's tragus-concha bowl 957. Thus, all of the EEG sensor electrodes 12, 13, 72, 73, 82, 83, will be naturally in close contact with the skin of the wearer's external ear canal 904; while the taVNS stimulating electrode 31, the auricular SCOS sensor 512, the PPG unit 555, and the auriculotemporal nerve stimulating electrode 312 will be naturally in close contact with the skin of the wearer's external ear canal 904 or the skin of the wearer's tragus-concha bowl 957; and, at the same time, the greater auricular nerve (GAN) stimulating electrode 314 and the optional reference electrode 84 will be naturally in close contact with the skin of the wearer's tragus-concha bowl 957. At the same time, the taVNS stimulating electrode 31 will be naturally in close contact with the skin of the wearer's vagus innervated auricular skin since the skin of the external ear canal 904 and the skin of the tragus-concha bowl 957 are part of the vagus innervated auricular skin. Likewise, at the same time, the auriculotemporal nerve stimulating electrode 312 will be naturally in close contact with the skin of the wearer's auriculotemporal nerve innervated auricular skin since the skin of the external ear canal 904 and the skin of the tragus-concha bowl 957 are part of the auriculotemporal nerve innervated auricular skin. Meanwhile, the GAN stimulating electrode 314 will be naturally in close contact with the skin of the wearer's tragus-concha bowl 957 (including cavum concha 907) since the skin of cavum concha 907 is part of GAN innervated auricular skin. (This is feasible due to the proximity of the innervation patterns of the auricular branch of vagus nerve, the auriculotemporal nerve and the greater auricular nerve in the auricular area, and by careful selection of the locations for 31, 312, 314, to match the locations of their respective target skin on the wearer's ear, as described hereinbefore.) This unique design and set-ups will allow the wearer 900 to install or to remove all of the EEG sensor electrodes 12, 13, 72, 73, 82, 83, the optional reference electrode 84, the auricular SCOS sensor 512, the PPG unit 555, the taVNS stimulating electrodes 31, the auriculotemporal nerve stimulating electrode 312 and the GAN stimulating electrode 314 as easily as placing or removing the tubular-body portion 25 of the modified earbud housing 61 in (or from) the wearer's 900 external ear canal 904 and the tragus-concha bowl 957. Thus, this automatic detection-therapy system 101 is fully wearable, user-installable, user-removable, freely ambulatory and very convenient for wearers (users).

In other embodiments of an automatic detection-therapy system 101 for cluster headache, all or part of the following (or part of their components): an auricular EEG recording module 20, an auricular SCOS unit 511, a PPG unit 555, a processing unit 50, 401, a taVNS unit 30, an auriculotemporal nerve stimulation unit 302, a GAN stimulating electrode 314 and a network interface 53 may be configured to be housed in an auricular housing 11. The auricular housing 11 may be selected from a behind-the-ear-hearing-aid-style housing 63. The behind-the-ear-hearing-aid-style housing includes an in-the-ear portion 27 and a behind-the-ear portion 26. The in-the-ear portion 27 is essentially the same as the tubular-body portion 25 of a modified earbud housing 61 as aforementioned. (FIG. 13). The in-the-ear portion 27 (of the behind-the-ear-hearing-aid-style housing 63) includes a tubular-shaped structure 66 and a body-structure 67. The tubular-shaped structure 66 is equivalent to an elongated modified “ear-tip” and “nozzle” of an earbud (or an AirPod) and will be inserted into a wearer's external ear canal 904 when in use. The body-structure 67 is equivalent to a “body” (or a “shell”) of an earbud (or an AirPod). The body-structure 67 will be placed immediately at the opening of the wearer's external ear canal 904 and sit inside the wearer's tragus-concha bowl 957 when in use. All of the EEG sensor electrodes 12, 13, 72, 73, 82, 83, may be configured to be placed on a surface 91 and partially embedded in the surface 91 with slight protrusion at the surface 91 of the tubular-shaped structure 66. The taVNS stimulating electrode 31, the auricular SCOS sensor 512, the PPG unit 555, and the auriculotemporal nerve stimulating electrode 312 may be configured to be placed on the surface and partially embedded in the surface with slight protrusion at the surface of one of: the tubular-shaped structure 66 and the body-structure 67. (Alternatively, the auricular SCOS sensor 512 may be located slightly below the surface 91 of the tubular-shaped structure 66 or body-structure 67 so that there is a tiny air gap between the auricular SCOS sensor 512 and the skin of the wearer's external ear canal 904 or the skin of the wearer's tragus-concha bowl 957 when the tubular-shaped structure 66 and the body-structure 67 are placed in the wearer's ear 902.) The GAN stimulating electrode 314 and the optional reference electrode 84 are configured to be place at a surface (and partially embedded in the surface with slight protrusion at the surface) of the body-structure 67. In some embodiments, both the tubular-shaped structure 66 and the body-structure 67 are configured to be made of elastic flexible and adaptable material. The material of the tubular-shaped structure 66 is configured to have appropriate elasticity flexibility and adaptability so that when the tubular-shaped structure 66 is inserted into the wearer's external ear canal 904, the tubular-shaped structure 66 will naturally adapt to the contour of the wearer's external ear canal 904 and snugly fill the interior of the wearer's external ear canal 904. Similarly, the material of the body-structure 67 is configured to have appropriate elasticity flexibility and adaptability so that when the body-structure 67 is placed inside the wearer's tragus-concha bowl 957, the body-structure 67 will naturally adapt to the contour of the wearer's tragus-concha bowl 957 and snugly fill the interior of the wearer's tragus-concha bowl 957. Thus, all of the EEG sensor electrodes 12, 13, 72, 73, 82, 83, will be naturally in close contact with the skin of the wearer's external ear canal 904; while the taVNS stimulating electrode 31, the auricular SCOS sensor 512, the PPG unit 555, and the auriculotemporal nerve stimulating electrode 312 will be naturally in close contact with the skin of the wearer's external ear canal 904 or the skin of the wearer's tragus-concha bowl 957; and, at the same time, the GAN stimulating electrode 314 and the optional reference electrode 84 will be naturally in close contact with the skin of the wearer's tragus-concha bowl 957. At the same time, the taVNS stimulating electrode 31 will be naturally in close contact with the skin of the wearer's vagus innervated auricular skin since the skin of the external ear canal 904 and the skin of the tragus-concha bowl 957 are part of the vagus innervated auricular skin. Likewise, at the same time, the auriculotemporal nerve stimulating electrode 312 will be naturally in close contact with the skin of the wearer's auriculotemporal nerve innervated auricular skin since the skin of the external ear canal 904 and the skin of the tragus-concha bowl 957 are part of the auriculotemporal nerve innervated auricular skin. Similarly, at the same time, the GAN stimulating electrode 314 will be naturally in close contact with the skin of the wearer's tragus-concha bowl 957 since the skin of cavum concha 907 is part of the GAN innervated auricular skin. (This is feasible due to the proximity of the innervation patterns of the auricular branch of vagus nerve, the auriculotemporal nerve and the greater auricular nerve in the auricular area, and by careful selection of the locations for 31, 312, 314, to match the locations of their respective target skin on the wearer's ear, as described hereinbefore.) This unique design and set-up will allow the wearer 900 to attach or to remove all of the EEG sensor electrodes 12, 13, 72, 73, 82, 83, the optional reference electrode 84, the auricular SCOS sensor 512, the PPG unit 555, the taVNS stimulating electrodes 31, the auriculotemporal nerve stimulating electrode 312 and the GAN stimulating electrode 314 as easily as placing or removing the in-the-ear portion 27 of the behind-the-ear-hearing-aid-style housing 63 in (or from) the wearer's 900 external ear canal 904 and the tragus-concha bowl 957. These features will enable the automatic detection-therapy system 101 be fully wearable, self-installable, self-removable, freely ambulatory and very convenient for wearers (users). Alternatively, the auriculotemporal nerve stimulation unit 302 may have a clip electrode 319 to be clipped manually to the wearer's anterior-superior helix 909 of the ear 902 for the auriculotemporal nerve stimulating unit 902 to deliver the electric stimuli via the clip electrode 319 (FIG. 28).

In some embodiments for an automatic detection-therapy system 101 for cluster headache, all or part of the following (or part of their components): an auricular EEG recording module 20, a processing unit 50, 401, an auricular SCOS unit 511, a PPG unit 555, a taVNS unit 30, an auriculotemporal nerve stimulation unit 302, a greater auricular nerve (GAN) stimulation unit 304, and a network interface 53 may be configured to be housed in an auricular housing 11. The auricular housing 11 may comprise a modified in-the-ear-housing 62 that includes a tubular-shaped structure 66 (to be inserted into a wearer's external ear canal 904 when in use) and a body-structure 67 (to be placed in a tragus-concha bowl 957 of the wearer's ear when in use). (As aforementioned, the modified earbud housing 61 includes a tubular-shaped structure 66 and a body-structure 67. The behind-the-ear-hearing-aid-style housing 63 also includes a tubular-shaped structure 66 and a body-structure 67.) In some embodiments, the modified in-the-ear housing 62 may be made with or may comprise elastic flexible and adaptable material. The material for the modified in-the-ear housing 62 is configured to have appropriate elasticity, flexibility and adaptability so that when it is placed in the wearer's external ear canal 904 and the tragus-conch bowl 957, it will naturally adapt to the contours of the wearer's external ear canal 904 and the contour of the wearer's tragus-concha bowl 957 and snugly fill the interior of the wearer's external ear canal 904 and the interior of the tragus-concha bowl 957. All of the EEG sensor electrodes 12, 13, 72, 73, 82, 83, may be configured to be placed on a surface 91 and partially embedded in the surface 91 with slight protrusion at the surface 91 of the tubular-shaped structure 66. The taVNS stimulating electrode 31, the auricular SCOS sensor 512, the PPG unit 555 and the auriculotemporal nerve stimulating electrode 312 may be configured to be placed on the surface and partially embedded in the surface with slight protrusion at the surface of one of: the tubular-shaped structure 66 and the body-structure 67. The GAN stimulating electrode 314 and the optional reference electrode 84 are configured to be placed at a surface (and partially embedded in the surface with slight protrusion at the surface) of the body-structure 67. The material of the modified in-the-ear housing 62 (including the tubular-shaped structure 66 and the body structure 67) is configured to have appropriate elasticity, flexibility and adaptability so that when the modified in-the-ear housing 62 is placed in a wearer's external ear canal 904 and the tragus-concha bowl 957, all of the EEG sensor electrodes 12, 13, 72, 73, 82, 83, will be naturally in close contact with the skin of the wearer's external ear canal 904, while the auricular SCOS sensor 512, the PPG unit 555, the taVNS stimulating electrode 31 and the auriculotemporal nerve stimulating electrode 312 will be naturally in close contact with the skin of the wearer's external ear canal 904 or the skin of the wearer's tragus-concha bowl 957, and, at the same time, the GAN stimulating electrode 314 and the optional reference electrode 84 will be naturally in close contact with the skin of the wearer's tragus-concha bowl 957. Thus, at the same time, the taVNS stimulating electrode 31 will be naturally in close contact with the skin of the wearer's vagus innervated auricular skin since the skin of the external ear canal 904 and the skin of the tragus-concha bowl 957 are part of the vagus innervated auricular skin. Likewise, at the same time, the auriculotemporal nerve stimulating electrode 312 will be naturally in close contact with the skin of the wearer's auriculotemporal nerve innervated auricular skin since the skin of the external ear canal 904 and the skin of the tragus-concha bowl 957 are part of the auriculotemporal nerve innervated auricular skin. Similarly, at the same time, the GAN stimulating electrode 314 will be naturally in close contact with the skin of the wearer's tragus-concha bowl 957 since the skin of cavum concha 907 is part of the GAN innervated auricular skin. (This is feasible due to the proximity of the innervation patterns of the auricular branch of vagus nerve, the auriculotemporal nerve and the greater auricular nerve in the auricular area, and by careful selection of the locations for 31, 312, 314, to match the locations of their respective target skin on the wearer's ear, as described hereinbefore.) This unique design and set-up will allow the wearer 900 to attach or to remove all of the EEG sensor electrodes 12, 13, 72, 73, 82, 83, the optional reference electrode 84, the auricular SCOS sensor 512, the PPG unit 555, the taVNS stimulating electrodes 31, the auriculotemporal nerve stimulating electrode 312 and the GAN stimulating electrode 314 as easily as placing or removing the modified in-the-ear housing 62 in (or from) the wearer's 900 external ear canal 904 and the tragus-concha bowl 957.

In some embodiments for an auricular EEG monitoring system 100 and/or an automatic detection-therapy system 101, a standalone tubular-shaped structure 66 may be used for housing purpose. The housing setups and arrangements for the standalone tubular-shaped structure 66 are essentially the same as the tubular-shaped structure 66 described hereinbefore.

As aforementioned, all of these 3 types of housing (i.e. a modified earbud housing 61, a behind-the-ear-hearing-aid-style housing 63 and a modified in-the-ear housing 62) comprise a tubular-shaped structure 66. In preferred embodiments for an auricular EEG monitoring system 100 and/or an automatic detection-therapy system 101, all of the EEG sensor electrodes 12, 13, 72, 73, 82, 83, may be housed at the surface 91 of the tubular-shaped structure 66 and partially embedded with slight protrusion at the surface 91 of the tubular-shaped structure 66. One or more of the EEG sensor electrodes 12, 13, 72, 73, 82, 83, is/are configured to be located at the upper surface 91 (upper surface at approximately 90 degrees above horizontal level 92, as shown by electrodes 12, 13, in FIGS. 19, 22, 25) of the tubular-shaped structure 66. In preferred embodiments, one or more of the EEG sensor electrodes 12, 13, 72, 73, 82, 83, is/are configured to be located at between 0 and 90 degrees, and more preferably at approximately 45 degrees (plus or minus fifteen degrees or 30-60 degrees) above the horizontal level 92 of the tubular-shaped structure 66 and is/are configured to face forward-upward direction 93 (e.g., as shown by electrodes 72, 82, in FIGS. 19, 21, 24). In preferred embodiments, one or more of the EEG sensor electrodes is/are configured to be located at between 90 and 180 degrees, and more preferably at approximately 135 degrees (plus or minus fifteen degrees or 120-150 degrees) above the horizontal level 92 of the tubular-shaped structure 66 and is/are configured to face backward-upward direction 94 (e.g., as shown by electrodes 73, 83, in FIGS. 19, 23, 26). These arrangements will enable the EEG sensor electrodes 12, 13, 72, 73, 82, 83, to have one of the best locations and directions from the external ear canal 904 to record the wearer's EEG activities. (Upper surface, horizontal level, forward, backward, and upward all refer to directions relative to the head 901 of the wearer 900 with the wearer in an upright position after the tubular-shaped structure 66 has been inserted into a wearer's external ear canal 904.)

In some embodiments for an automatic detection-therapy system 101 for cluster headache, a separate client device 400 may be used for housing of some of the components of the automatic detection-therapy system 101. All of the EEG sensor electrodes 12, 13, 72, 73, 82, 83, may be configured as wireless EEG sensor electrodes. The optional reference electrode 84 may be configured as wireless optional reference electrode 84. The EEG recording module 20 may be configured to comprise a wireless EEG amplifier 21. Thus, all of the wireless EEG sensor electrodes 12, 13, 72, 73, 82, 83 and the optional wireless reference electrode 84 may be housed in an auricular housing 11 while the EEG recording module 20 and the processing unit may be configured to be housed remotely in one of: a watch-type client device, a smart phone-type client device or a tablet-type client device. Wireless EEG sensor electrodes may be used for all of the EEG sensor electrodes 12, 13, 72, 73, 82, 83, and the optional reference electrode 84. (Wireless dry EEG electrodes such as that described by Ryan Kaveh et al. in Nature Communications on Aug. 2, 2024. Wireless dry EEG electrodes are also available from Zeto, Inc. headquarter in Santa Clara, California.) There are well-known wired or wireless EEG amplifiers available, such as EEG Electroencephalogram Smart Amplifier (Part #: EEG100D), or preferably BioNomadix 2Ch Wireless EEG Amplifier (Part #: BN-EEG2), both being made by the same company BIOPAC Systems, Inc. (Goleta, California). By using wireless EEG sensor electrodes 12, 13, 72, 73, 82, 83, wireless optional reference electrode 84 and wireless EEG amplifier 21, the EEG recording module 20 together with the processing unit 50, 401, may be housed remotely in a watch-type, smart phone-type or tablet-type client device 400 and communicate with all of the EEG sensor electrodes 12, 13, 72, 73, 82, 83, and the optional reference electrode 84 wirelessly.

In some embodiments, an automatic detection-therapy system 101 for cluster headache may comprises a switch 88 and a timer 89 to enable the wearer 900 to turn on the system 101 manually and setting a duration, an interval or a schedule, etc. Preferably, the switch 88 may be configured as a multi-mode switch 88 and the timer 89 may be configured as a multi-mode timer 89. In some embodiments, a multi-mode switch 88 may be configured to enable the wearer to set the system 101 at an automatic mode or a manual mode. The functions and setups of the automatic mode for system 101 are as described hereinbefore. In some embodiments for an automatic detection-therapy system 101 for cluster headache, the multi-mode switch 88 may be configured to allow the wearer 900 to choose at least one of the following switch selections: turning on or off an auricular EEG monitoring system 100, turning on or off an auricular SCOS unit 511, turning on or off a PPG unit 555, turning on or off a taVNS unit 30, turning on or off an auriculotemporal nerve stimulation unit 302, turning on or off a greater auricular nerve (GAN) stimulation unit 304, turning on or off a supraorbital nerve stimulation unit 301, turning on or off an occipital nerve stimulation unit 303, turning on or off an infraorbital nerve stimulation unit 305 or various combinations thereof: The multi-mode timer 89 may be configured to function together with the multi-mode switch 88 to set each switch selection at a desirable timing course such as duration, interval, countdown, daily/weekly schedule, 10 minute/30 minutes/60 minutes (or other duration) selection, continuous operation, manual turning on and manual shutdown etc. Furthermore, the multi-mode switch 88 and multi-mode timer 89 may be configured as programmable. In some embodiments, the multi-mode switch 88 functions and the multi-mode timer 89 functions may be configured to be incorporated into a wearer's client device 400 and can be easily set up as programmable. In the manual mode, the wearer 900 can use the taVNS unit 30 or the auriculotemporal nerve stimulation unit 302 or the greater auricular nerve (GAN) stimulation unit 304 or various combinations thereof for prophylactic purpose against cluster headache or for health maintenance purpose. Studies have shown that the taVNS unit 30 is effective not only for therapy of cluster headache and impending cluster headache, but also for prophylaxis against cluster headache. Studies have also shown that neuromodulating electric stimulation from the taVNS unit 30, the supraorbital nerve stimulation unit 301, the auriculotemporal nerve stimulation unit 302, the occipital nerve stimulation unit 303, GAN stimulation unit 304 and the infraorbital nerve stimulation unit 305 (and various combinations thereof) are effective not only for therapy of cluster headache, impending cluster headache and cluster headache prophylaxis but also for other neuropsychiatric diseases and for health maintenance. The aforementioned programmable set up for the system 101 will enable the system 101 to be used for prophylactic purpose and health maintenance purpose. The multi-mode switch 88, multi-mode timer 89 and the programmable design may enable the wearer the option to choose from one of the following: neuromodulation from one neuromodulation component (single neuromodulation), simultaneous neuromodulation from two neuromodulation components (double neuromodulation) and simultaneous neuromodulation from three neuromodulation components (triple neuromodulation). Thus, when prompted, the neuromodulation unit 300 is configured to generate neuromodulating electric stimulation to a wearer 900 according to the choice of the stimulation mode selected by the wearer 900. The choices of the stimulation mode include the following: single neuromodulation by one component of the neuromodulation unit 300, double neuromodulation by various combinations of two components of the neuromodulation unit 300, triple neuromodulation by various combinations of three components of the neuromodulation unit 300, etc.

There are many conventional smart watches or health trackers that contain monitoring devices 71 to monitor various body functions, including electrocardiogram (ECG), heart rate, blood oxygen, sleep, body temperature, and motion etc. (as known in the art). However, none of them can monitor the most important part of the human body, which is the brain. The auricular EEG monitoring system 100 and/or the automatic detection-therapy system 101 can easily combine with other monitoring devices 71 by placing their monitoring sensors in the external ear 902 or the external ear canal 904. Thus, system 100 and/or system 101 can monitor not only the brain (through EEG), it can also monitor other body functions, such as ECG, heart rate, blood oxygen, motion, sleep and body temperature, etc.

### A Third Preferred Embodiment

According to a further aspect consistent with the principles of the invention, a wearable and ambulatory novel neurovascular monitoring system 500 is disclosed. The neurovascular monitoring system 500 comprises an auricular electroencephalogram (EEG) monitoring system 100, a cephalic blood flow monitoring system 530, a network interface 53 and a processing unit 50, 401. The neurovascular monitoring system 500 is configured to simultaneous record and monitor cerebral electrical activities (through the auricular EEG monitoring system 100) and cerebral blood flow and extracranial blood flow (through the cephalic blood flow monitoring system 530), as described hereinbefore. (This neurovascular monitoring system 500 is very useful to monitor patients at high risk of developing a stroke, such as patients having atrial fibrillation or other heart diseases and patients having severe stenosis of carotid artery, middle cerebral artery, anterior cerebral artery and vertebro-basial artery, etc.)

In some embodiments, a neurovascular monitoring system 500 comprises an auricular electroencephalogram (EEG) monitoring system 100. The auricular EEG monitoring system 100 comprises an auricular electroencephalogram (EEG) recording module 20. The auricular EEG recording module 20 may have a plurality (at least two, but preferably more than two) of miniature wired or wireless EEG sensor electrodes 12, 13, 72, 73, 82, 83 and a wired or wireless EEG amplifier 21. Optionally, the EEG recording module 20 may include an optional reference electrode 84. (The optional reference electrode 84 would be desirable but is not always necessary. Instead, average of all of the EEG sensor electrodes 12, 13, 72, 73, 82, 83, can be used as a reference, i.e. common average reference). The EEG sensor electrodes 12, 13, 72, 73, 82, 83, and the optional reference electrode 84 may be configured to contact separate areas selected from at least one of the following: external ear canal 904 of a first ear of the wearer, external ear 902 of the first ear of the wearer or peri-auricular area 903 around the first ear of the wearer. The optional reference electrode 84 of the EEG recording module 20 may be placed in the tragus-concha bowl 957 or at the mastoid (mastoid process) of the peri-auricular area 903. (Tragus-concha bowl 957 and mastoid are known to be good locations for EEG reference electrode.) The EEG recording module 20 may be configured to record and generate EEG data of the wearer 900 using electrical activities of the wearer 900 that are picked up via all of the EEG sensor electrodes 12, 13, 72, 73, 82, 83, and the optional reference electrode 84 (if the optional reference electrode is included). All of the EEG sensor electrodes 12, 13, 72, 73, 82, 83 and the optional reference electrode 84 are in wired or wireless electronic communication with the EEG amplifier 21 of the EEG recording module 20. The auricular EEG monitoring system 100 may further comprise a processing unit 50, 401. The auricular EEG recording module 20 may be in electronic communication with the processing unit 50, 401, through wire, Bluetooth or other wireless connection means. The auricular EEG recording module 20 may be configured to collect or record EEG data of the wearer 900 via all of the EEG sensor electrodes 12, 13, 72, 73, 82, 83 and the optional reference electrode 84 (if the optional reference electrode is incorporated). The processing unit 50, 401 is configured to converts raw EEG data into quantitative EEG (qEEG) data through digital signal processing (DSP) techniques that transform, analyze, and mathematically quantify brain electrical activity. These EEG and quantitative EEG (qEEG) data are transmitted or otherwise communicated to the processing unit 50, 401. With the help of various EEG analysis algorithms, together with machine learning, deep learning and artificial intelligence, the processing unit 50, 401, may be configured to analyze the EEG and qEEG data of the wearer 900 for detection of presence of EEG and qEEG signals suggestive of significant EEG status change. The processing unit 50, 401, may be further configured to detect cessation of EEG and qEEG signals suggestive of the significant EEG status change. More specifically, the processing unit 50,401, is configured to analyze the EEG and qEEG data to detect cerebral ischemia within 28-100 seconds of cerebral ischemic penumbra (before structural damage) indicating impending stroke by detecting at least one of the following: increased delta/theta waves with an increase of relative delta power more than a predetermined level (e.g. an increase of 15% or more), reduced alpha/beta frequencies and elevated ratios such as an increase of delta/alpha ratio (DAR) or (delta+theta)/(alpha+beta) ratio (DTABR) more than a predetermined level (e.g. an increase of 15% or more), and interhemispheric differences in relative power (.e.g. an interhemispheric difference of 15% or more). (Please note: detecting cerebral ischemic penumbra within 28-100 seconds is more valuable and significantly sooner than detecting cerebral structural damages by CT scan or MRI scan. In stroke management, timing is critically important because thrombolytic therapy (tPA/Alteplase) for ischemic stroke should be given as soon as possible, ideally within 3 hours of symptom onset and at least within 4.5 hours of symptom onset. The standard goal is to start treatment within 60 minutes of emergency room arrival because earlier treatment yields better outcomes. Quantitative EEG shows rapid changes during impending stroke and acute ischemic stroke. Changes detectable by quantitative EEG includes increased delta/theta waves and reduced alpha/beta frequencies, elevated ratios, such as the (delta+theta)/(alpha+beta) ratio (DTABR) or delta/alpha ratio (DAR), and interhemispheric differences in relative power, or an increase in the delta/alpha ratio, etc. Using qEEG to monitor stroke or impending stroke can save valuable time as compared with using CT or MRI scan to monitor stroke or impending stroke. Similarly, qEEG can detect changes of delayed cerebral ischemia before it is clinically obvious and is very valuable when monitoring patients with brain trauma and cerebral inflammation.)

In some embodiments, a neurovascular monitoring system 500 may further comprise a cephalic blood flow monitoring system 530. The cephalic blood flow monitoring system 530 may be selected from at least one of: various types of laser speckle blood flow devices, photoplethysmography (PPG) and ultrasound-doppler based blood flow monitors. Examples of laser speckle blood flow devices may include a Speckle Contrast Optical Spectroscope unit (SCOS unit) 510. Other examples of laser speckle blood flow devices include Diffuse Correlation Spectroscopy (DCS), Laser Speckle Contrast Imaging (LSCI), etc. An auricular Speckle Contrast Optical Spectroscope unit (auricular SCOS unit) 511 is created by placing a SCOS unit 510 in an auricular housing 11, such as a modified earbud housing 61 or an in-the-ear housing 62 or a behind-the-ear-hearing-aid-style housing 63. The auricular SCOS unit 511 comprises a light source (laser) 519 that pass through a focusing lens 518 and an expander 513 and mirrors 514 and then tissue (or sample) 515. The light is then collected by a SCOS sensor (detector) 512 that is integrated with a CMOS camera 516 and the data are transmitted to a data processing unit (computer) 517 for analysis and to get the blood flow data at different depths of the tissue. (FIG. 34). (A CMOS camera 516 refers to Complementary Metal-Oxide-Semiconductor camera that is a digital imaging device using a sensor to convert light into electrical signals to create images.) The auricular SCOS unit 511 comprises an auricular Speckle Contrast Optical Spectroscope sensor (auricular SCOS sensor) 512. The auricular SCOS sensor 512 may be configured to be located on a surface of a wearer's tragus-concha bowl 957 or the wearer's external ear canal 904 when in use. The auricular SCOS sensor 512 is configured to collect or record the wearer's “blurriness” (speckle contrast) of laser light scattered by moving red blood cells and, with the help of the data processing unit 517, to determine flow speed and volume, providing a faster and more accurate alternative to diffuse correlation spectroscopy (DCS). The auricular SCOS unit 511 is configured to collect or record the wearer's cerebral (intracranial) and extracranial blood flow data. In some embodiments, a neurovascular monitoring system 500 may comprise an auricular EEG monitoring system 100 (configured to record EEG data of a wearer 900 from an ear 902 of the wearer) and a cephalic blood flow monitoring system 530 (configured to record cerebral and extracranial blood flow data of the wearer from the ear 902 of the wearer.) The cephalic blood flow monitoring system 530 includes an auricular SCOS unit 511. The auricular EEG monitoring system 100 and the cephalic blood flow monitoring system 530 are configured to simultaneously record (collect) the wearer's EEG data and cerebral (intracranial) and extracranial blood flow data.

In some embodiments, the auricular Speckle Contrast Optical Spectroscope unit (auricular SCOS unit) 511 comprises an auricular SCOS sensor 512 that may be placed at a surface of an auricular housing 11. The EEG sensor electrodes 12, 13, 72, 73, 82, 83 and the optional reference electrode 84 may also be placed on the surface of the auricular housing 11. Examples of auricular housing 11 include a modified earbud housing 61, a modified in-the-ear housing 62, a behind-the-ear-hearing-aid style housing 63 or a tubular-shaped structure 66, as described hereinbefore. The auricular housing 11 includes a tubular-shaped structure 66 (to be inserted into a wearer's external ear canal 904 when in use) and a body-structure (to be placed at immediate opening of the wearer's external ear canal 904 and be placed in the wearer's tragus-concha bowl 957 when in use) (similar to descriptions hereinbefore). The auricular SCOS sensor 512 is configured to be placed on a surface and partially embedded in the surface with slight protrusion at the surface of the tubular-shaped structure 66 or the body-structure 67 of the auricular housing 11. (Alternatively, the auricular SCOS sensor 512 may be located slightly below the surface of the tubular-shaped structure 66 or the body-structure 67 so that there is a tiny air gap between the auricular SCOS sensor 512 and the skin of the wearer's external ear canal 904 or skin of the wearer's tragus-concha bowl 957 when the auricular housing 11 is placed in the wearer's ear 902.) Meanwhile, all of the EEG sensor electrodes 12, 13, 72, 73, 82, 83 are configured to be placed on the surface (and partially embedded in the surface with slight protrusion at the surface) of the tubular-shaped structure 66, while the optional reference electrode 84 (of the EEG recording module 20) is configured to be placed on the surface (and partially embedded in the surface with slight protrusion at the surface) of the body-structure 67. The tubular-shaped structure 66 and the body-structure 67 are configured to be made of elastic flexible and adaptable material. The material for the tubular-shaped structure 66 is configured to have appropriate elasticity flexibility and adaptability so that when the tubular-shaped structure 66 is placed in the wearer's external ear canal 904, the tubular-shaped structure 66 will naturally adapt to the contour of the wearer's external ear canal 904 and snugly fill the interior of the wearer's external ear canal 904 and so that all of the EEG sensor electrodes 12, 13, 72, 73, 82, 83 will be naturally in close contact with the skin of the wearer's external ear canal 904. Similarly, the material for the body-structure 67 is configured to have appropriate elasticity, flexibility and adaptability so that when the body-structure is placed in the wearer's tragus-concha bowl 957, the body-structure 957 will naturally adapt to the contour of the wearer's tragus-concha bowl 957 and snugly fill the interior of the wearer's tragus-concha bowl 957, and so that the optional reference electrode 84 will be naturally in close contact with the skin of the wearer's tragus-concha bowl 957 and, at the same time, the auricular SCOS sensor 512 will be naturally snugly in contact with the skin of the wearer's external ear canal 904 or skin of the wearer's tragus-concha bowl 957. This housing designs will enable the neurovascular monitoring system 500 (including the auricular EEG monitoring system 100 and the cephalic blood flow monitoring system 530) user-installable, user-removable, wearable and freely ambulatory and very convenient for long-term use.

In some modified embodiments, the auricular housing 11 comprises a tubular-shaped structure 66 (standalone tubular-shaped structure 66). The tubular-shaped structure 66 is configured to be inserted into an external ear canal 904 of a wearer 900 when in use. The auricular Speckle Contrast Optical Spectroscope unit (auricular SCOS unit) 511 comprises an auricular SCOS sensor 512 that is placed on a surface of the tubular-shaped structure 66. All of the EEG sensor electrodes 12, 13, 72, 73, 82, 83 may also be placed on the surface of the tubular-shaped structure 66. The auricular SCOS sensor 512 and all of the EEG sensor electrodes 12, 13, 72, 73, 82, 83, are configured to be partially embedded in the surface with slight protrusion at the surface of the tubular-shaped structure 66. (Alternatively, the auricular SCOS sensor 512 may be located slightly below the surface of the tubular-shaped structure 66 so that there is a tiny air gap between the auricular SCOS sensor 512 and the skin of the wearer's external ear canal 904 when the tubular-shaped structure 66 is inserted into the wearer's external ear canal 904.) The tubular-shaped structure 66 is configured to be made of elastic flexible adaptable material. The material for the tubular-shaped structure 66 is configured to have appropriate elasticity flexibility and adaptability so that when the tubular-shaped structure 66 is inserted into the wearer's external ear canal 904, the tubular-shaped structure 66 will naturally adapt to the contour of the wearer's external ear canal 904 and fill the interior of the wearer's external ear canal 904 and so that the auricular SCOS sensor 512 and all of the EEG sensor electrodes 12, 13, 72, 73, 82, 83, will be naturally snugly in contact with the skin of the wearer's external ear canal 904.

As aforementioned, all of these 4 types of housing (i.e. a modified earbud housing 61, a behind-the-ear-hearing-aid-style housing 63, a modified in-the-ear housing 62 and a standalone tubular-shaped structure 66) comprises a tubular-shaped structure 66. In preferred embodiments for a neurovascular monitoring system 500, all of the EEG sensor electrodes 12, 13, 72, 73, 82, 83, may be housed at the surface 91 of the tubular-shaped structure 66 and partially embedded with slight protrusion at the surface 91 of the tubular-shaped structure 66. One or more of the EEG sensor electrodes 12, 13, 72, 73, 82, 83, is/are configured to be located at the upper surface 91 (upper surface at approximately 90 degrees above horizontal level 92, as shown by electrodes 12, 13, in FIGS. 19, 22, 25) of the tubular-shaped structure 66. In preferred embodiments, one or more of the EEG sensor electrodes 12, 13, 72, 73, 82, 83, is/are configured to be located at between 0 and 90 degrees, and more preferably at approximately 45 degrees (plus or minus fifteen degrees) above the horizontal level 92 of the tubular-shaped structure 66 and is/are configured to face forward-upward direction 93 (e.g., as shown by electrodes 72, 82, in FIGS. 19, 21, 24). In preferred embodiments, one or more of the EEG sensor electrodes is/are configured to be located at between 90 and 180 degrees, and more preferably at approximately 135 degrees (plus or minus fifteen degrees) above the horizontal level 92 of the tubular-shaped structure 66 and is/are configured to face backward-upward direction 94 (e.g., as shown by electrodes 73, 83, in FIGS. 19, 23, 26) (Upper surface, horizontal level, forward, backward, and upward all refer to directions relative to the head 901 of the wearer 900 with the wearer in an upright position after the tubular-shaped structure 66 has been inserted into a wearer's external ear canal 904.) This design and arrangement will enable the EEG sensor electrodes 12, 13, 72, 73, 82, 83, to have one of the best arrangements (multi-dimensional locations) and directions from the wearer's external ear canal 904 to record the wearer's EEG.

In some embodiments, a neurovascular monitoring system 500 may further comprise a network interface 53 and a processing unit 50, 401. The processing unit 50, 401, is in wired or wireless electronic communication with the auricular EEG monitoring system 100, the cephalic blood flow monitoring system 530 including the auricular SCOS unit 511, and the network interface 53. The processing unit 50, 401, is configured to analyze the wearer's EEG and qEEG data recorded by (transmitted from) the auricular EEG monitoring system 100 to assess the wearer's EEG status. The processing unit 50, 401, is also configured to analyze the wearer's cerebral blood flow data recorded by (transmitted from) the auricular SCOS unit 511 to assess the wearer's cerebral blood flow status. In addition, the processing unit 50, 401, is configured to analyze the wearer's EEG and qEEG data recorded by (transmitted from) the auricular EEG monitoring system 100 to detect any significant change of the wearer's EEG status. The processing unit 50, 401, is further configured to analyze the wearer's cerebral blood flow data recorded by (transmitted from) the auricular SCOS unit 511 to detect any significant change of the wearer's cerebral blood flow status. Artificial intelligence (AI) and analysis algorithms (known in the art) may be used to identify or detect subtle patterns in cerebral blood flow changes (changes in blood flow data) and brain EEG activity changes (changes in EEG and qEEG data) that precede a stroke (i.e. impending stroke) or precede worsening of an existing stroke. The changes in EEG and qEEG that are detectable by the auricular EEG monitoring system 100 when monitoring stroke include: increased delta/theta waves and reduced alpha/beta frequencies, increase relative delta power, elevated ratios, such as the (delta+theta)/(alpha+beta) ratio (DTABR) or delta/alpha ratio (DAR), and interhemispheric differences in relative power, or an increase in the delta/alpha ratio, etc. For example, an increase of relative delta power of 10% or an increase of delta/alpha ratio of 10% indicate a significant change while an increase of relative delta power or 22% or more or an increase of delta/alpha ratio of 22% or more indicate a major change. For monitoring of patients with traumatic brain injuries, AI and analysis algorithms (known in the art) may also be used to identify or detect EEG and qEEG data changes and cerebral blood flow data changes that precede a secondary brain injury (i.e. impending secondary brain injury). (Secondary brain injury is the delayed, indirect damage to brain tissue that evolves hours or days after a primary injury or an initial head trauma. The secondary brain injury is caused by secondary physiological responses like swelling, inflammation, and reduced blood flow and oxygenation, rather than the initial impact.) Artificial intelligence (AI) and analysis algorithms (known in the art) may also be used to identify or detect EEG and qEEG data changes and cerebral blood flow data changes that precede worsening of brain damage (i.e. impending worsening of brain damage) in cerebral inflammation or cerebral infection patients. The changes in EEG and qEEG that are detectable by the auricular EEG monitoring system 100 when monitoring brain trauma or cerebral inflammation include: increased delta/theta waves and reduced alpha/beta frequencies, increase relative delta power, elevated ratios, such as the (delta+theta)/(alpha+beta) ratio (DTABR) or delta/alpha ratio (DAR), and interhemispheric differences in relative power, or an increase in the delta/alpha ratio, etc. For example, an increase of relative delta power of 10% or an increase of delta/alpha ratio of 10% indicate a significant change while an increase of relative delta power or 22% or more or an increase of delta/alpha ratio of 22% or more indicate a major change. (Worsening of brain damage may occur due to excessive immune response leading to brain edema or disruption of blood-brain barrier or increase intracranial pressure with reduced cerebral blood flow.)

In some embodiments, a neurovascular monitoring system 500 for monitoring of stroke and impending stroke may comprise an auricular EEG monitoring system 100, a cephalic blood flow monitoring system 530, a network interface 53 and a processing unit 50, 401. The processing unit 50, 401, is configured to analyze the EEG and qEEG data to assess the wearer's EEG status and to detect presence of at least one of: an increase of relative delta power more than a predetermined level (e.g. an increase of 20% or more) and an increase of delta/alpha ratio more than a predetermined level (e.g. an increase of 20% or more). The processing unit 50, 401, is also configured to analyze the cerebral blood flow data recorded by the auricular SCOS unit 511 to assess the wearer's cerebral blood flow status and to detect presence of a decrease of cerebral blood flow more than a predetermined level (for example, a decrease of cerebral blood flow of 20% or more). When the processing unit 50, 401, detect at least one of the following: presence of the qEEG data showing an increase of relative delta power more than a predetermined level (e.g. an increase of 20% or more), an increase of delta/alpha ratio more than a predetermined level (e.g. an increase of 20% or more), and decrease of cerebral blood flow more than a predetermined level (e.g. a decrease of cerebral blood flow of 20% or more), the processing unit 50, 401, is configured to generate signals to the network interface 53 to prompt the network interface 53 to generate notification to at least one of: a client device 400 of the wearer 900 and a client device of a healthcare provider 950 of the wearer to take appropriate actions since these changes are suggestive or indicative of impending stroke or impending worsening of an existing stroke. The processing unit 50, 401, is further configured to generate signals to the network interface 53 to prompt the network interface 53 to generate a display of the wearer's EEG status and blood flow status on at least one of: the client device 400 of the wearer 900 and the client device 400 of the healthcare provider 950 of the wearer.

Similarly, in some embodiments, a neurovascular monitoring system 500 for monitoring of traumatic brain injury patients or patients with cerebral inflammation or infection, the processing unit 50, 401, is configured to analyze the EEG and qEEG data to assess the wearer's EEG status and to detect presence of EEG and qEEG data showing at least one of: an increase of relative delta power over a predetermined level (e.g. an increase of 15% or more) and an increase of delta/alpha ratio over a predetermined level (e.g. an increase of 15% or more). The processing unit 50, 401, is also configured to analyze the cerebral blood flow data recorded by the auricular SCOS unit 511 to assess the wearer's cerebral blood flow status and to detect presence of a decrease of cerebral blood flow more than a predetermined level (for example, a decrease of cerebral blood flow of 15% or more). When the processing unit 50, 401, detect at least one of the following: presence of the EEG and qEEG data showing an increase of relative delta power more than a predetermined level (e.g. an increase of 15% or more), an increase of delta/alpha ratio more than a predetermined level (e.g. an increase of 15% or more), and decrease of cerebral blood flow more than a predetermined level (e.g. a decrease of cerebral blood flow of 15% or more), the processing unit 50, 401, is configured to generate signals to the network interface 53 to prompt the network interface 53 to generate notification to at least one of: a client device 400 of the wearer 900 and a client device of a healthcare provider 950 of the wearer to take appropriate actions since these changes are suggestive or indicative of impending secondary brain injury or impending worsening of brain damage. This neurovascular monitoring system 500 is also very useful in neurocritical care and coma management because non-convulsive seizure is a common complication of coma management. Non-convulsive seizure is often not recognized clinically. The auricular EEG monitoring system 100 (of this neurovascular monitoring system 500) will enable the clinician to easily detect seizures and non-convulsive seizures. The network interface 53 is configured to generate a display of the wearer's EEG status and the cerebral blood flow status on at least one of: a client device 400 of the wearer 900 or a client device 400 of a healthcare provider 950 of the wearer. The client device 400 of the healthcare provider may include monitoring devices in a hospital (including intensive care unit) or in a clinic. The network interface 53 may also be in electronic communication with a speaker 15, 404A on a client device 400 of the wearer 900 or a speaker 15, 404B on a client device 400 of the wearer's healthcare provider 950. The speaker 15, 404A is configured to generate an audible notification when the processing unit 50, 401, detects at least one of the following: significant change of the wearer's EEG status (significant change of the wearer's EEG status refers to an increase of relative delta power more than a predetermined level or an increase of delta/alpha ratio more than a predetermined level) and significant change of the wearer's cerebral blood flow status (significant change of the wearer's cerebral blood flow status refers to a decrease of cerebral blood flow more than a predetermined level). Similarly, the vibrator 17, 404B is configured to generate a tactile notification when the processing unit 50, 401, detects at least one of the following: significant changes of the wearer's EEG status (e.g. an increase of relative delta power more than a predetermined level or an increase of delta/alpha ratio more than a predetermined level) and significant change of the wearer's cerebral blood flow status (e.g. a decrease of cerebral blood flow more than a predetermined level).

In some embodiments, a neurovascular monitoring system 500 may comprise an auricular EEG monitoring system 100 and a cephalic blood flow monitoring system 530 having an auricular SCOS unit 511. The processing unit 50, 401, is configured to analyze the EEG data recorded by the auricular EEG monitoring system 100 to detect at least one of the following: presence of EEG status change suggestive of stroke, presence of EEG status change suggestive of impending stroke, presence of EEG status change suggestive of impending secondary brain injury, presence of EEG status change suggestive of impending worsening of brain damage, presence of EEG status change suggestive of seizure, and presence of EEG status change suggestive of non-convulsive seizure. The processing unit 50, 401, is further configured to analyze the cerebral blood flow data recorded by the cephalic blood flow monitoring system 530 to detect at least one of the following: presence of cerebral blood flow status change suggestive of stroke, presence of cerebral blood flow status change suggestive of impending stroke, presence of cerebral blood flow status change suggestive of impending secondary brain injury, and presence of cerebral blood flow status change suggestive of impending worsening of brain damage. When the processing unit 50, 401, detects at least one of the following: presence of EEG status change suggestive of stroke, presence of EEG status change suggestive of impending stroke, presence of EEG status change suggestive of impending secondary brain injury, presence of EEG status change suggestive of impending worsening of brain damage, presence of EEG status change suggestive of seizure, presence of EEG status change suggestive of non-convulsive seizure, presence of cerebral blood flow status change suggestive of stroke, presence of cerebral blood flow status change suggestive of impending stroke. presence of cerebral blood flow status change suggestive of impending secondary brain injury, and presence of cerebral blood flow status change suggestive of impending worsening of brain damage, the processing unit 50, 401, is configured to send signals to the network interface 53 to prompt the network interface 53 to send warning notification to at least one of the following: a client device 400 of the wearer 900 and a client device 400 of the wearer's healthcare provider 950. The processing unit 50, 401, is also configured to analyze the EEG data recorded by the auricular EEG monitoring system 100 to detect presence of significant EEG status changes. As used herein, the significant EEG status changes include at least one of the following: EEG status change suggestive of stroke, EEG status change suggestive of impending stroke, EEG status change suggestive of seizure, EEG status change suggestive of non-convulsive seizure, EEG status change suggestive of impending secondary brain injury and EEG status change suggestive of impending worsening of brain damage. The processing unit 50, 401, is also configured to analyze the cerebral blood flow data recorded by the cephalic blood flow monitoring system 530 to detect presence of significant cerebral blood flow status changes. (As used herein, significant change refers to a change more than a predetermined level, as described hereinbefore.)

In some embodiments, a neurovascular monitoring system 500 may comprise an auricular EEG monitoring system 100 having a first EEG recording module 20 and a second EEG recording module 20. The neurovascular monitoring system 500 may further comprise a cephalic blood flow monitoring system 530 having a first auricular SCOS unit 511 and a second auricular SCOS unit 511. The first EEG recording module 20 and the first auricular SCOS unit may be configured to be linked to a first ear 902 of a wearer 900, while the second EEG recording module 20 and the second auricular SCOS unit 511 may be configured to be linked to a second ear 902 of the wearer. The first EEG recording module 20 and the first auricular SCOS unit 511 are as described hereinbefore. The second EEG recording module 20 may comprise a plurality of EEG sensor electrodes 12, 13, 72, 73, 82, 83, configured to be housed in a second auricular housing 11, and the second auricular housing 11 is configured to be placed in the wearer's second ear 902 when in use. Each EEG sensor electrode 12, 13, 72, 73, 82, 83, of the second EEG recording module 20 is in electronic communication with the second EEG recording module 20. The second EEG recording module 20 is configured to record EEG data of the wearer 900. The second EEG recording module 20 is in electronic communication with a processing unit 50, 401, that is configured to convert the raw EEG data into quantitative EEG (qEEG) data through digital signal processing techniques. The second EEG recording module 20 is in electronic communication with the processing unit 50, 401. The second auricular SCOS unit 511 comprises a second auricular SCOS sensor 512. The second auricular SCOS sensor 512 is configured to be housed in the second auricular housing 11. The second auricular SCOS unit 511 is configured to record cerebral blood flow data from the wearer's second ear 902, and the second auricular SCOS unit 511 is in electronic communication with the processing unit 50, 401. The processing unit 50, 401, is configured to analyze the EEG and qEEG data recorded by the first EEG recording module 20 to detect at least one of the following: presence of EEG status change suggestive of stroke, presence of EEG status change suggestive of impending stroke, presence of EEG status change suggestive of impending secondary brain injury, presence of EEG status change suggestive of impending worsening of brain damage, presence of EEG status change suggestive of seizure, and presence of EEG status change suggestive of non-convulsive seizure. The processing unit 50, 401, is configured to analyze the cerebral blood flow data recorded by the first auricular SCOS unit 511 to detect at least one of the following: presence of cerebral blood flow status change suggestive of stroke, presence of cerebral blood flow status change suggestive of impending stroke, presence of cerebral blood flow status change suggestive of impending secondary brain injury, and presence of cerebral blood flow status change suggestive of impending worsening of brain damage. Similarly, the processing unit 50, 401, is configured to analyze the EEG data recorded by the second EEG recording module 20 to detect at least one of the following: presence of EEG status change suggestive of stroke, presence of EEG status change suggestive of impending stroke, presence of EEG status change suggestive of impending secondary brain injury, presence of EEG status change suggestive of impending worsening of brain damage, presence of EEG status change suggestive of seizure, and presence of EEG status change suggestive of non-convulsive seizure. Likewise, the processing unit 50, 401, is configured to analyze the cerebral blood flow data recorded by the second auricular SCOS unit 511 to detect at least one of the following: presence of cerebral blood flow status change suggestive of stroke, presence of cerebral blood flow status change suggestive of impending stroke, presence of cerebral blood flow status change suggestive of impending secondary brain injury, and presence of cerebral blood flow status change suggestive of impending worsening of brain damage. When the processing unit 50, 401, detects at least one of the following: presence of EEG status change suggestive of stroke by analyzing EEG and qEEG data recorded by the first EEG recording module 20, presence of EEG status change suggestive of impending stroke by analyzing EEG and qEEG data recorded by the first EEG recording module 20, presence of EEG status change suggestive of impending secondary brain injury by analyzing EEG and qEEG data recorded by the first EEG recording module 20, presence of EEG status change suggestive of impending worsening of brain damage by analyzing EEG and qEEG data recorded by the first EEG recording module 20, presence of EEG status change suggestive of seizure by analyzing EEG data recorded by the first EEG recording module 20, presence of EEG status change suggestive of non-convulsive seizure by analyzing EEG and qEEG data recorded by the first EEG recording module 20, presence of EEG status change suggestive of stroke by analyzing EEG and qEEG data recorded by the second EEG recording module 20, presence of EEG status change suggestive of impending stroke by analyzing EEG and qEEG data recorded by the second EEG recording module 20, presence of EEG status change suggestive of impending secondary brain injury by analyzing EEG and qEEG data recorded by the second EEG recording module 20, presence of EEG status change suggestive of impending worsening of brain damage by analyzing EEG and qEEG data recorded by the second EEG recording module 20, presence of EEG status change suggestive of seizure by analyzing EEG and qEEG data recorded by the second EEG recording module 20, presence of EEG status change suggestive of non-convulsive seizure by analyzing EEG and qEEG data recorded by the second EEG recording module 20, presence of cerebral blood flow status change suggestive of stroke by analyzing cerebral blood flow data recorded by the first auricular SCOS unit 511, presence of cerebral blood flow status change suggestive of impending stroke by analyzing cerebral blood flow data recorded by the first auricular SCOS unit 511, presence of cerebral blood flow status change suggestive of impending secondary brain injury by analyzing cerebral blood flow data recorded by the first auricular SCOS unit 511, presence of cerebral blood flow status change suggestive of impending worsening of brain damage by analyzing cerebral blood flow data recorded by the first auricular SCOS unit 511, presence of cerebral blood flow status change suggestive of stroke by analyzing cerebral blood flow data recorded by the second auricular SCOS unit 511, presence of cerebral blood flow status change suggestive of impending stroke by analyzing cerebral blood flow data recorded by the second auricular SCOS unit 511, presence of cerebral blood flow status change suggestive of impending secondary brain injury by analyzing cerebral blood flow data recorded by the second auricular SCOS unit 511, and presence of cerebral blood flow status change suggestive of impending worsening of brain damage by analyzing cerebral blood flow data recorded by the second auricular SCOS unit 511, the processing unit 50, 401, is configured to send signals to the network interface 53 to prompt the network interface 53 to send warning notification to at least one of: the client device 400 of the wearer 900 and the client device 400 of the wearer's healthcare provider 950. When the processing unit 50, 401, detects cessation of all of the above, the processing unit 50, 401, may be configured to send signals to the network interface 53 to prompt the network interface 53 to send notification to at least one of: the client device 400 of the wearer 900 and the client device of the wearer's healthcare provider 950. (As used herein, significant change refers to a change more than a predetermined level, as described hereinbefore.) In some embodiments, the processing unit 50, 401, may be configured to analyze the EEG and qEEG data recorded by the first EEG recording module 20 to detect at least one of the following: an increase of relative delta power more than a predetermined level, an increase of delta/alpha ratio more than a predetermined level and presence of one of: seizure and non-convulsive seizure. The processing unit 50, 401, may be also configured to analyze the EEG and qEEG data recorded by the second EEG recording module 20 to detect at least one of the following: an increase of relative delta power more than a predetermined level, an increase of delta/alpha ratio more than a predetermined level and presence of at least one of: seizure and non-convulsive seizure. The processing unit 50, 401, may be configured to analyze the cerebral blood flow data recorded by the first auricular SCOS unit 511 to detect presence of a decrease of cerebral blood flow more than a predetermined level. Similarly, the processing unit 50, 401, may be configured to analyze the cerebral blood flow data recorded by the second auricular SCOS unit to detect presence of a decrease of cerebral blood flow more than a predetermined level. The processing unit 50, 401, may be configured to generate signals to the network interface 53 to prompt the network interface 53 to generate a notification to at least one of: a client device 400 of the wearer 900 and a client device 400 of a healthcare provider 950 of the wearer when the processing unit detect presence of at least one of the following: presence of an increase of relative delta power more than a predetermined level, presence of an increase of delta/alpha ratio more than a predetermined level and presence of one of: seizure and non-convulsive seizure as recorded by the first EEG recording module 20, presence of an increase of relative delta power more than a predetermined level, presence of an increase of delta/alpha ratio more than a predetermined level and presence of one of: seizure and non-convulsive seizure as recorded by the second EEG recording module 20, presence of a decrease of cerebral blood flow more than a predetermined level as recorded by the first auricular SCOS unit 511, and presence of a decrease of cerebral blood flow more than a predetermined level as recorded by the second SCOS unit 511.

It should be noted that although both cerebral hypoperfusion and sleep or drowsiness can produce increase of slow waves (theta waves and delta waves) in EEG and qEEG, there are distinct features that the processing unit 50, 401, can use to differentiate between them. Studies have shown that quantitative EEG (qEEG) is a highly sensitive, though not perfectly specific, tool for detecting cerebral hypoperfusion, with studies indicating a sensitivity greater than 80% and specificity of roughly 70% between qEEG changes and actual blood flow reductions. It can detect ischemia before clinical symptoms or imaging (CT/MRI) reveal damage. There are features that the processing unit 50, 401, can use to differentiate between hypoperfusion-induced slow waves and sleep-induced slow waves, such as analyzing topography (location or where they appear) and coherence (how regions communicate), as sleep slow waves are generally global, while hypoperfusion-related slow waves are often focal or regional. They also differ in other electrophysiological signatures. When the qEEG data are combined with data from auricular SCOS unit 511 and data from PPG unit 555, the sensitivity and specificity for qEEG in detecting cerebral hypoperfusion are very high. The fact that quantitative EEG can detect cerebral ischemia and hypoperfusion before clinical symptoms and CT or MRI imaging offers significant advantages.

In some embodiments, the auricular Speckle Contrast Optical Spectroscope unit (auricular SCOS unit) 511 may be configured as a wireless auricular SCOS unit 511. (Wireless SCOS is known in the art. For example, Wireless SCOS Headset has been developed by researchers in Caltech and USC for monitoring of brain blood flow.) As described hereinbefore, all of the EEG sensor electrodes 12, 13, 72, 73, 82, 83, and the optional reference electrode 84 may be configured as wireless electrodes and the EEG amplifier 21 may be configured as wireless EEG amplifier 21. Thus, the wireless auricular SCOS unit 511, wireless EEG sensor electrodes 12, 13, 72, 73, 82, 83, and the wireless optional reference electrode 84 may all be housed in an auricular housing 11, while the processing unit 50, 401 and the wireless EEG amplifier 21 may be housed remotely in a client device 400 of the wearer 900, such as a watch-type client device or a smart phone-type client device 400 or a tablet-type client device 400. The wireless setups will enable this neurovascular monitoring system 500 even more wearable and more convenient for inpatient use and outpatient ambulatory use.

There are a few devices designed to monitor EEG and cerebral blood flow simultaneously, for example, Hybrid EEG-fNIRS Headbands and Nuroflux. The Hybrid EEG-fNIRS headbands use wearable headband to do scalp EEG and functional near-infrared spectroscopy. Although it is wearable, the headband is too bulky cumbersome and nonaesthetic and not suitable for long-term ambulatory use. Nuroflux can monitor EEG through scalp EEG and can monitor blood flow by using conductivity changes in electrocardiogram (EKG/ECG) signal propagation to infer dynamic blood flow states. Nuroflux also uses a headband to mount its elements and is similarly too bulky, cumbersome, nonaesthetic and not suitable for long-term ambulatory use. Both Hybrid EEG-fNIRS Headbands and Nuroflux are primarily designed for inpatient use for monitoring of stroke or traumatic brain injury patients. When EEG and blood flow data are simultaneously assessed, they can offer a more complete picture of cerebral physiology. The dual approach can enhance the capability in understanding various neurological conditions and improve the treatment strategies. The neurovascular monitoring system 500 as hereby disclosed is a much smaller device, located inside the ear and both the auricular EEG monitoring system 100 and the auricular SCOS unit 511 can be easily installed by the wearer himself/herself simply by placing the device in the ear 902 and can be equally easily removed by simply removing the device from the ear 902. This neurovascular monitoring system 500 is wearable, ambulatory, very convenient for the wearer and suitable for both inpatient and outpatient long-term use. This system 500 is especially very suitable for long-term monitoring in outpatient setting for patients with carotid stenosis (or stenosis of other arteries, like the middle cerebral artery, anterior cerebral artery or vertebra-basial artery) to watch and to warn of any worsening of the arterial stenosis, impending stroke or future risk of stroke. When cerebral blood flow drops below a critical threshold (around 30% of normal), detectable EEG changes often appear before patients have physical symptoms. Patients with critical or near-critical stenosis of cerebral arteries might not have symptoms, nevertheless the neurovascular monitoring system 500 would be able to detect EEG changes and blood flow changes to warm the patient or patient's healthcare provider to take appropriate actions.

In some embodiments, a cephalic blood flow monitoring apparatus for migraine and cluster headache detection may comprise a cephalic blood flow monitoring system 530, a network interface 53 and a processing unit 50, 401. The cephalic blood flow monitoring system 530 may be configured to record cerebral and extracranial blood flow data of a wearer. The cephalic blood flow monitoring system 530 may comprise an auricular SCOS unit 511 and a PPG unit 555, as described hereinbefore. The processing unit 50, 401, may be in electronic communication with the cephalic blood flow monitoring system 530 and the network interface 53. The processing unit 50, 401, is configured to analyze the cerebral and extracranial blood flow data recorded by the cephalic blood flow monitoring system 530 to detect presence or cessation of cerebral and extracranial blood flow data suggestive of at least one of: migraine and cluster headache. The processing unit 50, 401, is also configured to analyze the cerebral and extracranial blood flow data recorded by the cephalic blood flow monitoring system 530 to detect presence or cessation of cerebral and extracranial blood flow data suggestive of at least one of: impending migraine and impending cluster headache. When the processing unit 50, 401, detects presence of cerebral and extracranial blood flow data suggestive of at least one of: migraine and cluster headache, the processing unit 50, 401, is configured to generate signals to the network interface 53 to prompt the network interface 53 to send notification to at least one of: a client device 400 of the wearer 900 and a client device 400 of a healthcare provider 950 of the wearer. When the processing unit 50, 401, detects presence of cerebral and extracranial blood flow data suggestive of at least one of: impending migraine and impending cluster headache, the processing unit 50, 401, is configured to generate signals to the network interface 53 to prompt the network interface 53 to send notification to at least one of: the client device 400 of the wearer 900 and the client device 400 of the healthcare provider 950 of the wearer. When the processing unit 50, 401, detects both of the following: cessation of cerebral and extracranial blood flow data suggestive of at least one of migraine and cluster headache and cessation of cerebral and extracranial blood flow data suggestive of at least one of impending migraine and impending cluster headache, the processing unit 50, 401, is further configured to generate signals to the network interface 53 to prompt the network interface 53 to send notification to at least one of: the client device 400 of the wearer 900 and the client device 400 of the healthcare provider 950 of the wearer.

While some exemplary shapes and sizes have been provided for elements of the auricular EEG monitoring system 100 and automatic detection-therapy system 101 it should be understood to one of ordinary skill in the art that the elements described herein may be configured in a plurality of sizes and shapes including “T” shaped, “X” shaped, square shaped, rectangular shaped, cylinder shaped, cuboid shaped, hexagonal prism shaped, triangular prism shaped, or any other geometric or non-geometric shape, including combinations of shapes. It is not intended herein to mention all the possible alternatives, equivalent forms or ramifications of the invention. It is understood that the terms and proposed shapes used herein are merely descriptive, rather than limiting, and that various changes, such as to size and shape, may be made without departing from the spirit or scope of the invention.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. An automatic detection-therapy system for migraine, comprising:
a neurovascular monitoring system, comprising an auricular electroencephalogram (EEG) monitoring system and a cephalic blood flow monitoring system, wherein the auricular EEG monitoring system is configured to record EEG data of a wearer from at least an ear of the wearer, wherein the cephalic blood flow monitoring system is configured to record cerebral and extracranial blood flow data of the wearer, wherein the neurovascular monitoring system is configured to simultaneously record the wearer's EEG data and cerebral and extracranial blood flow data;
a first neuromodulation unit configured to give neuromodulating electric stimulation to the wearer when activated; and
a processing unit, wherein the processing unit is in electronic communication with the auricular EEG monitoring system, the cephalic blood flow monitoring system and the first neuromodulation unit;
wherein the processing unit is configured to convert the EEG data recorded by the auricular EEG monitoring system into quantitative EEG (qEEG) data, wherein the processing unit is configured to analyze the EEG data and qEEG data recorded by the auricular EEG monitoring system to detect presence or cessation of EEG and qEEG signals suggestive of migraine, wherein the processing unit is configured to analyze the cerebral and extracranial blood flow data recorded by the cephalic blood flow monitoring system to detect presence or cessation of cerebral and extracranial blood flow data suggestive of migraine;
wherein when the processing unit detects at least one of the following: presence of EEG and qEEG signals suggestive of migraine and presence of cerebral and extracranial blood flow data suggestive of migraine, the processing unit is configured to immediately send signals to the first neuromodulation unit to automatically start sending neuromodulating electric stimulation to the wearer;
wherein the processing unit is configured to analyze the EEG and qEEG data recorded by the auricular EEG monitoring system to detect presence or cessation of EEG and qEEG signals suggestive of impending migraine, wherein the processing unit is also configured to analyze the cerebral and extracranial blood flow data recorded by the cephalic blood flow monitoring system to detect presence or cessation of cerebral and extracranial blood flow data suggestive of impending migraine;
wherein when the processing unit detects at least one of the following: presence of EEG and qEEG signals suggestive of impending migraine and presence of cerebral and extracranial blood flow data suggestive of impending migraine, the processing unit is configured to immediately send signals to the first neuromodulation unit to automatically start sending neuromodulating electric stimulation to the wearer; and
wherein when the processing unit detects all of the following: cessation of EEG and qEEG signals suggestive of migraine, cessation of EEG and qEEG signals suggestive of impending migraine, cessation of cerebral and extracranial blood flow data suggestive of migraine and cessation of cerebral and extracranial blood flow data suggestive of impending migraine, the processing unit is further configured to immediately send signals to the first neuromodulation unit to automatically stop sending neuromodulating electric stimulation to the wearer.

2. The automatic detection-therapy system for migraine of claim 1, wherein the first neuromodulation unit comprises at least one of the following components: a first transcutaneous auricular vagus nerve stimulation unit (first taVNS unit), a supraorbital nerve stimulation unit, an infraorbital nerve stimulation unit, a first auriculotemporal nerve stimulation unit, an occipital nerve stimulation unit and a first greater auricular nerve stimulation unit, wherein the processing unit is in electronic communication with the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit, wherein the processing unit is configured to analyze the EEG and qEEG data recorded by the auricular EEG monitoring system to detect presence or cessation of EEG and qEEG signals suggestive of migraine, wherein the processing unit is further configured to analyze the EEG and qEEG data recorded by the auricular EEG monitoring system to detect presence or cessation of EEG and qEEG signals suggestive of impending migraine, wherein the processing unit is configured to analyze the cerebral and extracranial blood flow data recorded by the cephalic blood flow monitoring system to detect presence or cessation of cerebral and extracranial blood flow data suggestive of migraine, wherein the processing unit is further configured to analyze the cerebral and extracranial blood flow data recorded by the cephalic blood flow monitoring system to detect presence or cessation of cerebral and extracranial blood flow data suggestive of impending migraine,
wherein when the processing unit detects at least one of the following: presence of EEG and qEEG signals suggestive of migraine and presence of cerebral and extracranial blood flow data suggestive of migraine, the processing unit is configured to start sending neuromodulating electric stimulation from at least one of the following: the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit;
wherein when the processing unit detects at least one of the following: presence of EEG and qEEG signals suggestive of impending migraine and presence of cerebral and extracranial blood flow data suggestive of impending migraine, the processing unit is configured to start sending neuromodulating electric stimulation from at least one of the following: the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit; and
wherein when the processing unit detects all of the following: cessation of EEG and qEEG signals suggestive of migraine, cessation of EEG and qEEG signals suggestive of impending migraine, cessation of cerebral and extracranial blood flow data suggestive of migraine and cessation of cerebral and extracranial blood flow data suggestive of impending migraine, the processing unit is configured to stop sending neuromodulating electric stimulation from any of the following: the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit.

3. The automatic detection-therapy system for migraine of claim 1, wherein the auricular electroencephalogram (EEG) monitoring system comprises a first EEG recording module having a plurality of EEG sensor electrodes, wherein the first EEG recording module is in electronic communication with each EEG sensor electrode of the first EEG recording module, wherein the first EEG recording module is configured to record EEG data of the wearer, wherein all of the EEG sensor electrodes of the first EEG recording module are configured to be housed in a first auricular housing having a first tubular-shaped structure, wherein the first tubular-shaped structure is configured to be inserted into an external ear canal of the wearer's first ear when in use, wherein all of the EEG sensor electrodes of the first EEG recording module are configured to be located on a surface and partially embedded in the surface with protrusion at the surface of the first tubular-shaped structure, wherein one or more EEG sensor electrode(s) are located at the upper surface of the first tubular-shaped structure, wherein one or more EEG sensor electrode(s) are located above the horizontal level of the first tubular-shaped structure and are facing forward-upward, wherein one or more EEG sensor electrode(s) are located above the horizontal level of the first tubular-shaped structure and are facing backward-upward, wherein the first tubular-shaped structure comprises an elastic flexible and adaptable material, and wherein the elastic flexible and adaptable material of the first tubular-shaped structure is configured to have appropriate elasticity flexibility and adaptability so that when the first tubular-shaped structure is inserted into the external ear canal of the wearer's first ear the first tubular-shaped structure will naturally adapt to the contour of the external ear canal of the wearer's first ear and snugly fill the interior of the external ear canal of the wearer's first ear, so that all of the EEG sensor electrodes of the first EEG recording module are naturally in close contact with the skin of the external ear canal of the wearer's first ear when the first tubular-shaped structure is inserted into the external ear canal of the wearer's first ear.

4. The automatic detection-therapy system for migraine of claim 1, wherein the cephalic blood flow monitoring system comprises one of: a speckle contrast optical spectroscopy unit (SCOS unit) and an auricular speckle contrast optical spectroscope unit (auricular SCOS unit), wherein the auricular SCOS unit is configured as a SCOS unit placed in an ear of the wearer when in use, wherein the SCOS unit is configured to record the wearer's cerebral and extracranial blood flow data from the wearer's scalp, wherein the auricular SCOS unit is configured to record the wearer's cerebral and extracranial blood flow data from the wearer's ear.

5. The automatic detection-therapy system for migraine of claim 1, wherein the auricular EEG monitoring system comprises a first EEG recording module having a plurality of EEG sensor electrodes and a first reference electrode, wherein all of the EEG sensor electrodes and the first reference electrode of the first EEG recording module are configured to be located in a first ear of the wearer, wherein the first EEG recording module is configured to record EEG data of the wearer, wherein the cephalic blood flow monitoring system comprises a first auricular speckle contrast optical spectroscope unit (first auricular SCOS unit) having a first auricular SCOS sensor, wherein the first neuromodulation unit comprises at least one of the following: a first transcutaneous auricular vagus nerve stimulation (taVNS) unit having a first taVNS stimulating electrode for stimulation of the auricular branch of vagus nerve of the wearer's first ear, a first auriculotemporal nerve stimulation unit having a first auriculotemporal nerve stimulating electrode for stimulation of the auriculotemporal nerve of the wearer's first ear, and a first greater auricular nerve stimulation unit having a greater auricular nerve stimulating electrode for stimulation of the greater auricular nerve of the wearer's first ear; wherein all of the EEG sensor electrodes, the first reference electrode, the first auricular SCOS sensor, the first taVNS stimulating electrode, the first auriculotemporal nerve stimulating electrode and the first greater auricular nerve stimulating electrode are configured to be housed in a first auricular housing, wherein the first auricular housing includes a first tubular-shaped structure and a first body-structure, wherein the first tubular-shaped structure is configured to be inserted into an external ear canal of the wearer's first ear when in use, wherein the first body-structure is configured to be placed at immediate opening of the external ear canal of the wearer's first ear and be placed inside a tragus-concha bowl of the wearer's first ear when in use, wherein all of the EEG sensor electrodes are configured to be located on a surface and partially embedded in the surface with protrusion at the surface of the first tubular-shaped structure, wherein the first greater auricular nerve stimulating electrode and the first reference electrode are configured to be located on a surface and partially embedded in the surface with protrusion at the surface of the first body-structure, wherein the first auricular SCOS sensor, the first taVNS stimulating electrode and the first auriculotemporal nerve stimulating electrode are configured to be located on the surface and partially embedded in the surface with protrusion at the surface of one of: the first tubular-shaped structure and the first body-structure, wherein the first tubular-shaped structure is configured to be made of elastic flexible adaptable material and the material is configured to have appropriate elasticity flexibility and adaptability so that the first tubular-shaped structure will naturally adapt to the contour of the external ear canal of the wearer's first ear and snugly fill the interior of the external ear canal of the wearer's first ear when the first tubular-shaped structure is inserted into the external ear canal of the wearer's first ear and so that all of the EEG sensor electrodes will be naturally in close contact with the skin of the external ear canal of the wearer's first ear; wherein the first body-structure is configured to be made of elastic flexible adaptable material and the material is configured to have appropriate elasticity flexibility and adaptability so that the first body-structure will naturally adapt to the contour of the tragus-concha bowl of the wearer's first ear and snugly fill the interior of the tragus-concha bowl of the wearer's first ear when the first body-structure is placed inside the tragus-concha bowl of the wearer's first ear and so that the first greater auricular nerve stimulating electrode and the first reference electrode will be naturally in close contact with the skin of the tragus-concha bowl of the wearer's first ear, and, at the same time, the first taVNS stimulating electrode, the first auriculotemporal nerve stimulating electrode and the first auricular SCOS sensor will be naturally in close contact with one of: the skin of the tragus-concha bowl of the wearer's first ear and the skin of the external ear canal of the wearer's first ear, and so that the first taVNS stimulating electrode will be naturally in close contact with the vagus innervated auricular skin of the wearer's first ear, provided by carefully selecting a location for the first taVNS stimulating electrode on the first tubular-shaped structure or the first body-structure to match one of the innervation locations of the vagus innervated auricular skin on the external ear canal of the wearer's first ear or the tragus-concha bowl of the wearer's first ear, and, at the same time, the first auriculotemporal nerves stimulating electrode will be naturally in close contact with auriculotemporal nerve innervated auricular skin of the wearer's first ear, provided by carefully selecting a location for the first auriculotemporal nerve stimulating electrode on the first tubular-shaped structure or the first body-structure to match one of the innervation locations of the auriculotemporal nerve innervated skin on the external ear canal of the wearer's first ear or the tragus-concha bowl of the wearer's first ear, and, at the same time, the first greater auricular nerve stimulating electrode will be naturally in close contact with greater auricular nerve innervated auricular skin of the wearer's first ear, provided by carefully selecting a location for the first greater auricular nerve stimulating electrode on the first body-structure to match one of the innervation locations of the greater auricular nerve innervated skin on tragus-concha bowl of the wearer's first ear.

6. The automatic detection-therapy system for migraine of claim 5, further comprising a network interface, wherein the network interface is in electronic communication with the processing unit, wherein the network interface is configured to generate a notification to at least one of: a client device of the wearer and a client device of a healthcare provider of the wearer when the processing unit detects at least one of the following: presence of EEG and qEEG signals suggestive of migraine and presence of cerebral and extracranial blood flow data suggestive of migraine, wherein the network interface is further configured to generate a notification to at least one of: the client device of the wearer and the client device of the healthcare provider of the wearer when the processing unit detects at least one of the following: presence of EEG and qEEG signals suggestive of impending migraine and presence of cerebral and extracranial blood flow data suggestive of impending migraine, wherein the network interface is configured to generate a notification to at least one of the following: the client device of the wearer and the client device of the wearer's healthcare provider when the processing unit detects all of the following: cessation of EEG and qEEG signals suggestive of migraine, cessation of EEG and qEEG signals suggestive of impending migraine, cessation of cerebral and extracranial blood flow data suggestive of migraine and cessation of cerebral and extracranial blood flow data suggestive of impending migraine; wherein all of the EEG sensor electrodes of the first EEG recording module are configured as wireless EEG sensor electrodes, wherein the first reference electrode is configured as a first wireless reference electrode, wherein the first EEG recording module comprises a wireless EEG amplifier, wherein the first auricular SCOS unit is configured as a first wireless auricular SCOS unit, and wherein all of the wireless EEG sensor electrodes of the first EEG recording module, the wireless first reference electrode and the first wireless auricular SCOS unit are housed in the first auricular housing while the wireless EEG amplifier, the first EEG recording module and the processing unit are housed remotely in a client device.

7. The automatic detection-therapy system for migraine of claim 2, wherein the cephalic blood flow monitoring system includes a first auricular speckle contrast optical spectroscopy unit (first auricular SCOS unit) and a second auricular speckle contrast optical spectroscopy unit (second auricular SCOS unit), wherein the first auricular SCOS unit is configured to be located in a first ear of the wearer when in use, wherein the second auricular SCOS unit is configured to be located in a second ear of the wearer when in use, wherein the first auricular SCOS unit is configured to record cerebral and extracranial blood flow data of the wearer from the wearer's first ear, wherein the second auricular SCOS unit is configured to record cerebral and extracranial blood flow data of the wearer from the wearer's second ear, wherein the first auricular SCOS unit is in electronic communication with the processing unit, wherein the second auricular SCOS unit is in electronic communication with the processing unit; wherein the auricular EEG monitoring system comprises a first EEG recording module and a second EEG recording module, wherein the first EEG recording module comprises a plurality of EEG sensor electrodes, wherein the first EEG recording module is in electronic communication with each EEG sensor electrode of the first EEG recording module, wherein each EEG sensor electrode of the first EEG recording module is configured to contact separate areas of the wearer's skin selected from at least one of the following: an external ear canal of the wearer's first ear, an external ear of the wearer's first ear, and a peri-auricular area around the wearer's first ear, wherein the first EEG recording module is in electronic communication with the processing unit, wherein the first EEG recording module is configured to record EEG data of the wearer from the wearer's first ear; wherein the second EEG recording module comprises a plurality of EEG sensor electrodes, wherein the second EEG recording module is in electronic communication with each EEG sensor electrode of the second EEG recording module, wherein each EEG sensor electrode of the second EEG recording module is configured to contact separate areas of the wearer's skin selected from at least one of the following: an external ear canal of the second ear of the wearer, an external ear of the wearer's second ear, and a peri-auricular area around the wearer's second ear, wherein the second EEG recording module is configured to record EEG data of the wearer, and wherein the second EEG recording module is in electronic communication with the processing unit.

8. The automatic detection-therapy system for migraine of claim 7, further comprising a second neuromodulation unit, wherein the second neuromodulation unit comprises at least one of the following: a second transcutaneous auricular vagus nerve stimulation unit (second taVNS unit), a second auriculotemporal nerve stimulation unit and a second greater auricular nerve stimulation unit, wherein the second taVNS unit includes a second taVNS stimulating electrode configured to contact vagus innervated auricular skin of the wearer's second ear, wherein the second auriculotemporal nerve stimulation unit includes a second auriculotemporal nerve stimulating electrode configured to contact auriculotemporal nerve innervated auricular skin of the wearer's second ear, wherein the second greater auricular nerve stimulation unit includes a second greater auricular nerve stimulating electrode configured to contact greater auricular nerve innervated auricular skin of the wearer's second ear, wherein the processing unit is configured to analyze the EEG and qEEG data recorded by the auricular EEG monitoring system, including EEG and qEEG data recorded by the first EEG recording module and EEG and qEEG data recorded by the second EEG recording module, to detect presence or cessation of EEG and qEEG signals suggestive of migraine, wherein the processing unit is further configured to analyze the EEG and qEEG data recorded by the auricular EEG monitoring system, including EEG and qEEG data recorded by the first EEG recording module and EEG and qEEG data recorded by the second EEG recording module, to detect presence or cessation of EEG and qEEG signals suggestive of impending migraine, wherein the processing unit is configured to analyze the cerebral and extracranial blood flow data recorded by the cephalic blood flow monitoring system, including cerebral and extracranial blood flow data recorded by the first auricular SCOS unit and cerebral and extracranial blood flow data recorded by the second auricular SCOS unit, to detect presence or cessation of cerebral and extracranial blood flow data suggestive of migraine, wherein the processing unit is further configured to analyze the cerebral and extracranial blood flow data recorded by the cephalic blood flow monitoring system, including cerebral and extracranial blood flow data recorded by the first auricular SCOS unit and cerebral and extracranial blood flow data recorded by the second auricular SCOS unit, to detect presence or cessation of cerebral and extracranial blood flow data suggestive of impending migraine,
wherein when the processing unit detects at least one of the following: presence of EEG and qEEG signals suggestive of migraine by analyzing the EEG and qEEG data recorded by the first EEG recording module, presence of EEG and qEEG signals suggestive of migraine by analyzing the EEG and qEEG data recorded by the second EEG recording module, presence of cerebral and extracranial blood flow data suggestive of migraine by analyzing cerebral and extracranial blood flow data recorded by the first auricular SCOS unit, and presence of cerebral and extracranial blood flow data suggestive of migraine by analyzing cerebral and extracranial blood flow data recorded by the second auricular SCOS unit, the processing unit is configured to start sending predetermined neuromodulating electric stimulation from at least one of the following: the first taVNS unit, the first auriculotemporal nerve stimulation unit, the first greater auricular nerve stimulation unit, the second taVNS unit, the second auriculotemporal nerve stimulation unit, the second greater auricular nerve stimulation unit, the supraorbital nerve stimulation unit, the infraorbital nerve stimulation unit and the occipital nerve stimulation unit;
wherein when the processing unit detects at least one of the following: presence of EEG and qEEG signals suggestive of impending migraine by analyzing EEG and qEEG data recorded by the first EEG recording module, presence of EEG and qEEG signals suggestive of impending migraine by analyzing EEG and qEEG data recorded by the second EEG recording module, presence of cerebral and extracranial blood flow data suggestive of impending migraine by analyzing cerebral and extracranial blood flow data recorded by the first auricular SCOS unit, and presence of cerebral and extracranial blood flow data suggestive of impending migraine by analyzing cerebral and extracranial blood flow data recorded by the second auricular SCOS unit, the processing unit is configured to start sending predetermined neuromodulating electric stimulation from at least one of the following: the first taVNS unit, the first auriculotemporal nerve stimulation unit, the first greater auricular nerve stimulation unit, the second taVNS unit, the second auriculotemporal nerve stimulation unit, the second greater auricular nerve stimulation unit, the supraorbital nerve stimulation unit, the infraorbital nerve stimulation unit and the occipital nerve stimulation unit; and
wherein when the processing unit detects all of the following: cessation of EEG and qEEG signals suggestive of migraine by analyzing EEG and qEEG data recorded by the first EEG recording module, cessation of EEG and qEEG signals suggestive of impending migraine by analyzing EEG and qEEG data recorded by the first EEG recording module, cessation of cerebral and extracranial blood flow data suggestive of migraine by analyzing cerebral and extracranial blood flow data recorded by the first auricular SCOS unit, cessation of cerebral and extracranial blood flow data suggestive of impending migraine by analyzing cerebral and extracranial blood flow data recorded by the first auricular SCOS unit, cessation of EEG and qEEG signals suggestive of migraine by analyzing EEG and qEEG data recorded by the second EEG recording module, cessation of EEG and qEEG signals suggestive of impending migraine by analyzing EEG and qEEG data recorded by the second EEG recording module, cessation of cerebral and extracranial blood flow data suggestive of migraine by analyzing cerebral and extracranial blood flow data recorded by the second auricular SCOS unit, and cessation of cerebral and extracranial blood flow suggestive of impending migraine by analyzing cerebral and extracranial blood flow data recorded by the second auricular SCOS unit, the processing unit is further configured to stop sending neuromodulating electric stimulation from any of the following: the first taVNS unit, the first auriculotemporal nerve stimulation unit, the first greater auricular nerve stimulation unit, the second taVNS unit, the second auriculotemporal nerve stimulation unit, the second greater auricular nerve stimulation unit, the supraorbital nerve stimulation unit, the infraorbital nerve stimulation unit and the occipital nerve stimulation unit.

9. The automatic detection-therapy system for migraine of claim 2, further comprising at least one of the following: a multi-mode timer, a multi-mode switch, a multifunctional timer-switch and a programmable multifunctional timer-switch, to enable the wearer to do at least one of the following: to select manual or automatic control, to select various time courses, and to select at least one of the following neuromodulation components:
the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit including various combinations thereof, wherein when prompted the first neuromodulation unit is configured to generate neuromodulating electric stimulation to the wearer according to the selection of the stimulation mode selected by the wearer, and wherein the selections of the stimulation mode include the following: single neuromodulation by one component of the neuromodulation unit, double neuromodulation by various combinations of two components of the neuromodulation unit, and triple neuromodulation by various combinations of three components of the neuromodulation unit.

10. The automatic detection-therapy system for migraine of claim 4, wherein the cephalic blood flow monitoring system further comprises a photoplethysmography (PPG) unit, wherein the PPG unit is configured to record the wearer's cerebral and extracranial blood flow data and the wearer's blood pressure data, wherein the PPG unit is in electronic communication with the processing unit, wherein the processing unit is configured to analyze the cerebral and extracranial blood flow data recorded by the auricular SCOS unit and the cerebral and extracranial blood flow data and blood pressure data recorded by the PPG unit to assess the wearer's cerebral and extracranial blood flow data and blood pressure data.

11. The automatic detection-therapy system for migraine of claim 10, wherein the processing unit includes a closed-loop control system, wherein the closed-loop control system within the processing unit is in electronic communication with the auricular EEG monitoring system, the cephalic blood flow monitoring system and the neuromodulation unit, wherein the closed-loop control system receives real-time continuous input of the wearer's EEG and qEEG data from the auricular EEG monitoring system and real-time continuous input of the wearer's cerebral and extracranial blood flow data and blood pressure data from the cephalic blood flow monitoring system, wherein the closed-loop control system of the processing unit is configured to analyze these real-time EEG and qEEG data, cerebral and extracranial blood flow data and blood pressure data, using controlling algorithms, to continuously adjust the actuating outputs to the neuromodulation unit, wherein the actuating outputs includes turning on or turning off at least one component of the neuromodulation unit and adjusting the stimulating parameters of the neuromodulation unit during the time when the neuromodulation unit is turned on.

12. An automatic detection-therapy system for cluster headache, comprising:
a cephalic blood flow monitoring system, wherein the cephalic blood flow monitoring system is configured to record cerebral and extracranial blood flow data of a wearer;
a first neuromodulation unit configured to give neuromodulating electric stimulation to the wearer when activated; and
a processing unit, wherein the processing unit is in electronic communication with the cephalic blood flow monitoring system and the first neuromodulation unit;
wherein the processing unit is configured to analyze the cerebral and extracranial blood flow data recorded by the cephalic blood flow monitoring system to detect presence or cessation of cerebral and extracranial blood flow data suggestive of cluster headache;
wherein when the processing unit detects presence of cerebral and extracranial blood flow data suggestive of cluster headache, the processing unit is configured to immediately send signals to the first neuromodulation unit to automatically start sending neuromodulating electric stimulation to the wearer;
wherein the processing unit is configured to analyze the cerebral and extracranial blood flow data recorded by the cephalic blood flow monitoring system to detect presence or cessation of cerebral and extracranial blood flow data suggestive of impending cluster headache;
wherein when the processing unit detects presence of cerebral and extracranial blood flow data suggestive of impending cluster headache, the processing unit is configured to immediately send signals to the first neuromodulation unit to automatically start sending neuromodulating electric stimulation to the wearer; and
wherein when the processing unit detects both of the following: cessation of cerebral and extracranial blood flow data suggestive of cluster headache and cessation of cerebral and extracranial blood flow data suggestive of impending cluster headache, the processing unit is further configured to immediately send signals to the first neuromodulation unit to automatically stop sending neuromodulating electric stimulation to the wearer.

13. The automatic detection-therapy system for cluster headache of claim 12, wherein the first neuromodulation unit comprises at least one of the following components: a first transcutaneous auricular vagus nerve stimulation unit (first taVNS unit), a supraorbital nerve stimulation unit, an infraorbital nerve stimulation unit, a first auriculotemporal nerve stimulation unit, an occipital nerve stimulation unit and a first greater auricular nerve stimulation unit, wherein the processing unit is in electronic communication with the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit, wherein each component of the first neuromodulation unit is configured to give neuromodulating electric stimulation to the wearer when prompted.

14. The automatic detection-therapy system for cluster headache of claim 13, wherein the cephalic blood flow monitoring system comprises one of: a speckle contrast optical spectroscopy unit (SCOS unit) and an auricular speckle contrast optical spectroscope unit (auricular SCOS unit), wherein the auricular SCOS unit is configured as a SCOS unit placed in an ear of the wearer when in use, wherein the SCOS unit is configured to record the wearer's cerebral and extracranial blood flow data from the wearer's scalp, wherein the auricular SCOS unit is configured to record the wearer's cerebral and extracranial blood flow data from the wearer's ear.

15. The automatic detection-therapy system for cluster headache of claim 14, further comprising an auricular electroencephalogram (EEG) monitoring system configured to record EEG data of the wearer from at least an ear of the wearer, wherein the auricular EEG monitoring system comprises a first EEG recording module in electronic communication with the processing unit, wherein the first EEG recording module comprises a plurality of EEG sensor electrodes, wherein the first EEG recording module is in electronic communication with each EEG sensor electrode of the first EEG recording module, wherein the first EEG recording module is configured to record EEG data of the wearer, wherein the processing unit is configured to convert the EEG data recorded by the auricular EEG monitoring system into quantitative EEG (qEEG) data, wherein the processing unit is configured to analyze the EEG and qEEG data recorded by the auricular EEG monitoring system to detect presence or cessation of EEG and qEEG signals suggestive of cluster headache, wherein the processing unit is further configured to analyze the EEG and qEEG data recorded by the auricular EEG monitoring system to detect presence or cessation of EEG and qEEG signals suggestive of impending cluster headache, wherein the processing unit is configured to analyze the cerebral and extracranial blood flow data recorded by the cephalic blood flow monitoring system to detect presence or cessation of cerebral and extracranial blood flow data suggestive of cluster headache, wherein the processing unit is further configured to analyze the cerebral and extracranial blood flow data recorded by the cephalic blood flow monitoring system to detect presence or cessation of cerebral and extracranial blood flow data suggestive of impending cluster headache,
wherein when the processing unit detects at least one of the following: presence of EEG and qEEG signals suggestive of cluster headache and presence of cerebral and extracranial blood flow data suggestive of cluster headache, the processing unit is configured to start sending neuromodulating electric stimulation from at least one of the following: the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit;
wherein when the processing unit detects at least one of the following: presence of EEG and qEEG signals suggestive of impending cluster headache and presence of cerebral and extracranial blood flow data suggestive of impending cluster headache, the processing unit is configured to start sending neuromodulating electric stimulation from at least one of the following: the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit; and
wherein when the processing unit detects all of the following: cessation of EEG and qEEG signals suggestive of cluster headache, cessation of EEG and qEEG signals suggestive of impending cluster headache, cessation of cerebral and extracranial blood flow data suggestive of cluster headache and cessation of cerebral and extracranial blood flow data suggestive of impending cluster headache, the processing unit is configured to stop sending neuromodulating electric stimulation from any of the following: the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit.

16. The automatic detection-therapy system for cluster headache of claim 15, wherein all of the EEG sensor electrodes of the first EEG recording module are configured to be housed in a first auricular housing having a first tubular-shaped structure, wherein the first tubular-shaped structure is configured to be inserted into an external ear canal of the wearer's first ear when in use, wherein all of the EEG sensor electrodes of the first EEG recording module are configured to be located on a surface and partially embedded in the surface with protrusion at the surface of the first tubular-shaped structure, wherein one or more EEG sensor electrode(s) are located at the upper surface of the first tubular-shaped structure, wherein one or more EEG sensor electrode(s) are located above the horizontal level of the first tubular-shaped structure and are facing forward-upward, wherein one or more EEG sensor electrode(s) are located above the horizontal level of the first tubular-shaped structure and are facing backward-upward, wherein the first tubular-shaped structure comprises an elastic flexible and adaptable material, and wherein the elastic flexible and adaptable material of the first tubular-shaped structure is configured to have appropriate elasticity flexibility and adaptability so that when the first tubular-shaped structure is inserted into the external ear canal of the wearer's first ear the first tubular-shaped structure will naturally adapt to the contour of the external ear canal of the wearer's first ear and snugly fill the interior of the external ear canal of the wearer's first ear, so that all of the EEG sensor electrodes of the first EEG recording module are naturally in close contact with the skin of the external ear canal of the wearer's first ear when the first tubular-shaped structure is inserted into the external ear canal of the wearer's first ear.

17. The automatic detection-therapy system for cluster headache of claim 15, wherein the auricular EEG monitoring system comprises a first EEG recording module having a plurality of EEG sensor electrodes and a first reference electrode, wherein all of the EEG sensor electrodes and the first reference electrode of the first EEG recording module are configured to be located in a first ear of the wearer, wherein the first EEG recording module is configured to record EEG data of the wearer, wherein the cephalic blood flow monitoring system comprises a first auricular speckle contrast optical spectroscope unit (first auricular SCOS unit) having a first auricular SCOS sensor, wherein the first neuromodulation unit comprises at least one of the following: a first transcutaneous auricular vagus nerve stimulation (taVNS) unit having a first taVNS stimulating electrode for stimulation of the auricular branch of vagus nerve of the wearer's first ear, a first auriculotemporal nerve stimulation unit having a first auriculotemporal nerve stimulating electrode for stimulation of the auriculotemporal nerve of the wearer's first ear, and a first greater auricular nerve stimulation unit having a greater auricular nerve stimulating electrode for stimulation of the greater auricular nerve of the wearer's first ear; wherein all of the EEG sensor electrodes, the first reference electrode, the first auricular SCOS sensor, the first taVNS stimulating electrode, the first auriculotemporal nerve stimulating electrode and the first greater auricular nerve stimulating electrode are configured to be housed in a first auricular housing, wherein the first auricular housing includes a first tubular-shaped structure and a first body-structure, wherein the first tubular-shaped structure is configured to be inserted into an external ear canal of the wearer's first ear when in use, wherein the first body-structure is configured to be placed at immediate opening of the external ear canal of the wearer's first ear and be placed inside a tragus-concha bowl of the wearer's first ear when in use, wherein all of the EEG sensor electrodes are configured to be located on a surface and partially embedded in the surface with protrusion at the surface of the first tubular-shaped structure, wherein the first greater auricular nerve stimulating electrode and the first reference electrode are configured to be located on a surface and partially embedded in the surface with protrusion at the surface of the first body-structure, wherein the first auricular SCOS sensor, the first taVNS stimulating electrode and the first auriculotemporal nerve stimulating electrode are configured to be located on the surface and partially embedded in the surface with protrusion at the surface of one of: the first tubular-shaped structure and the first body-structure, wherein the first tubular-shaped structure is configured to be made of elastic flexible adaptable material and the material is configured to have appropriate elasticity flexibility and adaptability so that the first tubular-shaped structure will naturally adapt to the contour of the external ear canal of the wearer's first ear and snugly fill the interior of the external ear canal of the wearer's first ear when the first tubular-shaped structure is inserted into the external ear canal of the wearer's first ear and so that all of the EEG sensor electrodes will be naturally in close contact with the skin of the external ear canal of the wearer's first ear; wherein the first body-structure is configured to be made of elastic flexible adaptable material and the material is configured to have appropriate elasticity flexibility and adaptability so that the first body-structure will naturally adapt to the contour of the tragus-concha bowl of the wearer's first ear and snugly fill the interior of the tragus-concha bowl of the wearer's first ear when the first body-structure is placed inside the tragus-concha bowl of the wearer's first ear, and so that the first greater auricular nerve stimulating electrode and the first reference electrode will be naturally in close contact with the skin of the tragus-concha bowl of the wearer's first ear, and, at the same time, the first taVNS stimulating electrode, the first auriculotemporal nerve stimulating electrode and the first auricular SCOS sensor will be naturally in close contact with one of: the skin of the tragus-concha bowl of the wearer's first ear and the skin of the external ear canal of the wearer's first ear, and so that the first taVNS stimulating electrode will be naturally in close contact with the vagus innervated auricular skin of the wearer's first ear, provided by carefully selecting a location for the first taVNS stimulating electrode on the first tubular-shaped structure or the first body-structure to match one of the innervation locations of the vagus innervated auricular skin on the external ear canal of the wearer's first ear or the tragus-concha bowl of the wearer's first ear, and, at the same time, the first auriculotemporal nerves stimulating electrode will be naturally in close contact with auriculotemporal nerve innervated auricular skin of the wearer's first ear, provided by carefully selecting a location for the first auriculotemporal nerve stimulating electrode on the first tubular-shaped structure or the first body-structure to match one of the innervation locations of the auriculotemporal nerve innervated skin on the external ear canal of the wearer's first ear or the tragus-concha bowl of the wearer's first ear, and, at the same time, the first greater auricular nerve stimulating electrode will be naturally in close contact with greater auricular nerve innervated auricular skin of the wearer's first ear, provided by carefully selecting a location for the first greater auricular nerve stimulating electrode on the first body-structure to match one of the innervation locations of the greater auricular nerve innervated skin on tragus-concha bowl of the wearer's first ear.

18. The automatic detection-therapy system for cluster headache of claim 17, further comprising a network interface, wherein the network interface is in electronic communication with the processing unit, wherein the network interface is configured to generate a notification to at least one of: a client device of the wearer and a client device of a healthcare provider of the wearer when the processing unit detects at least one of the following: presence of EEG and qEEG signals suggestive of cluster headache and presence of cerebral and extracranial blood flow data suggestive of cluster headache, wherein the network interface is further configured to generate a notification to at least one of: the client device of the wearer and the client device of the healthcare provider of the wearer when the processing unit detects at least one of the following: presence of EEG and qEEG signals suggestive of impending cluster headache and presence of cerebral and extracranial blood flow data suggestive of impending cluster headache, wherein the network interface is configured to generate a notification to at least one of the following: the client device of the wearer and the client device of the wearer's healthcare provider when the processing unit detects all of the following: cessation of EEG and qEEG signals suggestive of cluster headache, cessation of EEG and qEEG signals suggestive of impending cluster headache, cessation of cerebral and extracranial blood flow data suggestive of cluster headache and cessation of cerebral and extracranial blood flow data suggestive of impending cluster headache; wherein all of the EEG sensor electrodes of the first EEG recording module are configured as wireless EEG sensor electrodes, wherein the first reference electrode is configured as a first wireless reference electrode, wherein the first EEG recording module comprises a wireless EEG amplifier, wherein the first auricular SCOS unit is configured as a first wireless auricular SCOS unit, and wherein all of the wireless EEG sensor electrodes of the first EEG recording module, the wireless first reference electrode and the first wireless auricular SCOS unit are housed in the first auricular housing while the wireless EEG amplifier, the first EEG recording module and the processing unit are housed remotely in a client device.

19. The automatic detection-therapy system for cluster headache of claim 15, wherein the cephalic blood flow monitoring system includes a first auricular speckle contrast optical spectroscopy unit (first auricular SCOS unit) and a second auricular speckle contrast optical spectroscopy unit (second auricular SCOS unit), wherein the first auricular SCOS unit is configured to be located in a first ear of the wearer when in use, wherein the second auricular SCOS unit is configured to be located in a second ear of the wearer when in use, wherein the first auricular SCOS unit is configured to record cerebral and extracranial blood flow data of the wearer from the wearer's first ear, wherein the second auricular SCOS unit is configured to record cerebral and extracranial blood flow data of the wearer from the wearer's second ear, wherein the first auricular SCOS unit is in electronic communication with the processing unit, wherein the second auricular SCOS unit is in electronic communication with the processing unit; wherein the auricular EEG monitoring system comprises a first EEG recording module and a second EEG recording module, wherein the first EEG recording module comprises a plurality of EEG sensor electrodes, wherein the first EEG recording module is in electronic communication with each EEG sensor electrode of the first EEG recording module, wherein each EEG sensor electrode of the first EEG recording module is configured to contact separate areas of the wearer's skin selected from at least one of the following: an external ear canal of the wearer's first ear, an external ear of the wearer's first ear, and a peri-auricular area around the wearer's first ear, wherein the first EEG recording module is configured to record EEG data of the wearer from the wearer's first ear, wherein the first EEG recording module is in electronic communication with the processing unit; wherein the second EEG recording module comprises a plurality of EEG sensor electrodes, wherein the second EEG recording module is in electronic communication with each EEG sensor electrode of the second EEG recording module, wherein each EEG sensor electrode of the second EEG recording module is configured to contact separate areas of the wearer's skin selected from at least one of the following: an external ear canal of the second ear of the wearer, an external ear of the wearer's second ear, and a peri-auricular area around the wearer's second ear, and wherein the second EEG recording module is configured to record EEG data of the wearer, and wherein the second EEG recording module is in electronic communication with the processing unit.

20. The automatic detection-therapy system for cluster headache of claim 15, further comprising a second neuromodulation unit, wherein the second neuromodulation unit comprises at least one of the following: a second transcutaneous auricular vagus nerve stimulation unit (second taVNS unit), a second auriculotemporal nerve stimulation unit and a second greater auricular nerve stimulation unit, wherein the second taVNS unit includes a second taVNS stimulating electrode configured to contact vagus innervated auricular skin of the wearer's second ear, wherein the second auriculotemporal nerve stimulation unit includes a second auriculotemporal nerve stimulating electrode configured to contact auriculotemporal nerve innervated auricular skin of the wearer's second ear, wherein the second greater auricular nerve stimulation unit includes a second greater auricular nerve stimulating electrode configured to contact greater auricular nerve innervated auricular skin of the wearer's second ear, wherein the processing unit is configured to analyze the EEG and qEEG data recorded by the auricular EEG monitoring system, including EEG and qEEG data recorded by the first EEG recording module and EEG and qEEG data recorded by the second EEG recording module, to detect presence or cessation of EEG and qEEG signals suggestive of cluster headache, wherein the processing unit is further configured to analyze the EEG and qEEG data recorded by the auricular EEG monitoring system, including EEG and qEEG data recorded by the first EEG recording module and EEG and qEEG data recorded by the second EEG recording module, to detect presence or cessation of EEG and qEEG signals suggestive of impending cluster headache, wherein the processing unit is configured to analyze the cerebral and extracranial blood flow data recorded by the cephalic blood flow monitoring system, including cerebral and extracranial blood flow data recorded by the first auricular SCOS unit and cerebral and extracranial blood flow data recorded by the second auricular SCOS unit, to detect presence or cessation of cerebral and extracranial blood flow data suggestive of cluster headache, wherein the processing unit is further configured to analyze the cerebral and extracranial blood flow data recorded by the cephalic blood flow monitoring system, including cerebral and extracranial blood flow data recorded by the first auricular SCOS unit and cerebral and extracranial blood flow data recorded by the second auricular SCOS unit, to detect presence or cessation of cerebral and extracranial blood flow data suggestive of impending cluster headache,
wherein when the processing unit detects at least one of the following: presence of EEG and qEEG signals suggestive of cluster headache by analyzing the EEG and qEEG data recorded by the first EEG recording module, presence of EEG and qEEG signals suggestive of cluster headache by analyzing the EEG and qEEG data recorded by the second EEG recording module, presence of cerebral and extracranial blood flow data suggestive of cluster headache by analyzing cerebral and extracranial blood flow data recorded by the first auricular SCOS unit, and presence of cerebral and extracranial blood flow data suggestive of cluster headache by analyzing cerebral and extracranial blood flow data recorded by the second auricular SCOS unit, the processing unit is configured to start sending predetermined neuromodulating electric stimulation from at least one of the following: the first taVNS unit, the first auriculotemporal nerve stimulation unit, the first greater auricular nerve stimulation unit, the second taVNS unit, the second auriculotemporal nerve stimulation unit, the second greater auricular nerve stimulation unit, the supraorbital nerve stimulation unit, the infraorbital nerve stimulation unit and the occipital nerve stimulation unit;
wherein when the processing unit detects at least one of the following: presence of EEG and qEEG signals suggestive of impending cluster headache by analyzing EEG and qEEG data recorded by the first EEG recording module, presence of EEG and qEEG signals suggestive of impending cluster headache by analyzing EEG and qEEG data recorded by the second EEG recording module, presence of cerebral and extracranial blood flow data suggestive of impending cluster headache by analyzing cerebral and extracranial blood flow data recorded by the first auricular SCOS unit, and presence of cerebral and extracranial blood flow data suggestive of impending cluster headache by analyzing cerebral and extracranial blood flow data recorded by the second auricular SCOS unit, the processing unit is configured to start sending predetermined neuromodulating electric stimulation from at least one of the following: the first taVNS unit, the first auriculotemporal nerve stimulation unit, the first greater auricular nerve stimulation unit, the second taVNS unit, the second auriculotemporal nerve stimulation unit, the second greater auricular nerve stimulation unit, the supraorbital nerve stimulation unit, the infraorbital nerve stimulation unit and the occipital nerve stimulation unit; and
wherein when the processing unit detects all of the following: cessation of EEG and qEEG signals suggestive of cluster headache by analyzing EEG and qEEG data recorded by the first EEG recording module, cessation of EEG and qEEG signals suggestive of impending cluster headache by analyzing EEG and qEEG data recorded by the first EEG recording module, cessation of cerebral and extracranial blood flow data suggestive of cluster headache by analyzing cerebral and extracranial blood flow data recorded by the first auricular SCOS unit, cessation of cerebral and extracranial blood flow data suggestive of impending cluster headache by analyzing cerebral and extracranial blood flow data recorded by the first auricular SCOS unit, cessation of EEG and qEEG signals suggestive of cluster headache by analyzing EEG data recorded by the second EEG recording module, cessation of EEG and qEEG signals suggestive of impending cluster headache by analyzing EEG and qEEG data recorded by the second EEG recording module, cessation of cerebral and extracranial blood flow data suggestive of cluster headache by analyzing cerebral and extracranial blood flow data recorded by the second auricular SCOS unit, and cessation of cerebral and extracranial blood flow suggestive of impending cluster headache by analyzing cerebral and extracranial blood flow data recorded by the second auricular SCOS unit, the processing unit is further configured to stop sending neuromodulating electric stimulation from any of the following: the first taVNS unit, the first auriculotemporal nerve stimulation unit, the first greater auricular nerve stimulation unit, the second taVNS unit, the second auriculotemporal nerve stimulation unit, the second greater auricular nerve stimulation unit, the supraorbital nerve stimulation unit, the infraorbital nerve stimulation unit and the occipital nerve stimulation unit.

21. The automatic detection-therapy system for cluster headache of claim 13, further comprising at least one of the following: a multi-mode timer, a multi-mode switch, a multifunctional timer-switch and a programmable multifunctional timer-switch, to enable the wearer to do at least one of the following: to select manual or automatic control, to select various time courses, and to select at least one of the following neuromodulation components: the first taVNS unit, the supraorbital nerve stimulation unit, the infraorbital nerve stimulation unit, the first auriculotemporal nerve stimulation unit, the occipital nerve stimulation unit and the first greater auricular nerve stimulation unit including various combinations thereof, wherein when prompted the first neuromodulation unit is configured to generate neuromodulating electric stimulation to the wearer according to the selection of the stimulation mode selected by the wearer, and wherein the selections of the stimulation mode include the following: single neuromodulation by one component of the neuromodulation unit, double neuromodulation by various combinations of two components of the neuromodulation unit, and triple neuromodulation by various combinations of three components of the neuromodulation unit.

22. The automatic detection-therapy system for cluster headache of claim 15, wherein the cephalic blood flow monitoring system further comprises a photoplethysmography (PPG) unit, wherein the PPG unit is configured to record the wearer's cerebral and extracranial blood flow data and the wearer's blood pressure data, wherein the PPG unit is in electronic communication with the processing unit, wherein the processing unit is configured to analyze the cerebral and extracranial blood flow data recorded by the auricular SCOS unit and the cerebral and extracranial blood flow data and blood pressure data recorded by the PPG unit to assess the wearer's cerebral and extracranial blood flow data and blood pressure data.

23. The automatic detection-therapy system for cluster headache of claim 22, wherein the processing unit includes a closed-loop control system, wherein the closed-loop control system within the processing unit is in electronic communication with the auricular EEG monitoring system, the cephalic blood flow monitoring system and the neuromodulation unit, wherein the closed-loop control system receives real-time continuous input of the wearer's EEG and qEEG data from the auricular EEG monitoring system and real-time continuous input of the wearer's cerebral and extracranial blood flow data and blood pressure data from the cephalic blood flow monitoring system, wherein the closed-loop control system of the processing unit is configured to analyze these real-time EEG and qEEG data, cerebral and extracranial blood flow data and blood pressure data, using controlling algorithms, to continuously adjust the actuating outputs to the neuromodulation unit, wherein the actuating outputs includes turning on or turning off at least one component of the neuromodulation unit and adjusting the stimulating parameters of the neuromodulation unit during the time when the neuromodulation unit is turned on.

24. A neurovascular monitoring system, comprising:
an auricular electroencephalogram (EEG) monitoring system comprising a first EEG recording module having a plurality of EEG sensor electrodes, wherein the plurality of EEG sensor electrodes are configured to be housed in a first auricular housing, wherein the first auricular housing is configured to be placed in a wearer's first ear when in use, wherein each EEG sensor electrode of the first EEG recording module is in electronic communication with the first EEG recording module, wherein the first EEG recording module is configured to record EEG data of the wearer;
a cephalic blood flow monitoring system comprising a first laser speckle blood flow unit, wherein the first laser speckle blood flow unit is configured as a first auricular speckle contrast optical spectroscopy unit (first auricular SCOS unit) having a first auricular SCOS sensor, wherein the first auricular SCOS unit is configured to record cerebral blood flow data of the wearer, wherein the first auricular SCOS sensor is configured to be housed in the first auricular housing, wherein the neurovascular monitoring system is configured to simultaneously record the wearer's EEG data from the wearer's first ear and the wearer's cerebral blood flow data from the wearer's first ear;
a network interface in electronic communication with at least one of: a client device of the wearer and a client device of a healthcare provider of the wearer; and
a processing unit in electronic communication with the auricular EEG monitoring system, the cephalic blood flow monitoring system and the network interface,
wherein the processing unit is configured to convert the EEG data recorded by the auricular EEG monitoring system into quantitative EEG (qEEG) data, wherein the processing unit is configured to analyze the qEEG data recorded by the auricular EEG monitoring system to detect presence of at least one of: an increase of relative delta power more than a predetermined level and an increase of delta/alpha ratio more than a predetermined level, wherein the processing unit is configured to analyze the cerebral blood flow data recorded by the cephalic blood flow monitoring system to detect a decrease of cerebral blood flow more than a predetermined level, wherein when the processing unit detects at least one of the following: qEEG data showing an increase of relative delta power more than the predetermined level, qEEG data showing an increase of delta/alpha ratio more than the predetermined level and cerebral blood flow data showing a decrease of cerebral blood flow more than the predetermined level, the network interface is configured to generate a notification to at least one of: a client device of the wearer and a client device of a healthcare provider of the wearer, and wherein the processing unit is further configured to generate signals to the network interface to prompt the network interface to generate a display of the wearer's EEG status and cerebral blood flow status on at least one of: the client device of the wearer and the client device of the healthcare provider of the wearer.

25. The neurovascular monitoring system of claim 24, wherein the first auricular housing comprises a first tubular-shaped structure and a first body-structure, wherein the first tubular-shaped structure is configured to be inserted into an external ear canal of the wearer's first ear when in use, wherein the body-structure is configured to be placed at the immediate opening of the external ear canal of the wearer's first ear and be placed inside a tragus-concha bowl of the wearer's first ear when in use, wherein the first EEG recording module further comprises a reference electrode, wherein all of the EEG sensor electrodes of the first EEG recording module are located on a surface of the first tubular-shaped structure and partially embedded in the surface with protrusion at the surface of the first tubular-shaped structure, wherein the first tubular-shaped structure is configured to be made of elastic flexible adaptable material and the material is configured to have appropriate elasticity, flexibility and adaptability so that the first tubular-shaped structure will naturally adapt to the contour of the external ear canal of the wearer's first ear and snugly fill the interior of the external ear canal of the wearer's first ear when the first tubular-shaped structure is inserted into the external ear canal of the wearer's first ear and so that all of the EEG sensor electrodes will be naturally snugly in contact with the skin of the external ear canal of the wearer's first ear; wherein the reference electrode is configured to be located on a surface and partially embedded in the surface with protrusion at the surface of the first body-structure, wherein the first body-structure is configured to be made of elastic flexible adaptable material and the material is configured to have appropriate elasticity, flexibility and adaptability so that the first body-structure will naturally adapt to the contour of the tragus-concha bowl of the wearer's first ear and snugly fill the interior of the tragus-concha bowl of the wearer's first ear when the first body-structure is placed inside the tragus-concha bowl of the wearer's first ear and so that the reference electrode will be naturally snugly in contact with the skin of the tragus-concha bowl of the wearer's first ear; wherein the first auricular SCOS sensor is configured to be located on the surface and partially embedded in the surface with protrusion at the surface of one of: the tubular-shaped structure and the body-structure, wherein the tubular-shaped structure and the body-structure are configured to be made of elastic flexible and adaptable material so that the first auricular SCOS sensor will be naturally contacting the skin of the external ear canal or the skin of the tragus-concha bowl of the wearer's first ear when the tubular-shaped structure and the body-structure are placed in the wearer's first ear.

26. The neurovascular monitoring system of claim 24, wherein all of the EEG sensor electrodes of the first EEG recording module are configured as wireless EEG sensor electrodes, wherein the first EEG recording module comprises a wireless EEG amplifier, wherein the first auricular SCOS unit is configured as a first wireless auricular SCOS unit, wherein all of the EEG sensor electrodes of the first EEG recording module and the first wireless auricular SCOS unit are housed in the auricular housing while the wireless EEG amplifier and the processing unit are housed remotely in a client device.

27. The neurovascular monitoring system of claim 24, wherein the first auricular housing comprises a tubular-shaped structure, wherein all of the EEG sensor electrodes of the first EEG recording module are configured to be placed on a surface of the tubular-shaped structure, wherein the tubular-shaped structure is configured to be inserted into an external ear canal of the wearer's first ear when in use, wherein all of the EEG sensor electrodes of the first EEG recording module are configured to be located on a surface and partially embedded in the surface with protrusion at the surface of the tubular-shaped structure, wherein one or more EEG sensor electrode(s) are located at the upper surface of the tubular-shaped structure, wherein one or more EEG sensor electrode(s) are located above the horizontal level of the tubular-shaped structure and are facing forward-upward, wherein one or more EEG sensor electrode(s) are located above the horizontal level of the tubular-shaped structure and are facing backward-upward, wherein the tubular-shaped structure comprises an elastic flexible and adaptable material, and wherein the elastic flexible and adaptable material of the tubular-shaped structure is configured to have appropriate elasticity flexibility and adaptability so that the tubular-shaped structure will naturally adapt to the contour of the external ear canal of the wearer's first ear and snugly fill the interior of the external ear canal of the wearer's first ear when the tubular-shaped structure is inserted into the external ear canal of the wearer's first ear, and so that all of the EEG sensor electrodes of the first EEG recording module are naturally in close contact with the skin of the external ear canal of the wearer's first ear when the tubular-shaped structure is inserted into the external ear canal of the wearer's first ear.

28. The neurovascular monitoring system of claim 24, wherein the auricular EEG monitoring system further comprises a second EEG recording module, wherein the second EEG recording module comprises a plurality of EEG sensor electrodes configured to be housed in a second auricular housing, wherein the second auricular housing is configured to be placed in the wearer's second ear when in use, wherein each EEG sensor electrode of the second EEG recording module is in electronic communication with the second EEG recording module, wherein the second EEG recording module is configured to record EEG data of the wearer from the wearer's second ear, wherein the second EEG recording module is in electronic communication with the processing unit, wherein the processing unit is configured to convert the EEG data recorded by the second EEG recording module into quantitative EEG (qEEG) data, wherein the cephalic blood flow monitoring system further comprises a second auricular spectral contrast optical spectroscope unit (second auricular SCOS unit), wherein the second auricular SCOS unit comprises a second auricular SCOS sensor, wherein the second auricular SCOS sensor is configured to be housed in the second auricular housing, wherein the second auricular SCOS unit is configured to record cerebral blood flow data from the wearer's second ear, and wherein the second auricular SCOS unit is in electronic communication with the processing unit.

29. The neurovascular monitoring system of claim 28, wherein the processing unit is configured to analyze the EEG and qEEG data recorded by the first EEG recording module to detect at least one of the following: an increase of relative delta power more than a predetermined level, an increase of delta/alpha ratio more than a predetermined level and presence of one of: seizure and non-convulsive seizure, wherein the processing unit is configured to analyze the EEG and qEEG data recorded by the second EEG recording module to detect at least one of the following: an increase of relative delta power more than a predetermined level, an increase of delta/alpha ratio more than a predetermined level and presence of at least one of: seizure and non-convulsive seizure, wherein the processing unit is configured to analyze the cerebral blood flow data recorded by the first auricular SCOS unit to detect presence of a decrease of cerebral blood flow more than a predetermined level, wherein the processing unit is configured to analyze the cerebral blood flow data recorded by the second auricular SCOS unit to detect presence of a decrease of cerebral blood flow more than a predetermined level, wherein the processing unit is configured to generate signals to the network interface to prompt the network interface to generate a notification to at least one of: a client device of the wearer and a client device of a healthcare provider of the wearer when the processing unit detect presence of at least one of the following: presence of an increase of relative delta power more than a predetermined level, presence of an increase of delta/alpha ratio more than a predetermined level and presence of one of: seizure and non-convulsive seizure as recorded by the first EEG recording module, presence of an increase of relative delta power more than a predetermined level, presence of an increase of delta/alpha ratio more than a predetermined level and presence of one of: seizure and non-convulsive seizure as recorded by the second EEG recording module, presence of a decrease of cerebral blood flow more than a predetermined level as recorded by the first auricular SCOS unit, and presence of a decrease of cerebral blood flow more than a predetermined level as recorded by the second SCOS unit.

30. A cephalic blood flow monitoring apparatus for migraine and cluster headache detection, comprising:
a cephalic blood flow monitoring system configured to record cerebral and extracranial blood flow data of a wearer;
a network interface
a processing unit in electronic communication with the cephalic blood flow monitoring system and the network interface, wherein the processing unit is configured to analyze the cerebral and extracranial blood flow data recorded by the cephalic blood flow monitoring system to detect presence or cessation of cerebral and extracranial blood flow data suggestive of at least one of: migraine and cluster headache, wherein the processing unit is also configured to analyze the cerebral and extracranial blood flow data recorded by the cephalic blood flow monitoring system to detect presence or cessation of cerebral and extracranial blood flow data suggestive of at least one of: impending migraine and impending cluster headache;
wherein when the processing unit detects presence of cerebral and extracranial blood flow data suggestive of at least one of: migraine and cluster headache, the processing unit is configured to generate signals to the network interface to prompt the network interface to send notification to at least one of: a client device of the wearer and a client device of a healthcare provider of the wearer;
wherein when the processing unit detects presence of cerebral and extracranial blood flow data suggestive of at least one of: impending migraine and impending cluster headache, the processing unit is configured to generate signals to the network interface to prompt the network interface to send notification to at least one of: the client device of the wearer and the client device of the healthcare provider of the wearer; and
wherein when the processing unit detects both of the following: cessation of cerebral and extracranial blood flow data suggestive of at least one of migraine and cluster headache and cessation of cerebral and extracranial blood flow data suggestive of at least one of impending migraine and impending cluster headache, the processing unit is further configured to generate signals to the network interface to prompt the network interface to send notification to at least one of: the client device of the wearer and the client device of the healthcare provider of the wearer.

31. The cephalic blood flow monitoring apparatus for migraine and cluster headache detection of claim 30, wherein the cephalic blood flow monitoring system comprises a photoplethysmography (PPG) unit and an auricular speckle contrast optical spectroscopy unit (auricular SCOS unit), wherein the processing unit is in electronic communication with the PPG unit and the auricular SCOS unit, wherein the PPG unit and the auricular SCOS unit are both configured to be housed in an auricular housing to be placed in an ear of the wearer when in use, wherein the PPG unit is configured to record the wearer's cerebral and extracranial blood flow data and blood pressure data from the wearer's ear, and wherein the auricular SCOS unit is configured to record the wearer's cerebral and extracranial blood flow data from the wearer's ear.

32. An automatic detection-therapy system for migraine, comprising:
a cephalic blood flow monitoring system, wherein the cephalic blood flow monitoring system is configured to record cerebral and extracranial blood flow data of a wearer;
a first neuromodulation unit configured to give neuromodulating electric stimulation to the wearer when activated; and
a processing unit, wherein the processing unit is in electronic communication with the cephalic blood flow monitoring system and the first neuromodulation unit;
wherein the processing unit is configured to analyze the cerebral and extracranial blood flow data recorded by the cephalic blood flow monitoring system to detect presence or cessation of cerebral and extracranial blood flow data suggestive of migraine;
wherein when the processing unit detects presence of cerebral and extracranial blood flow data suggestive of migraine, the processing unit is configured to immediately send signals to the first neuromodulation unit to automatically start sending neuromodulating electric stimulation to the wearer;
wherein the processing unit is configured to analyze the cerebral and extracranial blood flow data recorded by the cephalic blood flow monitoring system to detect presence or cessation of cerebral and extracranial blood flow data suggestive of impending migraine;
wherein when the processing unit detects presence of cerebral and extracranial blood flow data suggestive of impending migraine, the processing unit is configured to immediately send signals to the first neuromodulation unit to automatically start sending neuromodulating electric stimulation to the wearer; and
wherein when the processing unit detects both of the following: cessation of cerebral and extracranial blood flow data suggestive of migraine and cessation of cerebral and extracranial blood flow data suggestive of impending migraine, the processing unit is further configured to immediately send signals to the first neuromodulation unit to automatically stop sending neuromodulating electric stimulation to the wearer.
